(19) United States
(12) Patent Application Publication
(10) Pub. No.: US 2013/0172324 A1
(43) Pub. Date: Jul. 4, 2013

(54) METHOD FOR TREATING CARDIOVASCULAR DISEASES USING RHO KINASE INHIBITOR COMPOUNDS

(71) Applicant: Inspire Pharmaceuticals, Inc., Whitehouse Station, NJ (US)

(72) Inventors: John W. LAMPE, Dedham, MA (US); Tomas NAVRATIL, Carrboro, NC (US); Ward M. PETERSON, Morrisville, NC (US); Jose L. BOYER, Chapel Hill, NC (US); Emilee H. FULCHER, Cary, NC (US); Scott D. SORENSEN, Morrisville, NC (US)

(73) Assignee: INSPIRE PHARMACEUTICALS, INC., Whitehouse Station, NJ (US)

(21) Appl. No.: 13/774,869

(22) Filed: Feb. 22, 2013

Related U.S. Application Data
(62) Division of application No. 12/492,579, filed on Jun. 26, 2009.
(60) Provisional application No. 61/076,059, filed on Jun. 26, 2008.
(60) Provisional application No. 61/169,239, filed on Apr. 14, 2009.
(60) Provisional application No. 61/169,639, filed on Apr. 15, 2009.
(60) Provisional application No. 61/169,635, filed on Apr. 15, 2009.

(51) Int. Cl. A61K 31/454; A61K 31/5377; A61K 31/55; A61K 31/497; A61K 31/496; A61K 31/4709; A61K 31/4725; A61K 31/506; A61K 31/416; A61K 31/4545

(52) U.S. Cl. CPC A61K 31/454; A61K 31/416; A61K 31/5377; A61K 31/55; A61K 31/4545; A61K 31/496; A61K 31/4709; A61K 31/4725; A61K 31/506; A61K 31/497
USPC 514/217.07; 514/322; 514/406; 514/234.5; 514/217.09; 514/318; 514/253.09; 514/312; 514/310; 514/235.2; 514/274; 514/256; 514/255.05; 514/309

(57) ABSTRACT

This invention is directed to methods of preventing or treating diseases or conditions associated with excessive cell proliferation, remodeling, inflammation, and vasoconstriction. Particularly, this invention is directed to methods of treating cardiovascular diseases or conditions such as stent restenosis and thrombosis, vascular thrombosis, cerebral vasospasm, atherosclerosis, systemic hypertension, cardiac hypertrophy, and sexual dysfunction. The method comprises identifying a subject in need of the treatment, and administering to the subject an effective amount of a novel Rho kinase inhibitor compound to treat the disease.

This application is a divisional of U.S. application Ser. No. 12/492,579, filed Jun. 26, 2009, which claims the benefit of U.S. Provisional Application Nos. 61/076,059, filed Jun. 26, 2008; 61/169,239, filed Apr. 14, 2009; 61/169,639, filed Apr. 15, 2009; and 61/169,635, filed Apr. 15, 2009; which are incorporated herein by reference in their entirety.

### TECHNICAL FIELD

This invention relates to methods of preventing or treating diseases or conditions associated with excessive cell proliferation, remodeling, inflammation, and vasoconstriction. Particularly, this invention relates to methods of treating cardiovascular diseases or conditions such as stent restenosis and thrombosis, vascular thrombosis, cerebral vasospasm, atherosclerosis, systemic hypertension, cardiac hypertrophy, and sexual dysfunction, using novel Rho kinase inhibitor compounds.

### BACKGROUND OF THE INVENTION

### Rho Kinase as a Target

The Rho family of small GTP binding proteins can be activated by several extracellular stimuli such as growth factors, hormones and mechanic stress and function as a molecular signaling switch by cycling between an inactive GDP-bound form and an active GTP-bound form to elicit cellular responses. Rho kinase (ROCK) functions as a key downstream mediator of Rho and exists as two isoforms (ROCK 1 and ROCK 2) that are ubiquitously expressed. ROCKs are serine/threonine kinases that regulate the function of a number of substrates including cytoskeletal proteins such as adducin, moesin, Na⁺—H⁺ exchanger 1 (NHE1), LIM-kinase and vimentin, contractile proteins such as the myosin light chain phosphatase binding subunit (MYPT-1), CPI-17, myosin light chain and calponin, microtubule associated proteins such as Tau and MAP-2, neuronal growth cone associate proteins such as CRMP-2, signaling proteins such as PTEN and transcription factors such as serum response factor (Loirand et al, Circ Res 98:322-334 (2006)). ROCK is also required for cellular transformation induced by RhoA. As a key intermediary of multiple signaling pathways, ROCK regulates a diverse array of cellular phenomena including cytoskeletal rearrangement, actin stress fiber formation, proliferation, chemotaxis, cytokinesis, cytokine and chemokine secretion, endothelial or epithelial cell junction integrity, apoptosis, transcriptional activation and smooth muscle contraction. As a result of these cellular actions, ROCK regulates physiologic processes such as vasoconstriction, bronchoconstriction, tissue remodeling, inflammation, edema, platelet aggregation and proliferative disorders.

One well documented example of ROCK activity is in smooth muscle contraction. In smooth muscle cells ROCK mediates calcium sensitization and smooth muscle contraction. Agonists (noradrenaline, acetylcholine, endothelin, etc.) that bind to G protein coupled receptors produce contraction by increasing both the cytosolic Ca²⁺ concentration and the Ca²⁺ sensitivity of the contractile apparatus. The Ca²⁺-sensitizing effect of smooth muscle constricting agents is ascribed to ROCK-mediated phosphorylation of MYPT-1, the regulatory subunit of myosin light chain phosphatase (MLCP), which inhibits the activity of MLCP resulting in enhanced phosphorylation of the myosin light chain and smooth muscle contraction (WO 2005/003101A2, WO 2005/034866A2).

Prototype non-potent Rho-kinase inhibitors, Y27632 or fasudil, have been used in several animal models. Y27632 has shown favorable activity in animal models of cardiovascular diseases such as hypertension (Uehata et al Nature 389:990-994, 1997), atherosclerosis (Mallat et al. Circ Res 93:884-888, 2003) and heart failure (Shimokawa et al. Arterioscler Thromb Vasc Biol 25:1767-1775, 2005). Y27632 has additionally shown favorable activity in animal models of penile erectile dysfunction (Chitaley et al. Nat Med 7:119-22, 2001), cardiovascular disorders such as arteriosclerosis/restenosis, coronary and cerebral vasospasm, pulmonary hypertension, stroke, ischemia/reperfusion injury and cardiac hypertrophy (Shimokawa et al. Arterioscler Thromb Vasc Biol 25:1767-1775, 2005); erectile dysfunction (Park K et al. J Sex Med 3:996-1003, 2006); neuronal degeneration (Asano T et al. Br J Pharmacol 103:1935-1938, 1991). In addition, fasudil has been shown to provide benefits for controlling cerebral vasospasms and ischemia following subarachnoid hemorrhage in humans.

### Stent Restenosis and Thrombosis

Coronary artery disease is the leading cause of mortality and morbidity in developed countries. Coronary artery stenting of blocked arteries using bare metal stents was first developed, producing a significant decrease in the rates of angiographic restenosis and target lesion revascularization (Fischman D L et al. N Engl J Med 331: 496-501, 1994; Serruys P W et al. N Engl J Med 331: 489-95, 1994; Cutlip D E et al., J Am Coll Cardiol 40: 2082-9, 2002). Drug-eluting stents (DES) were then developed in an effort to further reduce the need for target lesion revascularization. DES consist of a metallic stent backbone, a polymer covering, and an anti-restenotic drug that is mixed within the polymer and is released over a period as short as days to as long as one year after implantation to modify the local healing response of the stented tissue. The clinical trials of sirolimus-eluting and paclitaxel-eluting stents have demonstrated a marked reduction in the incidence of restenosis and target lesion revascularization compared to bare metal stents (Costa M A and Simon DI Circulation 111: 2257-73, 2005; Roiron C et al., Heart. 92: 641-9, 2006).

Although drug-eluting stents were regarded as a major medical advance when they first appeared, new evidence suggests a high risk for in-stent thrombosis. A stent is a foreign object in the body, and the body responds to the stent's presence in a variety of ways. Macrophages accumulate around the stent, and nearby smooth muscle cells proliferate. These physiological changes, which can cause restenosis, are limited by the drugs released by the stent, but these drugs also limit re-endothelialization. This lack of healing can make the stent an exposed surface on which a life-threatening clot can form.

Stent occlusion due to thrombosis may occur during the procedure, in the following days, or later. Treatment with antiplatelet drugs such as aspirin and clopidogrel appears to be the most important factor reducing this risk of hospitalization, urgent care and death due to in-stent thrombosis, however, systemic administration of antiplatelet drugs may lead to other side effects such as minor and major bleeding due to uncontrolled antithrombotic activity. In addition, since both aspirin and clopidogrel are irreversible antiplatelet drugs with effects that persist up to more than five days after discontinuation of treatment, emergency surgical procedures due to accident or disease are prone to severe peri- and post-surgical bleeding. Discontinuation of the antiplatelet therapy for scheduled surgical procedures or early cessation of treatment with these drugs increases the risk of in-stent thrombosis and myocardial infarction.

Rho kinase signaling pathways are implicated in cell proliferation, motility and migration of vascular smooth muscle cells responsible for restenosis of stented vessels. Rho kinase signaling pathways are also implicated in important platelet functions that may lead to in-stent thrombus formation, such as the platelet shape change that precedes the aggregation of platelets stimulated with thrombin receptor agonists and other aggregating agents (Benjamin Z S et al. J Biol Chem 274: 28293-28300, 1999) and the formation of stable aggregates of platelets stimulated with thrombin receptor agonists (Missy K et al., Thromb Haemost 85: 514-20, 2001; Nishioka H et al. Biochem Biophys Res Commun. 280: 970-5, 2001).

### Vascular Thrombosis

Platelets function in the body to limit blood loss in the event of vascular damage. Normally, platelets circulate throughout the body with other cellular components of blood, bathed in a mixture of various plasma proteins, many of which play key roles in the clotting process. Upon exposure of vascular sub-endothelium, a complex series of events occurs to limit the loss of blood from the damaged vessel. Circulating platelets contacting components of the exposed sub-endothelium: 1) bind and adhere, 2) spread across the exposed surface, 3) activate as evidenced by release of granule contents, 4) aggregate and recruit other circulating platelets from the blood stream, and 5) form an efficient plug, clot, and/or thrombus stemming the flow of blood from the vessel.

In response to vascular injury, such as atherosclerotic plaque rupture in a coronary vessel, circulating platelets are exposed to a variety of matrix elements that are prothrombotic. Platelets can strongly adhere to two specific matrix components, collagen and von Willebrand factor. At low blood flow shear rates, adhesion to collagen predominates, whereas at higher shear rates—for example, those that would occur in stenosed vessels—the initial platelet adhesion is primarily mediated by binding to von Willebrand factor. Adhesion to either collagen and/or von Willebrand factor initiates signals leading to platelet activation, platelet spreading on the matrix, secretion of prothrombotic substances such as thrombin, adenosine diphosphate (ADP) and thromboxane A₂, and upregulation of the adhesive function of GP IIb-IIIa, which can bind fibrinogen and von Willebrand factor, resulting in platelet aggregate or thrombus formation. In contrast to the coagulation cascade, a process defined in part by the conversion of fibrinogen to fibrin, platelets coalesce about the damaged area and are held together by bridging molecules that bind to specific receptors on the platelet surface. The initial bridging between platelets and the sub-endothelium is dependent on the interaction between the glycoprotein Ib (GPIb) receptor on the surface of the platelet and von Willebrand Factor (VWF) in the subendothelium (i.e., immobilized VWF). This interaction in itself is unique, since normal platelets circulating in the blood often contact soluble VWF, but are not activated, nor do they bind to the soluble VWF. In vitro experimentation has confirmed that immobilization of the soluble VWF to a surface facilitates binding and activation of platelets. Upon activation of the platelet, an additional receptor, glycoprotein IIb/IIIa (GPIIb/IIIa), is altered enabling the binding of several plasma proteins, thereby promoting platelet/platelet binding.

Hyperactive platelets can induce thrombus formation at inopportune times resulting in reduced blood supply to various organs and tissues. A prime example is thrombus formation induced by blood flowing through a stenotic (narrowed) vessel supplying the heart. Reduction of the flow of blood to the heart muscle leads to infarction and eventually heart attack (cardiac cell death). Cerebral ischemia (transient ischemic attack; stroke) occurs when an embolus or thrombus occludes blood vessels feeding the brain.

Other pathological states exist that are caused by platelet activation as a result of an inappropriate antibody-mediated process. Heparin-induced thrombocytopenia (HIT) is characterized by a dramatic loss in platelet numbers and thrombus formation at sites of pre-existing pathology. From 1% to 5% of all patients receiving unfractionated heparin as an anticoagulant to promote blood flow produce an antibody that binds to heparin in complex with a platelet granule protein. The binding of the antibody to the heparin/protein complex on the surface of the platelet induces rapid platelet activation and localized thrombus formation. This in turn leads to infarction of the affected area.

Rho kinase signaling pathways are implicated in important platelet functions that may lead to thrombus formation. For example, the pathways are implicated in the platelet shape change that precedes the aggregation of platelets stimulated with thrombin receptor agonists and other aggregating agents (Benjamin Z S et al. J Biol Chem 274: 28293-28300, 1999). The pathways are also implicated in the formation of stable aggregates of platelets stimulated with thrombin receptor agonists (Missy K et al., Thromb Haemost 85: 514-20, 2001; Nishioka H et al. Biochem Biophys Res Commun. 280: 970-5, 2001).

### Cerebral Vasospasm

Cerebral aneurysm rupture and subarachnoid hemorrhage (SAH) inflict disability and death upon thousands of individuals each year with mortality rates as high as 50% and the majority of survivors left with moderate to severe disability (Hop J W et al. Stroke, 28:660-664, 1997). Traumatic brain injury is the leading cause of SAH. SAH can lead to cerebral vasospasm, characterized as a delayed and sustained arterial constriction that can ultimately lead to brain cell damage, in the form of cerebral ischemia and infarction, due to interrupted blood supply. In addition to vasospasm in large diameter arteries, enhanced constriction of resistance arteries within the cerebral vasculature may contribute to decreased cerebral blood flow and the development of delayed neurological deficits following SAH. In vitro, elevation of intravascular pressure within a physiological range (60 to 100 mmHg) constricts small diameter cerebral arteries in the absence of other vasoactive stimuli. In cerebral arteries, increased intravascular pressure leads to vascular smooth muscle membrane potential depolarization and increased global cytosolic free Ca²⁺ concentration ([Ca²⁺]ᵢ), a key mediator of vascular smooth muscle contraction. Subjects at risk of vasospasm are currently administered a variety of preventative medications including L-type voltage-dependent calcium channel (L-type VDCC) inhibitors (e.g., nimodipine), phenylephrine, dopamine, as well as a combination of mannitol and hyperventilation; however, current therapies in the treatment of this phenomenon are less than ideal (Macdonald R L et al. Stroke, 22:971-982, 1991).

A key determinant of smooth muscle calcium sensitivity is Rho kinase. Certain inhibitors of Rho kinase (not compounds of Formula I or II of the present invention) have been shown to induce relaxation of smooth muscle via inhibition of calcium sensitivity (Yoshii et al. Am. J. Respir. Cell Mol. Biol, 20:1190-1200, 1999). Fasudil, a known Rho kinase inhibitor, inhibits vascular smooth muscle contraction in vitro and is utilized clinically in Japan to improve subarachnoid hemorrhage-postoperative cerebral vasospasm and corresponding cerebral ischemia symptoms.

### Atherosclerosis

Atherosclerosis is the underlying disease process responsible for vascular conditions that causes the death of over one third of the population of the Western world. It is a chronic inflammatory response in the walls of arteries, in large part due to the accumulation of macrophage white blood cells and promoted by low density lipoproteins without adequate removal of fats and cholesterol from the macrophages by functional high density lipoproteins (HDL). It is caused by the formation of multiple plaques within the arteries, resulting in a hardening or “furring” of the arteries (Maton A et al. Human Biology and Health, ISBN 0-13-981176-1, 1993). As the disease progresses, there is a migration of the endothelial cells over the ensuing plaques. These plaques are composed of cholesterol, activated platelets, macrophages and accumulated lipoproteins. The combination of these plaques, inflammation and endothelial cell migration leads to this hardening or “furring” of the arteries and loss elasticity of the vessels.

Current therapies for atherosclerosis include the use of statins, as well as aspirin. Exercise and a strict diet are also used to combat this disease. Dietary changes to achieve benefit have been more controversial, generally far less effective and less widely adhered to with success. New therapies would be a welcome addition to the current treatments.

Rho kinase signaling pathways are implicated in cell proliferation, motility and migration of vascular cells responsible for thickening of the blood vessels. Rho kinase signaling pathways are also implicated in the platelet shape change that precedes the aggregation of platelets stimulated with thrombin receptor agonists and other aggregating agents (Benjamin Z S et al. J Biol Chem 274:28293-28300, 1999). Rho kinase signaling pathways are further implicated in the formation of stable aggregates of platelets stimulated with thrombin receptor agonists (Missy K et al., Thromb Haemost 85:514-20, 2001; Nishioka H et al. Biochem Biophys Res Commun. 280:970-5, 2001), that are associated with plaque formation. In addition to cell migration upon vessel wall injury and platelet activation, Rho kinase activation is also involved in the proinflammatory mediators that trigger the ensuing inflammatory cascade.

### Systemic Hypertension

In the United States, the treatment of hypertension is the leading reason for physician office visits in non-pregnant adults as well as the most common reason for the use of prescribed drugs (Chemy, D K et al. Advance data from vital and health statistics; no 337. Hyattsville, Md.: National Center for Health Statistics, 2003). According to NHANES and United State Census bureau data from 1999-2000, an estimated 58 to 65 million American adults, approximately 29 to 31 percent incidence, suffer from hypertension (Hajjar, I et al. JAMA, 290:199-206, 2003; Fields, L E et al. Hypertension, 44:398-404, 2004). The prevalence of hypertension is estimated to increase with a rising incidence of obesity and a growing elderly population, as over fifty percent of hypertensive persons are older than 65 (Kaplan, N M. Clinical hypertension. 8th ed. Philadelphia: Lippincott Williams & Wilkins, 2002). Since elevated blood pressure imposes an increased workload on the heart, hypertensive patients often suffer from various cardiovascular disorders, such as angina pectoris, cardiac hypertrophy, coronary vascular diseases, ischemic heart injury, and, in more severe cases, myocardial infarction and heart failure. In addition, hypertension is often concomitant with the development of renal disorders and the occurrence of cerebrovascular conditions, such as cerebral infarction, cerebral hemorrhage, and subarachnoid hemorrhage. Reducing arterial blood pressure is thus critical in the prevention and even the treatment of such life-threatening conditions.

The pathogenesis of hypertension is poorly understood and a variety of factors are related to the condition including increased angiotensin II activity, genetic factors, and enhanced beta-adrenergic responsiveness (Staessen, J A et al. Lancet, 361: 1629-41, 2003). Furthermore, a number of risk factors are associated with the condition, such as high alcohol consumption, sodium intake, obesity, race, and personality traits (Thompson, D et al. Arch Intern Med, 1999; 159:2177-83, 1999; de Simone, G et al. Hypertension, 47:162-7, 2006; Khot, U N et al. JAMA, 290:898-907, 2003). As the blood pressure increases, this leads to an additional inflammatory cascade which can accelerate the organ dysfunctions.

Various therapeutic strategies have been designed for the treatment of hypertension and its associated complications. These treatment strategies focus on blood pressure control, which has demonstrated 35 to 40 percent mean reductions in stroke incidence, 20 to 25 percent in myocardial infarction and more than 50 percent in heart failure in clinical trials performed on antihypertensive therapy (Neal, B et al. Lancet, 356:1955-64, 2000). Though such modalities are generally effective in reducing blood pressure in patients, they do not reduce some of the concomitant inflammation associated with elevated blood pressure and organ dysfunction. In addition, the current treatments are frequently associated with serious debilitating side effects, such as potassium depletion, hyperglycemia, depression, carbohydrate intolerance, tachychardia, allergic skin rashes, and in more severe cases vomiting, fever, diarrhea, angina, and cardiac failure. Thus, additional therapeutic modalities for reducing or preventing hypertension and its associated conditions are desirable.

Rho kinase signaling pathways are implicated in vascular smooth muscle contraction, motility and migration. In smooth muscle cells, Rho kinase mediates calcium sensitization and smooth muscle contraction. Rho kinase signaling pathways are also implicated in the platelet shape change that precedes the aggregation of platelets stimulated with thrombin receptor agonists and other aggregating agents (Benjamin Z S et al. J Biol Chem, 274:28293-28300, 1999). Additionally, Rho kinase signaling pathways are implicated in the down regulation of pro-inflammatory pathways (Riento K et al. Nat Rev Mol Cell Biol, 4:446-456, 2003).

### Cardiac Hypertrophy

Cardiac hypertrophy, which is an adaptive response to hemodynamic or non-hemodynamic stimuli, may occur as the result of a variety of ailments including, but not limited to, high blood pressure, valvular heart disease, myocardial infarction, and cardiomyopathy, and leads to an enlarged heart. The presence of cardiac hypertrophy (on ECG or echocardiography) is important clinically because it is associated with increases in the incidence of heart failure, ventricular arrhythmias, death following myocardial infarction, decreased LV ejection fraction, sudden cardiac death, aortic root dilation, and a cerebrovascular event. Cardiac hypertrophy also carries an increased risk for cardiac events such as angina, myocardial infarction, heart failure, serious ventricular arrhythmias and cardiovascular death.

One of the hallmarks of cardiac hypertrophy is an increase in the mass of the left ventricle. However, this can be secondary to an increase in wall thickness, an increase in cavity size, or both. Cardiac hypertrophy as a consequence of hypertension usually presents with an increase in wall thickness, with or without an increase in cavity size. The normal LV mass in men is 135 g and the mass index is 71 g/m2; in women, the values are 99 g and 62 g/m2, respectively. LVH is usually defined as two standard deviations above normal. The current echocardiographic criteria for LVH are ≧134 and ≧110 g/m2 in men and women respectively, although there is a relatively wide range of published cutoff values (Albergel (1995) Am. J. Cardiol. 75:498; RB. Devereux (1984) J. Am. Coll. Cardiol. 4:1222). In clinical practice, however, the presence of LVH is more commonly defined by wall thickness values obtained from M-mode or 2D images from the parasternal views. Hypertension associated cardiac hypertrophy may also result in interstitial fibrosis (Van Hoeven (1990) Circulation 82:848). Both factors contribute to an increase in left ventricular stiffness, resulting in diastolic dysfunction and an elevation in left ventricular end diastolic pressure.

Clinical experience has suggested that antihypertensive agents alone are not an effective treatment of abdominal aortic aneurysm. Calcium channel blockers, which are often prescribed to patients diagnosed with hypertension in order to decrease blood pressure, may increase risk in patients with abdominal aortic aneurysm (Wilmink et al. (2002) J. Vase. Surg. 36:751-757).

Many of the hormones and neurotransmitters that are implicated in the initiation and exacerbation of myocardial hypertrophy, including angiotensin II and endothelin, bind to cell membrane receptors which couple to a subset of intracellular heterotrimeric G proteins, the G(q) subclass. Direct evidence for the importance of this subclass is provided by the phenotype of transgenic mice, which selectively overexpress the carboxyl-terminal peptide of the alpha subunit G(q) (SA. Akhter (1998) Science 280:574). This peptide competes with endogenously expressed G proteins, thereby inhibiting intracellular signaling of coupled cell surface receptors. In response to surgically induced pressure overload, transgenic animals develop significantly less myocardial hypertrophy compared to control mice. This effect of angiotensin II may be related in part to the promotion of myocardial fibrosis (Cuspidi (2006) Transplant 21:20).

Rho-kinase has been identified in the signaling pathway within the cardiovascular field as potential therapeutic targets (H. Shimokawa (2002) J. Cardiovascular Pharm. 39: 319-327) that is involved in smooth muscle function. Angiotensin II induces cardiac hypertrophy, by directly stimulating cardiomyocyte growth and by increasing ventricular afterload. Rho-kinase can affect cell growth and motility, focal adhesions and cytokinesis (M. Amano (1997) Science 275:1308-1311), and has been implicated in cardiovascular disease (see review (H. Shimokawa, (2002) J. Cardiovasc. Pharmacol. 39:319-327)). Data suggest that some of the cardiac effects of angiotensin II can be mediated by Rho/Rho-kinase signaling. Activation of angiotensin II type 1 receptors (AT1) by angiotensin II has been shown to activate Rho, which, in turn, induces protein synthesis in cardiomyocytes, leading to hypertrophy (R. Aikawa (2000) Mol. Cell. Biochem. 212:177-182; H. Aoki (1998) Circ. Res. 82:666-676). Angiotensin II also promotes inflammation by up-regulating the expression monocyte chemotactic protein (MCP-1), macrophage colony-stimulating factor (M-CSF), vascular cell adhesion molecule-1 (VCAM-1), intercellular adhesion molecule-1 (ICAM-1) and E-selectin, and by promoting monocyte/macrophage migration (D. Tham (2002) Physiol. Genomics 11:21-30). Rho-kinase has been shown to mediate angiotensin II-induced MCP-1 expression, macrophage infiltration (R. Aikawa (2000) Mol. Cell. Biochem. 212:177-182; H. Aoki (1998) Circ. Res. 82:666-676; Y. Funakoshi (2001) Hypertension 38:100-104; K. Miyata (2000) Vasc. Biol. 20:2351-2358), and connective tissue growth factor production, thus contributing to fibrosis (D. Iwanciw (2003) Vasc. Biol. 23:1782-1787). Recent data indicate that inhibition of Rho-kinase can prevent angiotensin II-induced expression of plasminogen activator inhibitor-1, and attenuate cardiac remodeling in rats (N. Kobayashi (2002) Cardiovasc. Res. 55:757-767; N. Kobayashi (2002) J. Pharmacol. Exp. Ther. 301:459-466).

### Sexual Dysfunction

The human sexual response in both males and females results from an interplay of physiological, psychological, and hormonal factors. One common aspect of the sexual response in males and females, however, is the vasoactive response, which results in engorgement of the sexual tissues of the genitalia with blood as a result of vascular smooth muscle relaxation in response to sexual stimulation. Thus, blood pressure and blood flow inside the penis and clitoris increase when smooth muscles of the pudental vasculature relax.

This arterial influx of blood causes enlargement of the penile or clitoral corpora cavernosa and results in erection. In the penis, venous outflow is reduced by enlargement of the corpus cavernosum, permitting sustained high cavernosal blood pressure and maintained rigidity.

Relaxation of penile or clitoral smooth muscle and the accompanying vasodilation are triggered by the central nervous system and reinforced locally by reflex mechanisms. Most of the time, however, the body keeps the erectile tissue in a flaccid (non-erect) state by maintaining the smooth muscle tissues in the contracted state. Vasoconstrictors, such as norepinephrine (noradrenaline) and endothelin-1, help maintain the cavernosal smooth muscle tissue in a contracted state to keep blood flow low.

Impotence (erectile dysfunction in men) is generally defined as an inability to achieve and sustain an erection sufficient for satisfactory sexual performance and intercourse.

Impotence can be due to psychological disturbances, neurological abnormalities, or other physiological disturbances including hormonal deficiencies or a combination of causes. Male impotence is estimated to affect 40% of men age 40 in the U.S., increasing with age to about 50% by 50 years, and is as high as 67% by the age of 70. In the United States, it is estimated that up to 30 million males may suffer from impotence.

Females can also have sexual dysfunction that increases with age and is associated with the onset of menopause and increased risk of vascular disorders. Thus, similar to men, sexual arousal in women is accompanied, at least in part, by increased blood flow which engorges the clitoris. Blood flow to the vagina also increases resulting in increased vaginal lubrication. Thus, female sexual dysfunction can result from an inability to attain or maintain vaginal lubrication and clitoral engorgement throughout the period of sexual activity (see e.g. Berman, J. R., et al, Eur. Urology 38, 20-29, 2000). Previous work in the area of erectile dysfunction has focused on processes that result in smooth muscle relaxation. One mechanism which causes erection of the penis involves release of nitric oxide (NO), enabling relaxation of blood vessels in the cavernosal circulation during sexual stimulation. For example, the compound sildenafil (Viagra) is a type 5 phosphodiesterase inhibitor that potentiates the effects of local release of NO, thereby resulting in vascular smooth muscle relaxation. Studies have found sildenafil to have an overall 60% efficacy rate in the promotion of NO-mediated cavernosal vasorelaxation (Virag, R., Urology 54, 1073-77, 1999). Still, in those patients with severe erectile dysfunction (such as that resulting from diabetes or prostate surgery), sildenafil treatment was associated with a modest satisfaction rate (Jarow, I P et al., J. Urologyb 102, 722-725, 1999). Moreover, only 30% of patents studied chose sildenafil treatment alone (Virag, R., 1999).

There is a need for an effective or improved method for treating stent restenosis and thrombosis, vascular thrombosis, cerebral vasospasm, atherosclerosis, systemic hypertension, cardiac hypertrophy, and sexual dysfunction.

### SUMMARY OF THE INVENTION

The present invention is directed to methods of preventing or treating diseases or conditions associated with excessive cell proliferation, remodeling, inflammation, and vasoconstriction. Particularly, this invention is directed to methods of treating diseases or disorders associated with cardiovascular conditions such as stent restenosis and thrombosis, vascular thrombosis, cerebral vasospasm, atherosclerosis, systemic hypertension, cardiac hypertrophy, and sexual dysfunction. The method comprises identifying a subject in need of the treatment, and administering to the subject an effective amount of a novel Rho kinase inhibitor compound of Formula I or II to treat the disease.

The active compound is delivered to a subject by systemic administration or local administration.

The present invention is also directed to a drug-eluting stent, wherein the stent is coated with one or more compounds of Formula I or II, or a pharmaceutically acceptable hydrate, solvate or salt thereof, wherein a therapeutically effective amount of the compound is eluted to the local environment when the stent is placed in a blood vessel.

### BRIEF DESCRIPTION OF THE DRAWINGS

FIG. 1 shows the murine eosinophil chemotaxis. The data reported are mean number of migrated eosinophils per high power view field±SEM. Average of at least 2 view fields per well, each treatment ran in triplicate.

FIG. 2 shows the human eosinophil chemotaxis. The data reported are mean number of migrated eosinophils per high power view field±SEM. Average of at least 3 view fields per well, each treatment ran in duplicate.

FIG. 3 shows percent of FBS induced proliferation. Each compound was tested at 30 μM and challenged with 10% FBS with an n=3. * indicates n=5.

FIG. 4A shows the dose response curves for a representative compound, 2.039, to induce relaxation in 100 nM norepinephrine precontracted rings. Data are reported as a percent of the maximal norepinephrine response and are mean±SEM of 5 replicates. FIG. 4B shows the tension (in grams) recorded from an aortal ring. Addition of 100 nM norepinephrine induced a contractile response recorded as an increase in tension that was fully reversed upon addition of 100 μM compound 2.039.

FIG. 5 shows the % inhibition of ATP-stimulated IL-1β Secretion in Human Monocytes by Rho kinase Inhibitors. Data represent the mean±SD of at least n=2 experiments.

FIG. 6 shows the dose response curves for representative compounds, 1.123 and 2.039, to induce relaxation in 1 μM carbachol precontracted tracheal rings. Data are reported as a percent of the maximal carbachol response and are mean±SEM of at least 4 replicates.

FIG. 7 shows the dose response curve for the representative compound, 1.091, to induce relaxation in rat tracheal rings pretreated with either vehicle alone or the inflammatory cytokines, IL-1β and TNF-α. Data are reported as a percent of the maximal carbachol (300 nM carbachol) response.

FIG. 8 shows the bronchodilator dosing paradigm.

FIG. 9 shows the dose response effect of Compound 1.091 on airway hyperreactivity when dosed intratracheally using the bronchodilator dosing paradigm. Linear AUC values from compound treated mice were reported as a percent of linear AUC values from vehicle-treated ovalbumin-sensitized/ovalbumin-challenged (Ova) mice. *, p<0.05 using statistical analysis described in Example 23.

FIG. 10 shows the anti-inflammatory dosing paradigm.

FIG. 11 shows the eosinophils per mL in ova-sensitized, ova-challenged mice treated with Compound 2.038 or Compound 1.131 and normal mice.

FIG. 12 shows the dose response effect of Compound 1.091 on eosinophil influx when dosed intratracheally (i.t.) to ova-sensitized, ova-challenged mice, *, p<0.05 when compared to ova-sensitized, ova-challenged mice (Ova) using Student's t-test.

FIG. 13 shows the concentration of IL-5 (pg/mL) in BALF of (1) ova-sensitized, ova-challenged mice, (2) ova-sensitized, ova-challenged mice treated with Compound 2.038 (15 μmol/kg/oral), and (3) normal, saline-sensitized mice. Dashed line indicates the lower limit of detection for the cytokine of interest. Data represent mean±SEM, n=10 for ova-sensitized, ova-challenged mice, treated or untreated; n=5 for normal mice.

FIG. 14 shows the concentration of Eotaxin (pg/mL) in BALF of (1) ova-sensitized, ova-challenged mice, (2) ova-sensitized, ova-challenged mice treated with Compound 2.038 (15 μmol/kg/oral), and (3) normal, saline-sensitized mice. Dashed line indicates the lower limit of detection for the cytokine of interest. Data represent mean±SEM, n=10 for ova-sensitized, ova-challenged mice, treated or untreated; n=5 for normal mice.

FIG. 15 shows the concentration of IL-13 (pg/mL) in BALF of (1) ova-sensitized, ova-challenged mice, (2) ova-sensitized, ova-challenged mice treated with Compound 2.038 (15 μmol/kg/oral), and (3) normal, saline-sensitized mice. Dashed line indicates the lower limit of detection for the cytokine of interest. Data represent mean±SEM, n=10 for ova-sensitized, ova-challenged mice, treated or untreated; n=5 for normal mice.

FIG. 16 shows the dose response effect of Compound 1.091 on airway hyperreactivity when dosed using the anti-inflammatory dosing paradigm on Days 27 to 30. *, p<0.05 using statistical analysis described in Example 23.

FIG. 17 shows the dose-dependent inhibition of LPS-induced neutrophilia by Compound 1.091 when dosed intratracheally to mice. Data are reported as cells/ml and are mean±SEM. *, p<0.05 when compared to mice treated with LPS alone using Student's t-test.

FIG. 18 shows the reduction of IL-1β levels in BALF from LPS-challenged mice upon intratracheal administration of Compound 1.091 or Compound 2.059. Data are reported as pg/mL of IL-1β and are mean±SEM.

FIGS. 19A and 19B show [³H]-thymidine incorporation in primary human LAM-derived cells. Cells were treated with vehicle alone (control) or with 10 μM of Compound 1.132, Compound 2.066 or Compound 1.161. Experiments were performed on two separate cell lines, LAM1 cells (FIG. 19A) and LAM2 cells (FIG. 19B). Data are reported as counts per minute (cpm) of incorporated [³H]-thymidine are mean±SEM.

### DETAILED DESCRIPTION OF THE INVENTION

### Definitions

When present, unless otherwise specified, the following terms are generally defined as, but are not limited to, the following:

Halo substituents are taken from fluorine, chlorine, bromine, and iodine.

“Alkyl” refers to groups of from 1 to 12 carbon atoms inclusively, either straight chained or branched, more preferably from 1 to 8 carbon atoms inclusively, and most preferably 1 to 6 carbon atoms inclusively.

“Alkenyl” refers to groups of from 2 to 12 carbon atoms inclusively, either straight or branched containing at least one double bond but optionally containing more than one double bond.

“Alkynyl” refers to groups of from 2 to 12 carbon atoms inclusively, either straight or branched containing at least one triple bond but optionally containing more than one triple bond, and additionally optionally containing one or more double bonded moieties.

“Alkoxy” refers to the group alkyl-O— wherein the alkyl group is as defined above including optionally substituted alkyl groups as also defined above.

“Alkenoxy” refers to the group alkenyl-O— wherein the alkenyl group is as defined above including optionally substituted alkenyl groups as also defined above.

“Alkynoxy” refers to the group alkynyl-O— wherein the alkynyl group is as defined above including optionally substituted alkynyl groups as also defined above.

“Aryl” refers to an unsaturated aromatic carbocyclic group of from 6 to 14 carbon atoms inclusively having a single ring (e.g., phenyl) or multiple condensed rings (e.g., naphthyl or anthryl). Preferred aryls include phenyl, naphthyl and the like.

“Arylalkyl” refers to aryl-alkyl-groups preferably having from 1 to 6 carbon atoms inclusively in the alkyl moiety and from 6 to 10 carbon atoms inclusively in the aryl moiety. Such arylalkyl groups are exemplified by benzyl, phenethyl and the like.

“Arylalkenyl” refers to aryl-alkenyl-groups preferably having from 2 to 6 carbon atoms in the alkenyl moiety and from 6 to 10 carbon atoms inclusively in the aryl moiety.

“Arylalkynyl” refers to aryl-alkynyl-groups preferably having from 2 to 6 carbon atoms inclusively in the alkynyl moiety and from 6 to 10 carbon atoms inclusively in the aryl moiety.

“Cycloalkyl” refers to cyclic alkyl groups of from 3 to 12 carbon atoms inclusively having a single cyclic ring or multiple condensed rings which can be optionally substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, or multiple ring structures such as adamantyl, and the like.

“Cycloalkenyl” refers to cyclic alkenyl groups of from 4 to 12 carbon atoms inclusively having a single cyclic ring or multiple condensed rings and at least one point of internal unsaturation, which can be optionally substituted with from 1 to 3 alkyl groups. Examples of suitable cycloalkenyl groups include, for instance, cyclobut-2-enyl, cyclopent-3-enyl, cyclooct-3-enyl and the like.

“Cycloalkylalkyl” refers to cycloalkyl-alkyl-groups preferably having from 1 to 6 carbon atoms inclusively in the alkyl moiety and from 6 to 10 carbon atoms inclusively in the cycloalkyl moiety. Such cycloalkylalkyl groups are exemplified by cyclopropylmethyl, cyclohexylethyl and the like.

“Cycloalkylalkenyl” refers to cycloalkyl-alkenyl-groups preferably having from 2 to 6 carbon atoms inclusively in the alkenyl moiety and from 6 to 10 carbon atoms inclusively in the cycloalkyl moiety. Such cycloalkylalkenyl groups are exemplified by cyclohexylethenyl and the like.

“Cycloalkylalkynyl” refers to cycloalkyl-alkynyl-groups preferably having from 2 to 6 carbon atoms inclusively in the alkynyl moiety and from 6 to 10 carbon atoms inclusively in the cycloalkyl moiety. Such cycloalkylalkynyl groups are exemplified by cyclopropylethynyl and the like.

“Heteroaryl” refers to a monovalent aromatic heterocyclic group of from 1 to 10 carbon atoms inclusively and 1 to 4 heteroatoms inclusively selected from oxygen, nitrogen and sulfur within the ring. Such heteroaryl groups can have a single ring (e.g., pyridyl or furyl) or multiple condensed rings (e.g., indolizinyl or benzothienyl).

“Heteroarylalkyl” refers to heteroaryl-alkyl-groups preferably having from 1 to 6 carbon atoms inclusively in the alkyl moiety and from 6 to 10 atoms inclusively in the heteroaryl moiety. Such heteroarylalkyl groups are exemplified by pyridylmethyl and the like.

“Heteroarylalkenyl” refers to heteroaryl-alkenyl-groups preferably having from 2 to 6 carbon atoms inclusively in the alkenyl moiety and from 6 to 10 atoms inclusively in the heteroaryl moiety.

“Heteroarylalkynyl” refers to heteroaryl-alkynyl-groups preferably having from 2 to 6 carbon atoms inclusively in the alkynyl moiety and from 6 to 10 atoms inclusively in the heteroaryl moiety.

“Heterocycle” refers to a saturated or unsaturated group having a single ring or multiple condensed rings, from 1 to 8 carbon atoms inclusively and from 1 to 4 hetero atoms inclusively selected from nitrogen, sulfur or oxygen within the ring. Such heterocyclic groups can have a single ring (e.g., piperidinyl or tetrahydrofuryl) or multiple condensed rings (e.g., indolinyl, dihydrobenzofuran or quinuclidinyl). Preferred heterocycles include piperidinyl, pyrrolidinyl and tetrahydrofuryl.

“Heterocycle-alkyl” refers to heterocycle-alkyl-groups preferably having from 1 to 6 carbon atoms inclusively in the alkyl moiety and from 6 to 10 atoms inclusively in the heterocycle moiety. Such heterocycle-alkyl groups are exemplified by morpholino-ethyl, pyrrolidinylmethyl, and the like.

“Heterocycle-alkenyl” refers to heterocycle-alkenyl-groups preferably having from 2 to 6 carbon atoms inclusively in the alkenyl moiety and from 6 to 10 atoms inclusively in the heterocycle moiety.

“Heterocycle-alkynyl” refers to heterocycle-alkynyl-groups preferably having from 2 to 6 carbon atoms inclusively in the alkynyl moiety and from 6 to 10 atoms inclusively in the heterocycle moiety.

Examples of heterocycles and heteroaryls include, but are not limited to, furan, thiophene, thiazole, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, pyrrolidine, indoline and the like.

Unless otherwise specified, positions occupied by hydrogen in the foregoing groups can be further substituted with substituents exemplified by, but not limited to, hydroxy, oxo, nitro, methoxy, ethoxy, alkoxy, substituted alkoxy, trifluoromethoxy, haloalkoxy, fluoro, chloro, bromo, iodo, halo, methyl, ethyl, propyl, butyl, alkyl, alkenyl, alkynyl, substituted alkyl, trifluoromethyl, haloalkyl, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, phenyl, aryl, substituted aryl, aryloxy, arylalkyl, arylalkenyl, arylalkynyl, pyridyl, imidazolyl, heteroaryl, substituted heteroaryl, heteroaryloxy, heteroarylalkyl, heteroarylalkenyl, heteroarylalkynyl, cyclopropyl, cyclobutyl, cyclopentyl, cyclohexyl, cycloalkyl, cycloalkenyl, cycloalkylalkyl, substituted cycloalkyl, cycloalkyloxy, pyrrolidinyl, piperidinyl, morpholino, heterocycle, (heterocycle)oxy, and (heterocycle)alkyl; and preferred heteroatoms are oxygen, nitrogen, and sulfur. It is understood that where open valences exist on these substituents they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further understood that the above substitutions can be made provided that replacing the hydrogen with the substituent does not introduce unacceptable instability to the molecules of the present invention, and is otherwise chemically reasonable.

The term “heteroatom-containing substituent” refers to substituents containing at least one non-halogen heteroatom. Examples of such substituents include, but are not limited to, hydroxy, oxo, nitro, methoxy, ethoxy, alkoxy, substituted alkoxy, trifluoromethoxy, haloalkoxy, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, aryloxy, pyridyl, imidazolyl, heteroaryl, substituted heteroaryl, heteroaryloxy, heteroarylalkyl, heteroarylalkenyl, heteroarylalkynyl, cycloalkyloxy, pyrrolidinyl, piperidinyl, morpholino, heterocycle, (heterocycle)oxy, and (heterocycle)alkyl; and preferred heteroatoms are oxygen, nitrogen, and sulfur. It is understood that where open valences exist on these substituents they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further understood that the above substitutions can be made provided that replacing the hydrogen with the substituent does not introduce unacceptable instability to the molecules of the present invention, and is otherwise chemically reasonable.

“Pharmaceutically acceptable salts” are salts that retain the desired biological activity of the parent compound and do not impart undesired toxicological effects. Pharmaceutically acceptable salt forms include various polymorphs as well as the amorphous form of the different salts derived from acid or base additions. The acid addition salts can be formed with inorganic or organic acids. Illustrative but not restrictive examples of such acids include hydrochloric, hydrobromic, sulfuric, phosphoric, citric, acetic, propionic, benzoic, napthoic, oxalic, succinic, maleic, fumaric, malic, adipic, lactic, tartaric, salicylic, methanesulfonic, 2-hydroxyethanesulfonic, toluenesulfonic, benzenesulfonic, camphorsulfonic, and ethanesulfonic acids. The pharmaceutically acceptable base addition salts can be formed with metal or organic counterions and include, but are not limited to, alkali metal salts such as sodium or potassium; alkaline earth metal salts such as magnesium or calcium; and ammonium or tetraalkyl ammonium salts, i.e., NX₄⁺ (wherein X is C₁₋₄.

“Tautomers” are compounds that can exist in one or more forms, called tautomeric forms, which can interconvert by way of a migration of one or more hydrogen atoms in the compound accompanied by a rearrangement in the position of adjacent double bonds. These tautomeric forms are in equilibrium with each other, and the position of this equilibrium will depend on the exact nature of the physical state of the compound. It is understood that where tautomeric forms are possible, the current invention relates to all possible tautomeric forms.

“Solvates” are addition complexes in which a compound of Formula I or Formula II is combined with a pharmaceutically acceptable cosolvent in some fixed proportion. Cosolvents include, but are not limited to, water, methanol, ethanol, 1-propanol, isopropanol, 1-butanol, isobutanol, tert-butanol, acetone, methyl ethyl ketone, acetonitrile, ethyl acetate, benzene, toulene, xylene(s), ethylene glycol, dichloromethane, 1,2-dichloroethane, N-methylformamide, N,N-dimethylformamide, N-methylacetamide, pyridine, dioxane, and diethyl ether. Hydrates are solvates in which the cosolvent is water. It is to be understood that the definitions of compounds in Formula I and Formula II encompass all possible hydrates and solvates, in any proportion, which possess the stated activity.

“Inflammation” generally refers to a localized reaction of tissue, characterized by the influx of immune cells, which occurs in reaction to injury or infection.

“An effective amount” is the amount effective to treat a disease by ameliorating the pathological condition or reducing the symptoms of the disease. “An effective amount” is the amount effective to improve at least one of the parameters relevant to measurement of the disease.

The inventors of the present invention have discovered that compounds of Formula I or II, which are Rho kinase inhibitors, are effective in reducing cell proliferation, decreasing remodeling that is defined by cell migration and/or proliferation, reducing inflammation via the inhibition of leukocytes chemotaxis and the inhibition of cytokine and chemokine secretion, lowering or preventing tissue or organ edema via the increase of endothelial cell junction integrity, and reducing vasoconstriction via the disruption of acto-myosin-based cytoskeleton within smooth muscle cells, thereby reducing smooth muscle tone and contractibility. By having the above properties, compounds of Formula I or II are useful in a method of preventing or treating cardiovascular diseases or conditions associated with excessive cell proliferation, remodeling, inflammation, and vasoconstriction.

The invention provides a method of reducing excessive cell proliferation, a method of decreasing remodeling that is defined by cell migration and/or proliferation, a method of reducing inflammation via inhibition of leukocytes chemotaxis and via decreasing cytokine and chemokine secretion, and a method of reducing vasoconstriction via disruption of acto-myosin-based cytoskeleton within smooth muscle cells and thus reducing smooth muscle tone and contractibility. A method of reducing undesired platelet activation and aggregation by preventing platelet shape change and platelet aggregation. By resolving one or more of the above-described pathophysiologies, the present invention provides a method of treating of stent restenosis and thrombosis, vascular thrombosis, cerebral vasospasm, atherosclerosis, systemic hypertension, cardiac hypertrophy, and sexual dysfunction.

The present method comprises the steps of identifying a subject in need of treatment for the above conditions, and administering to the subject an effective amount of Rho kinase inhibitor compound of Formula I or II.

### Rho Kinase Inhibitor Compounds

The rho kinase inhibitor compounds useful for this invention include compounds of general Formula I and Formula II, and/or tautomers thereof, and/or pharmaceutically-acceptable salts, and/or solvates, and/or hydrates thereof. Compounds of general Formula I and Formula II can be prepared according to the methods disclosed in co-pending application US2008/0214614, which is incorporated herein by reference.

A compound according to Formula I or Formula II can exist in several diastereomeric forms. The general structures of Formula I and Formula II include all diastereomeric forms of such materials, when not specified otherwise. Formula I and Formula II also include mixtures of compounds of these Formulae, including mixtures of enantiomers, diastereomers and/or other isomers in any proportion.

### A. Formula I

Compounds of Formula I are as follows:

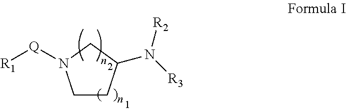

wherein: R₁ is aryl or heteroaryl, optionally substituted;
Q is C═O, SO₂, or (CR₄R₅)ₙ₃;
n₁ is 1, 2, or 3;
n₂ is for 2;
n₃ is 0, 1, 2, or 3;
wherein the ring represented by

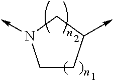

is optionally substituted by alkyl, halo, oxo, OR₆, NR₆R₇, or SR₆;
R₂ is selected from the following heteroaryl systems, optionally substituted:

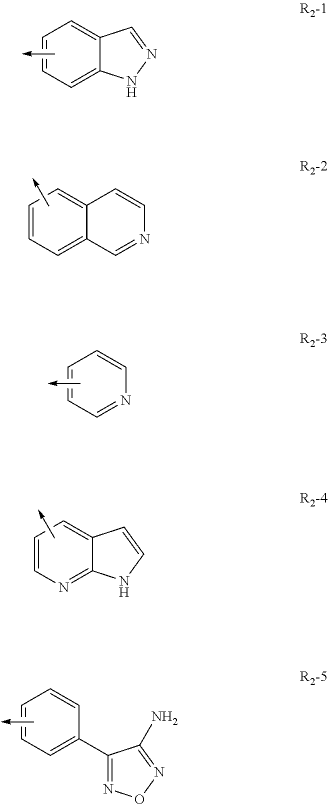

R₃-R₇ are independently H, alkyl, alkenyl, alkynyl, cycloalkyl, cycloalkenyl, cycloalkylalkyl, cycloalkylalkenyl, or cycloalkylalkynyl optionally substituted.

In Formula I, a preferred R₁ is substituted aryl, a more preferred R₁ is substituted phenyl, the preferred Q is (CR₄R₅)ₙ₃, the more preferred Q is CH₂, the preferred n₁ is 1 or 2, the preferred n₂ is 1, the preferred n₃ is 1 or 2, and the preferred R₃-R₇ are H.

In Formula I, a preferred R₂ substituent is halo, alkyl, cycloalkyl, hydroxyl, alkoxy, cycloalkyloxy, amino, alkylamino, or R₂ is unsubstituted. A more preferred R₂ substituent is halo, methyl, ethyl, isopropyl, cyclopropyl, hydroxyl, methoxy, ethoxy, amino, methylamino, dimethylamino, or R₂ is unsubstituted.
[1] One embodiment of the invention is represented by Formula I, in which R₂ is 5-indazolyl or 6-indazolyl (R₂-1), optionally substituted.
[1a] In embodiment 1, R₂-1 is substituted by one or more alkyl or halo substituents.
[1b] In embodiment 1, R₂-1 is substituted by one or more amino, alkylamino, hydroxyl, or alkoxy substituents.
[1c] In embodiment 1, R₂-1 is unsubstituted.
[2] In another embodiment, the invention is represented by Formula I in which R₂ is 5-isoquinolinyl or 6-isoquinolinyl (R₂-2), optionally substituted.
[2a] In embodiment 2, R₂-2 is substituted by one or more alkyl or halo substituents.
[2b] In embodiment 2, R₂-2 is substituted by one or more amino, alkylamino, hydroxyl, or alkoxy substituents.
[2c] In embodiment 2, R₂-2 is unsubstituted.
[3] In another embodiment, the invention is represented by Formula I in which R₂ is 4-pyridyl or 3-pyridyl (R₂-3), optionally substituted.
[3a] In embodiment 3, R₂-3 is substituted by one or more alkyl or halo substituents.
[3b] In embodiment 3, R₂-3 is substituted by one or more amino, alkylamino, hydroxyl, or alkoxy substituents.
[3c] In embodiment 3, R₂-3 is unsubstituted.
[4] In another embodiment, the invention is represented by Formula I in which R₂ is 7-azaindol-4-yl or 7-azaindol-5-yl (R₂-4), optionally substituted.
[4a] In embodiment 4, R₂-4 is substituted by one or more alkyl or halo substituents.
[4b] In embodiment 4, R₂-4 is substituted by one or more amino, alkylamino, hydroxyl, or alkoxy substituents.
[4c] In embodiment 4, R₂-4 is unsubstituted.
[5] In another embodiment, the invention is represented by Formula I in which R₂ is 4-(3-amino-1,2,5-oxadiazol-4-yl)phenyl or 3-(3-amino-1,2,5-oxadiazol-4-yl)phenyl (R₂-5), optionally substituted.
[5a] In embodiment 5, R₂-5 is unsubstituted.
[6] In another embodiment, the invention is represented by Formula I in which R₂ is one of the groups R₂-1-R₂-5, substituted by one or more alkyl, halo, amino, alkylamino, hydroxyl, or alkoxy substituents.
[6a] In embodiment 6, R₂ is substituted by one or more alkyl or halo substituents.
[6b] In embodiment 6, R₂ is substituted by one or more amino, alkylamino, hydroxyl, or alkoxy substituents.
[7] In another embodiment, the invention is represented by Formula I in which R₂ is one of the groups R₂-1-R₂-5, and is unsubstituted.
[8] In another embodiment, the invention is represented by Formula I in which R₃ is H.
[9] In another embodiment, the invention is represented by Formula I in which Q is (CR₄R₅)ₙ₃, and n₃ is 1 or 2.
[10] In another embodiment, the invention is represented by Formula I in which Q is (CH₂)ₙ₃, and n₃ is 1.
[11] In another embodiment, the invention is represented by Formula I in which R₁ is aryl or heteroaryl substituted with one or more alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituents, optionally further substituted.
Compounds exemplifying embodiment 11 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table A.
[12] In another embodiment, the invention is represented by Formula I in which R₁ is aryl or heteroaryl substituted with one or more heteroatom-containing substituents, with the proviso that if the R₁ substituent is acyclic and is connected to R₁ by a carbon atom, then this substituent contains at least one nitrogen or sulfur atom, with the second proviso that if the substituent is acyclic and is connected to R₁ by an oxygen or nitrogen atom, then this substituent contains at least one additional oxygen, nitrogen or sulfur atom, and with the third proviso that if the substituent is connected to R₁ by a sulfone linkage “—SO₂-”, then R₂ is not nitrogen- or oxygen-substituted R₂-2.
[12a] In embodiment 12, the heteroatom-containing substituent is connected to R₁ by an oxygen or nitrogen atom.
[12b] In embodiment 12, the heteroatom-containing substituent is connected to R₁ by a sulfide linkage, “−5-”.
Compounds exemplifying embodiment 12 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table A.
[13] In another embodiment, the invention is represented by Formula I in which R₁ is aryl or heteroaryl substituted with one or more alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituents, which are further substituted with one or more heteroatom-containing substituents, with the proviso that if the R₁ substituent is acyclic and its heteroatom-containing substituent falls on the carbon by which it is attached to R₁, then the heteroatom-containing substituent contains at least one nitrogen or sulfur atom.
Compounds exemplifying embodiment 13 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, 1.122, and 1.123, shown below in Table A.
[14] In another embodiment, the invention is represented by Formula I in which R₁ is aryl or heteroaryl substituted with one or more alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituents, optionally further substituted, and R₂ is 5-indazolyl (R₂-1) or 5-isoquinolinyl (R₂-2), optionally substituted.
[14a] In embodiment 14, R₂ is 5-indazolyl (R₂-1), optionally substituted by one or more alkyl, halo, amino, alkylamino, hydroxyl, or alkoxy substituents.
[14b] In embodiment 14, R₂ is 5-isoquinolinyl (R₂-2), optionally substituted by one or more alkyl, halo, amino, alkylamino, hydroxyl, or alkoxy substituents.
[14c] In embodiment 14, R₂ is unsubstituted.
Compounds exemplifying embodiment 14 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table A.
[15] In another embodiment, the invention is represented by Formula I in which R₁ is aryl or heteroaryl substituted with one or more heteroatom-containing substituents, and R₂ is 5-indazolyl (R₂-1) or 5-isoquinolinyl (R₂-2), optionally substituted, with the proviso that if the R₁ substituent is acyclic and is connected to R₁ by a carbon atom, then this substituent contains at least one nitrogen or sulfur atom, with the second proviso that if the substituent is acyclic and is connected to R₁ by an oxygen or nitrogen atom, then this substituent contains at least one additional oxygen, nitrogen or sulfur atom, and with the third proviso that if the substituent is connected to R₁ by a sulfone linkage “—SO₂-”, then R₂ is not nitrogen- or oxygen-substituted R₂-2.
[15a] In embodiment 15, R₂ is 5-indazolyl (R₂-1), optionally substituted by one or more alkyl, halo, amino, alkylamino, hydroxyl, or alkoxy substituents.
[15b] In embodiment 15, R₂ is 5-isoquinolinyl (R₂-2), optionally substituted by one or more alkyl, halo, amino, alkylamino, hydroxyl, or alkoxy substituents.
[15c] In embodiment 15, R₂ is unsubstituted.
[15d] In embodiment 15, the heteroatom-containing substituent is connected to R₁ by an oxygen or nitrogen atom.
[15e] In embodiment 15, the heteroatom-containing substituent is connected to R₁ by a sulfide linkage, “−5-”.
Compounds exemplifying embodiment 15 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table A.
[16] In another embodiment, the invention is represented by Formula I in which R₁ is aryl or heteroaryl substituted with one or more alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituents, at least one of which is further substituted with one or more heteroatom-containing substituents, and R₂ is 5-indazolyl (R₂-1) or 5-isoquinolinyl (R₂-2), optionally substituted, with the proviso that if the R₁ substituent is acyclic and its heteroatom-containing substituent falls on the carbon by which it is attached to R₁, then the heteroatom-containing substituent contains at least one nitrogen or sulfur atom.
[16a] In embodiment 16, R₂ is 5-indazolyl (R₂-1), optionally substituted by one or more alkyl, halo, amino, alkylamino, hydroxyl, or alkoxy substituents.
[16b] In embodiment 16, R₂ is 5-isoquinolinyl (R₂-2), optionally substituted by one or more alkyl, halo, amino, alkylamino, hydroxyl, or alkoxy substituents.
[16c] In embodiment 16, R₂ is unsubstituted.
Compounds exemplifying embodiment 16 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, 1.122, and 1.123, shown below in Table A.
The inventors have discovered certain compounds of Formula I that have properties that render them particularly useful for treating the conditions addressed by the invention. In particular, these preferred compounds can be described as compounds of Formula I in which R₂, R₃, n₁, and n₂ are limited to the combinations shown in Formulae Ia, Ib, and Ic:

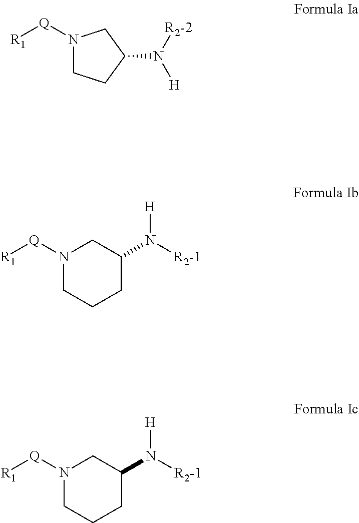

In Formulae Ia, Ib, and Ic, the stereochemistry of the central pyrrolidine or piperidine ring is limited to the R, R, and S configurations respectively, as drawn. Further, the group R₁ in these Formulae is limited to phenyl, thiophene, and 6,5- or 6,6-fused bicyclic heteroaryl rings. The group R₁ is either unsubstituted or is optionally substituted with 1, 2 or 3 substituents independently selected from halogen, methyl, ethyl, hydroxyl, methoxy, or ethoxy.
In Formula Ia, Ib, and Ic, Q is C═O, SO₂, or (CR₄R₅)ₙ₃; where R₄ and R₅ are independently H, alkyl, cycloalkyl, optionally substituted. The preferred R₄ and R₅ are H or unsubstituted alkyl. The preferred Q is CH₂.
In Formula Ia, Ib, and Ic, a preferred R₂ substituent is halo, alkyl, cycloalkyl, hydroxyl, alkoxy, cycloalkyloxy, amino, alkylamino, or R₂ is unsubstituted. A more preferred R₂ substituent is halo, methyl, ethyl, isopropyl, cyclopropyl, hydroxyl, methoxy, ethoxy, amino, methylamino, dimethylamino, or R₂ is unsubstituted.
In a more preferred form of Formulae Ia, Ib, and Ic, R₁ is phenyl or a 6,5-fused bicyclic heteroaryl ring, optionally substituted by 1 or 2 substituents, Q is CH₂, and the group R₂ is unsubstituted. The most preferred 6,5-fused bicyclic heteroaryl rings are benzofuran, benzothiophene, indole, and benzimidazole.
In another more preferred form, R₁ of Formulae Ia, Ib, and Ic is mono- or disubstituted when R₁ is phenyl, with 3-substituted, 4-substituted, 2,3-disubstituted, and 3,4-disubstituted being most preferred. When R₁ is bicyclic heteroaryl, an unsubstituted or monosubstituted R₁ is most preferred.
The inventors have found that certain members of Formulae Ia, Ib, and Ic, as defined above, are particularly useful in treating the conditions addressed in this invention. The compounds of the invention are multikinase inhibitors, with inhibitory activity against ROCK1 and ROCK2, in addition to several other kinases in individual compound cases. These kinase inhibitory properties endow the compounds of the invention not only with smooth muscle relaxant properties, but additionally with antiproliferative, antichemotactic, and cytokine secretion inhibitory properties that render them particularly useful in treating conditions with proliferative or inflammatory components as described in the invention.
[17] In particular, we have found that compounds in which R₂ is R₂-2 are particularly potent inhibitors of both ROCK1 and ROCK2, and that these agents inhibit the migration of neutrophils toward multiple chemotactic stimuli and inhibit the secretion of the cytokines IL-1β, TNF-α and IL-9 from LPS-stimulated human monocytes. Compounds in which R₁ is heteroaryl, particularly 6,5-fused bicyclic heteroaryl, are especially preferred. These compounds are of particular value in addressing conditions with an inflammatory component.
Compounds exemplifying embodiment 17 include compounds 2.025, 2.027, 2.046, 2.047, 2.048, 2.055, 2.056, 2.057, 2.061, 2.062, 2.065, 2.074, 2.075, 2.088, and 2.090.
[18] In another embodiment, we have found that compounds of Formula Ic are potent and selective inhibitors of ROCK2, with comparatively lower inhibitory potency against ROCK1. We have demonstrated that compounds of this class typically show good smooth muscle relaxation properties and that smooth muscle relaxation effects in this class are generally correlated with ROCK2 potency. Compounds in which R₁ is phenyl are particularly preferred. Compounds of this embodiment are of particular value in addressing conditions where relaxation of smooth muscle, in particular vascular and bronchial smooth muscle, is of highest importance.
Compounds exemplifying embodiment 18 include compounds 1.072, 1.078, 1.079, 1.080, 1.141, 1.142, 1.148, 1.149, 1.150, 1.151, 1.154, 1.155, 1.156, 1.163, 1.164, 1.166, 1.170, 1.171, 1.175, 1.179, 1.183, 1.227, 1.277, and 1.278.
[19] In another embodiment, the inventors have found that compounds of Formula Ib are potent mixed inhibitors of ROCK1 and ROCK2, display additional inhibitory activity against the kinases Akt3 and p70S6K, and that these compounds generally display potent antiproliferative activity in models of smooth muscle cell proliferation. Compounds of this class are of particular value in addressing conditions in which an antiproliferative component is desired in combination with a smooth muscle relaxing effect.
Compounds exemplifying embodiment 19 include compounds 1.073, 1.110, 1.131, 1.132, 1.133, 1.134, 1.135, 1.136, 1.137, 1.138, 1.143, 1.144, 1.145, 1.146, 1.172, 1.173, 1.177, 1.191, 1.192, 1.203, 1.210, 1.226, 1.241, 1.242, 1.245, 1.246, 1.252, and 1.254.
[20] In another embodiment, the inventors have found that certain compounds of Formulae Ia, Ib, and Ic distribute preferentially to the lung on oral dosing. In particular, compounds in which R₁ is a lipophilic bicyclic heteroaryl group are preferred for this dosing behavior.
Compounds of this type are especially useful for treating diseases of the lung by oral dosing while minimizing impact on other tissues.
Compounds exemplifying embodiment 20 include compounds 1.131, 1.137, 1.138, 1.143, 1.148, 1.149, 1.150, 1.166, 1.175, 1.177, 1.246, 1.252, 2.055, 2.056, 2.057, 2.065, 2.074, and 2.075.
[21] In another embodiment, the inventors have found that certain compounds of Formulae Ia, Ib, and Ic produce low plasma concentrations of the compound when dosed by the oral route. Compounds in which one substituent on R₁ is selected from the group methyl, ethyl, or hydroxyl are preferred for typically exhibiting this pharmacokinetic behavior. Compounds displaying this property are particularly useful for inhalation dosing, since a large portion of the material dosed in this way is typically swallowed, and it is advantageous for this swallowed portion to remain unabsorbed or to be cleared rapidly so as to minimize the impact of the compound on other tissues.
Compounds exemplifying embodiment 21 include compounds 1.078, 1.133, 1.135, 1.136, 1.145, 1.151, 1.154, 1.155, 1.156, 1.163, 1.171, 1.172, 1.173, 1.192, 1.242, 2.025, and 2.061.
Preparation of compounds of Formulae Ia, Ib, and Ic can be problematic using methods commonly known in the art. In particular, syntheses of compounds of Formulae Ib and Ic using transition metal mediated coupling reactions to form the critical bond between R₂-1 and the nitrogen atom are hampered by low yields when the indazole ring is not protected properly to allow a successful reaction. Specifically, the methods disclosed in UA2006/0167043 fail to provide the desired amino indazole products when the indazole is unprotected or is protected with a standard acyl protecting group such as pivalate or alkoxycarbonyl protecting groups. The inventors prepare compounds of Formulae Ia, Ib, and Ic according to the methods disclosed in the co-pending application US2008/0214614, which allows the successful protection, coupling, and deprotection of the indazole ring, thereby allowing the successful preparation of the compounds of Formulae Ib and Ic and the demonstration of their useful biological properties.

### B. Formula II

A preferred compound of Formula I is where R₁=Ar—X, shown below as Formula II:

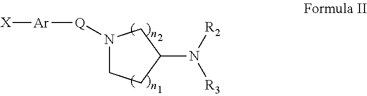

wherein:
Ar is a monocyclic or bicyclic aryl or heteroaryl ring, such as phenyl;
X is from 1 to 3 substituents on Ar, each independently in the form Y—Z, in which Z is attached to Ar;
Y is one or more substituents on Z, and each is chosen independently from H, halogen, or the heteroatom-containing substituents, including but not limited to OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀;
Each instance of Z is chosen independently from alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, heterocycle, (heterocycle)alkyl, (heterocycle)alkenyl, (heterocycle)alkynyl, or is absent;
R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents, including but not limited to OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, or NR₁₁C(═O)NR₁₂R₁₃;
R₉ and R₁₀ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents, including but not limited to OR₁₄, NR₁₄R₁₅, NO₂, SR₁₄, SOR₁₄, SO₂R₁₄, SO₂NR₁₄R₁₅, NR₁₄SO₂R₁₅, OCF₃, CONR₁₄R₁₅, NR₁₄C(═O)R₁₅, NR₁₄C(═O)OR₁₅, OC(═O)NR₁₄R₁₅, or NR₁₄C(═O)NR₁₅R₁₆; any two of the groups R₈, R₉ and R₁₀ are optionally joined with a link selected from the group consisting of bond, —O—, —S—, —SO—, —SO₂—, and —NR₁₇— to form a ring;
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle.
In Formula II, the preferred Y is H, halogen, OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀, the more preferred Y is H, halogen, OR₈, SR₈, SOR₈, SO₂R₈, SO₂NR₈R₉, NR₈SO₂R₉, CONR₈R₉, or NR₈C(═O)NR₉R₁₀, the preferred Z is alkyl, alkenyl, alkynyl, cycloalkyl, cycloalkenyl, cycloalkylalkyl, or is absent; the more preferred Z is alkyl, alkenyl, alkynyl, cycloalkyl, or is absent, the preferred Q is (CR₄R₅)ₙ₃, the more preferred Q is CH₂, the preferred n₁ is 1 or 2, the preferred n₂ is 1, the preferred n₃ is 1 or 2, the preferred R₃-R₇ are H, the preferred R₉ is H, alkyl, arylalkyl, cycloalkyl, cycloalkylalkyl, or heterocycle, the preferred R₉ substituents are H, halogen, OR₁₁, NR₁₁R₁₂, SR₁₁, SOR₁₁, SO₂R₁₁, SO₂NR₁₁R₁₂, NR₁₁SO₂R₁₂, CONR₁₁R₁₂, NR₁₁C(═O)R₁₂, and the preferred R₉-R₁₇ are H or alkyl.
In Formula II, a preferred R₂ substituent is halo, alkyl, cycloalkyl, hydroxyl, alkoxy, cycloalkyloxy, amino, alkylamino, or R₂ is unsubstituted. A more preferred R₂ substituent is halo, methyl, ethyl, isopropyl, cyclopropyl, hydroxyl, methoxy, ethoxy, amino, methylamino, dimethylamino, or R₂ is unsubstituted.
[1] One embodiment of the invention is represented by Formula II in which R₂ is 5-indazolyl or 6-indazolyl (R₂-1), optionally substituted.
[1a] In embodiment 1, R₂-1 is substituted by one or more alkyl or halo substituents.
[1b] In embodiment 1, R₂-1 is substituted by one or more amino, alkylamino, hydroxyl, or alkoxy substituents.
[1c] In embodiment 1, R₂-1 is unsubstituted.
[2] In another embodiment, the invention is represented by Formula II in which R₂ is 5-isoquinolinyl or 6-isoquinolinyl (R₂-2), optionally substituted.
[2a] In embodiment 2, R₂-2 is substituted by one or more alkyl or halo substituents.
[2b] In embodiment 2, R₂-2 is substituted by one or more amino, alkylamino, hydroxyl, or alkoxy substituents.
[2c] In embodiment 2, R₂-2 is unsubstituted.
[3] In another embodiment, the invention is represented by Formula II in which R₂ is 4-pyridyl or 3-pyridyl (R₂-3), optionally substituted.
[3a] In embodiment 3, R₂-3 is substituted by one or more alkyl or halo substituents.
[3b] In embodiment 3, R₂-3 is substituted by one or more amino, alkylamino, hydroxyl, or alkoxy substituents.
[3c] In embodiment 3, R₂-3 is unsubstituted.
[4] In another embodiment, the invention is represented by Formula II in which R₂ is 7-azaindol-4-yl or 7-azaindol-5-yl (R₂-4), optionally substituted.
[4a] In embodiment 4, R₂-4 is substituted by one or more alkyl or halo substituents.
[4b] In embodiment 4, R₂-4 is substituted by one or more amino, alkylamino, hydroxyl, or alkoxy substituents.
[4c] In embodiment 4, R₂-4 is unsubstituted.
[5] In another embodiment, the invention is represented by Formula II in which R₂ is 4-(3-amino-1,2,5-oxadiazol-4-yl)phenyl or 3-(3-amino-1,2,5-oxadiazol-4-yl)phenyl (R₂-5), optionally substituted.
[5a] In embodiment 5, R₂-5 is unsubstituted.
[6] In another embodiment, the invention is represented by Formula II in which R₂ is one of the groups R₂-1-R₂-5, substituted by one or more alkyl, halo, amino, alkylamino, hydroxyl, or alkoxy substituents.
[6a] In embodiment 6, R₂ is substituted by one or more alkyl or halo substituents.
[6b] In embodiment 6, R₂ is substituted by one or more amino, alkylamino, hydroxyl, or alkoxy substituents.
[7] In another embodiment, the invention is represented by Formula II in which R₂ is one of the groups R₂-1-R₂-5, and is unsubstituted.
[8] In another embodiment, the invention is represented by Formula II in which R₃ is H.
[9] In another embodiment, the invention is represented by Formula II in which Q is (CR₄R₅)₉₃, and n₃ is 1 or 2.
[10] In another embodiment, the invention is represented by Formula II in which Q is (CH₂)ₙ₃, and n₃ is 1.
[11] In another embodiment, the invention is represented by Formula II in which for at least one substituent X, Z is alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkylalkenyl, cycloalkylalkynyl, cycloalkenyl, cycloalkylalkyl, heterocycle, (heterocycle)alkyl, (heterocycle)alkenyl, or (heterocycle)alkynyl.
Compounds exemplifying embodiment 11 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table A.
[12] In another embodiment, the invention is represented by Formula II in which for at least one substituent X, Z is absent, and Y is a heteroatom-containing substituent, including but not limited to OR₈, NR₈R₉, SR₈, SOR₈, SO₂R₈, SO₂NR₈R₉, NR₈SO₂R₉, CONR₈R₉, NR₈C(═O)R₉, NR₈C(═O)OR₉, OC(═O)NR₈R₉, or NR₈C(═O)NR₉R₁₀, with the proviso that if the substituent Y is acyclic and is connected to Ar by a carbon atom, then this substituent contains at least one nitrogen or sulfur atom, with the second proviso that if the substituent Y is acyclic and is connected to Ar by an oxygen or nitrogen atom, then this substituent contains at least one additional oxygen, nitrogen or sulfur atom, and with the third proviso that if the substituent Y is connected to Ar by a sulfone linkage “—SO₂-”, then R₂ is not nitrogen- or oxygen-substituted R₂-2.
[12a] In embodiment 12, the heteroatom-containing substituent is connected to R₁ by an oxygen or nitrogen atom.
[12b] In embodiment 12, the heteroatom-containing substituent is connected to R₁ by a sulfide linkage, “—S—”.
Compounds exemplifying embodiment 12 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table A.
[13] In another embodiment, the invention is represented by Formula II in which for at least one substituent X, Z is alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl, and Y is a heteroatom-containing substituent, including but not limited to OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀, with the proviso that if Z is acyclic and Y falls on the carbon by which Z is attached to Ar, then Y contains at least one nitrogen or sulfur atom.
Compounds exemplifying embodiment 13 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, 1.122, and 1.123, shown below in Table A.
[14] In another embodiment, the invention is represented by Formula II in which for at least one substituent X, Z is alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl, and R₂ is 5-indazolyl (R₂-1) or 5-isoquinolinyl (R₂-2), optionally substituted.
[14a] In embodiment 14, R₂ is 5-indazolyl (R₂-1), optionally substituted by one or more alkyl, halo, amino, alkylamino, hydroxyl, or alkoxy substituents.
[14b] In embodiment 14, R₂ is 5-isoquinolinyl (R₂-2), optionally substituted by one or more alkyl, halo, amino, alkylamino, hydroxyl, or alkoxy substituents.
[14c] In embodiment 14, R₂ is unsubstituted.
Compounds exemplifying embodiment 14 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table A.
[15] In another embodiment, the invention is represented by Formula II in which for at least one substituent X, Z is absent, and Y is a heteroatom-containing substituent, including but not limited to OR₈, NR₈R₉, SR₈, SOR₈, SO₂R₈, SO₂NR₈R₉, NR₈SO₂R₉, CONR₈R₉, NR₈C(═O)R₉, NR₈C(═O)OR₉, OC(═O)NR₈R₉, or NR₈C(═O)NR₉R₁₀, and R₂ is 5-indazolyl (R₂-1) or 5-isoquinolinyl (R₂-2), optionally substituted, with the proviso that if the substituent Y is acyclic and is connected to Ar by a carbon atom, then this substituent contains at least one nitrogen or sulfur atom, with the second proviso that if the substituent Y is acyclic and is connected to Ar by an oxygen or nitrogen atom, then this substituent contains at least one additional oxygen, nitrogen or sulfur atom, and with the third proviso that if the substituent Y is connected to Ar by a sulfone linkage “—SO₂-”, then R₂ is not nitrogen- or oxygen-substituted R₂-2.
[15a] In embodiment 15, R₂ is 5-indazolyl (R₂-1), optionally substituted by one or more alkyl, halo, amino, alkylamino, hydroxyl, or alkoxy substituents.
[15b] In embodiment 15, R₂ is 5-isoquinolinyl (R₂-2), optionally substituted by one or more alkyl, halo, amino, alkylamino, hydroxyl, or alkoxy substituents.
[15c] In embodiment 15, R₂ is unsubstituted.
[15d] In embodiment 15, the heteroatom-containing substituent is connected to R₁ by an oxygen or nitrogen atom.
[15e] In embodiment 15, the heteroatom-containing substituent is connected to R₁ by a sulfide linkage, “—S-”.
Compounds exemplifying embodiment 15 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table A.
[16] In another embodiment, the invention is represented by Formula II in which for at least one substituent X, Z is alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl, and Y is a heteroatom-containing substituent, including but not limited to OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or

NR₈C(═O)NR₉R₁₀, and R₂ is 5-indazolyl (R₂-1) or 5-isoquinolinyl (R₂-2), optionally substituted, with the proviso that if Z is acyclic and Y falls on the carbon by which Z is attached to Ar, then Y contains at least one nitrogen or sulfur atom.

[16a] In embodiment 16, R₂ is 5-indazolyl (R₂-1), optionally substituted by one or more alkyl, halo, amino, alkylamino, hydroxyl, or alkoxy substituents.
[16b] In embodiment 16, R₂ is 5-isoquinolinyl (R₂-2), optionally substituted by one or more alkyl, halo, amino, alkylamino, hydroxyl, or alkoxy substituents.
[16c] In embodiment 16, R₂ is unsubstituted.
[16d] In embodiment 16, Ar is heteroaryl.
Compounds exemplifying embodiment 16 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, 1.122, and 1.123, shown below in Table A.
In Embodiments 11-16 of Formula II, the preferred Q is (CR₄R₅)ₙ₃, the more preferred Q is CH₂, the preferred n₁ is 1 or 2, the preferred n₂ is 1, the preferred n₃ is 1 or 2, and the preferred R₃ is H.
The inventors have discovered certain compounds of Formula II that have properties that render them particularly useful for treating the conditions addressed by the invention. In particular, these preferred compounds of Embodiments 14, 15 and 16 can be described as compounds of Formula II in which R₂, R₃, n₁, and n₂ are limited to the combinations shown in Formulae IIa, IIb, and IIc:

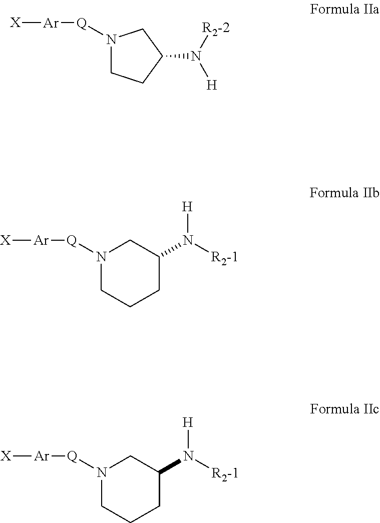

In Formulae IIa, IIb, and IIc, the stereochemistry of the central pyrrolidine or piperidine ring is limited to the R, R, and S configurations respectively, as drawn.
In Formula IIa, IIb, and IIc, Q is C═O, SO₂, or (CR₄R₅)ₙ₃; where R₄ and R₅ are independently H, alkyl, cycloalkyl, optionally substituted. The preferred R₄ and R₅ are H or unsubstituted alkyl. The preferred Q is CH₂.
In Formula IIa, IIb, and IIc, a preferred R₂ substituent is halo, alkyl, cycloalkyl, hydroxyl, alkoxy, cycloalkyloxy, amino, alkylamino, or R₂ is unsubstituted. A more preferred R₂ substituent is halo, methyl, ethyl, isopropyl, cyclopropyl, hydroxyl, methoxy, ethoxy, amino, methylamino, dimethylamino, or R₂ is unsubstituted.
In a more preferred form of Formulae IIa, IIb, and IIc, Ar is phenyl or a 6,5- or 6,6-fused bicyclic heteroaryl ring, substituted by 1 or 2 substituents X, and Q is CH₂. The most preferred 6,5-fused bicyclic heteroaryl rings are benzofuran, benzothiophene, indole, and benzimidazole.
In its more preferred form, Ar of Formulae IIa, IIb, and IIc is mono- or disubstituted when Ar is phenyl, with 3-substituted, 4-substituted, 2,3-disubstituted, and 3,4-disubstituted being most preferred. When Ar is bicyclic heteroaryl, a monosubstituted Ar is most preferred.
The inventors have found that certain members of Formulae IIa, IIb, and IIc, as defined above, are particularly useful in treating the conditions addressed in this invention. The compounds of the invention are multikinase inhibitors, with inhibitory activity against ROCK1 and ROCK2, in addition to several other kinases in individual compound cases. These kinase inhibitory properties endow the compounds of the invention not only with smooth muscle relaxant properties, but additionally with antiproliferative, antichemotactic, and cytokine secretion inhibitory properties that render them particularly useful in treating conditions with proliferative or inflammatory components as described in the invention.
[17] In particular, we have found that compounds in which R₂ is R₂-2 are particularly potent inhibitors of both ROCK1 and ROCK2, and that these agents inhibit the migration of neutrophils toward multiple chemotactic stimuli and inhibit the secretion of the cytokines IL-1β, TNF-α and IL-9 from LPS-stimulated human monocytes. Compounds in which Ar is heteroaryl, particularly 6,5-fused bicyclic heteroaryl, are especially preferred. These compounds are of particular value in addressing conditions with an inflammatory component.
Compounds exemplifying embodiment 17 include compounds 2.020, 2.021, 2.022, 2.026, 2.031, 2.033, 2.034, 2.038, 2.039, 2.040, 2.041, 2.043, 2.044, 2.054, 2.058, 2.059, 2.060, 2.063, 2.064, 2.066, 2.067, 2.068, 2.069, 2.070, 2.071, 2.072, 2.073, 2.076, 2.077, 2.078, 2.079, 2.080, 2.081, 2.082, 2.087, 2.092, 2.093, 2.094, 2.095, 2.096, 2.097, 2.098, 2.099, and 2.100.
[18] In another embodiment, we have found that compounds of Formula IIc are potent and selective inhibitors of ROCK2, with comparatively lower inhibitory potency against ROCK1.
We have demonstrated that compounds of this class typically show good smooth muscle relaxation properties and that smooth muscle relaxation effects in this class are generally correlated with ROCK2 potency. Compounds in which Ar is phenyl are particularly preferred, and compounds bearing one polar group X1 in the 3-position and a second group X2 in the 4-position are most preferred. Compounds of this embodiment are of particular value in addressing conditions where relaxation of smooth muscle, in particular vascular and bronchial smooth muscle, is of highest importance.
Compounds exemplifying embodiment 18 include compounds 1.075, 1.077, 1.090, 1.091, 1.094, 1.095, 1.107, 1.109, 1.117, 1.118, 1.124, 1.152, 1.153, 1.157, 1.158, 1.165, 1.168, 1.176, 1.181, 1.182, 1.184, 1.185, 1.186, 1.187, 1.195, 1.196, 1.197, 1.198, 1.199, 1.200, 1.201, 1.213, 1.214, 1.215, 1.217, 1.218, 1.219, 1.223, 1.224, 1.228, 1.229, 1.230, 1.233, 1.234, 1.236, 1.237, 1.238, 1.239, 1.240, 1.253, 1.255, 1.261, 1.269, 1.270, 1.272, 1.274, 1.275, 1.280, and 1.282.
[19] In another embodiment, the inventors have found that compounds of Formula IIb are potent mixed inhibitors of ROCK1 and ROCK2, display additional inhibitory activity against the kinases Akt3 and p70S6K, and that these compounds generally display potent antiproliferative activity in models of smooth muscle cell proliferation. Compounds of this class are of particular value in addressing conditions in which an antiproliferative component is desired in combination with a smooth muscle relaxing effect.
Compounds exemplifying embodiment 19 include compounds 1.074, 1.076, 1.092, 1.093, 1.096, 1.097, 1.106, 1.108, 1.113, 1.115, 1.116, 1.123, 1.125, 1.126, 1.127, 1.128, 1.129, 1.139, 1.140, 1.147, 1.159, 1.160, 1.161, 1.162, 1.174, 1.188, 1.189, 1.193, 1.194, 1.202, 1.205, 1.206, 1.207, 1.208, 1.211, 1.212, 1.221, 1.222, 1.225, 1.231, 1.232, 1.235, 1.244, 1.248, 1.249, 1.258, 1.259, 1.260, 1.262, 1.263, 1.264, 1.265, 1.266, 1.267, 1.268, 1.271, 1.273, 1.276, and 1.281.
[20] In another embodiment, the inventors have found that certain compounds of Formulae IIa, IIb, and IIc distribute preferentially to the lung on oral dosing. In particular, compounds in which Ar is a lipophilic bicyclic heteroaryl group are preferred for this dosing behavior.
Compounds of this type are especially useful for treating diseases of the lung by oral dosing while minimizing impact on other tissues.
Compounds exemplifying embodiment 20 include compounds 1.107, 1.109, 1.165, 1.106, 1.108, 2.058, 1.162, 1.264, 1.268, 1.271, 1.273, 1.217, 1.269, 2.059, 2.060, 2.066, and 2.072.
As discussed above for the compounds of Formulae Ia, Ib, and Ic, preparation of compounds of Formulae IIa, IIb, and IIc can be problematic using methods commonly known in the art. The inventors have disclosed and exemplified in US2008/0214614A1 methods to allow successful protection, coupling, and deprotection sequence that allows the successful preparation of the compounds of Formulae IIb and IIc and the demonstration of their useful biological properties.

The present compounds are useful for both oral and topical use, including use by the inhalation route. To be therapeutically effective in this way, the compounds must have both adequate potency and proper pharmacokinetic properties such as good permeability across the biological surface relevant to the delivery route. In general, compounds of Formulae I and II bearing polar functionality, particularly on Ar, have preferred absorption properties and are particularly suitable for topical use. In general, compounds bearing small lipophilic functional groups have good ROCK inhibitory potency.

R₁ substitution in Formula I and X in Formula II are important factors for pharmacokinetic properties and ROCK inhibitory potency. Specifically, compounds bearing polar functionality, especially those specified in the embodiments 11, 12, 13, 14, 15, and 16 in Formulae I and II, above, are particularly suitable for topical use with adequate ROCK inhibiting activity. Compounds bearing small lipophilic functional groups, as specified in the embodiments 11, 12, 13, 14, 15, and 16 in Formulae I and II, above, display ROCK inhibition with adequate permeability across biological surfaces. Compounds bearing substituents of both types are particularly preferred, and when R₁ (Formula I) or Ar (Formula II) is a phenyl ring, compounds with small lipophilic groups in the 4-position and polar functionality in the 3-position are most preferred.

Specific compounds illustrative of Formula I and Formula II are shown in the following Table A. The example compounds have been numbered in such a way that numbers of the form 1.nnn indicate compounds in which R₂ is R₂-1, numbers of the form 2.nnn indicate compounds in which R₂ is R₂-2, and so on in a similar fashion for the remaining compound numbers and groups R₂. In the following structures, hydrogens are omitted from the drawings for the sake of simplicity. Tautomers drawn represent all tautomers possible. Structures are drawn to indicate the preferred stereochemistry; where stereoisomers may be generated in these compounds, structures are taken to mean any of the possible stereoisomers alone or a mixture of stereoisomers in any ratio.

TABLE A
Exemplified Compounds.

| | | Select |
|---|---|---|
| Compound | Structure | Embodiments 1-16 |
| 1.001 | 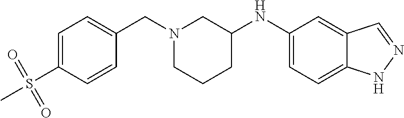 [SMILES: CS(=O)(=O)c1ccc(CN2CCCC(Nc3ccc4[nH]ncc4c3)C2)cc1] N-(1-(4-(methylsulfonyl)benzyl)piperidin-3-yl)-1H- indazol-5-amine | 1c, 7, 8, 9, 10, 12, 15c |
| 1.002 | 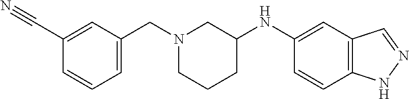 [SMILES: N#Cc1cccc(CN2CCCC(Nc3ccc4[nH]ncc4c3)C2)c1] 3-((3-(1H-indazol-5-ylamino)piperidin-1- yl)methyl)benzonitrile | 1c, 7, 8, 9, 10, 12, 15c |
| 1.003 | 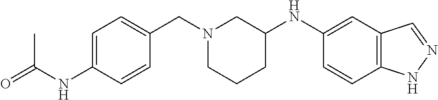 [SMILES: CC(=O)Nc1ccc(CN2CCCC(Nc3ccc4[nH]ncc4c3)C2)cc1] N-(4-((3-(1H-indazol-5-ylamino)piperidin-1- yl)methyl)phenyl)acetamide | 1c, 7, 8, 9, 10, 12a, 15c, 15d |
| 1.004 | 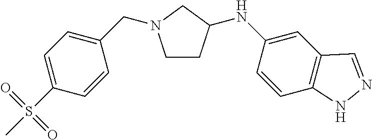 [SMILES: CS(=O)(=O)c1ccc(CN2CCC(Nc3ccc4[nH]ncc4c3)C2)cc1] N-(1-(4-(methylsulfonyl)benzyl)pyrrolidin-3-yl)-1H- indazol-5-amine | 1c, 7, 8, 9, 10, 12, 15c |
| 1.005 | 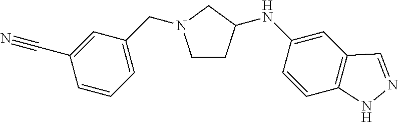 [SMILES: N#Cc1cccc(CN2CCC(Nc3ccc4[nH]ncc4c3)C2)c1] 3-((3-(1H-indazol-5-ylamino)pyrrolidin-1- yl)methyl)benzonitrile | 1c, 7, 8, 9, 10, 12, 15c |
| 1.006 | 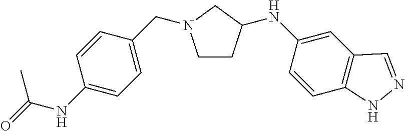 [SMILES: CC(=O)Nc1ccc(CN2CCC(Nc3ccc4[nH]ncc4c3)C2)cc1] N-(4-((3-(1H-indazol-5-ylamino)pyrrolidin-1- yl)methyl)phenyl)acetamide | 1c, 7, 8, 9, 10, 12a, 15c, 15d |
| 1.007 | 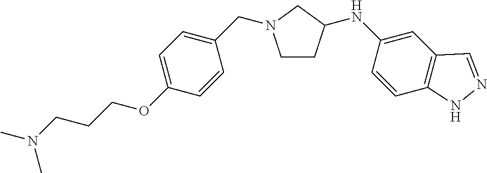 [SMILES: CN(C)CCCOc1ccc(CN2CCC(Nc3ccc4[nH]ncc4c3)C2)cc1] N-(1-(4-(3-(dimethylamino)propoxy)benzyl)pyrrolidin- 3-yl)-1H-indazol-5-amine | 1c, 7, 8, 9, 10, 12a, 15c, 15d |
| 1.008 | 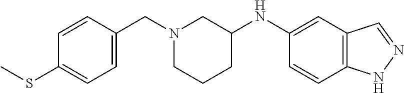 [SMILES: CSc1ccc(CN2CCCC(Nc3ccc4[nH]ncc4c3)C2)cc1] N-(1-(4-(methylthio)benzyl)piperidin-3-yl)-1H-indazol- 5-amine | 1c, 7, 8, 9, 10, 12b, 15c, 15e |
| 1.009 | 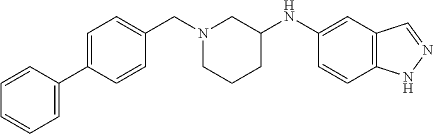 [SMILES: c1ccc(-c2ccc(CN3CCCC(Nc4ccc5[nH]ncc5c4)C3)cc2)cc1] N-(1-(biphenyl-4-ylmethyl)piperidin-3-yl)-1H-indazol-5- amine | 1c, 7, 8, 9, 10, 11, 14c |
| 1.010 | 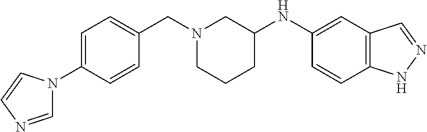 [SMILES: c1cn(-c2ccc(CN3CCCC(Nc4ccc5[nH]ncc5c4)C3)cc2)cn1] N-(1-(1H-imidazol-1-yl)benzyl)piperidin-3-yl)-1H- indazol-5-amine | 1c, 7, 8, 9, 10, 11, 14c |
| 1.011 | 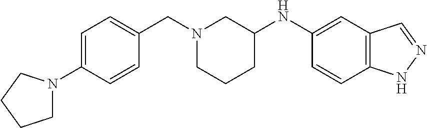 [SMILES: c1cc(N2CCCC2)ccc1CN1CCCC(Nc2ccc3[nH]ncc3c2)C1] N-(1-(4-(pyrrolidin-1-yl)benzyl)piperidin-3-yl)-1H- indazol-5-amine | 1c, 7, 8, 9, 10, 11, 14c |
| 1.012 | 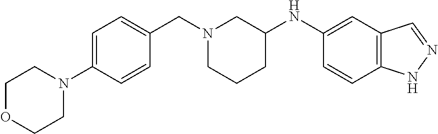 [SMILES: c1cc(N2CCOCC2)ccc1CN1CCCC(Nc2ccc3[nH]ncc3c2)C1] N-(1-(4-morpholinobenzyl)piperidin-3-yl)-1H-indazol-5- amine | 1c, 7, 8, 9, 10, 11, 14c |
| 1.013 | 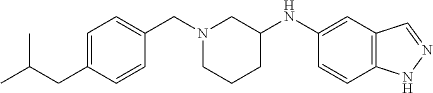 [SMILES: CC(C)Cc1ccc(CN2CCCC(Nc3ccc4[nH]ncc4c3)C2)cc1] N-(1-(4-isobutylbenzyl)piperidin-3-yl)-1H-indazol-5- amine | 1c, 7, 8, 9, 10 |
| 1.014 | 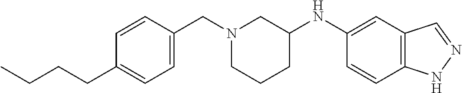 [SMILES: CCCCc1ccc(CN2CCCC(Nc3ccc4[nH]ncc4c3)C2)cc1] N-(1-(4-butylbenzyl)piperidin-3-yl)-1H-indazol-5-amine | 1c, 7, 8, 9, 10 |
| 1.015 | 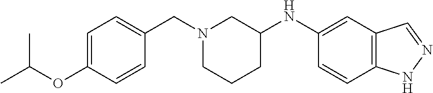 [SMILES: CC(C)Oc1ccc(CN2CCCC(Nc3ccc4[nH]ncc4c3)C2)cc1] N-(1-(4-isopropoxybenzyl)piperidin-3-yl)-1H-indazol-5- amine | 1c, 7, 8, 9, 10 |
| 1.016 | 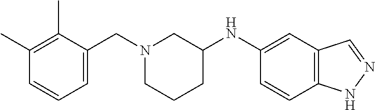 [SMILES: Cc1cccc(CN2CCCC(Nc3ccc4[nH]ncc4c3)C2)c1C] N-(1-(2,3-dimethylbenzyl)piperidin-3-yl)-1H-indazol-5- amine | 1c, 7, 8, 9, 10 |
| 1.017 | 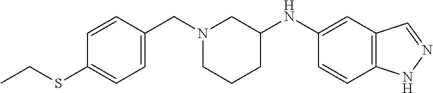 [SMILES: CCSc1ccc(CN2CCCC(Nc3ccc4[nH]ncc4c3)C2)cc1] N-(1-(4-(ethylthio)benzyl)piperidin-3-yl)-1H-indazol-5- amine | 1c, 7, 8, 9, 10, 12b, 15c, 15e |
| 1.018 | 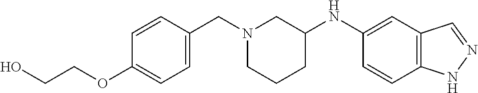 [SMILES: OCCOc1ccc(CN2CCCC(Nc3ccc4[nH]ncc4c3)C2)cc1] 2-(4-((3-(1H-indazol-5-ylamino)piperidin-1-yl)methyl) phenoxy)ethanol | 1c, 7, 8, 9, 10, 12a, 15c, 15d |
| 1.019 | 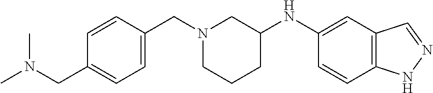 [SMILES: CN(C)Cc1ccc(CN2CCCC(Nc3ccc4[nH]ncc4c3)C2)cc1] N-(1-(4-((dimethylamino)methyl)benzyl)piperidin-3-yl)- 1H-indazol-5-amine | 1c, 7, 8, 9, 10, 13, 16c |
| 1.020 | 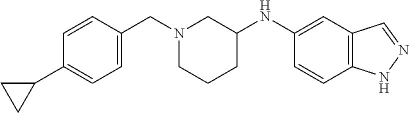 [SMILES: c1cc(C2CC2)ccc1CN1CCCC(Nc2ccc3[nH]ncc3c2)C1] N-(1-(4-cyclopropylbenzyl)piperidin-3-yl)-1H-indazol-5- amine | 1c, 7, 8, 9, 10, 11, 14c |
| 1.021 | 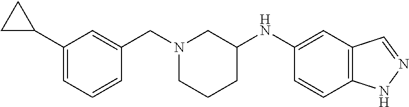 [SMILES: c1cc(CN2CCCC(Nc3ccc4[nH]ncc4c3)C2)cc(C2CC2)c1] N-(1-(3-cyclopropylbenzyl)piperidin-3-yl)-1H-indazol-5- amine | 1c, 7, 8, 9, 10, 11, 14c |
| 1.022 | 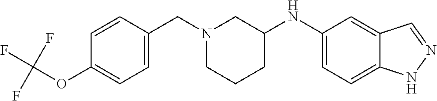 [SMILES: FC(F)(F)Oc1ccc(CN2CCCC(Nc3ccc4[nH]ncc4c3)C2)cc1] N-(1-(4-(trifluoromethoxy)benzyl)piperidin-3-yl)-1H- indazol-5-amine | 1c, 7, 8, 9, 10 |
| 1.023 | 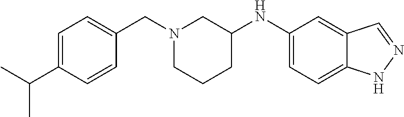 [SMILES: CC(C)c1ccc(CN2CCCC(Nc3ccc4[nH]ncc4c3)C2)cc1] N-(1-(4-isopropylbenzyl)piperidin-3-yl)-1H-indazol-5- amine | 1c, 7, 8, 9, 10 |
| 1.024 | 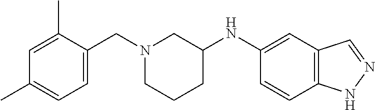 [SMILES: Cc1ccc(CN2CCCC(Nc3ccc4[nH]ncc4c3)C2)c(C)c1] N-(1-(2,4-dimethylbenzyl)piperidin-3-yl)-1H-indazol-5- amine | 1c, 7, 8, 9, 10 |
| 1.025 | 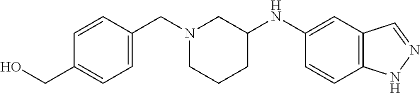 [SMILES: OCc1ccc(CN2CCCC(Nc3ccc4[nH]ncc4c3)C2)cc1] (4-((3-(1H-indazol-5-ylamino)piperidin-1- yl)methyl)phenyl)methanol | 1c, 7, 8, 9, 10 |
| 1.026 | 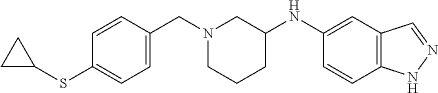 [SMILES: c1cc(SC2CC2)ccc1CN1CCCC(Nc2ccc3[nH]ncc3c2)C1] N-(1-(4-(cyclopropylthio)benzyl)piperidin-3-yl)-1H- indazol-5-amine | 1c, 7, 8, 9, 10, 12b, 15c, 15e |
| 1.027 | 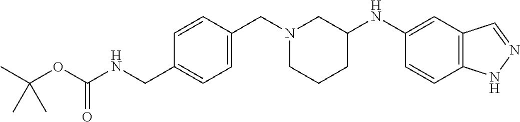 [SMILES: CC(C)(C)OC(=O)NCc1ccc(CN2CCCC(Nc3ccc4[nH]ncc4c3)C2)cc1] tert-butyl 4-((3-(1H-indazol-5-ylamino)piperidin-1- yl)methyl)benzylcarbamate | 1c, 7, 8, 9, 10, 13, 16c |
| 1.028 | 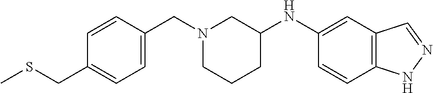 [SMILES: CSCc1ccc(CN2CCCC(Nc3ccc4[nH]ncc4c3)C2)cc1] N-(1-(4-(methylthiomethyl)benzyl)piperidin-3-yl)-1H- indazol-5-amine | 1c, 7, 8, 9, 10, 13, 16c |
| 1.029 | 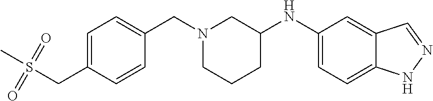 [SMILES: CS(=O)(=O)Cc1ccc(CN2CCCC(Nc3ccc4[nH]ncc4c3)C2)cc1] N-(1-(4-(methylsulfonylmethyl)benzyl)piperidin-3-yl)- 1H-indazol-5-amine | 1c, 7, 8, 9, 10, 13, 16c |
| 1.030 | 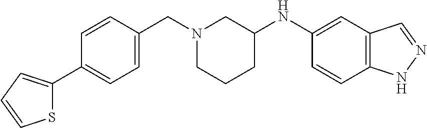 [SMILES: c1csc(-c2ccc(CN3CCCC(Nc4ccc5[nH]ncc5c4)C3)cc2)c1] N-(1-(4-(thiophen-2-yl)benzyl)piperidin-3-yl)-1H- indazol-5-amine | 1c, 7, 8, 9, 10, 11, 14c |
| 1.031 | 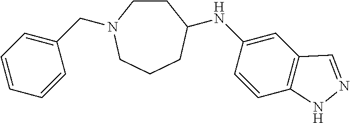 [SMILES: c1ccc(CN2CCCC(Nc3ccc4[nH]ncc4c3)CC2)cc1] N-(1-benzylazepan-4-yl)-1H-indazol-5-amine | 1c, 7, 8, 9, 10 |
| 1.032 | 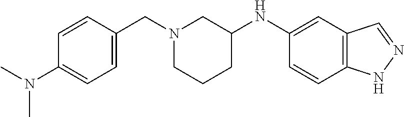 [SMILES: CN(C)c1ccc(CN2CCCC(Nc3ccc4[nH]ncc4c3)C2)cc1] N-(1-(4-(dimethylamino)benzyl)piperidin-3-yl)-1H- indazol-5-amine | 1c, 7, 8, 9, 10 |
| 1.033 | 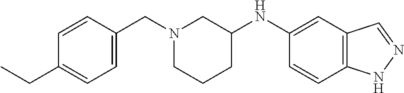 [SMILES: CCc1ccc(CN2CCCC(Nc3ccc4[nH]ncc4c3)C2)cc1] N-(1-(4-ethylbenzyl)piperidin-3-yl)-1H-indazol-5-amine | 1c, 7, 8, 9, 10 |
| 1.034 | 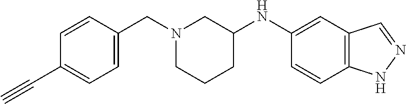 [SMILES: C#Cc1ccc(CN2CCCC(Nc3ccc4[nH]ncc4c3)C2)cc1] N-(1-(4-ethynylbenzyl)piperidin-3-yl)-1H-indazol-5- amine | 1c, 7, 8, 9, 10, 11, 14c |
| 1.035 | 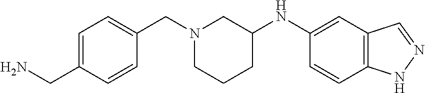 [SMILES: NCc1ccc(CN2CCCC(Nc3ccc4[nH]ncc4c3)C2)cc1] N-(1-(4-(aminomethyl)benzyl)piperidin-3-yl)-1H- indazol-5-amine | 1c, 7, 8, 9, 10, 13, 16c |
| 1.036 | 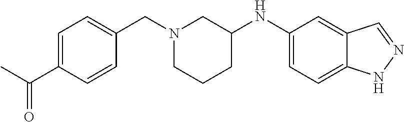 [SMILES: CC(=O)c1ccc(CN2CCCC(Nc3ccc4[nH]ncc4c3)C2)cc1] 1-(4-((3-(1H-indazol-5-ylamino)piperidin-1- yl)methyl)phenyl)ethanone | 1c, 7, 8, 9, 10 |
| 1.037 | 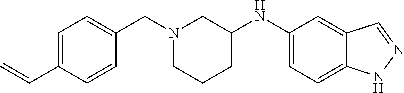 [SMILES: C=Cc1ccc(CN2CCCC(Nc3ccc4[nH]ncc4c3)C2)cc1] N-(1-(4-vinylbenzyl)piperidin-3-yl)-1H-indazol-5-amine | 1c, 7, 8, 9, 10, 11, 14c |
| 1.038 | 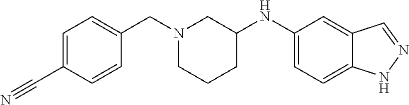 [SMILES: N#Cc1ccc(CN2CCCC(Nc3ccc4[nH]ncc4c3)C2)cc1] 4-((3-(1H-indazol-5-ylamino)piperidin-1- yl)methyl)benzonitrile | 1c, 7, 8, 9, 10, 12, 15c |
| 1.039 | 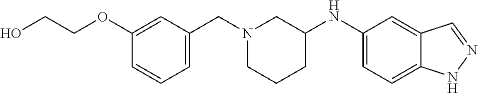 [SMILES: OCCOc1cccc(CN2CCCC(Nc3ccc4[nH]ncc4c3)C2)c1] 2-(3-((3-(1H-indazol-5-ylamino)piperidin-1-yl)methyl) phenoxy)ethanol | 1c, 7, 8, 9, 10, 12a, 15c, 15d |
| 1.040 | 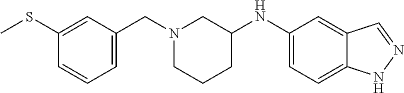 [SMILES: CSc1cccc(CN2CCCC(Nc3ccc4[nH]ncc4c3)C2)c1] N-(1-(3-(methylthio)benzyl)piperidin-3-yl)-1H-indazol- 5-amine | 1c, 7, 8, 9, 10, 12b, 15c, 15e |
| 1.041 | 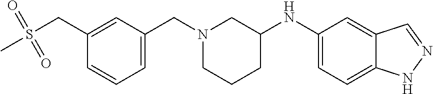 [SMILES: CS(=O)(=O)Cc1cccc(CN2CCCC(Nc3ccc4[nH]ncc4c3)C2)c1] N-(1-(3-(methylsulfonylmethyl)benzyl)piperidin-3-yl)- 1H-indazol-5-amine | 1c, 7, 8, 9, 10, 13, 16c |
| 1.042 | 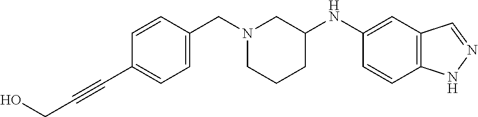 [SMILES: OCC#Cc1ccc(CN2CCCC(Nc3ccc4[nH]ncc4c3)C2)cc1] 3-(4-((3-(1H-indazol-5-ylamino)piperidin-1- yl)methyl)phenyl)prop-2-yn-1-ol | 1c, 7, 8, 9, 10, 13, 16c |
| 1.043 | 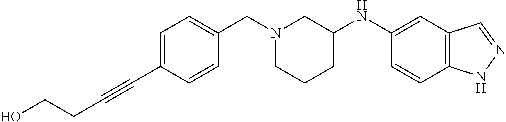 [SMILES: OCCC#Cc1ccc(CN2CCCC(Nc3ccc4[nH]ncc4c3)C2)cc1] 4-(4-((3-(1H-indazol-5-ylamino)piperidin-1- yl)methyl)phenyl)but-3-yn-1-ol | 1c, 7, 8, 9, 10, 13, 16c |
| 1.044 | 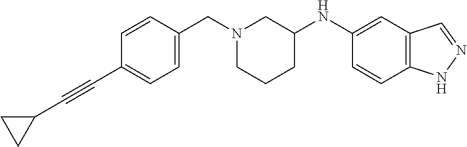 [SMILES: C(#CC1CC1)c1ccc(CN2CCCC(Nc3ccc4[nH]ncc4c3)C2)cc1] N-(1-(4-(cyclopropylethynyl)benzyl)piperidin-3-yl)-1H- indazol-5-amine | 1c, 7, 8, 9, 10, 11, 14c |
| 1.045 | 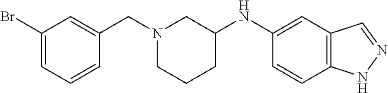 [SMILES: Brc1cccc(CN2CCCC(Nc3ccc4[nH]ncc4c3)C2)c1] N-(1-(3-bromobenzyl)piperidin-3-yl)-1H-indazol-5- amine | 1c, 7, 8, 9, 10 |
| 1.046 | 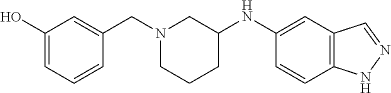 [SMILES: Oc1cccc(CN2CCCC(Nc3ccc4[nH]ncc4c3)C2)c1] 3-((3-(1H-indazol-5-ylamino)piperidin-1- yl)methyl)phenol | 1c, 7, 8, 9, 10 |
| 1.047 | 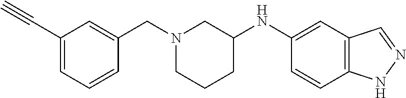 [SMILES: C#Cc1cccc(CN2CCCC(Nc3ccc4[nH]ncc4c3)C2)c1] N-(1-(3-ethynylbenzyl)piperidin-3-yl)-1H-indazol-5- amine | 1c, 7, 8, 9, 10, 11, 14c |
| 1.048 | 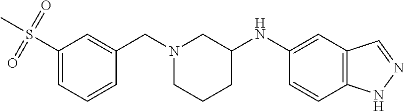 [SMILES: CS(=O)(=O)c1cccc(CN2CCCC(Nc3ccc4[nH]ncc4c3)C2)c1] N-(1-(3-(methylsulfonyl)benzyl)piperidin-3-yl)-1H- indazol-5-amine | 1c, 7, 8, 9, 10, 12, 15c |
| 1.049 | 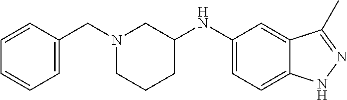 [SMILES: Cc1n[nH]c2ccc(NC3CCCN(Cc4ccccc4)C3)cc12] N-(1-benzylpiperidin-3-yl)-3-methyl-1H-indazol-5- amine | 1a, 6a, 8, 9, 10 |
| 1.050 | 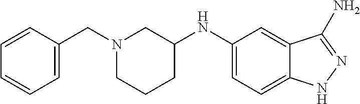 [SMILES: Nc1n[nH]c2ccc(NC3CCCN(Cc4ccccc4)C3)cc12] N5-(1-benzylpiperidin-3-yl)-1H-indazole-3,5-diamine | 1b, 6b, 8, 9, 10 |
| 1.051 | 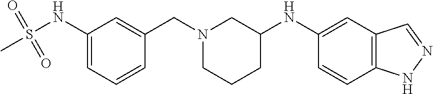 [SMILES: CS(=O)(=O)Nc1cccc(CN2CCCC(Nc3ccc4[nH]ncc4c3)C2)c1] N-(3-((3-(1H-indazol-5-ylamino)piperidin-1- yl)methyl)phenyl)methanesulfonamide | 1c, 7, 8, 9, 10, 12a, 15c, 15d |
| 1.052 | 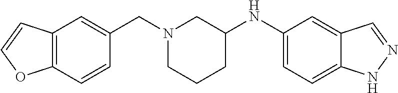 [SMILES: c1cc2cc(CN3CCCC(Nc4ccc5[nH]ncc5c4)C3)ccc2o1] N-(1-(benzofuran-5-ylmethyl)piperidin-3-yl)-1H- indazol-5-amine | 1c, 7, 8, 9, 10 |
| 1.053 | 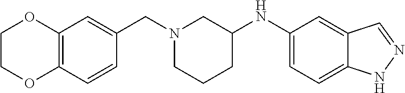 [SMILES: c1cc2c(cc1CN1CCCC(Nc3ccc4[nH]ncc4c3)C1)OCCO2] N-(1-((2,3-dihydrobenzo[b][1,4]dioxin-6- yl)methyl)piperidin-3-yl)-1H-indazol-5-amine | 1c, 7, 8, 9, 10 |
| 1.054 | 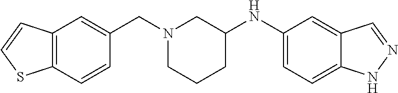 [SMILES: c1cc2cc(CN3CCCC(Nc4ccc5[nH]ncc5c4)C3)ccc2s1] N-(1-(benzo[b]thiophen-5-ylmethyl)piperidin-3-yl)-1H- indazol-5-amine | 1c, 7, 8, 9, 10 |
| 1.055 | 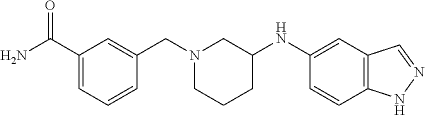 [SMILES: NC(=O)c1cccc(CN2CCCC(Nc3ccc4[nH]ncc4c3)C2)c1] 3-((3-(1H-indazol-5-ylamino)piperidin-1- yl)methyl)benzamide | 1c, 7, 8, 9, 10, 12, 15c |
| 1.056 | 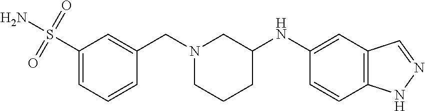 [SMILES: NS(=O)(=O)c1cccc(CN2CCCC(Nc3ccc4[nH]ncc4c3)C2)c1] 3-((3-(1H-indazol-5-ylamino)piperidin-1- yl)methyl)benzenesulfonamide | 1c, 7, 8, 9, 10, 12, 15c |
| 1.057 | 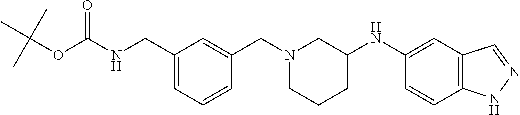 [SMILES: CC(C)(C)OC(=O)NCc1cccc(CN2CCCC(Nc3ccc4[nH]ncc4c3)C2)c1] tert-butyl 3-((3-(1H-indazol-5-ylamino)piperidin-1- yl)methyl)benzylcarbamate | 1c, 7, 8, 9, 10, 13, 16c |
| 1.058 | 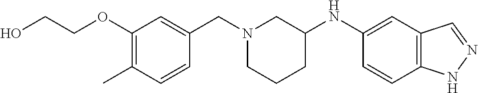 [SMILES: Cc1ccc(CN2CCCC(Nc3ccc4[nH]ncc4c3)C2)cc1OCCO] 2-(5-((3-(1H-indazol-5-ylamino)piperidin-1-yl)methyl)- 2-methylphenoxy)ethanol | 1c, 7, 8, 9, 10, 12a, 15c, 15d |
| 1.059 | 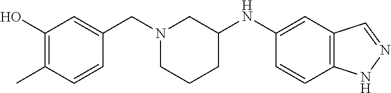 [SMILES: Cc1ccc(CN2CCCC(Nc3ccc4[nH]ncc4c3)C2)cc1O] 5-((3-(1H-indazol-5-ylamino)piperidin-1-yl)methyl)-2- methylphenol | 1c, 7, 8, 9, 10 |
| 1.060 | 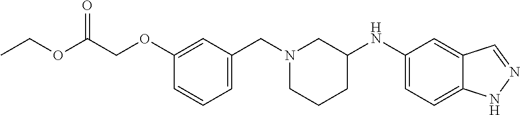 [SMILES: CCOC(=O)COc1cccc(CN2CCCC(Nc3ccc4[nH]ncc4c3)C2)c1] ethyl 2-(3-((3-(1H-indazol-5-ylamino)piperidin-1- yl)methyl)phenoxy)acetate | 1c, 7, 8, 9, 10, 12a, 15c, 15d |
| 1.061 | 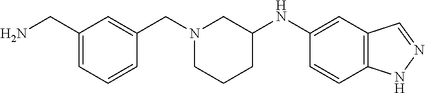 [SMILES: NCc1cccc(CN2CCCC(Nc3ccc4[nH]ncc4c3)C2)c1] N-(1-(3-(aminomethyl)benzyl)piperidin-3-yl)-1H- indazol-5-amine | 1c, 7, 8, 9, 10, 13, 16c |
| 1.062 | 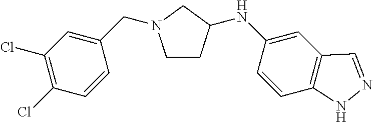 [SMILES: Clc1ccc(CN2CCC(Nc3ccc4[nH]ncc4c3)C2)cc1Cl] N-(1-(3,4-dichlorobenzyl)pyrrolidin-3-yl)-1H-indazol-5- amine | 1c, 7, 8, 9, 10 |
| 1.063 | 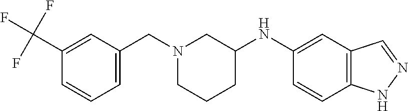 [SMILES: FC(F)(F)c1cccc(CN2CCCC(Nc3ccc4[nH]ncc4c3)C2)c1] N-(1-(3-(trifluoromethyl)benzyl)piperidin-3-yl)-1H- indazol-5-amine | 1c, 7, 8, 9, 10 |
| 1.064 | 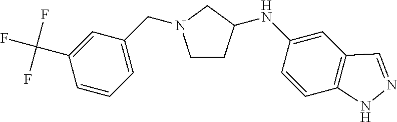 [SMILES: FC(F)(F)c1cccc(CN2CCC(Nc3ccc4[nH]ncc4c3)C2)c1] N-(1-(3-(trifluoromethyl)benzyl)pyrrolidin-3-yl)-1H- indazol-5-amine | 1c, 7, 8, 9, 10 |
| 1.065 | 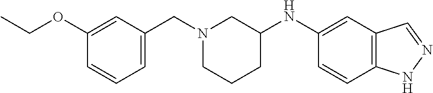 [SMILES: CCOc1cccc(CN2CCCC(Nc3ccc4[nH]ncc4c3)C2)c1] N-(1-(3-ethoxybenzyl)piperidin-3-yl)-1H-indazol-5- amine | 1c, 7, 8, 9, 10 |
| 1.066 | 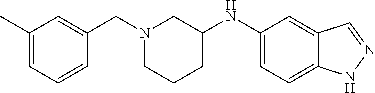 [SMILES: Cc1cccc(CN2CCCC(Nc3ccc4[nH]ncc4c3)C2)c1] N-(1-(3-methylbenzyl)piperidin-3-yl)-1H-indazol-5- amine | 1c, 7, 8, 9, 10 |
| 1.067 | 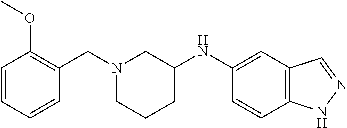 [SMILES: COc1ccccc1CN1CCCC(Nc2ccc3[nH]ncc3c2)C1] N-(1-(2-methoxybenzyl)piperidin-3-yl)-1H-indazol-5- amine | 1c, 7, 8, 9, 10 |
| 1.068 | 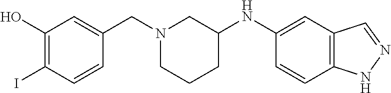 [SMILES: Oc1cc(CN2CCCC(Nc3ccc4[nH]ncc4c3)C2)ccc1I] 5-((3-(1H-indazol-5-ylamino)piperidin-1-yl)methyl)-2- iodophenol | 1c, 7, 8, 9, 10 |
| 1.069 | 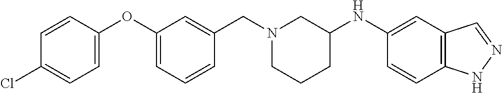 [SMILES: Clc1ccc(Oc2cccc(CN3CCCC(Nc4ccc5[nH]ncc5c4)C3)c2)cc1] N-(1-(3-(4-chlorophenoxy)benzyl)piperidin-3-yl)-1H- indazol-5-amine | 1c, 7, 8, 9, 10 |
| 1.070 | 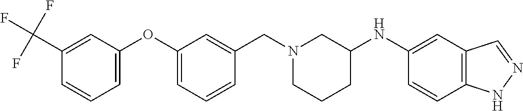 [SMILES: FC(F)(F)c1cccc(Oc2cccc(CN3CCCC(Nc4ccc5[nH]ncc5c4)C3)c2)c1] N-(1-(3-(3-(trifluoromethyl)phenoxy)benzyl)piperidin-3- yl)-1H-indazol-5-amine | 1c, 7, 8, 9, 10 |
| 1.071 | 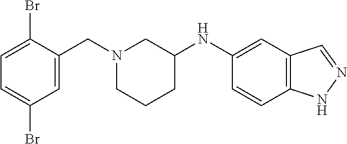 [SMILES: Brc1ccc(Br)c(CN2CCCC(Nc3ccc4[nH]ncc4c3)C2)c1] N-(1-(2,5-dibromobenzyl)piperidin-3-yl)-1H-indazol-5- amine | 1c, 7, 8, 9, 10 |
| 1.072 | 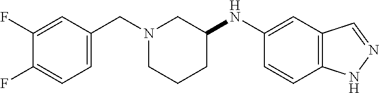 [SMILES: Fc1ccc(CN2CCC[C@H](Nc3ccc4[nH]ncc4c3)C2)cc1F] (S)-N-(1-(3,4-difluorobenzyl)piperidin-3-yl)-1H-indazol- 5-amine | 1c, 7, 8, 9, 10 |
| 1.073 | 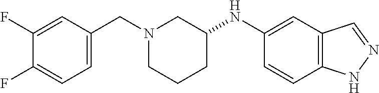 [SMILES: Fc1ccc(CN2CCC[C@@H](Nc3ccc4[nH]ncc4c3)C2)cc1F] (R)-N-(1-(3,4-difluorobenzyl)piperidin-3-yl)-1H-indazol- 5-amine | 1c, 7, 8, 9, 10 |
| 1.074 | 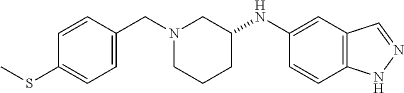 [SMILES: CSc1ccc(CN2CCC[C@@H](Nc3ccc4[nH]ncc4c3)C2)cc1] (R)-N-(1-(4-(methylthio)benzyl)piperidin-3-yl)-1H- indazol-5-amine | 1c, 7, 8, 9, 10, 12b, 15c, 15e |
| 1.075 | 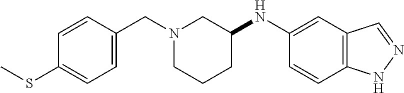 [SMILES: CSc1ccc(CN2CCC[C@H](Nc3ccc4[nH]ncc4c3)C2)cc1] (S)-N-(1-(4-(methylthio)benzyl)piperidin-3-yl)-1H- indazol-5-amine | 1c, 7, 8, 9, 10, 12b, 15c, 15e |
| 1.076 | 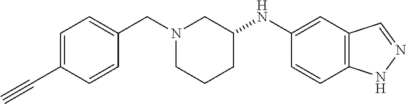 [SMILES: C#Cc1ccc(CN2CCC[C@@H](Nc3ccc4[nH]ncc4c3)C2)cc1] (R)-N-(1-(4-ethynylbenzyl)piperidin-3-yl)-1H-indazol-5- amine | 1c, 7, 8, 9, 10, 11, 14c |
| 1.077 | 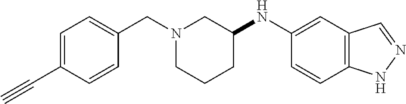 [SMILES: C#Cc1ccc(CN2CCC[C@H](Nc3ccc4[nH]ncc4c3)C2)cc1] (S)-N-(1-(4-ethynylbenzyl)piperidin-3-yl)-1H-indazol-5- amine | 1c, 7, 8, 9, 10, 11, 14c |
| 1.078 | 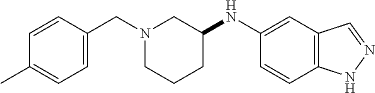 [SMILES: Cc1ccc(CN2CCC[C@H](Nc3ccc4[nH]ncc4c3)C2)cc1] (S)-N-(1-(4-methylbenzyl)piperidin-3-yl)-1H-indazol-5- amine | 1c, 7, 8, 9, 10 |
| 1.079 | 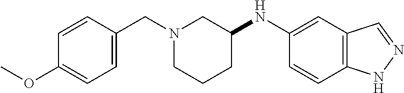 [SMILES: COc1ccc(CN2CCC[C@H](Nc3ccc4[nH]ncc4c3)C2)cc1] (S)-N-(1-(4-methoxybenzyl)piperidin-3-yl)-1H-indazol- 5-amine | 1c, 7, 8, 9, 10 |
| 1.080 | 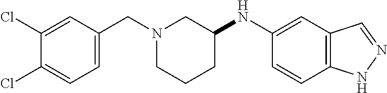 [SMILES: Clc1ccc(CN2CCC[C@H](Nc3ccc4[nH]ncc4c3)C2)cc1Cl] (S)-N-(1-(3,4-dichlorobenzyl)piperidin-3-yl)-1H- indazol-5-amine | 1c, 7, 8, 9, 10 |
| 1.082 | 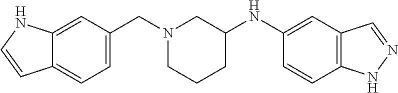 [SMILES: c1cc2ccc(CN3CCCC(Nc4ccc5[nH]ncc5c4)C3)cc2[nH]1] N-(1-((1H-indol-6-yl)methyl)piperidin-3-yl)-1H-indazol- 5-amine | 1c, 7, 8, 9, 10 |
| 1.083 | 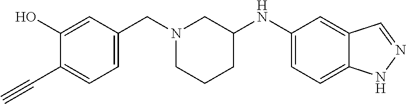 [SMILES: C#Cc1ccc(CN2CCCC(Nc3ccc4[nH]ncc4c3)C2)cc1O] 5-((3-(1H-indazol-5-ylamino)piperidin-1-yl)methyl)-2- ethynylphenol | 1c, 7, 8, 9, 10, 11, 14c |
| 1.084 | 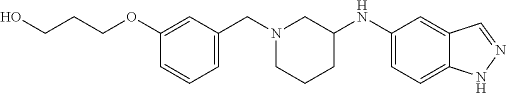 [SMILES: OCCCOc1cccc(CN2CCCC(Nc3ccc4[nH]ncc4c3)C2)c1] 3-(3-((3-(1H-indazol-5-ylamino)piperidin-1-yl)methyl) phenoxy)propan-1-ol | 1c, 7, 8, 9, 10, 12a, 15c, 15d |
| 1.085 | 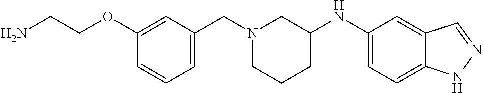 [SMILES: NCCOc1cccc(CN2CCCC(Nc3ccc4[nH]ncc4c3)C2)c1] N-(1-(3-(2-aminoethoxy)benzyl)piperidin-3-yl)-1H- indazol-5-amine | 1c, 7, 8, 9, 10, 12a, 15c, 15d |
| 1.086 | 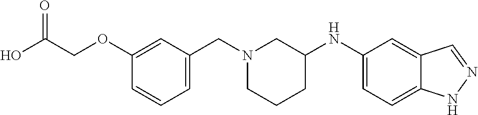 [SMILES: O=C(O)COc1cccc(CN2CCCC(Nc3ccc4[nH]ncc4c3)C2)c1] 2-(3-((3-(1H-indazol-5-ylamino)piperidin-1-yl)methyl) phenoxy)acetic acid | 1c, 7, 8, 9, 10, 12a, 15c, 15d |
| 1.087 | 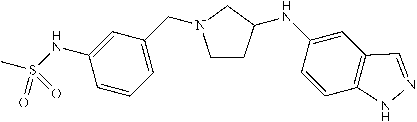 [SMILES: CS(=O)(=O)Nc1cccc(CN2CCC(Nc3ccc4[nH]ncc4c3)C2)c1] N-(3-((3-(1H-indazol-5-ylamino)pyrrolidin-1- yl)methyl)phenyl)methanesulfonamide | 1c, 7, 8, 9, 10, 12a, 15c, 15d |
| 1.088 | 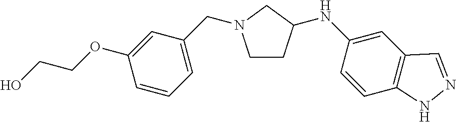 [SMILES: OCCOc1cccc(CN2CCC(Nc3ccc4[nH]ncc4c3)C2)c1] 2-(3-((3-(1H-indazol-5-ylamino)pyrrolidin-1-yl)methyl) phenoxy)ethanol | 1c, 7, 8, 9, 10, 12a, 15c, 15d |
| 1.089 | 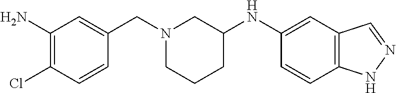 [SMILES: Nc1cc(CN2CCCC(Nc3ccc4[nH]ncc4c3)C2)ccc1Cl] N-(1-(3-amino-4-chlorobenzyl)piperidin-3-yl)-1H- indazol-5-amine | 1c, 7, 8, 9, 10 |
| 1.090 | 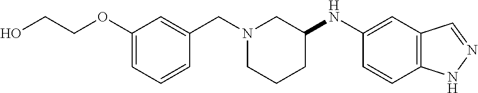 [SMILES: OCCOc1cccc(CN2CCC[C@H](Nc3ccc4[nH]ncc4c3)C2)c1] (S)-2-(3-((3-(1H-indazol-5-ylamino)piperidin-1- yl)methyl)phenoxy)ethanol | 1c, 7, 8, 9, 10, 12a, 15c, 15d |
| 1.091 | 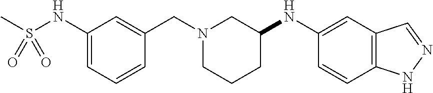 [SMILES: CS(=O)(=O)Nc1cccc(CN2CCC[C@H](Nc3ccc4[nH]ncc4c3)C2)c1] (S)-N-(3-((3-(1H-indazol-5-ylamino)piperidin-1- yl)methyl)phenyl)methanesulfonamide | 1c, 7, 8, 9, 10, 12a, 15c, 15d |
| 1.092 | 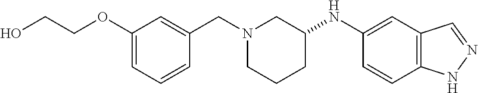 [SMILES: OCCOc1cccc(CN2CCC[C@@H](Nc3ccc4[nH]ncc4c3)C2)c1] (R)-2-(3-((3-(1H-indazol-5-ylamino)piperidin-1- yl)methyl)phenoxy)ethanol | 1c, 7, 8, 9, 10, 12a, 15c, 15d |
| 1.093 | 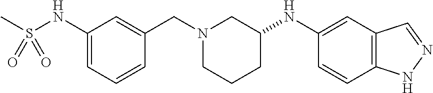 [SMILES: CS(=O)(=O)Nc1cccc(CN2CCC[C@@H](Nc3ccc4[nH]ncc4c3)C2)c1] (R)-N-(3-((3-(1H-indazol-5-ylamino)piperidin-1- yl)methyl)phenyl)methanesulfonamide | 1c, 7, 8, 9, 10, 12a, 15c, 15d |
| 1.094 | 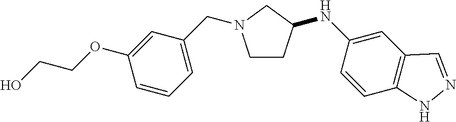 [SMILES: OCCOc1cccc(CN2CC[C@H](Nc3ccc4[nH]ncc4c3)C2)c1] (S)-2-(3-((3-(1H-indazol-5-ylamino)pyrrolidin-1- yl)methyl)phenoxy)ethanol | 1c, 7, 8, 9, 10, 12a, 15c, 15d |
| 1.095 | 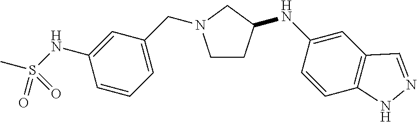 [SMILES: CS(=O)(=O)Nc1cccc(CN2CC[C@H](Nc3ccc4[nH]ncc4c3)C2)c1] (S)-N-(3-((3-(1H-indazol-5-ylamino)pyrrolidin-1- yl)methyl)phenyl)methanesulfonamide | 1c, 7, 8, 9, 10, 12a, 15c, 15d |
| 1.096 | 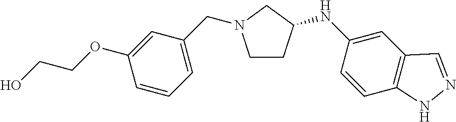 [SMILES: OCCOc1cccc(CN2CC[C@@H](Nc3ccc4[nH]ncc4c3)C2)c1] (R)-2-(3-((3-(1H-indazol-5-ylamino)pyrrolidin-1- yl)methyl)phenoxy)ethanol | 1c, 7, 8, 9, 10, 12a, 15c, 15d |
| 1.097 | 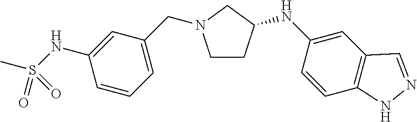 [SMILES: CS(=O)(=O)Nc1cccc(CN2CC[C@@H](Nc3ccc4[nH]ncc4c3)C2)c1] (R)-N-(3-((3-(1H-indazol-5-ylamino)pyrrolidin-1- yl)methyl)phenyl)methanesulfonamide | 1c, 7, 8, 9, 10, 12a, 15c, 15d |
| 1.098 | 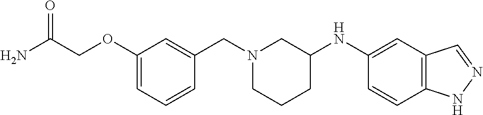 [SMILES: NC(=O)COc1cccc(CN2CCCC(Nc3ccc4[nH]ncc4c3)C2)c1] 2-(3-((3-(1H-indazol-5-ylamino)piperidin-1- yl)methyl)phenoxy)acetamide | 1c, 7, 8, 9, 10, 12a, 15c, 15d |
| 1.099 | 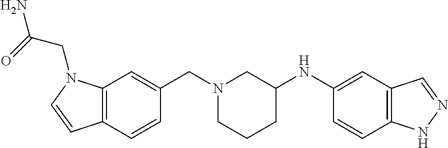 [SMILES: NC(=O)Cn1ccc2ccc(CN3CCCC(Nc4ccc5[nH]ncc5c4)C3)cc21] 2-(6-((3-(1H-indazolo-5-ylamino)piperidin-1-yl)methyl)- 1H-indol-1-yl)acetamide | 1c, 7, 8, 9, 10, 13, 16c |
| 1.100 | 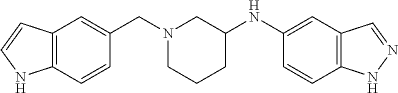 [SMILES: c1cc2cc(CN3CCCC(Nc4ccc5[nH]ncc5c4)C3)ccc2[nH]1] N-(1-((1H-indol-5-yl)methyl)piperidin-3-yl)-1H-indazol- 5-amine | 1c, 7, 8, 9, 10, 13, 16c |
| 1.101 | 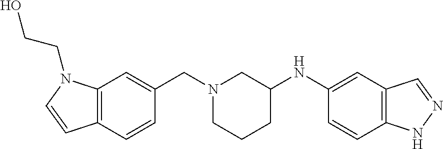 [SMILES: OCCn1ccc2ccc(CN3CCCC(Nc4ccc5[nH]ncc5c4)C3)cc21] 2-(6-((3-(1H-indazol-5-ylamino)piperidin-1-yl)methyl)- 1H-indol-1-yl)ethanol | 1c, 7, 8, 9, 10, 13, 16c |
| 1.102 | 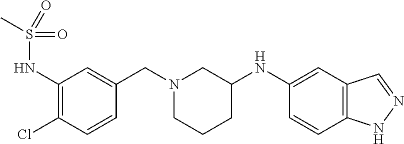 [SMILES: CS(=O)(=O)Nc1cc(CN2CCCC(Nc3ccc4[nH]ncc4c3)C2)ccc1Cl] N-(5-((3-(1H-indazol-5-ylamino)piperidin-1-yl)methyl)- 2-chlorophenyl)methanesulfonamide | 1c, 7, 8, 9, 10, 12a, 15c, 15d |
| 1.103 | 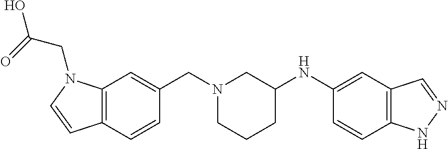 [SMILES: O=C(O)Cn1ccc2ccc(CN3CCCC(Nc4ccc5[nH]ncc5c4)C3)cc21] 2-(6-((3-(1H-indazol-5-ylamino)piperidin-1-yl)methyl)- 1H-indol-1-yl)acetic acid | 1c, 7, 8, 9, 10, 13, 16c |
| 1.104 | 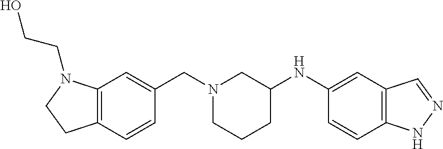 [SMILES: OCCN1CCc2ccc(CN3CCCC(Nc4ccc5[nH]ncc5c4)C3)cc21] 2-(6-((3-(1H-indazol-5-ylamino)piperidin-1- yl)methyl)indolin-1-yl)ethanol | 1c, 7, 8, 9, 10, 13, 16c |
| 1.105 | 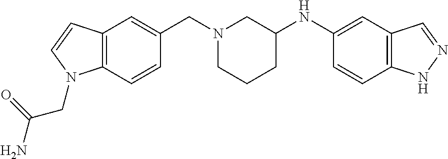 [SMILES: NC(=O)Cn1ccc2cc(CN3CCCC(Nc4ccc5[nH]ncc5c4)C3)ccc21] 2-(5-((3-(1H-indazol-5-ylamino)piperidin-1-yl)methyl)- 1H-indol-1-yl)acetamide | 1c, 7, 8, 9, 10, 13, 16c |
| 1.106 | 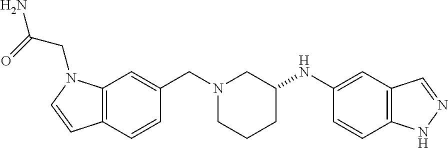 [SMILES: NC(=O)Cn1ccc2ccc(CN3CCC[C@@H](Nc4ccc5[nH]ncc5c4)C3)cc21] (R)-2-(6-((3-(1H-indazol-5-ylamino)piperidin-1- yl)methyl)-1H-indol-1-yl)acetamide | 1c, 7, 8, 9, 10, 13, 16c |
| 1.107 | 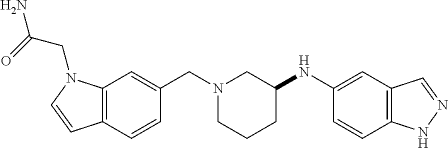 [SMILES: NC(=O)Cn1ccc2ccc(CN3CCC[C@H](Nc4ccc5[nH]ncc5c4)C3)cc21] (S)-2-(6-((3-(1H-indazol-5-ylamino)piperidin-1- yl)methyl)-1H-indol-1-yl)acetamide | 1c, 7, 8, 9, 10, 13, 16c |
| 1.108 | 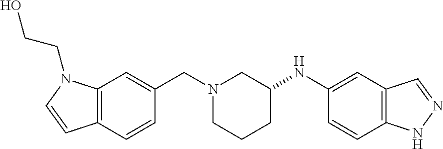 [SMILES: OCCn1ccc2ccc(CN3CCC[C@@H](Nc4ccc5[nH]ncc5c4)C3)cc21] (R)-2-(6-((3-(1H-indazol-5-ylamino)piperidin-1- yl)methyl)-1H-indol-1-yl)ethanol | 1c, 7, 8, 9, 10, 13, 16c |
| 1.109 | 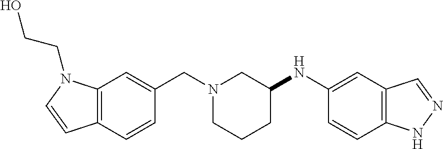 [SMILES: OCCn1ccc2ccc(CN3CCC[C@H](Nc4ccc5[nH]ncc5c4)C3)cc21] (S)-2-(6-((3-(1H-indazol-5-ylamino)piperidin-1- yl)methyl)-1H-indol-1-yl)ethanol | 1c, 7, 8, 9, 10, 13, 16c |
| 1.110 | 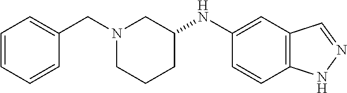 [SMILES: c1ccc(CN2CCC[C@@H](Nc3ccc4[nH]ncc4c3)C2)cc1] (R)-N-(1-benzylpiperidin-3-yl)-1H-indazol-5-amine | 1c, 7, 8, 9, 10 |
| 1.111 | 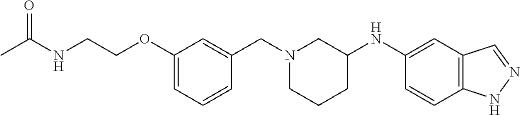 [SMILES: CC(=O)NCCOc1cccc(CN2CCCC(Nc3ccc4[nH]ncc4c3)C2)c1] N-(2-(3-((3-(1H-indazol-5-ylamino)piperidin-1- yl)methyl)phenoxy)ethyl)acetamide | 1c, 7, 8, 9, 10, 12a, 15c, 15d |
| 1.112 | 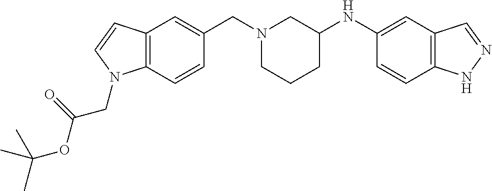 [SMILES: CC(C)(C)OC(=O)Cn1ccc2cc(CN3CCCC(Nc4ccc5[nH]ncc5c4)C3)ccc21] tert-butyl 2-(5-((3-(1H-indazol-5-ylamino)piperidin-1- yl)methyl)-1H-indol-1-yl)acetate | 1c, 7, 8, 9, 10, 13, 16c |
| 1.113 | 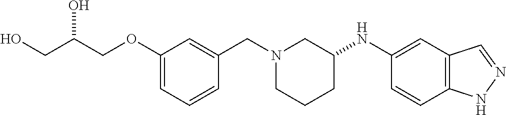 [SMILES: OC[C@H](O)COc1cccc(CN2CCC[C@@H](Nc3ccc4[nH]ncc4c3)C2)c1] (S)-3-(3-(((R)-3-(1H-indazol-5-ylamino)piperidin-1- yl)methyl)phenoxy)propane-1,2-diol | 1c, 7, 8, 9, 10, 12a, 15c, 15d |
| 1.114 | 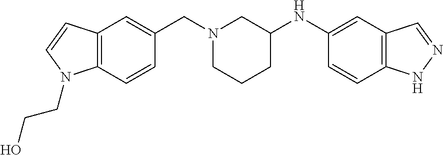 [SMILES: OCCn1ccc2cc(CN3CCCC(Nc4ccc5[nH]ncc5c4)C3)ccc21] 2-(5-((3-(1H-indazol-5-ylamino)piperidin-1-yl)methyl)- 1H-indol-1-yl)ethanol | 1c, 7, 8, 9, 10, 13, 16c |
| 1.115 | 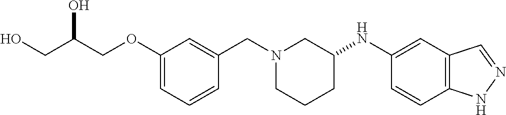 [SMILES: OC[C@@H](O)COc1cccc(CN2CCC[C@@H](Nc3ccc4[nH]ncc4c3)C2)c1] (R)-3-(3-(((R)-3-(1H-indazol-5-ylamino)piperidin-1- yl)methyl)phenoxy)propane-1,2-diol | 1c, 7, 8, 9, 10, 12a, 15c, 15d |
| 1.116 | 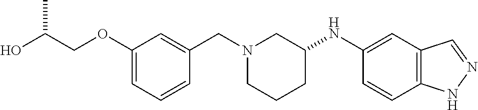 [SMILES: C[C@@H](O)COc1cccc(CN2CCC[C@@H](Nc3ccc4[nH]ncc4c3)C2)c1] (R)-1-(3-(((R)-3-(1H-indazol-5-ylamino)piperidin-1- yl)methyl)phenoxy)propan-2-ol | 1c, 7, 8, 9, 10, 12a, 15c, 15d |
| 1.117 | 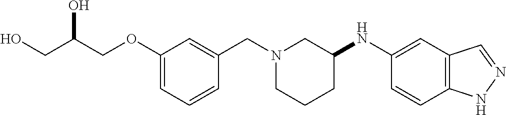 [SMILES: OC[C@@H](O)COc1cccc(CN2CCC[C@H](Nc3ccc4[nH]ncc4c3)C2)c1] (R)-3-(3-(((S)-3-(1H-indazol-5-ylamino)piperidin-1- yl)methyl)phenoxy)propane-1,2-diol | 1c, 7, 8, 9, 10, 12a, 15c, 15d |
| 1.118 | 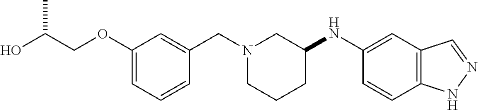 [SMILES: C[C@@H](O)COc1cccc(CN2CCC[C@H](Nc3ccc4[nH]ncc4c3)C2)c1] (R)-1-(3-(((S)-3-(1H-indazol-5-ylamino)piperidin-1- yl)methyl)phenoxy)propan-2-ol | 1c, 7, 8, 9, 10, 12a, 15c, 15d |
| 1.119 | 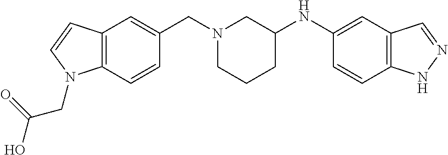 [SMILES: O=C(O)Cn1ccc2cc(CN3CCCC(Nc4ccc5[nH]ncc5c4)C3)ccc21] 2-(5-((3-(1H-indazol-5-ylamino)piperidin-1-yl)methyl)- 1H-indol-1-yl)acetic acid | 1c, 7, 8, 9, 10, 13, 16c |
| 1.120 | 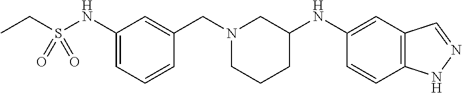 [SMILES: CCS(=O)(=O)Nc1cccc(CN2CCCC(Nc3ccc4[nH]ncc4c3)C2)c1] N-(3-((3-(1H-indazol-5-ylamino)piperidin-1- yl)methyl)phenyl)ethanesulfonamide | 1c, 7, 8, 9, 10, 12a, 15c, 15d |
| 1.121 | 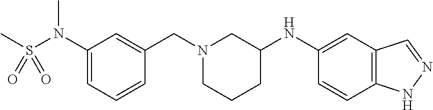 [SMILES: CN(c1cccc(CN2CCCC(Nc3ccc4[nH]ncc4c3)C2)c1)S(C)(=O)=O] N-(3-((3-(1H-indazol-5-ylamino)piperidin-1- yl)methyl)phenyl)-N-methylmethanesulfonamide | 1c, 7, 8, 9, 10, 12a, 15c, 15d |
| 1.122 | 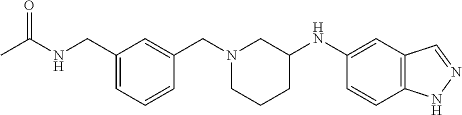 [SMILES: CC(=O)NCc1cccc(CN2CCCC(Nc3ccc4[nH]ncc4c3)C2)c1] N-(3-((3-(1H-indazol-5-ylamino)piperidin-1- yl)methyl)benzyl)acetamide | 1c, 7, 8, 9, 10, 13, 16c |
| 1.123 | 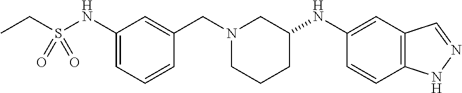 [SMILES: CCS(=O)(=O)Nc1cccc(CN2CCC[C@@H](Nc3ccc4[nH]ncc4c3)C2)c1] (R)-N-(3-((3-(1H-indazol-5-ylamino)piperidin-1- yl)methyl)phenyl)ethanesulfonamide | 1c, 7, 8, 9, 10, 12a, 15c, 15d |
| 1.124 | 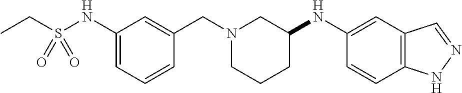 [SMILES: CCS(=O)(=O)Nc1cccc(CN2CCC[C@H](Nc3ccc4[nH]ncc4c3)C2)c1] (S)-N-(3-((3-(1H-indazol-5-ylamino)piperidin-1- yl)methyl)phenyl)ethanesulfonamide | 1c, 7, 8, 9, 10, 12a, 15c, 15d |
| 1.125 | 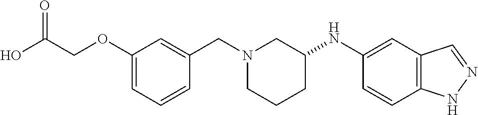 [SMILES: O=C(O)COc1cccc(CN2CCC[C@@H](Nc3ccc4[nH]ncc4c3)C2)c1] (R)-2-(3-((3-(1H-indazol-5-ylamino)piperidin-1- yl)methyl)phenoxy)acetic acid | 1c, 7, 8, 9, 10, 12a, 15c, 15d |
| 1.126 | 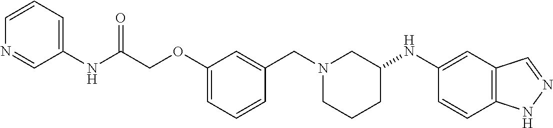 [SMILES: O=C(COc1cccc(CN2CCC[C@@H](Nc3ccc4[nH]ncc4c3)C2)c1)Nc1cccnc1] (R)-2-(3-((3-(1H-indazol-5-ylamino)piperidin-1- yl)methyl)phenoxy)-N-(pyridin-3-yl)acetamide | 1c, 7, 8, 9, 10, 12a, 15c, 15d |
| 1.127 | 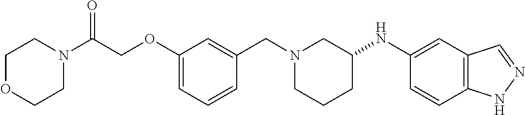 [SMILES: O=C(COc1cccc(CN2CCC[C@@H](Nc3ccc4[nH]ncc4c3)C2)c1)N1CCOCC1] (R)-2-(3-((3-(1H-indazol-5-ylamino)piperidin-1- yl)methyl)phenoxy)-1-morpholinoethanone | 1c, 7, 8, 9, 10, 12a, 15c, 15d |
| 1.128 | 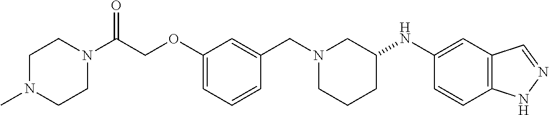 [SMILES: CN1CCN(C(=O)COc2cccc(CN3CCC[C@@H](Nc4ccc5[nH]ncc5c4)C3)c2)CC1] (R)-2-(3-((3-(1H-indazol-5-ylamino)piperidin-1- yl)methyl)phenoxy)-1-(4-methylpiperazin-1- yl)ethanone | 1c, 7, 8, 9, 10, 12a, 15c, 15d |
| 1.129 | 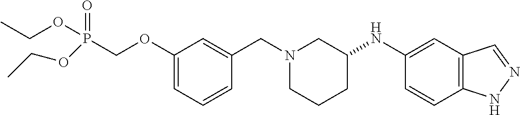 [SMILES: CCOP(=O)(COc1cccc(CN2CCC[C@@H](Nc3ccc4[nH]ncc4c3)C2)c1)OCC] (R)-diethyl (3-((3-(1H-indazol-5-ylamino)piperidin-1- yl)methyl)phenoxy)methylphosphonate | 1c, 7, 8, 9, 10, 12a, 15c, 15d |
| 1.130 | 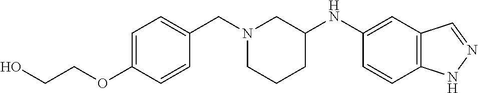 [SMILES: OCCOc1ccc(CN2CCCC(Nc3ccc4[nH]ncc4c3)C2)cc1] 2-(3-((4-(1H-indazol-5-ylamino)piperidin-1- yl)methyl)phenoxy)ethanol | 1c, 7, 8, 9, 10, 12a, 15c, 15d |
| 1.131 | 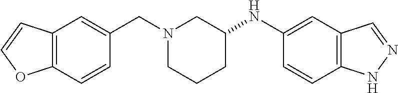 [SMILES: c1cc2cc(CN3CCC[C@@H](Nc4ccc5[nH]ncc5c4)C3)ccc2o1] (R)-N-(1-(benzofuran-5-ylmethyl)piperidin-3-yl)-1H- indazol-5-amine | 1c, 7, 8, 9, 10 |
| 1.132 | 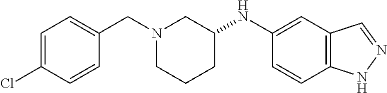 [SMILES: Clc1ccc(CN2CCC[C@@H](Nc3ccc4[nH]ncc4c3)C2)cc1] (R)-N-(1-(4-chlorobenzyl)piperidin-3-yl)-1H-indazol-5- amine | 1c, 7, 8, 9, 10 |
| 1.133 | 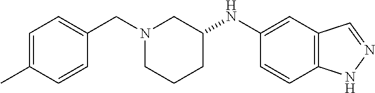 [SMILES: Cc1ccc(CN2CCC[C@@H](Nc3ccc4[nH]ncc4c3)C2)cc1] (R)-N-(1-(4-methylbenzyl)piperidin-3-yl)-1H-indazol-5- amine | 1c, 7, 8, 9, 10 |
| 1.134 | 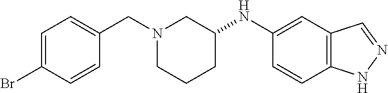 [SMILES: Brc1ccc(CN2CCC[C@@H](Nc3ccc4[nH]ncc4c3)C2)cc1] (R)-N-(1-(4-bromobenzyl)piperidin-3-yl)-1H-indazol-5- amine | 1c, 7, 8, 9, 10 |
| 1.136 | 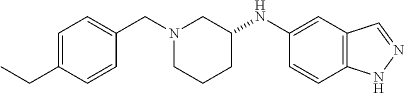 [SMILES: CCc1ccc(CN2CCC[C@@H](Nc3ccc4[nH]ncc4c3)C2)cc1] (R)-N-(1-(4-ethylbenzyl)piperidin-3-yl)-1H-indazol-5- amine | 1c, 7, 8, 9, 10 |
| 1.137 | 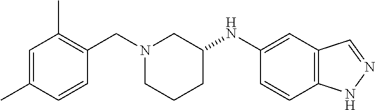 [SMILES: Cc1ccc(CN2CCC[C@@H](Nc3ccc4[nH]ncc4c3)C2)c(C)c1] (R)-N-(1-(2,4-dimethylbenzyl)piperidin-3-yl)-1H- indazol-5-amine | 1c, 7, 8, 9, 10 |
| 1.138 | 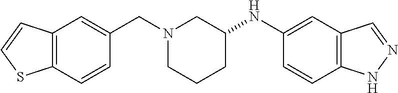 [SMILES: c1cc2cc(CN3CCC[C@@H](Nc4ccc5[nH]ncc5c4)C3)ccc2s1] (R)-N-(1-(benzo[b]thiophen-5-ylmethyl)piperidin-3-yl)- 1H-indazol-5-amine | 1c, 7, 8, 9, 10 |
| 1.139 | 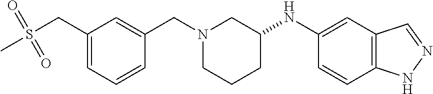 [SMILES: CS(=O)(=O)Cc1cccc(CN2CCC[C@@H](Nc3ccc4[nH]ncc4c3)C2)c1] (R)-N-(1-(3-(methylsulfonylmethyl)benzyl)piperidin-3- yl)-1H-indazol-5-amine | 1c, 7, 8, 9, 10, 12, 15c |
| 1.140 | 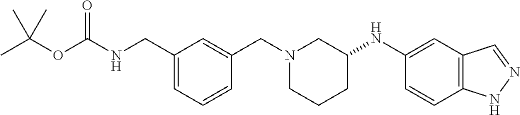 [SMILES: CC(C)(C)OC(=O)NCc1cccc(CN2CCC[C@@H](Nc3ccc4[nH]ncc4c3)C2)c1] (R)-tert-butyl 3-((3-(1H-indazol-5-ylamino)piperidin-1- yl)methyl)benzylcarbamate | 1c, 7, 8, 9, 10, 13, 16c |
| 1.141 | 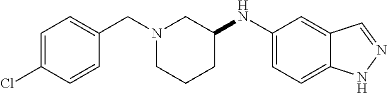 [SMILES: Clc1ccc(CN2CCC[C@H](Nc3ccc4[nH]ncc4c3)C2)cc1] (S)-N-(1-(4-chlorobenzyl)piperidin-3-yl)-1H-indazol-5- amine | 1c, 7, 8, 9, 10 |
| 1.142 | 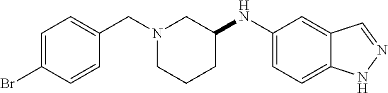 [SMILES: Brc1ccc(CN2CCC[C@H](Nc3ccc4[nH]ncc4c3)C2)cc1] (S)-N-(1-(4-bromobenzyl)piperidin-3-yl)-1H-indazol-5- amine | 1c, 7, 8, 9, 10 |
| 1.143 | 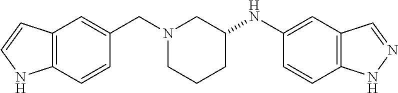 [SMILES: c1cc2cc(CN3CCC[C@@H](Nc4ccc5[nH]ncc5c4)C3)ccc2[nH]1] (R)-N-(1-((1H-indol-5-yl)methyl)piperidin-3-yl)-1H- indazol-5-amine | 1c, 7, 8, 9, 10, 13, 16c |
| 1.144 | 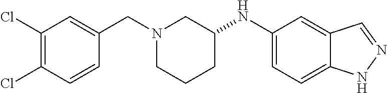 [SMILES: Clc1ccc(CN2CCC[C@@H](Nc3ccc4[nH]ncc4c3)C2)cc1Cl] (R)-N-(1-(3,4-dichlorobenzyl)piperidin-3-yl)-1H- indazol-5-amine | 1c, 7, 8, 9, 10 |
| 1.145 | 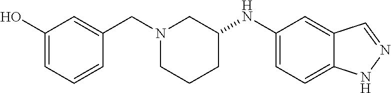 [SMILES: Oc1cccc(CN2CCC[C@@H](Nc3ccc4[nH]ncc4c3)C2)c1] (R)-3-((3-(1H-indazol-5-ylamino)piperidin-1- yl)methyl)phenol | 1c, 7, 8, 9, 10 |
| 1.146 | 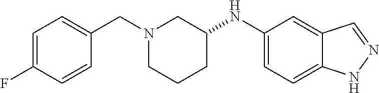 [SMILES: Fc1ccc(CN2CCC[C@@H](Nc3ccc4[nH]ncc4c3)C2)cc1] (R)-N-(1-(4-fluorobenzyl)piperidin-3-yl)-1H-indazol-5- amine | 1c, 7, 8, 9, 10 |
| 1.147 | 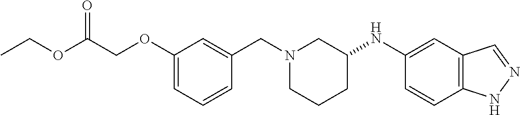 [SMILES: CCOC(=O)COc1cccc(CN2CCC[C@@H](Nc3ccc4[nH]ncc4c3)C2)c1] (R)-ethyl 2-(3-((3-(1H-indazol-5-ylamino)piperidin-1- yl)methyl)phenoxy)acetate | 1c, 7, 8, 9, 10, 12a, 15c, 15d |
| 1.148 | 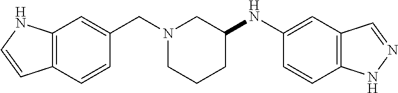 [SMILES: c1cc2ccc(CN3CCC[C@H](Nc4ccc5[nH]ncc5c4)C3)cc2[nH]1] (S)-N-(1-((1H-indol-6-yl)methyl)piperidin-3-yl)-1H- indazol-5-amine | 1c, 7, 8, 9, 10 |
| 1.149 | 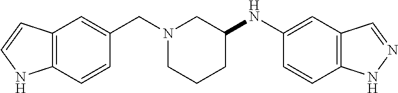 [SMILES: c1cc2cc(CN3CCC[C@H](Nc4ccc5[nH]ncc5c4)C3)ccc2[nH]1] (S)-N-(1-((1H-indol-5-yl)methyl)piperidin-3-yl)-1H- indazol-5-amine | 1c, 7, 8, 9, 10 |
| 1.150 | 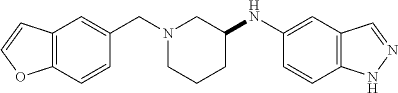 [SMILES: c1cc2cc(CN3CCC[C@H](Nc4ccc5[nH]ncc5c4)C3)ccc2o1] (S)-N-(1-(benzofuran-5-ylmethyl)piperidin-3-yl)-1H- indazol-5-amine | 1c, 7, 8, 9, 10 |
| 1.151 | 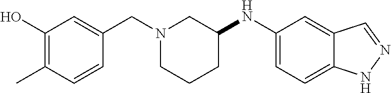 [SMILES: Cc1ccc(CN2CCC[C@H](Nc3ccc4[nH]ncc4c3)C2)cc1O] (S)-5-((3-(1H-indazol-5-ylamino)piperidin-1-yl)methyl)- 2-methylphenol | 1c, 7, 8, 9, 10 |
| 1.152 | 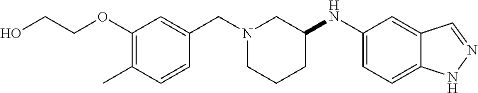 [SMILES: Cc1ccc(CN2CCC[C@H](Nc3ccc4[nH]ncc4c3)C2)cc1OCCO] (S)-2-(5-((3-(1H-indazol-5-ylamino)piperidin-1- yl)methyl)-2-methylphenoxy)ethanol | 1c, 7, 8, 9, 10, 12a, 15c, 15d |
| 1.153 | 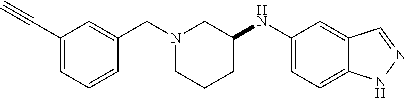 [SMILES: C#Cc1cccc(CN2CCC[C@H](Nc3ccc4[nH]ncc4c3)C2)c1] (S)-N-(1-(3-ethynylbenzyl)piperidin-3-yl)-1H-indazol-5- amine | 1c, 7, 8, 9, 10, 11, 14c |
| 1.154 | 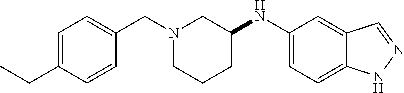 [SMILES: CCc1ccc(CN2CCC[C@H](Nc3ccc4[nH]ncc4c3)C2)cc1] (S)-N-(1-(4-ethylbenzyl)piperidin-3-yl)-1H-indazol-5- amine | 1c, 7, 8, 9, 10 |
| 1.155 | 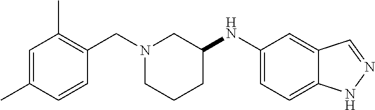 [SMILES: Cc1ccc(CN2CCC[C@H](Nc3ccc4[nH]ncc4c3)C2)c(C)c1] (S)-N-(1-(2,4-dimethylbenzyl)piperidin-3-yl)-1H- indazol-5-amine | 1c, 7, 8, 9, 10 |
| 1.156 | 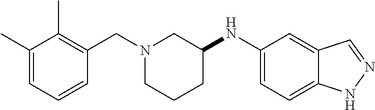 [SMILES: Cc1cccc(CN2CCC[C@H](Nc3ccc4[nH]ncc4c3)C2)c1C] (S)-N-(1-(2,3-dimethylbenzyl)piperidin-3-yl)-1H- indazol-5-amine | 1c, 7, 8, 9, 10 |
| 1.157 | 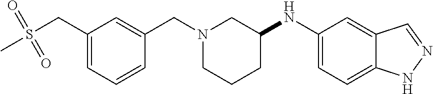 [SMILES: CS(=O)(=O)Cc1cccc(CN2CCC[C@H](Nc3ccc4[nH]ncc4c3)C2)c1] (S)-N-(1-(3-(methylsulfonylmethyl)benzyl)piperidin-3- yl)-1H-indazol-5-amine | 1c, 7, 8, 9, 10, 12, 15c |
| 1.158 | 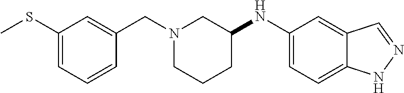 [SMILES: CSc1cccc(CN2CCC[C@H](Nc3ccc4[nH]ncc4c3)C2)c1] (S)-N-(1-(3-(methylthio)benzyl)piperidin-3-yl)-1H- indazol-5-amine | 1c, 7, 8, 9, 10, 12b, 15c, 15e |
| 1.159 | 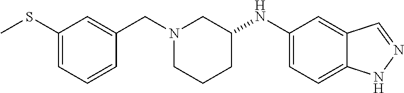 [SMILES: CSc1cccc(CN2CCC[C@@H](Nc3ccc4[nH]ncc4c3)C2)c1] (R)-N-(1-(3-(methylthio)benzyl)piperidin-3-yl)-1H- indazol-5-amine | 1c, 7, 8, 9, 10, 12b, 15c, 15e |
| 1.160 | 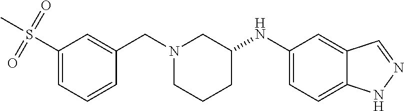 [SMILES: CS(=O)(=O)c1cccc(CN2CCC[C@@H](Nc3ccc4[nH]ncc4c3)C2)c1] (R)-N-(1-(3-(methylsulfonyl)benzyl)piperidin-3-yl)-1H- indazol-5-amine | 1c, 7, 8, 9, 10, 12, 15c |
| 1.161 | 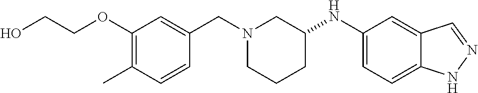 [SMILES: Cc1ccc(CN2CCC[C@@H](Nc3ccc4[nH]ncc4c3)C2)cc1OCCO] (R)-2-(5-((3-(1H-indazol-5-ylamino)piperidin-1- yl)methyl)-2-methylphenoxy)ethanol | 1c, 7, 8, 9, 10, 12a, 15c, 15d |
| 1.162 | 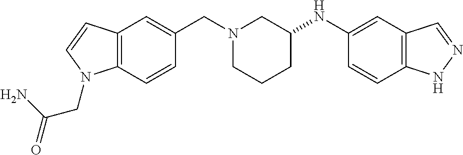 [SMILES: NC(=O)Cn1ccc2cc(CN3CCC[C@@H](Nc4ccc5[nH]ncc5c4)C3)ccc21] (R)-2-(5-((3-(1H-indazol-5-ylamino)piperidin-1- yl)methyl)-1H-indol-1-yl)acetamide | 1c, 7, 8, 9, 10, 13, 16c |
| 1.163 | 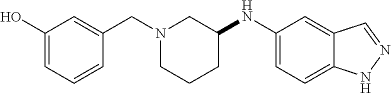 [SMILES: Oc1cccc(CN2CCC[C@H](Nc3ccc4[nH]ncc4c3)C2)c1] (S)-3-((3-(1H-indazol-5-ylamino)piperidin-1- yl)methyl)phenol | 1c, 7, 8, 9, 10 |
| 1.164 | 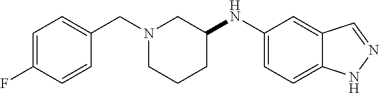 [SMILES: Fc1ccc(CN2CCC[C@H](Nc3ccc4[nH]ncc4c3)C2)cc1] (S)-N-(1-(4-fluorobenzyl)piperidin-3-yl)-1H-indazol-5- amine | 1c, 7, 8, 9, 10 |
| 1.165 | 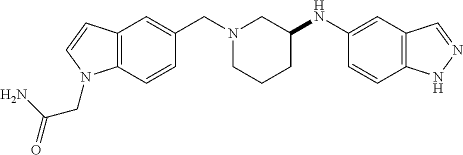 [SMILES: NC(=O)Cn1ccc2cc(CN3CCC[C@H](Nc4ccc5[nH]ncc5c4)C3)ccc21] (S)-2-(5-((3-(1H-indazol-5-ylamino)piperidin-1- yl)methyl)-1H-indazol-1-yl)acetamide | 1c, 7, 8, 9, 10, 13, 16c |
| 1.166 | 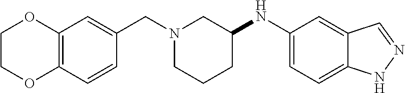 [SMILES: c1cc2c(cc1CN1CCC[C@H](Nc3ccc4[nH]ncc4c3)C1)OCCO2] (S)-N-(1-((2,3-dihydrobenzo[b][1,4]dioxin-6- yl)methyl)piperidin-3-yl)-1H-indazol-5-amine | 1c, 7, 8, 9, 10 |
| 1.167 | 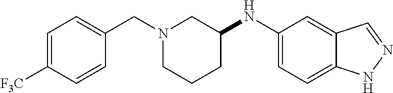 [SMILES: FC(F)(F)c1ccc(CN2CCC[C@H](Nc3ccc4[nH]ncc4c3)C2)cc1] (S)-N-(1-(4-(trifluoromethyl)benzyl)piperidin-3-yl)-1H- indazol-5-amine | 1c, 7, 8, 9, 10 |
| 1.168 | 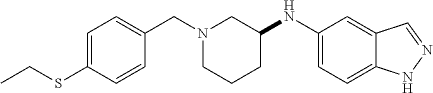 [SMILES: CCSc1ccc(CN2CCC[C@H](Nc3ccc4[nH]ncc4c3)C2)cc1] (S)-N-(1-(4-(ethylthio)benzyl)piperidin-3-yl)-1H- indazol-5-amine | 1c, 7, 8, 9, 10, 12b, 15c, 15e |
| 1.169 | 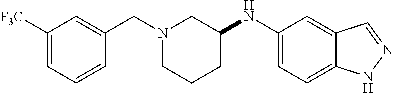 [SMILES: FC(F)(F)c1cccc(CN2CCC[C@H](Nc3ccc4[nH]ncc4c3)C2)c1] (S)-N-(1-(3-trifluoromethyl)benzyl)piperidin-3-yl)-1H- indazol-5-amine | 1c, 7, 8, 9, 10 |
| 1.170 | 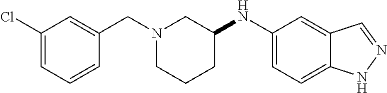 [SMILES: Clc1cccc(CN2CCC[C@H](Nc3ccc4[nH]ncc4c3)C2)c1] (S)-N-(1-(3-chlorobenzyl)piperidin-3-yl)-1H-indazol-5- amine | 1c, 7, 8, 9, 10 |
| 1.171 | 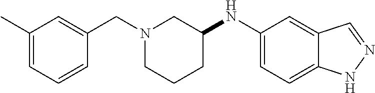 [SMILES: Cc1cccc(CN2CCC[C@H](Nc3ccc4[nH]ncc4c3)C2)c1] (S)-N-(1-(3-methylbenzyl)piperidin-3-yl)-1H-indazol-5- amine | 1.171 |
| 1.172 | 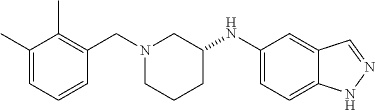 [SMILES: Cc1cccc(CN2CCC[C@@H](Nc3ccc4[nH]ncc4c3)C2)c1C] (R)-N-(1-(2,3-dimethylbenzyl)piperidin-3-yl)-1H- indazol-5-amine | 1.172 |
| 1.173 | 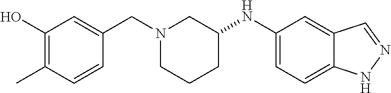 [SMILES: Cc1ccc(CN2CCC[C@@H](Nc3ccc4[nH]ncc4c3)C2)cc1O] (R)-5-((3-(1H-indazol-5-ylamino)piperidin-1-yl)methyl)- 2-methylphenol | 1.173 |
| 1.174 | 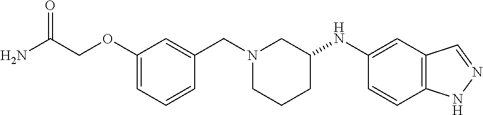 [SMILES: NC(=O)COc1cccc(CN2CCC[C@@H](Nc3ccc4[nH]ncc4c3)C2)c1] (R)-2-(3-((3-(1H-indazol-5-ylamino)piperidin-1- yl)methyl)phenoxy)acetamide | 1.174 |
| 1.175 | 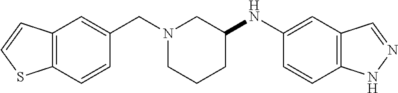 [SMILES: c1cc2cc(CN3CCC[C@H](Nc4ccc5[nH]ncc5c4)C3)ccc2s1] (S)-N-(1-benzo[b]thiophen-5-ylmethyl)piperidin-3-yl)- 1H-indazol-5-amine | 1.175 |
| 1.176 | 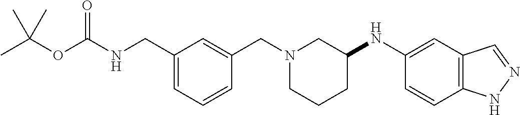 [SMILES: CC(C)(C)OC(=O)NCc1cccc(CN2CCC[C@H](Nc3ccc4[nH]ncc4c3)C2)c1] (S)-tert-butyl 3-((3-(1H-indazol-5-ylamino)piperidin-1- yl)methyl)benzylcarbamate | 1.176 |
| 1.177 | 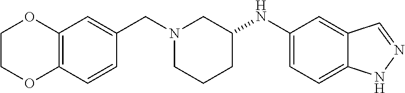 [SMILES: c1cc2c(cc1CN1CCC[C@@H](Nc3ccc4[nH]ncc4c3)C1)OCCO2] (R)-N-(1-((2,3-dihydrobenzo[b][1,4]dioxin-6- yl)methyl)piperidin-3-yl)-1H-indazol-5-amine | 1.177 |
| 1.178 | 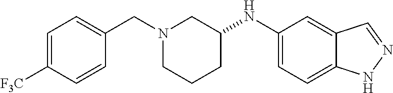 [SMILES: FC(F)(F)c1ccc(CN2CCC[C@@H](Nc3ccc4[nH]ncc4c3)C2)cc1] (R)-N-(1-(4-trifluoromethyl)benzyl)piperidin-3-yl)-1H- indazol-5-amine | 1.178 |
| 1.179 | 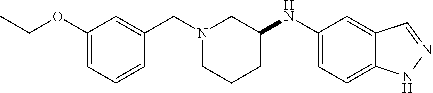 [SMILES: CCOc1cccc(CN2CCC[C@H](Nc3ccc4[nH]ncc4c3)C2)c1] (S)-N-(1-(3-ethoxybenzyl)piperidin-3-yl)-1H-indazol-5- amine | 1.179 |
| 1.180 | 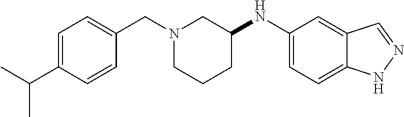 [SMILES: CC(C)c1ccc(CN2CCC[C@H](Nc3ccc4[nH]ncc4c3)C2)cc1] (S)-N-(1-(4-isopropylbenzyl)piperidin-3-yl)-1H-indazol- 5-amine | 1.180 |
| 1.181 | 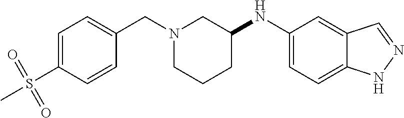 [SMILES: CS(=O)(=O)c1ccc(CN2CCC[C@H](Nc3ccc4[nH]ncc4c3)C2)cc1] (S)-N-(1-(4-(methylsulfonyl)benzyl)piperidin-3-yl)-1H- indazol-5-amine | 1.181 |
| 1.182 | 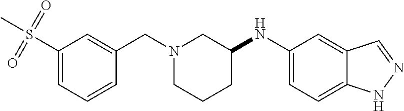 [SMILES: CS(=O)(=O)c1cccc(CN2CCC[C@H](Nc3ccc4[nH]ncc4c3)C2)c1] (S)-N-(1-(3-(methylsulfonyl)benzyl)piperidin-3-yl)-1H- indazol-5-amine | 1.182 |
| 1.183 | 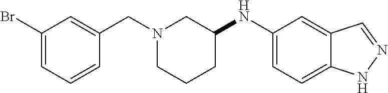 [SMILES: Brc1cccc(CN2CCC[C@H](Nc3ccc4[nH]ncc4c3)C2)c1] (S)-N-(1-(3-bromobenzyl)piperidin-3-yl)-1H-indazol-5- amine | 1.183 |
| 1.184 | 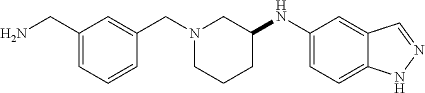 [SMILES: NCc1cccc(CN2CCC[C@H](Nc3ccc4[nH]ncc4c3)C2)c1] (S)-N-(1-(3-(aminomethyl)benzyl)piperidin-3-yl)-1H- indazol-5-amine | 1.184 |
| 1.185 | 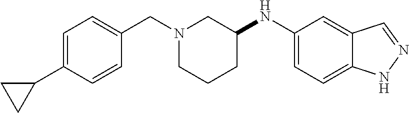 [SMILES: c1cc(C2CC2)ccc1CN1CCC[C@H](Nc2ccc3[nH]ncc3c2)C1] (S)-N-(1-(4-cyclopropylbenzyl)piperidin-3-yl)-1H- indazol-5-amine | 1.185 |
| 1.186 | 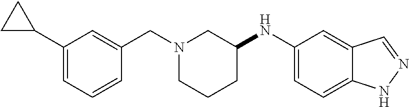 [SMILES: c1cc(CN2CCC[C@H](Nc3ccc4[nH]ncc4c3)C2)cc(C2CC2)c1] (S)-N-(1-(3-cyclopropylbenzyl)piperidin-3-yl)-1H- indazol-5-amine | 1.186 |
| 1.187 | 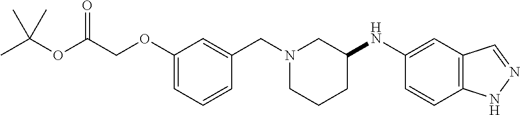 [SMILES: CC(C)(C)OC(=O)COc1cccc(CN2CCC[C@H](Nc3ccc4[nH]ncc4c3)C2)c1] (S)-tert-buyl 2-(3-((3-(1H-indazol-5-ylamino)piperidin- 1-yl)methyl)phenoxy)acetate | 1.187 |
| 1.188 | 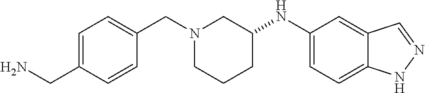 [SMILES: NCc1ccc(CN2CCC[C@@H](Nc3ccc4[nH]ncc4c3)C2)cc1] (R)-N-(1-(4-(aminoethyl)benzyl)piperidin-3-yl)-1H- indazol-5-amine | 1.188 |
| 1.189 | 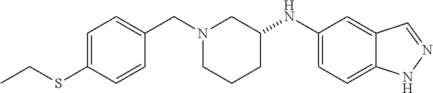 [SMILES: CCSc1ccc(CN2CCC[C@@H](Nc3ccc4[nH]ncc4c3)C2)cc1] (R)-N-(1-(4-(ethylthio)benzyl)piperidin-3-yl)-1H- indazol-5-amine | 1.189 |
| 1.190 | 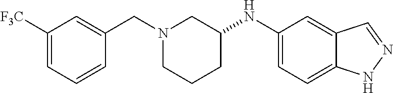 [SMILES: FC(F)(F)c1cccc(CN2CCC[C@@H](Nc3ccc4[nH]ncc4c3)C2)c1] (R)-N-(1-(3-(trifluoromethyl)benzyl)piperidin-3-yl)-1H- indazol-5-amine | 1.190 |
| 1.191 | 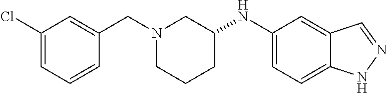 [SMILES: Clc1cccc(CN2CCC[C@@H](Nc3ccc4[nH]ncc4c3)C2)c1] (R)-N-(1-(3-chlorobenzyl)piperidin-3-yl)-1H-indazol-5- amine | 1c, 7, 8, 9, 10 |
| 1.192 | 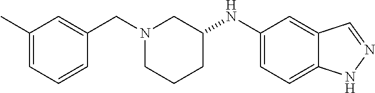 [SMILES: Cc1cccc(CN2CCC[C@@H](Nc3ccc4[nH]ncc4c3)C2)c1] (R)-N-(1-(3-methylbenzyl)piperidin-3-yl)-1H-indazol-5- amine | 1c, 7, 8, 9, 10 |
| 1.193 | 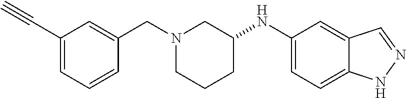 [SMILES: C#Cc1cccc(CN2CCC[C@@H](Nc3ccc4[nH]ncc4c3)C2)c1] (R)-N-(1-(3-ethynylbenzyl)piperidin-3-yl)-1H-indazol-5- amine | 1c, 7, 8, 9, 10, 11, 14c |
| 1.194 | 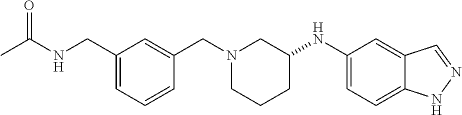 [SMILES: CC(=O)NCc1cccc(CN2CCC[C@@H](Nc3ccc4[nH]ncc4c3)C2)c1] (R)-N-(1H-indazol-5-ylamino)piperidin-1- yl)methyl)benzyl)acetamide | 1c, 7, 8, 9, 10, 13, 16c |
| 1.195 | 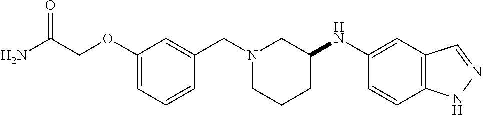 [SMILES: NC(=O)COc1cccc(CN2CCC[C@H](Nc3ccc4[nH]ncc4c3)C2)c1] (S)-2-(3-((3-(1H-indazol-5-ylamino)piperidin-1- yl)methyl)phenyl-1H-indazol-5-ylamino)piperidin-1- yl)methyl)phenoxy)acetamide | 1c, 7, 8, 9, 10, 12a, 15c, 15d |
| 1.196 | 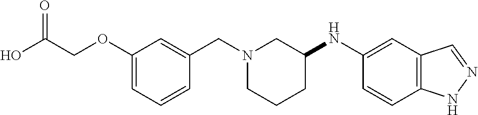 [SMILES: O=C(O)COc1cccc(CN2CCC[C@H](Nc3ccc4[nH]ncc4c3)C2)c1] (S)-2-(3-((3-(1H-indazol-5-ylamino)piperidin-1- yl)methyl)phenoxy)acetic acid | 1c, 7, 8, 9, 10, 12a, 15c, 15d |
| 1.197 | 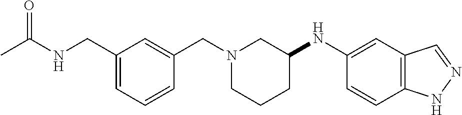 [SMILES: CC(=O)NCc1cccc(CN2CCC[C@H](Nc3ccc4[nH]ncc4c3)C2)c1] (S)-N-(1H-indazol-5-ylamino)piperidin-1- yl)methyl)benzyl)acetamide | 1c, 7, 8, 9, 10, 13, 16c |
| 1.198 | 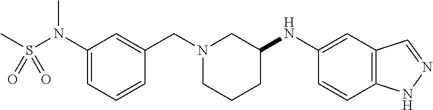 [SMILES: CN(c1cccc(CN2CCC[C@H](Nc3ccc4[nH]ncc4c3)C2)c1)S(C)(=O)=O] (S)-N-(3-((3-(1H-indazol-5-ylamino)piperidin-1- yl)methyl)phenyl)-N-methylmethanesulfonamide | 1c, 7, 8, 9, 10, 12a, 15c, 15d |
| 1.199 | 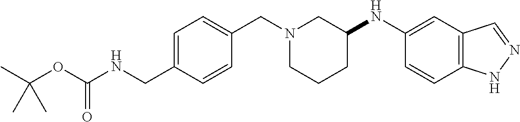 [SMILES: CC(C)(C)OC(=O)NCc1ccc(CN2CCC[C@H](Nc3ccc4[nH]ncc4c3)C2)cc1] (S)-tert-butyl 4-((3-(1H-indazol-5-ylamino)piperidin-1- yl)methyl)benzylcarbamate | 1c, 7, 8, 9, 10, 13, 16c |
| 1.200 | 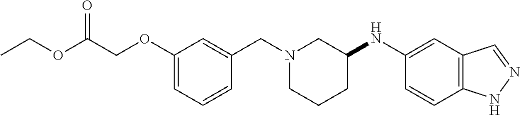 [SMILES: CCOC(=O)COc1cccc(CN2CCC[C@H](Nc3ccc4[nH]ncc4c3)C2)c1] (S)-ethyl 2-(3-((3-(1H-indazol-5-ylamino)piperidin-1- yl)methyl)phenoxy)acetate | 1c, 7, 8, 9, 10, 12a, 15c, 15d |
| 1.201 | 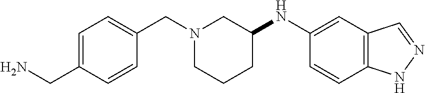 [SMILES: NCc1ccc(CN2CCC[C@H](Nc3ccc4[nH]ncc4c3)C2)cc1] (S)-N-(1-(4-(aminomethyl)benzyl)piperidin-3-yl)-1H- indazol-5-amine | 1c, 7, 8, 9, 10, 13, 16c |
| 1.202 | 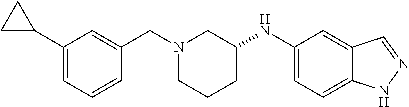 [SMILES: c1cc(CN2CCC[C@@H](Nc3ccc4[nH]ncc4c3)C2)cc(C2CC2)c1] (R)-N-(1-(3-cyclopropylbenzyl)piperidin-3-yl)-1H- indazol-5-amine | 1c, 7, 8, 9, 10, 11, 14c |
| 1.203 | 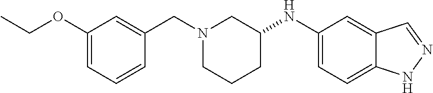 [SMILES: CCOc1cccc(CN2CCC[C@@H](Nc3ccc4[nH]ncc4c3)C2)c1] (R)-N-(1-(3-ethoxybenzyl)piperidin-3-yl)-1H-indazol-5- amine | 1c, 7, 8, 9, 10 |
| 1.204 | 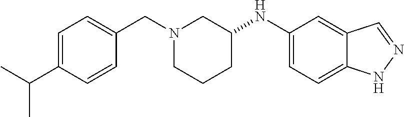 [SMILES: CC(C)c1ccc(CN2CCC[C@@H](Nc3ccc4[nH]ncc4c3)C2)cc1] (R)-N-(1-(4-isopropylbenzyl)piperidin-3-yl)-1H-indazol- 5-amine | 1c, 7, 8, 9, 10 |
| 1.205 | 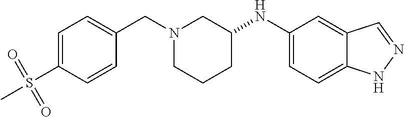 [SMILES: CS(=O)(=O)c1ccc(CN2CCC[C@@H](Nc3ccc4[nH]ncc4c3)C2)cc1] (R)-N-(1-(4-(methylsulfonyl)benzyl)piperidin-3-yl)-1H- indazol-5-amine | 1c, 7, 8, 9, 10, 12, 15c |
| 1.206 | 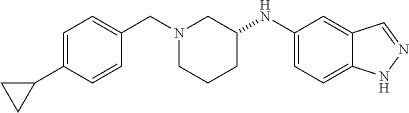 [SMILES: c1cc(C2CC2)ccc1CN1CCC[C@@H](Nc2ccc3[nH]ncc3c2)C1] (R)-N-(1-(4-cyclopropylbenzyl)piperidin-3-yl)-1H- indazol-5-amine | 1c, 7, 8, 9, 10, 11, 14c |
| 1.207 | 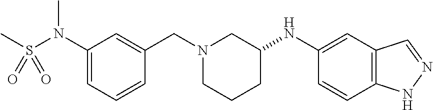 [SMILES: CN(c1cccc(CN2CCC[C@@H](Nc3ccc4[nH]ncc4c3)C2)c1)S(C)(=O)=O] (R)-N-(3-((3-(1H-indazol-5-ylamino)piperidin-1- yl)methyl)phenyl)-N-methylmethanesulfonamide | 1c, 7, 8, 9, 10, 12a, 15c, 15d |
| 1.208 | 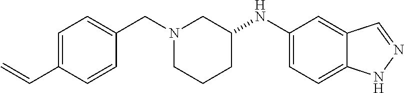 [SMILES: C=Cc1ccc(CN2CCC[C@@H](Nc3ccc4[nH]ncc4c3)C2)cc1] (R)-N-(1-(4-vinylbenzyl)piperidin-3-yl)-1H-indazol-5- amine | 1c, 7, 8, 9, 10, 11, 14c |
| 1.209 | 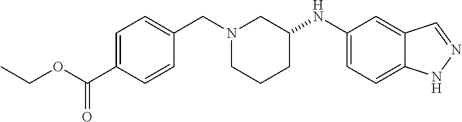 [SMILES: CCOC(=O)c1ccc(CN2CCC[C@@H](Nc3ccc4[nH]ncc4c3)C2)cc1] (R)-ethyl 4-((3-(1H-indazol-5-ylamino)piperidin-1- yl)methyl)benzoate | 1c, 7, 8, 9, 10 |
| 1.210 | 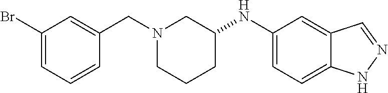 [SMILES: Brc1cccc(CN2CCC[C@@H](Nc3ccc4[nH]ncc4c3)C2)c1] (R)-N-(1-(3-bromobenzyl)piperidin-3-yl)-1H-indazol-5- amine | 1c, 7, 8, 9, 10 |
| 1.211 | 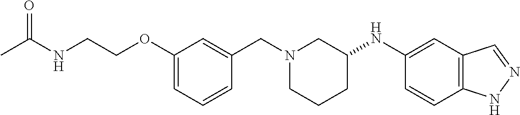 [SMILES: CC(=O)NCCOc1cccc(CN2CCC[C@@H](Nc3ccc4[nH]ncc4c3)C2)c1] (R)-N-(2-(3-((3-(1H-indazol-5-ylamino)piperidin-1- yl)methyl)phenoxy)ethyl)acetamide | 1c, 7, 8, 9, 10, 12a, 15c, 15d |
| 1.212 | 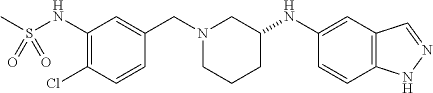 [SMILES: CS(=O)(=O)Nc1cc(CN2CCC[C@@H](Nc3ccc4[nH]ncc4c3)C2)ccc1Cl] (R)-N-(5-((3-(1H-indazol-5-ylamino)piperidin-1- yl)methyl)-2-chlorophenyl)methanesulfonamide | 1c, 7, 8, 9, 10, 12a, 15c, 15d |
| 1.213 | 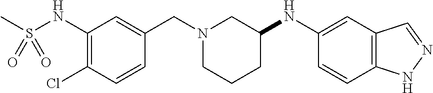 [SMILES: CS(=O)(=O)Nc1cc(CN2CCC[C@H](Nc3ccc4[nH]ncc4c3)C2)ccc1Cl] (S)-N-(5-((3-(1H-indazol-5-ylamino)piperidin-1- yl)methyl)-2-chlorophenyl)methanesulfonamide | 1c, 7, 8, 9, 10, 12a, 15c, 15d |
| 1.214 | 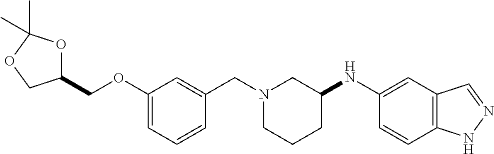 N-((S)-1-(3-(((S)-2,2-dimethyl-1,3-dioxolan-4- yl)methoxy)benzyl)piperidin-3-yl)-1H-indazol-5-amine | 1c, 7, 8, 9, 10, 12a, 15c, 15d |
| 1.215 | 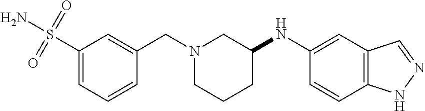 [SMILES: NS(=O)(=O)c1cccc(CN2CCC[C@H](Nc3ccc4[nH]ncc4c3)C2)c1] (S)-3-((3-(1H-indazol-5-ylamino)piperidin-1- yl)methyl)benzenesulfonamide | 1c, 7, 8, 9, 10, 12, 15c |
| 1.216 | 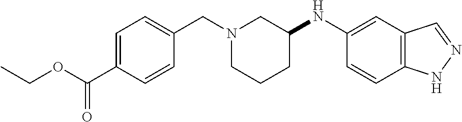 [SMILES: CCOC(=O)c1ccc(CN2CCC[C@H](Nc3ccc4[nH]ncc4c3)C2)cc1] (S)-ethyl 4-((3-(1H-indazol-5-ylamino)piperidin-1- yl)methyl)benzoate | 1c, 7, 8, 9, 10 |
| 1.217 | 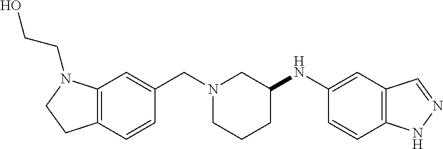 [SMILES: OCCN1CCc2ccc(CN3CCC[C@H](Nc4ccc5[nH]ncc5c4)C3)cc21] (S)-2-(6-((3-(1H-indazol-5-ylamino)piperidin-1- yl)methyl)indolin-1-yl)ethanol | 1c, 7, 8, 9, 10, 13, 16c |
| 1.218 | 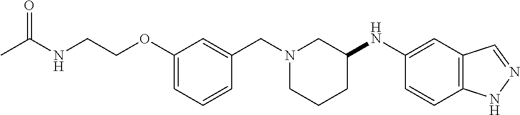 [SMILES: CC(=O)NCCOc1cccc(CN2CCC[C@H](Nc3ccc4[nH]ncc4c3)C2)c1] (S)-N-(2-(3-((3-(1H-indazol-5-ylamino)piperidin-1- yl)methyl)phenoxy)ethyl)acetamide | 1c, 7, 8, 9, 10, 12a, 15c, 15d |
| 1.219 | 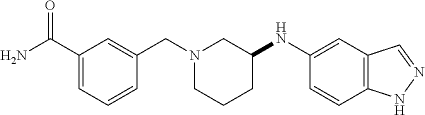 [SMILES: NC(=O)c1cccc(CN2CCC[C@H](Nc3ccc4[nH]ncc4c3)C2)c1] (S)-3-((3-(1H-indazol-5-ylamino)piperidin-1- yl)methyl)benzamide | 1c, 7, 8, 9, 10, 12, 15c |
| 1.221 | 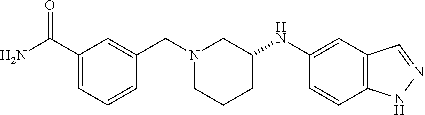 [SMILES: NC(=O)c1cccc(CN2CCC[C@@H](Nc3ccc4[nH]ncc4c3)C2)c1] (R)-3-((3-(1H-indazol-5-ylamino)piperidin-1- yl)methyl)benzamide | 1c, 7, 8, 9, 10, 12, 15c |
| 1.222 | 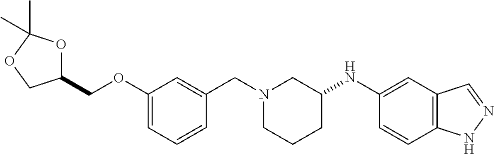 N-(((R)-1-(3-(((S)-2,2-dimethyl-1,3-dioxolan-4- yl)methoxy)benzyl)piperidin-3-yl)-1H-indazol-5-amine | 1c, 7, 8, 9, 10, 12a, 15c, 15d |
| 1.223 | 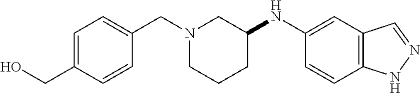 [SMILES: OCc1ccc(CN2CCC[C@H](Nc3ccc4[nH]ncc4c3)C2)cc1] (S)-(4-((3-(1H-indazol-5-ylamino)piperidin-1- yl)methyl)phenyl)methanol | 1c, 7, 8, 9, 10, 13, 16c |
| 1.224 | 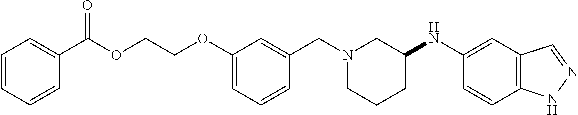 [SMILES: O=C(OCCOc1cccc(CN2CCC[C@H](Nc3ccc4[nH]ncc4c3)C2)c1)c1ccccc1] (S)-2-(3-((3-(1H-indazol-5-ylamino)piperidin-1- yl)methyl)phenoxy)ethyl benzoate | 1c, 7, 8, 9, 10, 12a, 15c, 15d |
| 1.225 | 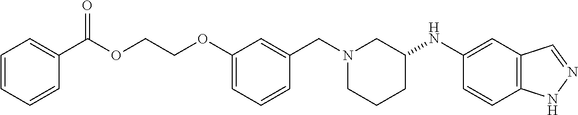 [SMILES: O=C(OCCOc1cccc(CN2CCC[C@@H](Nc3ccc4[nH]ncc4c3)C2)c1)c1ccccc1] (R)-2-(3-((3-(1H-indazol-5-ylamino)piperidin-1- yl)methyl)phenoxy)ethyl benzoate | 1c, 7, 8, 9, 10, 12a, 15c, 15d |
| 1.226 | 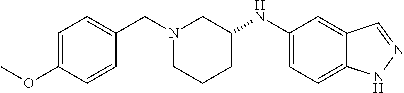 [SMILES: COc1ccc(CN2CCC[C@@H](Nc3ccc4[nH]ncc4c3)C2)cc1] (R)-N-(1-(4-methoxybenzyl)piperidin-3-yl)-1H-indazol- 5-amine | 1c, 7, 8, 9, 10 |
| 1.227 | 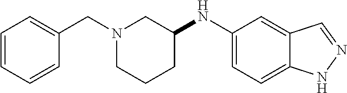 [SMILES: c1ccc(CN2CCC[C@H](Nc3ccc4[nH]ncc4c3)C2)cc1] (S)-N-(1-benzylpiperidin-3-yl)-1H-indazol-5-amine | 1c, 7, 8, 9, 10 |
| 1.228 | 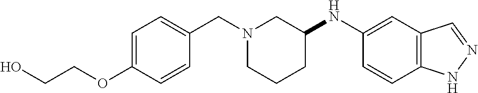 [SMILES: OCCOc1ccc(CN2CCC[C@H](Nc3ccc4[nH]ncc4c3)C2)cc1] (S)-2-(4-((3-(1H-indazol-5-ylamino)piperidin-1- yl)methyl)phenoxy)ethanol | 1c, 7, 8, 9, 10, 12a, 15c, 15d |
| 1.229 | 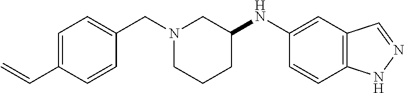 [SMILES: C=Cc1ccc(CN2CCC[C@H](Nc3ccc4[nH]ncc4c3)C2)cc1] (S)-N-(1-(4-vinylbenzyl)piperidin-3-yl)-1H-indazol-5- amine | 1c, 7, 8, 9, 10, 11, 14c |
| 1.230 | 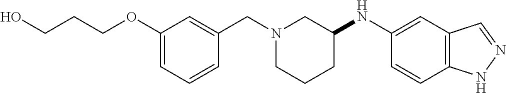 [SMILES: OCCCOc1cccc(CN2CCC[C@H](Nc3ccc4[nH]ncc4c3)C2)c1] (S)-3-(3-((3-(1H-indazol-5-ylamino)piperidin-1- yl)methyl)phenoxy)propan-1-ol | 1c, 7, 8, 9, 10, 12a, 15c, 15d |
| 1.231 | 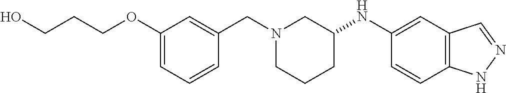 [SMILES: OCCCOc1cccc(CN2CCC[C@@H](Nc3ccc4[nH]ncc4c3)C2)c1] (R)-3-(3-((3-(1H-indazol-5-ylamino)piperidin-1- yl)methyl)phenoxy)propan-1-ol | 1c, 7, 8, 9, 10, 12a, 15c, 15d |
| 1.232 | 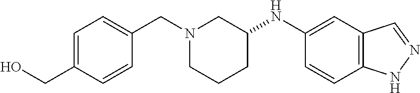 [SMILES: OCc1ccc(CN2CCC[C@@H](Nc3ccc4[nH]ncc4c3)C2)cc1] (R)-(4-((3-(1H-indazol-5-ylamino)piperidin-1- yl)methyl)phenyl)methanol | 1c, 7, 8, 9, 10 |
| 1.233 | 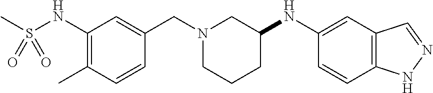 [SMILES: Cc1ccc(CN2CCC[C@H](Nc3ccc4[nH]ncc4c3)C2)cc1NS(C)(=O)=O] (S)-N-(5-((3-(1H-indazol-5-ylamino)piperidin-1- yl)methyl)-2-methylphenyl)methanesulfonamide | 1c, 7, 8, 9, 10, 12a, 15c, 15d |
| 1.234 | 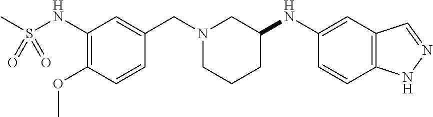 [SMILES: COc1ccc(CN2CCC[C@H](Nc3ccc4[nH]ncc4c3)C2)cc1NS(C)(=O)=O] (S)-N-(5-((3-(1H-indazol-5-ylamino)piperidin-1- yl)methyl)-2-methoxyphenyl)methanesulfonamide | 1c, 7, 8, 9, 10, 12a, 15c, 15d |
| 1.235 | 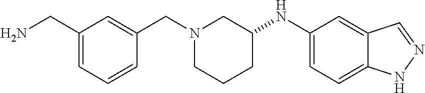 [SMILES: NCc1cccc(CN2CCC[C@@H](Nc3ccc4[nH]ncc4c3)C2)c1] (R)-N-(1-(3-(aminomethyl)benzyl)piperidin-3-yl)-1H- indazol-5-amine | 1c, 7, 8, 9, 10, 13, 16c |
| 1.236 | 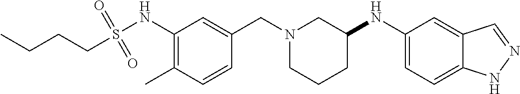 [SMILES: CCCCS(=O)(=O)Nc1cc(CN2CCC[C@H](Nc3ccc4[nH]ncc4c3)C2)ccc1C] (S)-N-(5-((3-(1H-indazol-5-ylamino)piperidin-1- yl)methyl)-2-methylphenyl)butane-1-sulfonamide | 1c, 7, 8, 9, 10, 12a, 15c, 15d |
| 1.237 | 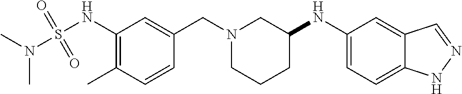 [SMILES: Cc1ccc(CN2CCC[C@H](Nc3ccc4[nH]ncc4c3)C2)cc1NS(=O)(=O)N(C)C] (S)-N-(2-((3-(1H-indazol-5-ylamino)piperidin-1- yl)methyl)-5-methylphenyl)-N′,N′ dimethylsulfamide | 1c, 7, 8, 9, 10, 12a, 15c, 15d |
| 1.238 | 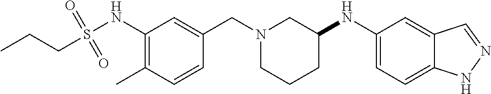 [SMILES: CCCS(=O)(=O)Nc1cc(CN2CCC[C@H](Nc3ccc4[nH]ncc4c3)C2)ccc1C] (S)-N-(5-((3-(1H-indazol-5-ylamino)piperidin-1- yl)methyl)-2-methylphenyl)propane-1-sulfonamide | 1c, 7, 8, 9, 10, 12a, 15c, 15d |
| 1.239 | 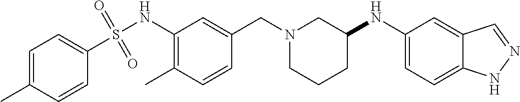 [SMILES: Cc1ccc(S(=O)(=O)Nc2cc(CN3CCC[C@H](Nc4ccc5[nH]ncc5c4)C3)ccc2C)cc1] (S)-N-(5-((3-(1H-indazol-5-ylamino)piperidin-1- yl)methyl)-2-methylphenyl)-4- methylbenzenesulfonamide | 1c, 7, 8, 9, 10, 12a, 15c, 15d |
| 1.240 | 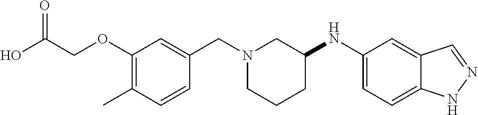 [SMILES: Cc1ccc(CN2CCC[C@H](Nc3ccc4[nH]ncc4c3)C2)cc1OCC(=O)O] (S)-2-(5-((3-(1H-indazol-5-ylamino)piperidin-1- yl)methyl)-2-methylphenyl-1H-indazol-5- ylamino)piperidin-1-yl)methyl)-2-methylphenoxy)acetic acid | 1c, 7, 8, 9, 10, 12a, 15c, 15d |
| 1.241 | 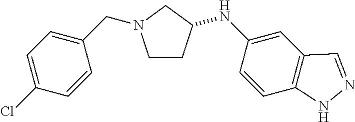 [SMILES: Clc1ccc(CN2CC[C@@H](Nc3ccc4[nH]ncc4c3)C2)cc1] (R)-N-(1-(4-chlorobenzyl)pyrrolidin-3-yl)-1H-indazol-5- amine | 1c, 7, 8, 9, 10 |
| 1.242 | 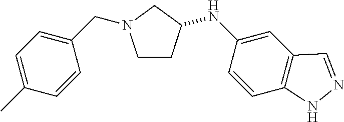 [SMILES: Cc1ccc(CN2CC[C@@H](Nc3ccc4[nH]ncc4c3)C2)cc1] (R)-N-(1-(4-methylbenzyl)pyrrolidin-3-yl)-1H-indazol-5- amine | 1c, 7, 8, 9, 10 |
| 1.243 | 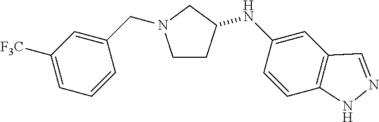 [SMILES: FC(F)(F)c1cccc(CN2CC[C@@H](Nc3ccc4[nH]ncc4c3)C2)c1] (R)-N-(1-(3-(trifluoromethyl)benzyl)pyrrolidin-3-yl)-1H- indazol-5-amine | 1c, 7, 8, 9, 10 |
| 1.244 | 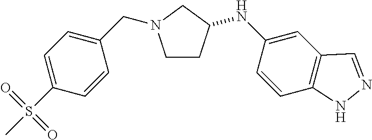 [SMILES: CS(=O)(=O)c1ccc(CN2CC[C@@H](Nc3ccc4[nH]ncc4c3)C2)cc1] (R)-N-(1-(4-(methylsulfonyl)benzyl)pyrrolidin-3-yl)-1H- indazol-5-amine | 1c, 7, 8, 9, 10, 12b, 15c, 15e |
| 1.245 | 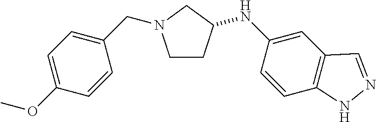 [SMILES: COc1ccc(CN2CC[C@@H](Nc3ccc4[nH]ncc4c3)C2)cc1] (R)-N-(1-(4-methoxybenzyl)pyrrolidin-3-yl)-1H-indazol- 5-amine | 1c, 7, 8, 9, 10 |
| 1.246 | 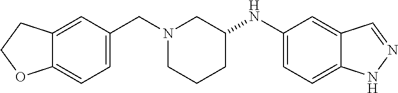 [SMILES: c1cc2c(cc1CN1CCC[C@@H](Nc3ccc4[nH]ncc4c3)C1)CCO2] (R)-N-(1-((2,3-dihydrobenzofuran-5- yl)methyl)piperidin-3-yl)-1H-indazol-5-amine | 1c, 7, 8, 9, 10 |
| 1.247 | 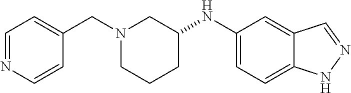 [SMILES: c1cc(CN2CCC[C@@H](Nc3ccc4[nH]ncc4c3)C2)ccn1] (R)-N-(1-(pyridin-4-ylmethyl)piperidin-3-yl)-1H-indazol- 5-amine | 1c, 7, 8, 9, 10 |
| 1.248 | 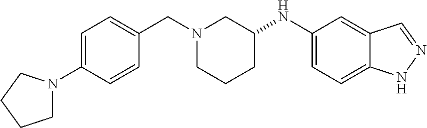 [SMILES: c1cc(N2CCCC2)ccc1CN1CCC[C@@H](Nc2ccc3[nH]ncc3c2)C1] (R)-N-(1-(4-(pyrrolidin-1-yl)benzyl)piperidin-3-yl)-1H- indazol-5-amine | 1c, 7, 8, 9, 10, 11, 14c |
| 1.249 | 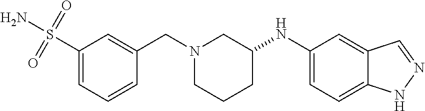 [SMILES: NS(=O)(=O)c1cccc(CN2CCC[C@@H](Nc3ccc4[nH]ncc4c3)C2)c1] (R)-3-((3-(1H-indazol-5-ylamino)piperidin-1- yl)methyl)benzenesulfonamide | 1c, 7, 8, 9, 10, 12b, 15c, 15e |
| 1.250 | 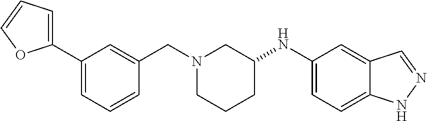 [SMILES: c1cc(CN2CCC[C@@H](Nc3ccc4[nH]ncc4c3)C2)cc(-c2ccco2)c1] (R)-N-(1-(3-(furan-2-yl)benzyl)piperidin-3-yl)-1H- indazol-5-amine | 1c, 7, 8, 9, 10, 11, 14c |
| 1.251 | 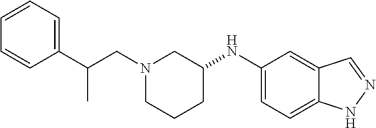 [SMILES: CC(CN1CCC[C@@H](Nc2ccc3[nH]ncc3c2)C1)c1ccccc1] N-((3R)-1-(2-phenylpropyl)piperidin-3-yl)-1H-indazol- 5-amine | 1c, 7, 8, 9 |
| 1.252 | 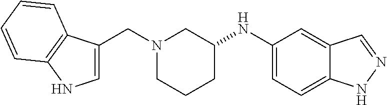 [SMILES: c1ccc2c(CN3CCC[C@@H](Nc4ccc5[nH]ncc5c4)C3)c[nH]c2c1] (R)-N-(1-((1H-indol-3-yl)methyl)piperidin-3-yl)-1H- indazol-5-amine | 1c, 7, 8, 9, 10 |
| 1.253 | 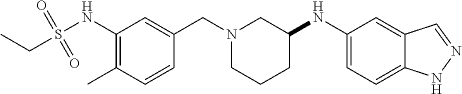 [SMILES: CCS(=O)(=O)Nc1cc(CN2CCC[C@H](Nc3ccc4[nH]ncc4c3)C2)ccc1C] (S)-N-(5-((3-(1H-indazol-5-ylamino)piperidin-1- yl)methyl)-2-methylphenyl)ethanesulfonamide | 1c, 7, 8, 9, 10, 12a, 15c, 15d |
| 1.254 | 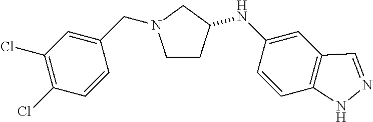 [SMILES: Clc1ccc(CN2CC[C@@H](Nc3ccc4[nH]ncc4c3)C2)cc1Cl] (R)-N-(1-(3,4-dichlorobenzyl)pyrrolidin-3-yl)-1H- indazol-5-amine | 1c, 7, 8, 9, 10 |
| 1.255 | 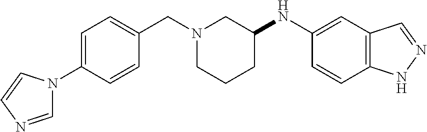 [SMILES: c1cn(-c2ccc(CN3CCC[C@H](Nc4ccc5[nH]ncc5c4)C3)cc2)cn1] (S)-N-(1-(1H-imidazol-1-yl)benzyl)piperidin-3-yl)-1H- indazol-5-amine-indazol | 1c, 7, 8, 9, 10, 11, 14c |
| 1.256 | 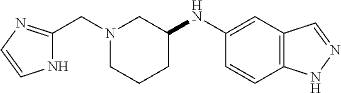 [SMILES: c1c[nH]c(CN2CCC[C@H](Nc3ccc4[nH]ncc4c3)C2)n1] (S)-N-(1-((1H-imidazol-2-yl)methyl)piperidin-3-yl)-1H- indazol-5-amine | 1c, 7, 8, 9, 10 |
| 1.257 | 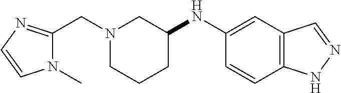 [SMILES: Cn1ccnc1CN1CCC[C@H](Nc2ccc3[nH]ncc3c2)C1] (S)-N-(1-((1-methyl-1H-imidazol-2-yl)methyl)piperidin- 3-yl)-1H-indazol-5-amine | 1c, 7, 8, 9, 10 |
| 1.258 | 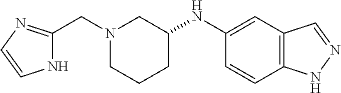 [SMILES: c1c[nH]c(CN2CCC[C@@H](Nc3ccc4[nH]ncc4c3)C2)n1] (R)-N-(5-((3-(1H-indazol-5-ylamino)piperidin-1- yl)methyl)-2-methylphenyl)methanesulfonamide | 1c, 7, 8, 9, 10, 12a, 15c, 15d |
| 1.259 | 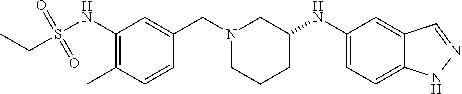 [SMILES: CCS(=O)(=O)Nc1cc(CN2CCC[C@@H](Nc3ccc4[nH]ncc4c3)C2)ccc1C] (R)-N-(5-((3-(1H-indazol-5-ylamino)piperidin-1- yl)methyl)-2-methylphenyl)ethanesulfonamide | 1c, 7, 8, 9, 10, 12a, 15c, 15d |
| 1.260 | 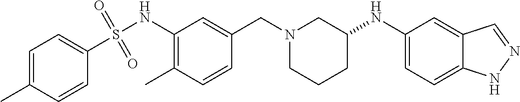 [SMILES: Cc1ccc(S(=O)(=O)Nc2cc(CN3CCC[C@@H](Nc4ccc5[nH]ncc5c4)C3)ccc2C)cc1] (R)-N-(5-((3-(1H-indazol-5-ylamino)piperidin-1- yl)methyl)-2-methylphenyl)-4- methylbenzenesulfonamide | 1c, 7, 8, 9, 10, 12a, 15c, 15d |
| 1.261 | 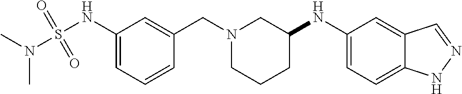 [SMILES: CN(C)S(=O)(=O)Nc1cccc(CN2CCC[C@H](Nc3ccc4[nH]ncc4c3)C2)c1] (S)-N-(3-((3-(1H-indazol-5-ylamino)piperidin-1- yl)methyl)phenyl)-N′,N′dimethylaminosulfamide | 1c, 7, 8, 9, 10, 12a, 15c, 15d |
| 1.262 | 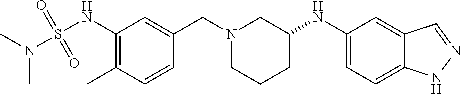 [SMILES: Cc1ccc(CN2CCC[C@@H](Nc3ccc4[nH]ncc4c3)C2)cc1NS(=O)(=O)N(C)C] (R)-N-(2-((3-(1H-indazol-5-ylamino)piperidin-1- yl)methyl)-5-methylphenyl)-N′,N′ dimethylaminosulfamide | 1c, 7, 8, 9, 10, 12a, 15c, 15d |
| 1.263 | 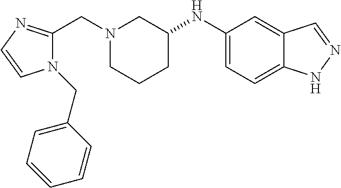 [SMILES: c1ccc(Cn2ccnc2CN2CCC[C@@H](Nc3ccc4[nH]ncc4c3)C2)cc1] (R)-N-(1-((1-benzyl-1H-imidazol-2-yl)methyl)piperidin- 3-yl)-1H-indazol-5-amine | 1c, 7, 8, 9, 10, 11, 14c |
| 1.264 | 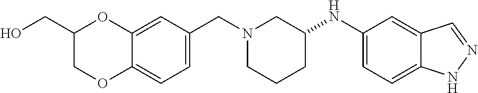 [SMILES: OCC1COc2ccc(CN3CCC[C@@H](Nc4ccc5[nH]ncc5c4)C3)cc2O1] (7-(((R)-3-(1H-indazol-5-ylamino)piperidin-1- yl)methyl)-2,3-dihydrobenzo[b][1,4]dioxin-2- yl)methanol | 1c, 7, 8, 9, 10, 13, 16c |
| 1.265 | 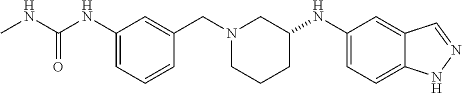 [SMILES: CNC(=O)Nc1cccc(CN2CCC[C@@H](Nc3ccc4[nH]ncc4c3)C2)c1] (R)-1-(3-((3-(1H-indazol-5-ylamino)piperidin-1- yl)methyl)phenyl)-3-methylurea | 1c, 7, 8, 9, 10, 12a, 15c, 15d |
| 1.266 | 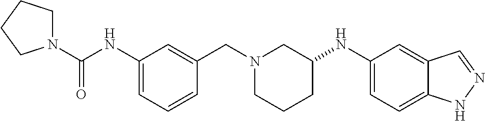 [SMILES: O=C(Nc1cccc(CN2CCC[C@@H](Nc3ccc4[nH]ncc4c3)C2)c1)N1CCCC1] (R)-N-(3-((3-(1H-indazol-5-ylamino)piperidin-1- yl)methyl)phenyl)pyrrolidine-1-carboxamide | 1c, 7, 8, 9, 10, 12a, 15c, 15d |
| 1.267 | 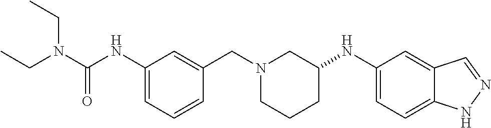 [SMILES: CCN(CC)C(=O)Nc1cccc(CN2CCC[C@@H](Nc3ccc4[nH]ncc4c3)C2)c1] (R)-3-(3-((3-(1H-indazol-5-ylamino)piperidin-1- yl)methyl)phenyl)-1,1-diethylurea | 1c, 7, 8, 9, 10, 12a, 15c, 15d |
| 1.268 | 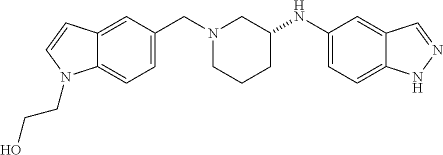 [SMILES: OCCn1ccc2cc(CN3CCC[C@@H](Nc4ccc5[nH]ncc5c4)C3)ccc21] (R)-2-(5-((3-(1H-indazol-5-ylamino)piperidin-1- yl)methyl)-1H-indol-1-yl)ethanol | 1c, 7, 8, 9, 10, 13, 16c |
| 1.269 | 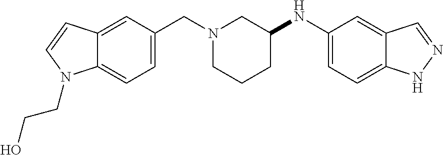 [SMILES: OCCn1ccc2cc(CN3CCC[C@H](Nc4ccc5[nH]ncc5c4)C3)ccc21] (S)-2-(5-((3-(1H-indazol-5-ylamino)piperidin-1- yl)methyl)-1H-indol-1-yl)ethanol | 1c, 7, 8, 9, 10, 13, 16c |
| 1.270 | 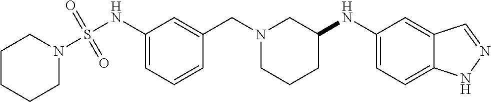 [SMILES: O=S(=O)(Nc1cccc(CN2CCC[C@H](Nc3ccc4[nH]ncc4c3)C2)c1)N1CCCCC1] (S)-N-(3-((3-(1H-indazol-5-ylamino)piperidin-1- yl)methyl)phenyl)piperidine-1-sulfonamide | 1c, 7, 8, 9, 10, 12a, 15c, 15d |
| 1.271 | 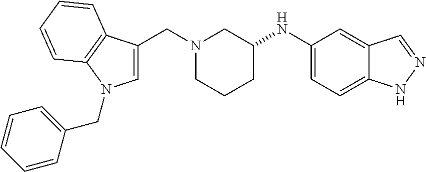 [SMILES: c1ccc(Cn2cc(CN3CCC[C@@H](Nc4ccc5[nH]ncc5c4)C3)c3ccccc32)cc1] (R)-N-(1-((1-benzyl-1H-indol-3-yl)methyl)piperidin-3- yl)-1H-indazol-5-amine | 1c, 7, 8, 9, 10, 11, 14c |
| 1.272 | 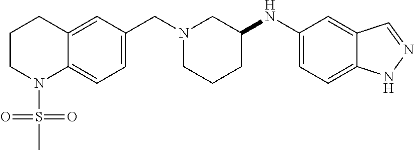 [SMILES: CS(=O)(=O)N1CCCc2cc(CN3CCC[C@H](Nc4ccc5[nH]ncc5c4)C3)ccc21] (S)-N-(1-((1-(methylsulfonyl)-1,2,3,4- tetrahydroquinolin-6-yl)methyl)piperidin-3-yl)-1H- indazol-5-amine | 1c, 7, 8, 9, 10, 12b, 15c, 15e |
| 1.273 | 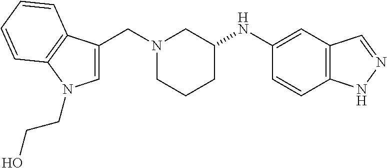 [SMILES: OCCn1cc(CN2CCC[C@@H](Nc3ccc4[nH]ncc4c3)C2)c2ccccc21] (R)-2-(3-((3-(1H-indazol-5-ylamino)piperidin-1- yl)methyl)-1H-indol-1-yl)ethanol | 1c, 7, 8, 9, 10, 13, 16c |
| 1.274 | 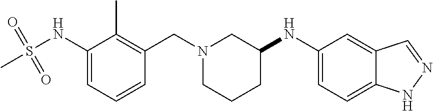 [SMILES: Cc1c(CN2CCC[C@H](Nc3ccc4[nH]ncc4c3)C2)cccc1NS(C)(=O)=O] (S)-N-(3-((3-(1H-indazol-5-ylamino)piperidin-1- yl)methyl)-2-methylphenyl)methanesulfonamide | 1c, 7, 8, 9, 10, 12a, 15c, 15d |
| 1.275 | 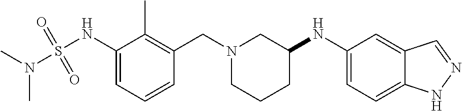 [SMILES: Cc1c(CN2CCC[C@H](Nc3ccc4[nH]ncc4c3)C2)cccc1NS(=O)(=O)N(C)C] (S)-N-(3-((3-(1H-indazol-5-ylamino)piperidin-1- yl)methyl)-2-methylphenyl)-N′,N′ dimethylaminosulfamide | 1c, 7, 8, 9, 10, 12a, 15c, 15d |
| 1.276 | 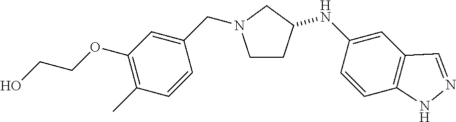 [SMILES: Cc1ccc(CN2CC[C@@H](Nc3ccc4[nH]ncc4c3)C2)cc1OCCO] (R)-2-(5-((3-(1H-indazol-5-ylamino)pyrrolidin-1- yl)methyl)-2-methylphenyl-1H-indazol-5- ylamino)pyrrolidin-1-yl)methyl)-2- methylphenoxy)ethanol | 1c, 7, 8, 9, 10, 12a, 15c, 15d |
| 1.277 | 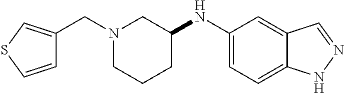 [SMILES: c1cc(CN2CCC[C@H](Nc3ccc4[nH]ncc4c3)C2)cs1] (S)-N-(1-(thiophen-3-ylmethyl)piperidin-3-yl)-1H- indazol-5-amine | 1c, 7, 8, 9, 10 |
| 1.278 | 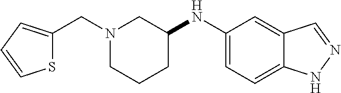 [SMILES: c1csc(CN2CCC[C@H](Nc3ccc4[nH]ncc4c3)C2)c1] (S)-N-(1-(thiophen-2-ylmethyl)piperidin-3-yl)-1H- indazol-5-amine | 1c, 7, 8, 9, 10 |
| 1.279 | 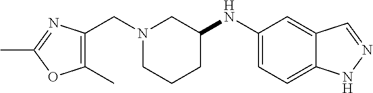 [SMILES: Cc1nc(CN2CCC[C@H](Nc3ccc4[nH]ncc4c3)C2)c(C)o1] (S)-N-(1-((2,5-dimethyloxazol-4-yl)methyl)piperidin-3- yl)-1H-indazol-5-amine | 1c, 7, 8, 9, 10 |
| 1.280 | 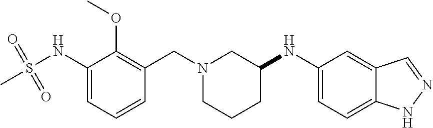 [SMILES: COc1c(CN2CCC[C@H](Nc3ccc4[nH]ncc4c3)C2)cccc1NS(C)(=O)=O] (S)-N-(3-((3-(1H-indazol-5-ylamino)piperidin-1- yl)methyl)-2-methoxyphenyl)methanesulfonamide | 1c, 7, 8, 9, 10, 12a, 15c, 15d |
| 1.281 | 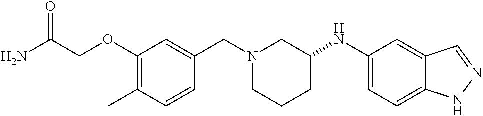 [SMILES: Cc1ccc(CN2CCC[C@@H](Nc3ccc4[nH]ncc4c3)C2)cc1OCC(N)=O] (R)-2-(5-((3-(1H-indazol-5-ylamino)piperidin-1- yl)methyl)-2-methylphenyl-1H-indazol-5- ylamino)piperidin-1-yl)methyl)-2- methylphenoxy)acetamide | 1c, 7, 8, 9, 10, 12a, 15c, 15d |
| 1.282 | 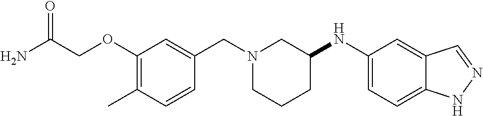 [SMILES: Cc1ccc(CN2CCC[C@H](Nc3ccc4[nH]ncc4c3)C2)cc1OCC(N)=O] (S)-2-(5-((3-(1H-indazol-5-ylamino)piperidin-1- yl)methyl)-2-methylphenyl-1H-indazol-5- ylamino)piperidin-1-yl)methyl)-2- methylphenoxy)acetamide | 1c, 7, 8, 9, 10, 12a, 15c, 15d |
| 2.001 | 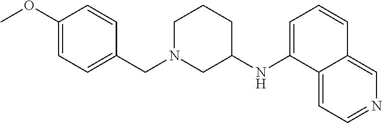 [SMILES: COc1ccc(CN2CCCC(Nc3cccc4cnccc34)C2)cc1] N-(1-(4-methoxybenzyl)piperidin-3-yl)isoquinolin-5- amine | 2c, 7, 8, 9, 10 |
| 2.002 | 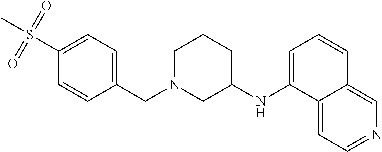 [SMILES: CS(=O)(=O)c1ccc(CN2CCCC(Nc3cccc4cnccc34)C2)cc1] N-(1-(4-(methylsulfonyl)benzyl)piperidin-3- yl)isoquinolin-5-amine | 2c, 7, 8, 9, 10, 12, 15c |
| 2.003 | 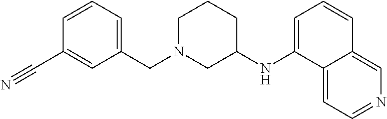 [SMILES: N#Cc1cccc(CN2CCCC(Nc3cccc4cnccc34)C2)c1] 3-((3-(isoquinolin-5-ylamino)piperidin-1- yl)methyl)benzonitrile | 2c, 7, 8, 9, 10, 12, 15c |
| 2.004 | 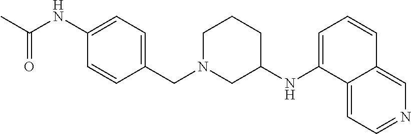 [SMILES: CC(=O)Nc1ccc(CN2CCCC(Nc3cccc4cnccc34)C2)cc1] N-(4-((3-(isoquinolin-5-ylamino)piperidin-1- yl)methyl)phenyl)acetamide | 2c, 7, 8, 9, 10, 12a, 15c, 15d |
| 2.005 | 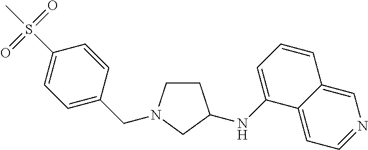 [SMILES: CS(=O)(=O)c1ccc(CN2CCC(Nc3cccc4cnccc34)C2)cc1] N-(1-(4-(methylsulfonyl)benzyl)pyrrolidin-3- yl)isoquinolin-5-amine | 2c, 7, 8, 9, 10, 12, 15c |
| 2.006 | 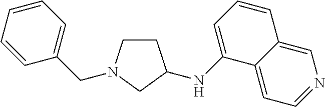 [SMILES: c1ccc(CN2CCC(Nc3cccc4cnccc34)C2)cc1] N-(1-benzylpyrrolidin-3-yl)isoquinolin-5-amine | 2c, 7, 8, 9, 10 |
| 2.007 | 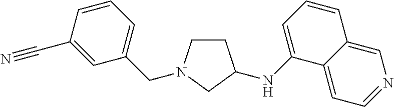 [SMILES: N#Cc1cccc(CN2CCC(Nc3cccc4cnccc34)C2)c1] 3-((3-(isoquinolin-5-ylamino)pyrrolidin-1- yl)methyl)benzonitrile | 2c, 7, 8, 9, 10, 12, 15c |
| 2.008 | 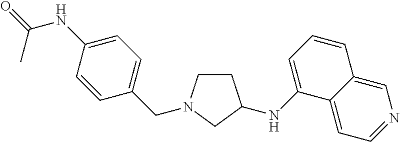 [SMILES: CC(=O)Nc1ccc(CN2CCC(Nc3cccc4cnccc34)C2)cc1] N-(4-((3-(isoquinolin-5-ylamino)pyrrolidin-1- yl)methyl)phenyl)acetamide | 2c, 7, 8, 9, 10, 12a, 15c, 15d |
| 2.009 | 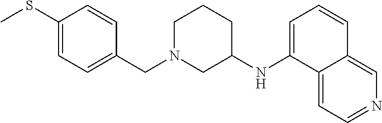 [SMILES: CSc1ccc(CN2CCCC(Nc3cccc4cnccc34)C2)cc1] N-(1-(4-(methylthio)benzyl)piperidin-3-yl)isoquinolin-5- amine | 2c, 7, 8, 9, 10, 12b, 15c, 15e |
| 2.010 | 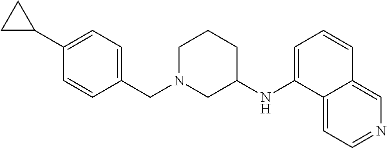 [SMILES: c1cc(NC2CCCN(Cc3ccc(C4CC4)cc3)C2)c2ccncc2c1] N-(1-(4-cyclopropylbenzyl)piperidin-3-yl)isoquinolin-5- amine | 2c, 7, 8, 9, 10, 11, 14c |
| 2.011 | 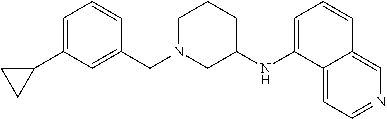 [SMILES: c1cc(CN2CCCC(Nc3cccc4cnccc34)C2)cc(C2CC2)c1] N-(1-(3-cyclopropylbenzyl)piperidin-3-yl)isoquinolin-5- amine | 2c, 7, 8, 9, 10, 11, 14c |
| 2.012 | 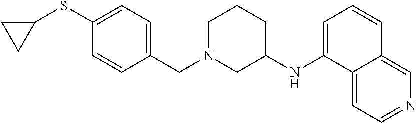 [SMILES: c1cc(NC2CCCN(Cc3ccc(SC4CC4)cc3)C2)c2ccncc2c1] N-(1-(4-(cyclopropylthio)benzyl)piperidin-3- yl)isoquinolin-5-amine | 2c, 7, 8, 9, 10, 12b, 15c, 15e |
| 2.013 | 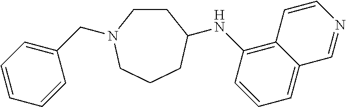 [SMILES: c1ccc(CN2CCCC(Nc3cccc4cnccc34)CC2)cc1] N-(1-benzylazepan-4-yl)isoquinolin-5-amine | 2c, 7, 8, 9, 10 |
| 2.014 | 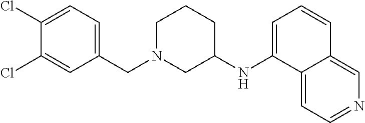 [SMILES: Clc1ccc(CN2CCCC(Nc3cccc4cnccc34)C2)cc1Cl] N-(1-(3,4-dichlorobenzyl)piperidin-3-yl)isoquinolin-5- amine | 2c, 7, 8, 9, 10 |
| 2.015 | 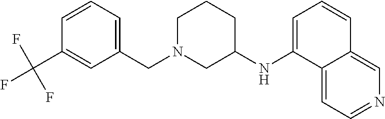 [SMILES: FC(F)(F)c1cccc(CN2CCCC(Nc3cccc4cnccc34)C2)c1] N-(1-(3-(trifluoromethyl)benzyl)piperidin-3- yl)isoquinolin-5-amine | 2c, 7, 8, 9, 10 |
| 2.016 | 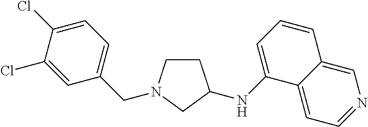 [SMILES: Clc1ccc(CN2CCC(Nc3cccc4cnccc34)C2)cc1Cl] N-(1-(3,4-dichlorobenzyl)pyrrolidin-3-yl)isoquinolin-5- amine | 2c, 7, 8, 9, 10 |
| 2.017 | 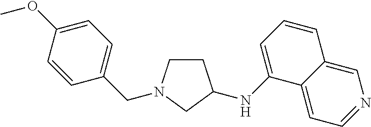 [SMILES: COc1ccc(CN2CCC(Nc3cccc4cnccc34)C2)cc1] N-(1-(4-methoxybenzyl)pyrrolidin-3-yl)isoquinolin-5- amine | 2c, 7, 8, 9, 10 |
| 2.018 | 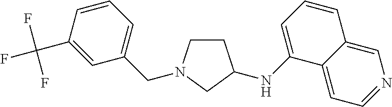 [SMILES: FC(F)(F)c1cccc(CN2CCC(Nc3cccc4cnccc34)C2)c1] N-(1-(3-(trifluoromethyl)benzyl)pyrrolidin-3- yl)isoquinolin-5-amine | 2c, 7, 8, 9, 10 |
| 2.019 | 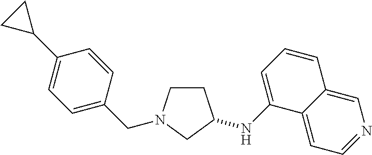 [SMILES: c1cc(N[C@H]2CCN(Cc3ccc(C4CC4)cc3)C2)c2ccncc2c1] (S)-N-(1-(4-cyclopropylbenzyl)pyrrolidin-3- yl)isoquionolin-5-amine | 2c, 7, 8, 9, 10, 11, 14c |
| 2.020 | 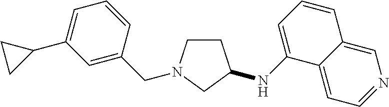 [SMILES: c1cc(CN2CC[C@@H](Nc3cccc4cnccc34)C2)cc(C2CC2)c1] (R)-N-(1-(3-cyclopropylbenzyl)pyrrolidin-3- yl)isoquinolin-5-amine | 2c, 7, 8, 9, 10, 11, 14c |
| 2.021 | 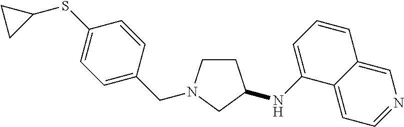 [SMILES: c1cc(N[C@@H]2CCN(Cc3ccc(SC4CC4)cc3)C2)c2ccncc2c1] (R)-N-(1-(4-(cyclopropylthio)benzyl)pyrrolidin-3- yl)isoquinolin-5-amine | 2c, 7, 8, 9, 10, 12b, 15c, 15e |
| 2.022 | 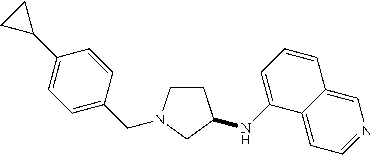 [SMILES: c1cc(N[C@@H]2CCN(Cc3ccc(C4CC4)cc3)C2)c2ccncc2c1] (R)-N-(1-(4-cyclopropylbenzyl)pyrrolidin-3- yl)isoquinolin-5-amine | 2c, 7, 8, 9, 10, 11, 14c |
| 2.023 | 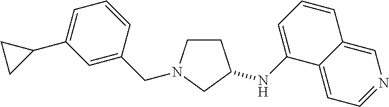 [SMILES: c1cc(CN2CC[C@H](Nc3cccc4cnccc34)C2)cc(C2CC2)c1] (S)-N-(1-(3-cyclopropylbenzyl)pyrrolidin-3- yl)isoquinolin-5-amine | 2c, 7, 8, 9, 10, 11, 14c |
| 2.024 | 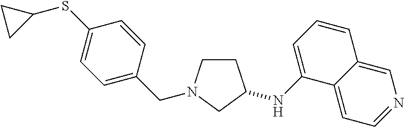 [SMILES: c1cc(N[C@H]2CCN(Cc3ccc(SC4CC4)cc3)C2)c2ccncc2c1] (S)-N-(1-(4-(cyclopropylthio)benzyl)pyrrolidin-3- yl)isoquinolin-5-amine | 2c, 7, 8, 9, 10, 12b, 15c, 15e |
| 2.025 | 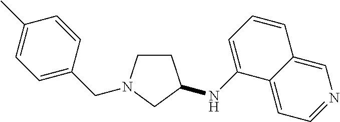 [SMILES: Cc1ccc(CN2CC[C@@H](Nc3cccc4cnccc34)C2)cc1] (R)-N-(1-(4-methylbenzyl)pyrrolidin-3-yl)isoquinolin-5- amine | 2c, 7, 8, 9, 10 |
| 2.026 | 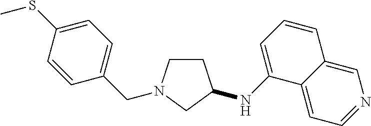 [SMILES: CSc1ccc(CN2CC[C@@H](Nc3cccc4cnccc34)C2)cc1] (R)-N-(1-(4-(methylthio)benzyl)pyrrolidin-3- yl)isoquinolin-5-amine | 2c, 7, 8, 9, 10, 12b, 15c, 15e |
| 2.027 | 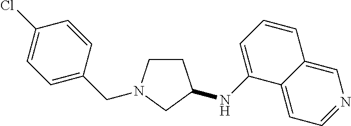 [SMILES: Clc1ccc(CN2CC[C@@H](Nc3cccc4cnccc34)C2)cc1] (R)-N-(1-(4-chlorobenzyl)pyrrolidin-3-yl)isoquinolin-5- amine | 2c, 7, 8, 9, 10 |
| 2.028 | 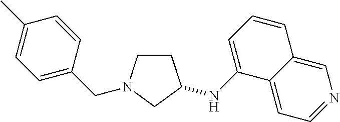 [SMILES: Cc1ccc(CN2CC[C@H](Nc3cccc4cnccc34)C2)cc1] (S)-N-(1-(4-methylbenzyl)pyrrolidin-3-yl)isoquinolin-5- amine | 2c, 7, 8, 9, 10 |
| 2.029 | 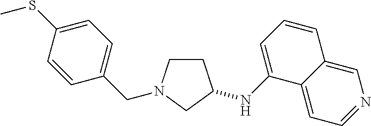 [SMILES: CSc1ccc(CN2CC[C@H](Nc3cccc4cnccc34)C2)cc1] (S)-N-(1-(4-(methylthio)benzyl)pyrrolidin-3- yl)isoquinolin-5-amine | 2c, 7, 8, 9, 10, 12b, 15c, 15e |
| 2.030 | 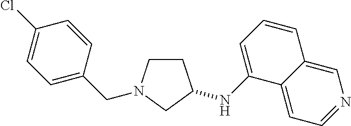 [SMILES: Clc1ccc(CN2CC[C@H](Nc3cccc4cnccc34)C2)cc1] (S)-N-(1-(4-chlorobenzyl)pyrrolidin-3-yl)isoquinolin-5- amine | 2c, 7, 8, 9, 10 |
| 2.031 | 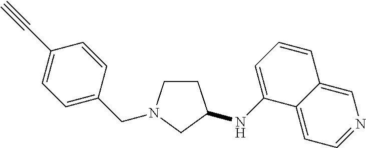 [SMILES: C#Cc1ccc(CN2CC[C@@H](Nc3cccc4cnccc34)C2)cc1] (R)-N-(1-(4-ethynylbenzyl)pyrrolidin-3-yl)isoquinolin-5- amine | 2c, 7, 8, 9, 10, 11, 14c |
| 2.032 | 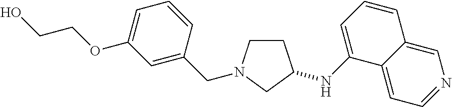 [SMILES: OCCOc1cccc(CN2CC[C@H](Nc3cccc4cnccc34)C2)c1] (S)-2-(3-((3-(isoquinolin-5-ylamino)pyrrolidin-1- yl)methyl)phenoxy)ethanol | 2c, 7, 8, 9, 10, 12a, 15c, 15d |
| 2.033 | 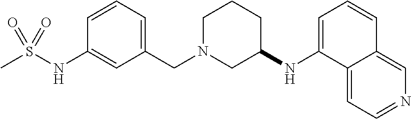 [SMILES: CS(=O)(=O)Nc1cccc(CN2CCC[C@@H](Nc3cccc4cnccc34)C2)c1] (R)-N-(3-((3-(isoquinolin-5-ylamino)piperidin-1- yl)methyl)phenyl)methanesulfonamide | 2c, 7, 8, 9, 10, 12a, 15c, 15d |
| 2.034 | 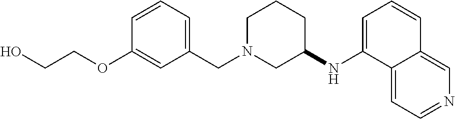 [SMILES: OCCOc1cccc(CN2CCC[C@@H](Nc3cccc4cnccc34)C2)c1] (R)-2-(3-((3-(isoquinolin-5-ylamino)piperidin-1- yl)methyl)phenoxy)ethanol | 2c, 7, 8, 9, 10, 12a, 15c, 15d |
| 2.035 | 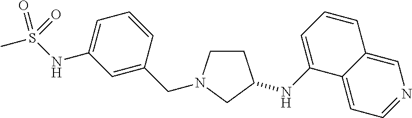 [SMILES: CS(=O)(=O)Nc1cccc(CN2CC[C@H](Nc3cccc4cnccc34)C2)c1] (S)-N-(3-((3-(isoquinolin-5-ylamino)pyrrolidin-1- yl)methyl)phenyl)methanesulfonamide | 2c, 7, 8, 9, 10, 12a, 15c, 15d |
| 2.036 | 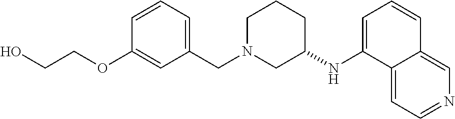 [SMILES: OCCOc1cccc(CN2CCC[C@H](Nc3cccc4cnccc34)C2)c1] (S)-2-(3-((3-(isoquinolin-5-ylamino)piperidin-1- yl)methyl)phenoxy)ethanol | 2c, 7, 8, 9, 10, 12a, 15c, 15d |
| 2.037 | 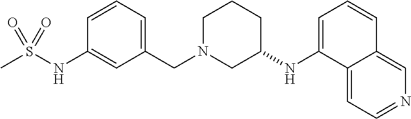 [SMILES: CS(=O)(=O)Nc1cccc(CN2CCC[C@H](Nc3cccc4cnccc34)C2)c1] (S)-N-(3-((3-(isoquinolin-5-ylamino)piperidin-1- yl)methyl)phenyl)methanesulfonamide | 2c, 7, 8, 9, 10, 12a, 15c, 15d |
| 2.038 | 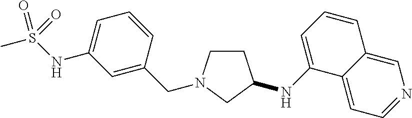 [SMILES: CS(=O)(=O)Nc1cccc(CN2CC[C@@H](Nc3cccc4cnccc34)C2)c1] (R)-N-(3-((3-(isoquinolin-5-ylamino)pyrrolidin-1- yl)methyl)phenyl)methanesulfonamide | 2c, 7, 8, 9, 10, 12a, 15c, 15d |
| 2.039 | 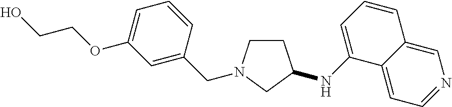 [SMILES: OCCOc1cccc(CN2CC[C@@H](Nc3cccc4cnccc34)C2)c1] (R)-2-(3-((3-(isoquinolin-5-ylamino)pyrrolidin-1- yl)methyl)phenoxy)ethanol | 2c, 7, 8, 9, 10, 12a, 15c, 15d |
| 2.040 | 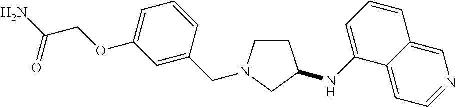 [SMILES: NC(=O)COc1cccc(CN2CC[C@@H](Nc3cccc4cnccc34)C2)c1] (R)-2-(3-((3-(isoquinolin-5-ylamino)pyrrolidin-1- yl)methyl)phenoxy)acetamide | 2c, 7, 8, 9, 10, 12a, 15c, 15d |
| 2.041 | 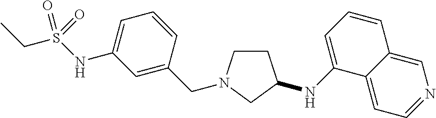 [SMILES: CCS(=O)(=O)Nc1cccc(CN2CC[C@@H](Nc3cccc4cnccc34)C2)c1] (R)-N-(3-((3-(isoquinolin-5-ylamino)pyrrolidin-1- yl)methyl)phenyl)ethanesulfonamide | 2c, 7, 8, 9, 10, 12a, 15c, 15d |
| 2.042 | 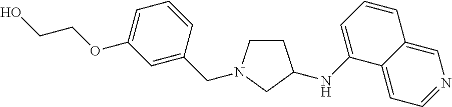 [SMILES: OCCOc1cccc(CN2CCC(Nc3cccc4cnccc34)C2)c1] 2-(3-((3-(isoquinolin-5-ylamino)pyrrolidin-1- yl)methyl)phenoxy)ethanol | 2c, 7, 8, 9, 10, 12a, 15c, 15d |
| 2.043 | 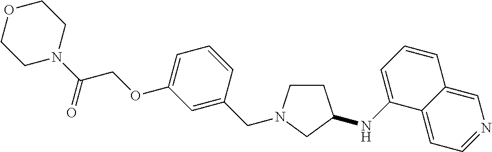 [SMILES: O=C(COc1cccc(CN2CC[C@@H](Nc3cccc4cnccc34)C2)c1)N1CCOCC1] (R)-2-(3-((3-(isoquinolin-5-ylamino)pyrrolidin-1- yl)methyl)phenoxy)-1-morpholinoethanone | 2c, 7, 8, 9, 10, 12a, 15c, 15d |
| 2.044 | 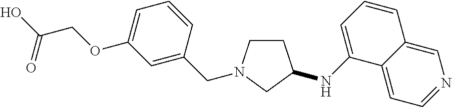 [SMILES: O=C(O)COc1cccc(CN2CC[C@@H](Nc3cccc4cnccc34)C2)c1] (R)-2-(3-((3-(isoquinolin-5-ylamino)pyrrolidin-1- yl)methyl)phenoxy)acetic acid | 2c, 7, 8, 9, 10, 12a, 15c, 15d |
| 2.045 | 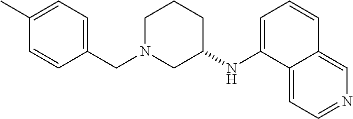 [SMILES: Cc1ccc(CN2CCC[C@H](Nc3cccc4cnccc34)C2)cc1] (S)-N-(1-(4-methylbenzyl)piperidin-3-yl)isoquinolin-5- amine | 2c, 7, 8, 9, 10 |
| 2.046 | 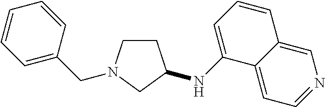 [SMILES: c1ccc(CN2CC[C@@H](Nc3cccc4cnccc34)C2)cc1] (R)-N-(1-benzylpyrrolidin-3-yl)isoquinolin-5-amine | 2c, 7, 8, 9, 10 |
| 2.047 | 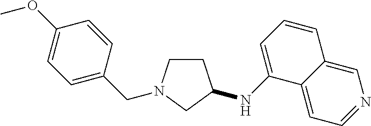 [SMILES: COc1ccc(CN2CC[C@@H](Nc3cccc4cnccc34)C2)cc1] (R)-N-(1-(4-methoxybenzyl)pyrrolidin-3-yl)isoquinolin- 5-amine | 2c, 7, 8, 9, 10 |
| 2.048 | 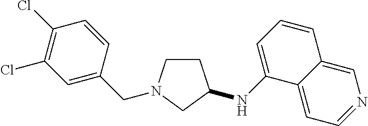 [SMILES: Clc1ccc(CN2CC[C@@H](Nc3cccc4cnccc34)C2)cc1Cl] (R)-N-(1-(3,4-dichlorobenzyl)pyrrolidin-3- yl)isoquinolin-5-amine | 2c, 7, 8, 9, 10 |
| 2.049 | 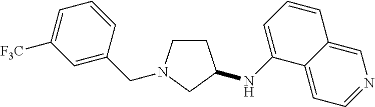 [SMILES: FC(F)(F)c1cccc(CN2CC[C@@H](Nc3cccc4cnccc34)C2)c1] (R)-N-(1-(3-(trifluoromethyl)benzyl)pyrrolidin-3- yl)isoquinolin-5-amine | 2c, 7, 8, 9, 10 |
| 2.050 | 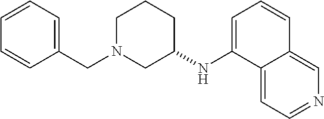 [SMILES: c1ccc(CN2CCC[C@H](Nc3cccc4cnccc34)C2)cc1] (S)-N-(1-benzylpiperidin-3-yl)isoquinolin-5-amine | 2c, 7, 8, 9, 10 |
| 2.051 | 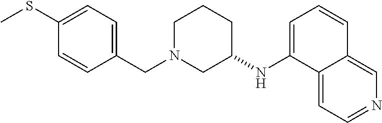 [SMILES: CSc1ccc(CN2CCC[C@H](Nc3cccc4cnccc34)C2)cc1] (S)-N-(1-(4-(methylthio)benzyl)piperidin-3- yl)isoquinolin-5-amine | 2c, 7, 8, 9, 10, 12b, 15c, 15e |
| 2.052 | 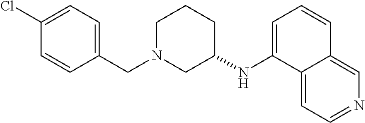 [SMILES: Clc1ccc(CN2CCC[C@H](Nc3cccc4cnccc34)C2)cc1] (S)-N-(1-(4-chlorobenzyl)piperidin-3-yl)isoquinolin-5- amine | 2c, 7, 8, 9, 10 |
| 2.053 | 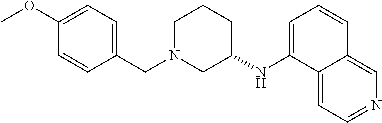 [SMILES: COc1ccc(CN2CCC[C@H](Nc3cccc4cnccc34)C2)cc1] (S)-N-(1-(4-methoxybenzyl)piperidin-3-yl)isoquinolin- 5-amine | 2c, 7, 8, 9, 10 |
| 2.054 | 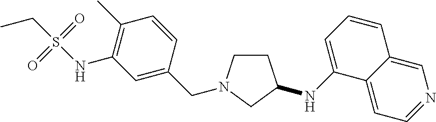 [SMILES: CCS(=O)(=O)Nc1cc(CN2CC[C@@H](Nc3cccc4cnccc34)C2)ccc1C] (R)-N-(5-((3-(isoquinolin-5-ylamino)pyrrolidin-1- yl)methyl)-2-methylphenyl)ethanesulfonamide | 2c, 7, 8, 9, 10, 12a, 15c, 15d |
| 2.055 | 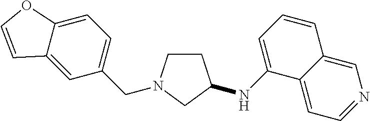 [SMILES: c1cc(N[C@@H]2CCN(Cc3ccc4occc4c3)C2)c2ccncc2c1] (R)-N-(1-(benzofuran-5-ylmethyl)pyrrolidin-3- yl)isoquinolin-5-amine | 2c, 7, 8, 9, 10 |
| 2.056 | 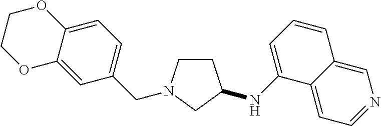 [SMILES: c1cc(N[C@@H]2CCN(Cc3ccc4c(c3)OCCO4)C2)c2ccncc2c1] (R)-N-(1-((2,3-dihydrobenzo[b][1,4]dioxin-6- yl)methyl)pyrrolidin-3-yl)isoquinolin-5-amine | 2c, 7, 8, 9, 10 |
| 2.057 | 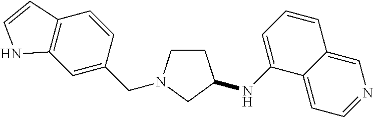 [SMILES: c1cc(N[C@@H]2CCN(Cc3ccc4cc[nH]c4c3)C2)c2ccncc2c1] (R)-N-(1-((1H-indol-6-yl)methyl)pyrrolidin-3- yl)isoquinolin-5-amine | 2c, 7, 8, 9, 10 |
| 2.058 | 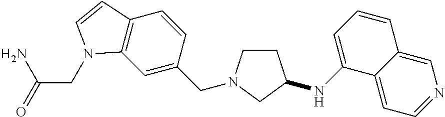 [SMILES: NC(=O)Cn1ccc2ccc(CN3CC[C@@H](Nc4cccc5cnccc45)C3)cc21] (R)-2-(6-((3-(isoquinolin-5-ylamino)pyrrolidin-1- yl)methyl)-1H-indol-1-yl)acetamide | 2c, 7, 8, 9, 10, 13, 16c |
| 2.059 | 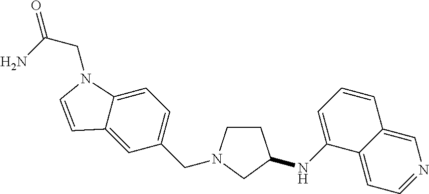 [SMILES: NC(=O)Cn1ccc2cc(CN3CC[C@@H](Nc4cccc5cnccc45)C3)ccc21] (R)-2-(5-((3-(isoquinolin-5-ylamino)pyrrolidin-1- yl)methyl)-1H-indol-1-yl)acetamide | 2c, 7, 8, 9, 10, 13, 16c |
| 2.060 | 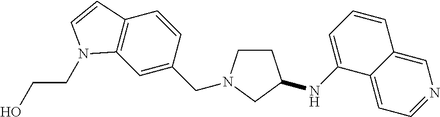 [SMILES: OCCn1ccc2ccc(CN3CC[C@@H](Nc4cccc5cnccc45)C3)cc21] (R)-2-(6-((3-(isoquinolin-5-ylamino)pyrrolidin-1- yl)methyl)-1H-indol-1-yl)ethanol | 2c, 7, 8, 9, 10, 13, 16c |
| 2.061 | 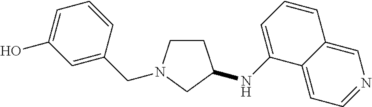 [SMILES: Oc1cccc(CN2CC[C@@H](Nc3cccc4cnccc34)C2)c1] (R)-3-((3-(isoquinolin-5-ylamino)pyrrolidin-1- yl)methyl)phenol | 2c, 7, 8, 9, 10 |
| 2.062 | 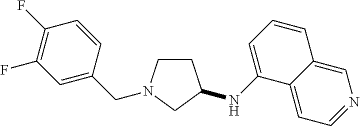 [SMILES: Fc1ccc(CN2CC[C@@H](Nc3cccc4cnccc34)C2)cc1F] (R)-N-(1-(3,4-difluorobenzyl)pyrrolidin-3-yl)isoquinolin- 5-amine | 2c, 7, 8, 9, 10 |
| 2.063 | 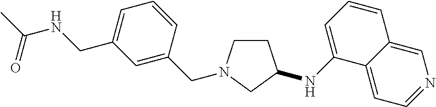 [SMILES: CC(=O)NCc1cccc(CN2CC[C@@H](Nc3cccc4cnccc34)C2)c1] (R)-N-(3-((3-(isoquinolin-5-ylamino)pyrrolidin-1- yl)methyl)benzyl)acetamide | 2c, 7, 8, 9, 10, 13, 16c |
| 2.064 | 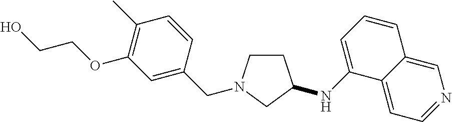 [SMILES: Cc1ccc(CN2CC[C@@H](Nc3cccc4cnccc34)C2)cc1OCCO] (R)-2-(5-((3-(isoquinolin-5-ylamino)pyrrolidin-1- yl)methyl)-2-methylphenoxy)ethanol | 2c, 7, 8, 9, 10, 12a, 15c, 15d |
| 2.065 | 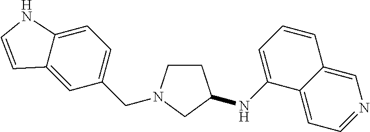 [SMILES: c1cc(N[C@@H]2CCN(Cc3ccc4[nH]ccc4c3)C2)c2ccncc2c1] (R)-N-(1-((1H-indol-5-yl)methyl)pyrrolidin-3- yl)isoquinolin-5-amine | 2c, 7, 8, 9, 10 |
| 2.066 | 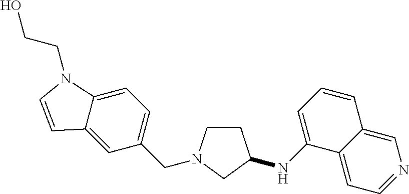 [SMILES: OCCn1ccc2cc(CN3CC[C@@H](Nc4cccc5cnccc45)C3)ccc21] (R)-2-(5-((3-(isoquinolin-5-ylamino)pyrrolidin-1- yl)methyl)-1H-indol-1-yl)ethanol | 2c, 7, 8, 9, 10, 13, 16c |
| 2.067 | 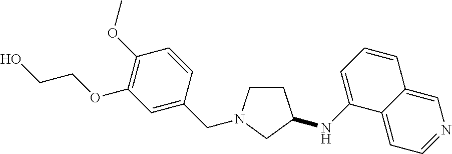 [SMILES: COc1ccc(CN2CC[C@@H](Nc3cccc4cnccc34)C2)cc1OCCO] (R)-2-(5-((3-(isoquinolin-5-ylamino)pyrrolidin-1- yl)methyl)-2-methoxyphenoxy)ethanol | 2c, 7, 8, 9, 10, 12a, 15c, 15d |
| 2.068 | 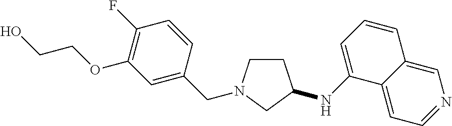 [SMILES: OCCOc1cc(CN2CC[C@@H](Nc3cccc4cnccc34)C2)ccc1F] (R)-2-(2-fluoro-5-((3-(isoquinolin-5-ylamino)pyrrolidin- 1-yl)methyl)phenoxy)ethanol | 2c, 7, 8, 9, 10, 12a, 15c, 15d |
| 2.069 | 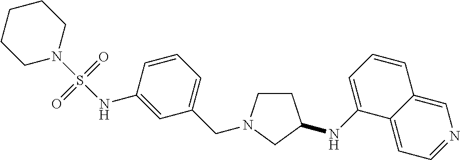 [SMILES: O=S(=O)(Nc1cccc(CN2CC[C@@H](Nc3cccc4cnccc34)C2)c1)N1CCCCC1] (R)-N-(3-((3-(isoquinolin-5-ylamino)pyrrolidin-1- yl)methyl)phenyl)piperidine-1-sulfonamide | 2c, 7, 8, 9, 10, 12a, 15c, 15d |
| 2.070 | 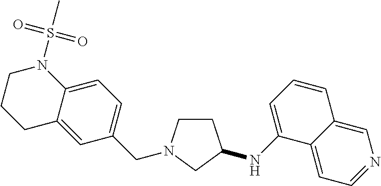 [SMILES: CS(=O)(=O)N1CCCc2cc(CN3CC[C@@H](Nc4cccc5cnccc45)C3)ccc21] (R)-N-(1-((1-(methylsulfonyl)-1,2,3,4- tetrahydroisoquinolin-6-yl)methyl)pyrrolidin-3- yl)isoquinolin-5-amine | 2c, 7, 8, 9, 10, 12b, 15c, 15e |
| 2.071 | 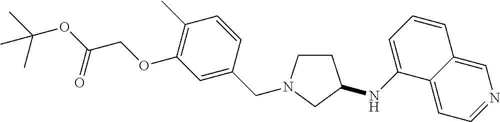 [SMILES: Cc1ccc(CN2CC[C@@H](Nc3cccc4cnccc34)C2)cc1OCC(=O)OC(C)(C)C] (R)-tert-butyl 2-(5-((3-(isoquinolin-5-ylamino)pyrrolidin- 1-yl)methyl)-2-methylphenoxy)acetate | 2c, 7, 8, 9, 10, 12a, 15c, 15d |
| 2.072 | 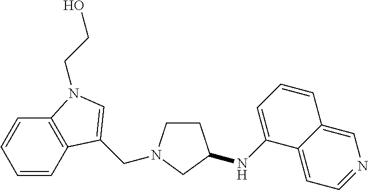 [SMILES: OCCn1cc(CN2CC[C@@H](Nc3cccc4cnccc34)C2)c2ccccc21] (R)-2-(3-((3-(isoquinolin-5-ylamino)pyrrolidin-1- yl)methyl)-1H-indol-1-yl)ethanol | 2c, 7, 8, 9, 10, 13, 16c |
| 2.073 | 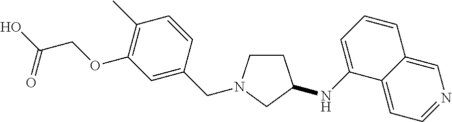 [SMILES: Cc1ccc(CN2CC[C@@H](Nc3cccc4cnccc34)C2)cc1OCC(=O)O] (R)-2-(5-((3-(isoquinolin-5-ylamino)pyrrolidin-1- yl)methyl)-2-methylphenoxy)acetic acid | 2c, 7, 8, 9, 10, 12a, 15c, 15d |
| 2.074 | 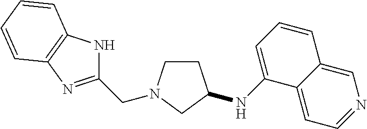 [SMILES: c1cc(N[C@@H]2CCN(Cc3nc4ccccc4[nH]3)C2)c2ccncc2c1] (R)-N-(1-((1H-benzo[d]imidazol-2-yl)methyl)pyrrolidin- 3-yl)isoquinolin-5-amine | 2c, 7, 8, 9, 10 |
| 2.075 | 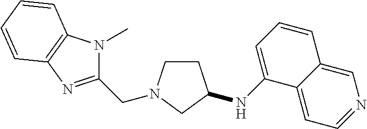 [SMILES: Cn1c(CN2CC[C@@H](Nc3cccc4cnccc34)C2)nc2ccccc21] (R)-N-(1-((1-methyl-1H-benzo[d]imidazol-2- yl)methyl)pyrrolidin-3-yl)isoquinolin-5-amine | 2c, 7, 8, 9, 10 |
| 2.076 | 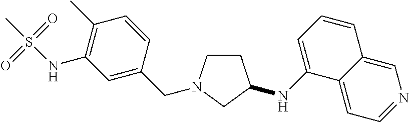 [SMILES: Cc1ccc(CN2CC[C@@H](Nc3cccc4cnccc34)C2)cc1NS(C)(=O)=O] (R)-N-(5-((3-(isoquinolin-5-ylamino)pyrrolidin-1- yl)methyl)-2-methylphenyl)methanesulfonamide | 2c, 7, 8, 9, 10, 12a, 15c, 15d |
| 2.077 | 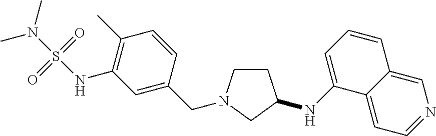 [SMILES: Cc1ccc(CN2CC[C@@H](Nc3cccc4cnccc34)C2)cc1NS(=O)(=O)N(C)C] (R)-N-(5-((3-(isoquinolin-5-ylamino)pyrrolidin-1- yl)methyl)-2-methylphenyl)-N′,N′ dimethylaminosulfamide | 2c, 7, 8, 9, 10, 12a, 15c, 15d |
| 2.078 | 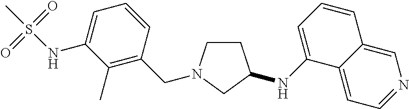 [SMILES: Cc1c(CN2CC[C@@H](Nc3cccc4cnccc34)C2)cccc1NS(C)(=O)=O] (R)-N-(3-((3-(isoquinolin-5-ylamino)pyrrolidin-1- yl)methyl)-2-methylphenyl)methanesulfonamide | 2c, 7, 8, 9, 10, 12a, 15c, 15d |
| 2.079 | 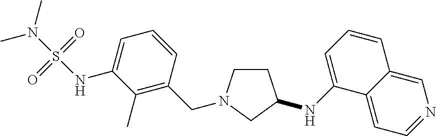 [SMILES: Cc1c(CN2CC[C@@H](Nc3cccc4cnccc34)C2)cccc1NS(=O)(=O)N(C)C] (R)-N-(3-((3-(isoquinolin-5-ylamino)pyrrolidin-1- yl)methyl)-2-methylphenyl)-N′,N′ dimethylaminosulfamide | 2c, 7, 8, 9, 10, 12a, 15c, 15d |
| 2.080 | 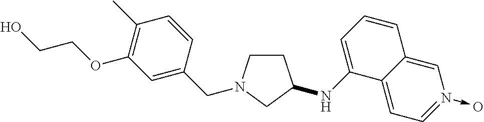 [SMILES: Cc1ccc(CN2CC[C@@H](Nc3cccc4c[n+]([O-])ccc34)C2)cc1OCCO] (R)-5-(1-(3-(2-hydroxyethoxy)-4- methylbenzyl)pyrrolidin-3-ylamino)isoquinoline 2-oxide | 2b, 6b, 8, 9, 10, 12a, 15b, 15d |
| 2.081 | 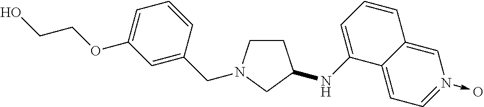 [SMILES: [O-][n+]1ccc2c(N[C@@H]3CCN(Cc4cccc(OCCO)c4)C3)cccc2c1] (R)-5-(1-(3-(2-hydroxyethoxy)benzyl)pyrrolidin-3- ylamino)isoquinoline 2-oxide | 2b, 6b, 8, 9, 10, 12a, 15b, 15d |
| 2.082 | 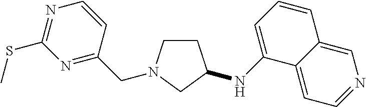 [SMILES: CSc1nccc(CN2CC[C@@H](Nc3cccc4cnccc34)C2)n1] (R)-N-(1-((2-(methylthio)pyrimidin-4- yl)methyl)pyrrolidin-3-yl)isoquinolin-5-amine | 2c, 7, 8, 9, 10, 12b, 15c, 15e |
| 2.083 | 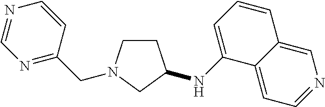 [SMILES: c1cc(N[C@@H]2CCN(Cc3ccncn3)C2)c2ccncc2c1] (R)-N-(1-(pyrimidin-4-ylmethyl)pyrrolidin-3- yl)isoquinolin-5-amine | 2c, 7, 8, 9, 10 |
| 2.084 | 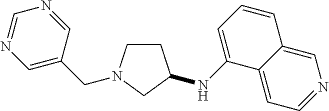 [SMILES: c1cc(N[C@@H]2CCN(Cc3cncnc3)C2)c2ccncc2c1] (R)-N-(1-(pyrimidin-5-ylmethyl)pyrrolidin-3- yl)isoquinolin-5-amine | 2c, 7, 8, 9, 10 |
| 2.085 | 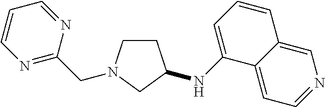 [SMILES: c1cnc(CN2CC[C@@H](Nc3cccc4cnccc34)C2)nc1] (R)-N-(1-(pyrimidin-2-ylmethyl)pyrrolidin-3- yl)isoquinolin-5-amine | 2c, 7, 8, 9, 10 |
| 2.086 | 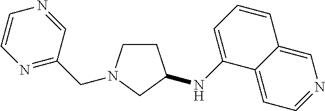 [SMILES: c1cc(N[C@@H]2CCN(Cc3cnccn3)C2)c2ccncc2c1] (R)-N-(1-(pyrazin-2-ylmethyl)pyrrolidin-3- yl)isoquinolin-5-amine | 2c, 7, 8, 9, 10 |
| 2.087 | 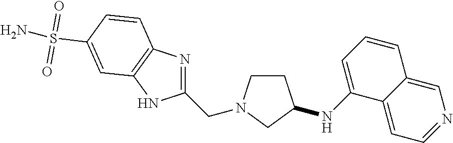 [SMILES: NS(=O)(=O)c1ccc2nc(CN3CC[C@@H](Nc4cccc5cnccc45)C3)[nH]c2c1] (R)-2-((3-(isoquinolin-5-ylamino)pyrrolidin-1- yl)methyl)-1H-benzo[d]imidazol-6-sulfonamide | 2c, 7, 8, 9, 10, 12b, 15c, 15e |
| 2.088 | 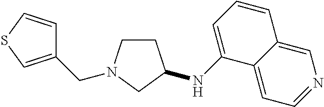 [SMILES: c1cc(N[C@@H]2CCN(Cc3ccsc3)C2)c2ccncc2c1] (R)-N-(1-(thiophen-3-ylmethyl)pyrrolidin-3- yl)isoquinolin-5-amine | 2c, 7, 8, 9, 10 |
| 2.089 | 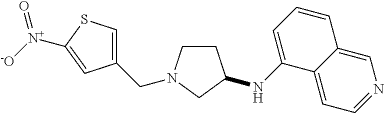 (R)-N-(1-((5-nitrothiophen-3-yl)methyl)pyrrolidin-3- yl)isoquinolin-5-amine | 2c, 7, 8, 9, 10 |
| 2.090 | 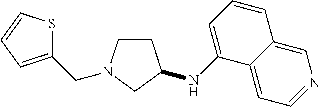 [SMILES: c1csc(CN2CC[C@@H](Nc3cccc4cnccc34)C2)c1] (R)-N-(1-(thiophen-2-ylmethyl)pyrrolidin-3- yl)isoquinolin-5-amine | 2c, 7, 8, 9, 10 |
| 2.091 | 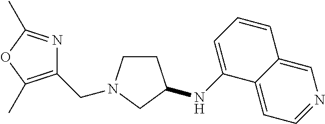 [SMILES: Cc1nc(CN2CC[C@@H](Nc3cccc4cnccc34)C2)c(C)o1] (R)-N-(1-((2,5-dimethyloxazol-4-yl)methyl)pyrrolidin-3- yl)isoquinolin-5-amine | 2c, 7, 8, 9, 10 |
| 2.092 | 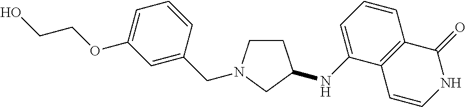 [SMILES: O=c1[nH]ccc2c(N[C@@H]3CCN(Cc4cccc(OCCO)c4)C3)cccc12] (R)-5-(1-(3-(2-hydroxyethoxy)benzyl)pyrrolidin-3- ylamino)isoquinolin-1(2H)-one | 2b, 6b, 8, 9, 10, 12a, 15b, 15d |
| 2.093 | 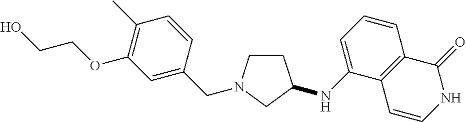 [SMILES: Cc1ccc(CN2CC[C@@H](Nc3cccc4c(=O)[nH]ccc34)C2)cc1OCCO] (R)-5-(1-(3-(2-hydroxyethoxy)-4- methylbenzyl)pyrrolidin-3-ylamino)isoquinolin-1(2H)- one | 2b, 6b, 8, 9, 10, 12a, 15b, 15d |
| 2.094 | 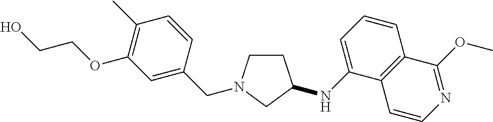 [SMILES: COc1nccc2c(N[C@@H]3CCN(Cc4ccc(C)c(OCCO)c4)C3)cccc12] (R)-2-(5-((3-(1-methoxyisoquinolin-5- ylamino)pyrrolidin-1-yl)methyl)-2- methylphenoxy)ethanol | 2b, 6b, 8, 9, 10, 12a, 15b, 15d |
| 2.095 | 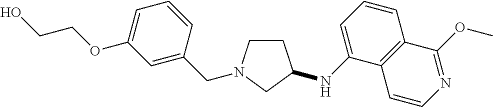 [SMILES: COc1nccc2c(N[C@@H]3CCN(Cc4cccc(OCCO)c4)C3)cccc12] (R)-2-(3-((3-(1-methoxyisoquinolin-5- ylamino)pyrrolidin-1-yl)methyl)phenoxy)ethanol | 2b, 6b, 8, 9, 10, 12a, 15b, 15d |
| 2.096 | 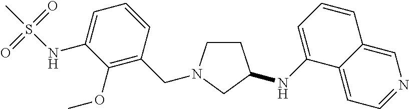 [SMILES: COc1c(CN2CC[C@@H](Nc3cccc4cnccc34)C2)cccc1NS(C)(=O)=O] (R)-N-(3-((3-(isoquinolin-5-ylamino)pyrrolidin-1- yl)methyl)-2-methoxyphenyl)methanesulfonamide | 2c, 7, 8, 9, 10, 12a, 15c, 15d |
| 2.097 | 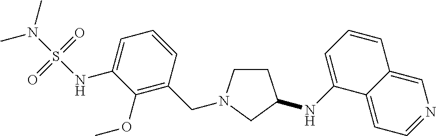 [SMILES: COc1c(CN2CC[C@@H](Nc3cccc4cnccc34)C2)cccc1NS(=O)(=O)N(C)C] (R)-N-(3-((3-isoquinolin-5-ylamino)pyrrolidin-1- yl)methyl)-2-methoxyphenyl)-N′,N′ dimethylsulfamide | 2c, 7, 8, 9, 10, 12a, 15c, 15d |
| 2.098 | 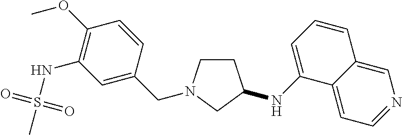 [SMILES: COc1ccc(CN2CC[C@@H](Nc3cccc4cnccc34)C2)cc1NS(C)(=O)=O] (R)-N-(5-((3-(isoquinolin-5-ylamino)pyrrolidin-1- yl)methyl)-2-methoxyphenyl)methanesulfonamide | 2c, 7, 8, 9, 10, 12a, 15c, 15d |
| 2.099 | 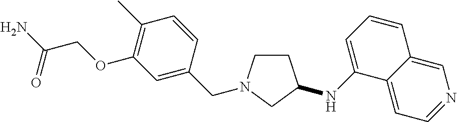 [SMILES: Cc1ccc(CN2CC[C@@H](Nc3cccc4cnccc34)C2)cc1OCC(N)=O] (R)-2-(5-((3-(isoquinolin-5-ylamino)pyrrolidin-1- yl)methyl)-2-methylphenoxy)acetamide | 2c, 7, 8, 9, 10, 12a, 15c, 15d |
| 2.100 | 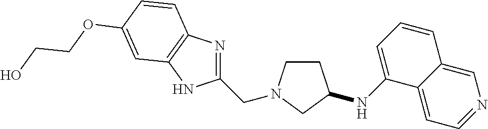 [SMILES: OCCOc1ccc2nc(CN3CC[C@@H](Nc4cccc5cnccc45)C3)[nH]c2c1] (R)-2-(2-((3-isoquinolin-5-ylamino)pyrrolidin-1- yl)methyl)-1H-benzo[d]imidazol-6-yloxy)ethanol | 2c, 7, 8, 9, 10, 12a, 15c, 15d |
| 3.001 | 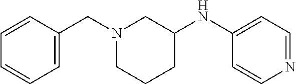 [SMILES: c1ccc(CN2CCCC(Nc3ccncc3)C2)cc1] N-(1-benzylpiperidin-3-yl)pyridin-4-amine | 3c, 7, 8, 9, 10 |
| 3.002 | 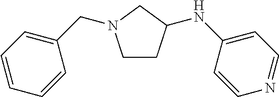 [SMILES: c1ccc(CN2CCC(Nc3ccncc3)C2)cc1] N-(1-benzylpyrrolidin-3-yl)pyridin-4-amine | 3c, 7, 8, 9, 10 |
| 4.001 | 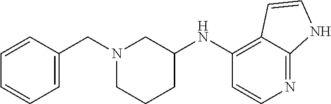 [SMILES: c1ccc(CN2CCCC(Nc3ccnc4[nH]ccc34)C2)cc1] N-(1-benzylpiperidin-3-yl)-1H-pyrrolo[2,3-b]pyridin-4- amine | 4c, 7, 8, 9, 10 |
| 4.002 | 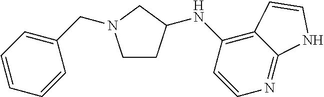 [SMILES: c1ccc(CN2CCC(Nc3ccnc4[nH]ccc34)C2)cc1] N-(1-benzylpyrrolidin-3-yl)-1H-pyrrolo[2,3-b]pyridin-4- amine | 4c, 7, 8, 9, 10 |
| 5.001 | 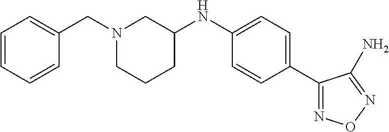 [SMILES: Nc1nonc1-c1ccc(NC2CCCN(Cc3ccccc3)C2)cc1] 4-(4-(1-benzylpiperidin-3-ylamino)phenyl)-1,2,5- oxadiazol-3-amine | 5a, 7, 8, 9, 10 |
| 5.002 | 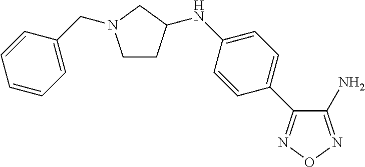 [SMILES: Nc1nonc1-c1ccc(NC2CCN(Cc3ccccc3)C2)cc1] 4-(4-(1-benzylpyrrolidin-3-ylamino)phenyl)-1,2,5- oxadiazol-3-amine | 5a, 7, 8, 9, 10 |

Preferred ROCK inhibitor compounds of this invention include, but are not limited to the ROCK inhibitor compounds of embodiments 5, 14, 15, 16, 17, 18, 19, 20, and 21 as described above, and their associated salts, tautomers, solvates, or hydrates. In particular, preferred Compounds include 1.074, 1.075, 1.076, 1.077, 1.079, 1.091, 1.093, 1.108, 1.109, 1.123, 1.124, 1.126, 1.131, 1.132, 1.133, 1.134, 1.135, 1.136, 1.137, 1.138, 1.141, 1.148, 1.149, 1.150, 1.152, 1.153, 1.155, 1.156, 1.157, 1.158, 1.161, 1.162, 1.163, 1.164, 1.165, 1.166, 1.171, 1.173, 1.175, 1.176, 1.186, 1.193, 1.195, 1.197, 1.200, 1.206, 1.212, 1.213, 1.215, 1.217, 1.219, 1.223, 1.233, 1.236, 1.237, 1.238, 1.239, 1.249, 1.252, 1.253, 1.258, 1.259, 1.260, 1.261, 1.262, 1.270, 1.273, 1.275, 1.277, 1.281, 2.025, 2.026, 2.031, 2.038, 2.039, 2.041, 2.046, 2.047, 2.054, 2.055, 2.057, 2.058, 2.059, 2.060, 2.061, 2.064, 2.065, 2.066, 2.067, 2.068, 2.069, 2.072, 2.073, 2.076, 2.077, 2.078, 2.079, 2.082, 2.096, 2.097, and 2.099.

### Pharmaceutical Formulations

The present invention provides a pharmaceutical formulation comprising compounds of Formula I or II and a pharmaceutically acceptable carrier. Pharmaceutically acceptable carriers can be selected by those skilled in the art using conventional criteria. Pharmaceutically acceptable carriers include, but are not limited to, saline solution, aqueous electrolyte solutions, isotonicity modifiers, water polyethers such as polyethylene glycol, polyvinyls such as polyvinyl alcohol and povidone, cellulose derivatives such as methylcellulose and hydroxypropyl methylcellulose, polymers of acrylic acid such as carboxypolymethylene gel, polysaccharides such as dextrans, and glycosaminoglycans such as sodium hyaluronate and salts such as sodium chloride and potassium chloride.

The pharmaceutical formulation useful for the present invention in general is an aqueous solution comprising water, suitable ionic or non-ionic tonicity modifiers, suitable buffering agents, and a compound of Formula I or II. In one embodiment, the compound is at 0.005 to 3% w/v, and the aqueous solution has a tonicity of 200-400 mOsm/kG and a pH of 4-9.

In one embodiment, the tonicity modifier is ionic such as NaCl, for example, in the amount of 0.5-0.9% w/v, preferably 0.6-0.9% w/v.

In another embodiment, the tonicity modifier is non-ionic, such as mannitol, dextrose, in the amount of at least 2%, or at least 2.5%, or at least 3%, and no more than 7.5%; for example, in the range of 3-5%, preferably 4-5% w/v.

The pharmaceutical formulation can be sterilized by filtering the formulation through a sterilizing grade filter, preferably of a 0.22-micron nominal pore size. The pharmaceutical formulation can also be sterilized by terminal sterilization using one or more sterilization techniques including but not limited to a thermal process, such as an autoclaving process, or a radiation sterilization process, or using pulsed light to produce a sterile formulation. In one embodiment, the pharmaceutical formulation is a concentrated solution of the active ingredient; the formulation can be serially diluted using appropriate acceptable sterile diluents prior to administration.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents such as those set forth above, and flavoring agents can be added to provide palatable oral preparations. These compositions can be preserved by the addition of an anti-oxidant such as ascorbic acid.

Pharmaceutical compositions of the invention can be in the form of oil-in-water emulsions. The oily phase can be a vegetable oil, for example olive oil or arachis oil, or a mineral oil, for example liquid paraffin or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions can also contain sweetening and flavoring agents.

Pharmaceutical compositions of the invention can be in the form of an aerosol suspension of respirable particles comprising the active compound, which the subject inhales. The respirable particles can be liquid or solid, with a particle size sufficiently small to pass through the mouth and larynx upon inhalation. In general, particles having a size of about 1 to 10 microns, preferably 1-5 microns, are considered respirable.

The pharmaceutical formulation for systemic administration such as injection and infusion is generally prepared in a sterile medium. The active ingredient, depending on the vehicle and concentration used, can either be suspended or dissolved in the vehicle. Adjuvants such as local anesthetics, preservatives and buffering agents can also be dissolved in the vehicle. The sterile injectable preparation can be a sterile injectable solution or suspension in a non-toxic acceptable diluent or solvent. Among the acceptable vehicles and solvents that can be employed are sterile water, saline solution, or Ringer's solution.

The pharmaceutical compositions for oral administration contain active compounds in the form of tablets, lozenges, aqueous or oily suspensions, viscous gels, chewable gums, dispersible powders or granules, emulsion, hard or soft capsules, or syrups or elixirs.

For oral use, an aqueous suspension is prepared by addition of water to dispersible powders and granules with a dispersing or wetting agent, suspending agent one or more preservatives, and other excipients. Suspending agents include, for example, sodium carboxymethylcellulose, methylcellulose and sodium alginate. Dispersing or wetting agents include naturally-occurring phosphatides, condensation products of an allylene oxide with fatty acids, condensation products of ethylene oxide with long chain aliphatic alcohols, condensation products of ethylene oxide with partial esters from fatty acids and a hexitol, and condensation products of ethylene oxide with partial esters derived from fatty acids and hexitol anydrides. Preservatives include, for example, ethyl, and n-propyl p-hydroxybenzoate. Other excipients include sweetening agents (e.g., sucrose, saccharin), flavoring agents and coloring agents. Those skilled in the art will recognize the many specific excipients and wetting agents encompassed by the general description above.

For oral application, tablets are prepared by mixing the active compound with nontoxic pharmaceutically acceptable excipients suitable for the manufacture of tablets. These excipients can be, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets can be uncoated or they can be coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil. Formulation for oral use can also be presented as chewable gums by embedding the active ingredient in gums so that the active ingredient is slowly released upon chewing.

The pharmaceutical compositions can be in the form of suppositories, which are prepared by mixing the active ingredient with a suitable non-irritating excipient that is solid at ordinary temperatures but liquid at the rectal temperature and will thus melt in the rectum to release the compound. Such excipients include cocoa butter and polyethylene glycols.

### Method of Treating Cardiovascular Diseases Using Rho Kinase Inhibitor Compounds

The present invention is useful in treating diseases associated with excessive cell proliferation, tissue remodeling, inflammation, and vasoconstriction. The present invention is particularly effective in treating cardiovascular disease such as stent restenosis and thrombosis, vascular thrombosis, cerebral vasospasm, atherosclerosis, systemic hypertension, cardiac hypertrophy, and sexual dysfunction.

### Stent Restenosis and Thrombosis

The inventors have discovered that compounds of Formula I or II are useful in suppression of proliferation ability of vascular smooth muscle cells (VSMC). Smooth muscle proliferation and remodeling play a role in the pathophysiology of thrombosis. Therefore, the inventors have discovered that compounds of Formula I or II provide a method to prevent restenosis and thrombosis of stented blood vessels, due to its anti-proliferative effects on vascular smooth muscle cells and the disaggregation of platelets and the inhibition of shape change that precedes aggregation.

The present invention is directed to a method of preventing or treating restenosis and thrombosis of stented blood vessels. The method comprises the steps of first identifying a subject with one or more blocked or narrowed blood vessels, then placing one or more Formula I or II compound-coated stents on one or more affected blood vessels, whereby the blood flow in the affected blood vessel is restored and to the restenosis and thrombosis of the stented vessel is prevented.

The present invention further provides stents coated with Rho kinase inhibitor compound of Formula I or II. Stents coated with such compounds that inhibit Rho kinase-mediated regulation of smooth muscle cell proliferation and motility, inhibit the platelet shape change that precedes aggregation and increase platelet dissagregation induced by thrombin represent a novel treatment for the prevention of restenosis and thrombosis following stent placement.

Indicia of efficacy for the prevention of restenosis and thrombosis following stent placement by the present method include demonstrable improvement in measurable signs, symptoms and other variables clinically relevant to stent restenosis and thrombosis. Such improvements include: inhibition of in-stent neointimal hyperplasia, decrease of neointimal coverage of the stent, decrease in reduction of in-stent lumen for up to 1 or 2 years as measured by quantitative angiography in the lesion zone and in the stent zone, reduction in angiographic restenosis, reduction of the rate of target vessel revascularization, reduction in major adverse cardiac events associated with target lesion revascularization, decrease of in-stent thrombosis, reduction of myocardial infarction, and decrease in the rate of target vessel revascularization.

### Rho Kinase Inhibitor Coated-Stents

Coating stents with pharmaceutical agents has an inherent advantage over systemic administration, due to the ability to precisely deliver a much lower dose of the drug to the target area thus achieving high tissue concentration while minimizing the risk of systemic toxicity.

The present invention is also directed to a drug-eluting stent, which is a stent coated with one or more Rho kinase inhibitor compounds of Formula I or II, or a pharmaceutically acceptable salt, solvate, or hydrate thereof. When the stent is placed in a narrowed or damaged arterial vessel, a therapeutically effective amount of the compound(s) is eluted continuously from the stent to the local environment of the stent. Local delivery to vasculature facilitates the achievement of high regional drug concentrations, achieves a continuous exposure of the tissue to the drug, and reduces potential adverse effects and systemic toxicity due to lower systemic doses. The drug can be targeted directly to the required site. A therapeutically effective amount of the Formula I or II compound is an amount that is effective in preventing restenosis and thrombosis and maintaining blood flow rate of the stented vessel, by decreasing in shear forces, relaxing vascular smooth muscle, and reducing narrowing of the vascular lumen restenosis.

The stent is coated with one or more compounds of Formula I or II. In one embodiment, the stent is coated with a carrier that comprises at least one Formula I or II compound. The carrier is usually a biocompatible and non-toxic polymer. The polymer is preferably a biodegradable polymer or a biostable polymer.

Biodegradable polymers suitable for this invention can be chosen from, but are not limited to, polycaprolactone, polylactic acid (D/L or L), poly(lactide-co-glycolide), poly(hydroxybutyrate), poly(hydroxybutyrate-covalerate), polydioxanone, polyorthoester, polyanhydride, poly(glycolic acid), poly(glycolic acid-cotrimethylene carbonate), polyphosphoester, polyphosphoester urethane, poly(amino acids), poly(trimethylene carbonate), poly(iminocarbonate), cyanoacrylates, polyalkylene oxalates, polyphosphazenes, and aliphatic polycarbonates. Alternately, natural biomolecules such as cellulose, starch, dextran, hyaluronic acid, and collagen can also be used.

Biostable polymers can be chosen from, but are not limited to, polyurethanes, polyesters, polyamides, polyolefins, polycaprolactam, polyvinyl chloride, polyvinyl alcohol, poly(ethylene-vinyl alcohol), polyethers, silicones, acrylate polymers and copolymers, polyvinylmethyl ether, polyimide, and polyacrylonitrile.

The concentration of the Formula I or II compounds in the stent is in general in the range of 0.001-20, preferably 0.01-10, and more preferably 0.1-5 μg/mm². Alternatively, the concentration of the Formula I or II compound in the stent is 1-500, preferably 10-100 μg/mm. Muni, et al. (American Heart Journal, 149:415-433, (2005)) have reported stent drug carriers, drug concentrations, stent sizes, and types of lesions; the article is incorporated herein by reference in its entirety.

In one embodiment, the elution of the Formula I or II compound is slow release and long-acting, i.e., the compound is eluted constantly and provides a local therapeutically effective amount at least until the epithelium damaged by the stent placement is healed. The local elution of the Formula I or II compound into the tissue surrounding the stent is preferably over a period of 3 to 6 months, and preferably 6 months. When the stent is coated with biodegradable polymers, the elution of the compound from the stent directly relates to the rate of degradation of the polymer.

Formula I or II compounds useful in this invention are compounds that do not require hepatic, renal or any other metabolic transformation to become pharmacologically active. The compound can be a prodrug if the conversion of the prodrug into the active species is carried out locally in the release area. For example, an ester prodrug can be converted into an active drug by tissue esterases such as endothelial esterases.

Applicants have discovered the therapeutic benefits of Formula I or II compound-eluting stents. The elution of Formula I or II compounds to local stented tissues can prevent the stenosis of stented arteries by relaxing the arterial smooth muscle, which results in an increase in blood flow rate of the stented artery and a decrease in shear forces that could promote thrombosis. Additionally, the inhibition of vascular smooth muscle contraction in stented arteries can decrease the risk of ischemia and thrombosis. Therefore, the use of stents coated with Formula I or II compound improves the therapeutic benefit of current stents by decreasing the incidence of thrombosis and restenosis and improving the flow rate of perfusion of the stented artery due to the relaxing activity of smooth muscle cells.

Formula I or II compound-eluting stents can be used as in situ antithrombotics to decrease the risk of stent thrombosis by a constant delivery of the Formula I or II compound for several months. This treatment decreases the risk of thrombosis by inhibiting the aggregation of platelets in the stented artery. Formula I or II compounds are useful to coat all types of stents, including coronary stents, cerebral arterial stents (basilar or vertebral arteries), other arterial stents (aortic, carotid, renal, peripheral, etc), and vein stents (portal, renal, including vein graft conduits). Peripheral artery is defined as an artery that carries blood to upper and lower extremities. Formula I or II compound-eluting stents are useful for saphenous vein grafts previously grafted in coronary arteries, which have reduced patency due to restenosis or thrombosis. Preferred stents for this invention are coronary stents.

Formula I or II compound-eluting stents are useful in preventing the thrombosis and restenosis observed on patients after placement of bare metal and other drug-eluting stents.

The present invention provides a method for treating blocked or narrowed arteries. The method comprises the step of placing a Formula I or II compound-eluting stent in a narrowed or blocked artery of a patient, whereby a therapeutically effective amount of the compound is eluted to the stented area, whereby the blood flow is resumed by the stent and the restenosis and thrombosis are prevented by the Formula I or II compound. The artery can be, for example, coronary artery, cerebral artery, or peripheral artery, which has been narrowed or blocked by a plaque or a plaque rupture, respectively. The inserted stent delivers Formula I or II compound locally to the stented area, and decreases the incidence of thrombosis and restenosis. The method optionally comprises the step of monitoring the patient to ensure patency of the stented artery. For example, when the stent is inserted into the coronary artery, the patient can be monitored by clinical symptoms of the cardiac function, e.g., electrocardiogram (EKG), to determine if the blood flow in the heart muscle is restored. When the stent is inserted into the carotid artery, the patient can be monitored by ultrasound to determine if the narrowed artery is restored, and by evaluation of clinical symptoms such as headache, facial droop, loss of coordination, vertigo and depressed mental status. When the stent is inserted into the cerebral arteries, the patient can be monitored by neurological examinations including clinical symptoms such as headache, facial droop, loss of coordination, vertigo and depressed mental status.

Preparation of Stents Coated with Formula I or II Compound

Stents are frequently made from stainless steel. Stents can be made of any biocompatible metal, including, but not limited to, steel, cobalt, titanium, tantalum, chromium, zirconium, niobium, tungsten, platinum, palladium, vanadium, silver, gold, molybdenum, nickel, or magnesium, and alloys thereof in any combination. Alternately, stents can be constructed of non-metallic biocompatible materials, such as bioabsorbable or biostable polymers. The preparation of drug-eluting stents has been described in Kavanagh, et al. (Pharmacology & Therapeutics, 102: 1-15, 2004), Doorty, et al. (Cardiovascular Pathology, 12: 105-110, 2003), Hossainy (U.S. Pat. No. 6,908,624). Both articles are incorporated herein by reference in their entirety.

In general, Formula I or II compounds of the present invention are preferably not attached directly (covalently of non-covalently) to the surface of an unmodified stent. In order to deliver the compounds of the present invention to the site of action, the stent is preferably coated with an organic or inorganic polymer (or polymers) or some other substance (such as an inorganic coating) that is able to retain the compound to be delivered and release it at a desired rate. The nature of this retention can be covalent or non-covalent, with the latter being preferred. In one embodiment, the stent is first modified by coating it with an inorganic substance or an organic or inorganic polymer which is capable of binding the compound to the stent surface. For example, when the Formula I or II compound bears a phosphate or other acidic moiety, the stent is first coated with a substance or a polymer that bears a basic moiety, and the compound is bound to the modified stent by an ionic interaction. When the Formula I or II compound bears a basic moiety, the stent is first coated with a substance or a polymer that bears an acidic moiety, and the compound is bound to the modified stent by an ionic interaction.

In another embodiment, the Formula I or II compound is first incorporated into a compatible polymer matrix, which is then used to coat a stent. The advantage of this approach is that the elution of the Formula I or II compound from the stent depends on the property of the polymer, thus one can select a suitable polymer, which provides controlled and sustained release of the Formula I or II compound to the site of action. The polymer can be hydrophilic, hydrophobic, biodegradable, or biostable, thus one can further select a polymer to optimize the desired therapeutic effect.

The present invention provides a composition comprising at least one biodegradable polymer and at least one Formula I or II compound, wherein said biodegradable polymer is selected from the group consisting of polycaprolactone, polylactic acid, poly(lactide-co-glycolide), poly(hydroxybutyrate), poly(hydroxybutyrate-covalerate), polydioxanone, polyorthoester, polyanhydride, poly(glycolic acid), poly(glycolic acid-cotrimethylene carbonate), polyphosphoester, polyphosphoester urethane, poly(amino acids), poly(trimethylene carbonate), poly(iminocarbonate), cyanoacrylates, polyalkylene oxalates, polyphosphazenes, aliphatic polycarbonates, cellulose, starch, dextran, hyaluronic acid, and collagen.

The present invention further provides a composition comprising at least one biostable polymer and at least one Formula I or II compound, wherein said biostable polymer is selected from the group consisting of polyurethanes, polyesters, polyamides, polyolefins, polycaprolactam, polyvinyl chloride, polyvinyl alcohol, poly(ethylene-vinyl alcohol), polyethers, silicones, acrylate polymers and copolymers, polyvinylmethyl ether, polyimide, and polyacrylonitrile.

When biodegradable polymers are used, the Formula I or II compound is incorporated into the polymer matrix and released in a controlled manner by a gradual degradation of the polymer matrix. This degradation can occur by various processes, including hydrolysis, metabolism, bulk erosion, or polymer surface erosion. When biostable polymers are used, the Formula I or II compound is uniformly distributed in the polymer or encapsulated within the polymer, from which the compound is eluted via diffusion processes or through pores of the polymer structure.

The Formula I or II compound can be incorporated into the polymer via processes known to those skilled in the art. These include, but are not limited to, encapsulation of the compound within a polymer matrix during polymer synthesis prior to application of the polymer to the stent, dissolving both polymer and the compound in an appropriate solvent and applying the solution to a stent, after which the solvent is allowed to evaporate and the stent is allowed to dry, or pre-coating a stent with a polymer, after which the therapeutic agent is applied as a solution in an appropriate solvent. Application methods can include, but are not limited to, spraying, dipping, or spin coating processes.

### Vascular Thrombosis

The inventors have discovered that Compounds of Formula I or II inhibit cell actin cytoskeleton reorganization, platelet adhesion, and platelet shape change, thus they inhibit the formation of stable platelet aggregates. Furthermore, the inventors have discovered that Compounds of Formula I or II are useful in regulating platelet function and preventing or treating vascular thrombosis.

The present invention is directed to a method of treating vascular thrombosis. The method comprises the steps of first identifying a subject suffering from vascular thrombosis, then administering to the subject an effective amount of a compound of Formula I or II to prevent vascular thrombosis.

Indicia of efficacy for preventing or treating vascular thrombosis by the present method include demonstrable improvement in measurable signs, symptoms and other variables clinically relevant to vascular thrombosis. Such improvements include: reduction in the incidence of heart attack or myocardial infarction, decrease in the incidence of unstable angina, decrease in the incidence of heart failure, decrease in the incidence of arrhythmia, decrease in the incidence of stroke, decrease in the incidence of peripheral vascular disease, decrease of pain during exercise (intermittent claudication). Formula I or II compounds are useful to prevent venous and arterial thrombosis. Preferred use of Formula I or II compounds is for arterial thrombosis

### Cerebral Vasospasm

The inventors have discovered that compounds of Formula I or II are useful in decreasing the calcium sensitivity of the smooth muscle, thus inhibiting vascular smooth muscle contraction. The inventors have further discovered that compounds of Formula I or II are useful in treating cerebral vasospasms.

The present invention is directed to a method of treating cerebral vasospasm. The method comprises the steps of first identifying a subject suffering from cerebral vasospasm, and then administering to the subject an effective amount of a compound of Formula I or II to treat cerebral vasospasm.

Indicia of efficacy for treating cerebral vasospasms by the present method include decrease in severe headache, decrease in nausea and/or vomiting, decrease in symptoms of meningeal irritation (eg, neck stiffness, low back pain, bilateral leg pain), decrease in photophobia and visual changes, improvements in consciousness as measured by the Glasgow coma scale, decreases convulsions, decrease in memory loss, decreased hemiparesis, decreased aphasia, decreased presence of creatine kinase-BB isoenzyme activity in the cerebrospinal fluid, decrease in blood present in the cerebrospinal fluid as measured by lumbar puncture, CT scan or MRI, improvement in vessel diameter by MR angiography and CT angiography, improvement in ability to vocalize words, improvement in ability to understand spoken or written words, survival, absence of hypodense lesions on CT that are consistent with infarction, improvements in the Fisher grade (an index of vasospasm risk based upon a CT-defined hemorrhage pattern), improvement in the Claassen grading system (an index of the risk of delayed cerebral ischemia due to vasospasm), and improvement in the Hunt and Hess grading system for neurological symptoms.

### Atherosclerosis

The inventors have discovered that compounds of Formula I or II, which inhibit Rho kinase activity, have properties that lead to a dilatory effect on arteries, thus relaxing the tissue and leading to higher blood flow, reduce thickening of the arteries and reduce the plaque induced inflammation. The inventors have therefore discovered that compounds of Formula I or II provide a method of preventing or treating atherosclerosis of blood vessels.

The present invention is directed to a method of treating atherosclerosis. The method comprises the steps of first identifying a subject suffering from atherosclerosis, then administering to the subject an effective amount of a compound of Formula I or II to treat atherosclerosis.

Indicia of efficacy for treating atherosclerosis by the present method include demonstrable improvements in measurable signs, symptoms and other variables clinically relevant to atherosclerosis. Improvements of the disease include a reduction in the number of instances clinical events such as heart attack, chest pain on exertion (angina), cerebrovascular disease leading to transient ischemic attack, stroke, permanent brain injury, abdominal aortic aneurysm, erectile dysfunction, blood clots, decreased pulse in the feet, and decrease in pain in calf muscles (claudication) upon normal activity such as walking. Improvements of the disease also include a reduction or elimination of oxidized phospholipids, a reduction in atherosclerotic plaque formation and rupture, a decrease in hypertension, and a decrease in inflammatory protein biosynthesis. Improvement in circulation via reduced arterial inflammation leads to better wound healing, increased blood flow to the intestines, kidneys, and other vital organs.

### Systemic Hypertension

The inventors have discovered that compounds of Formula I or II, which inhibit Rho kinase activity, have anti-proliferative effects on vascular smooth muscle cells and have ability to reduce proinflammatory mediators associated with leukocyte activation and migration. The inventors have therefore discovered that compounds of Formula I or II provide a method of treating arterial hypertension.

The present invention is directed to a method of treating systemic hypertension. The method comprises the steps of first identifying a subject suffering from systemic hypertension, then administering to the subject an effective amount of a compound of Formula I or II to treat systemic hypertension.

Indicia of efficacy for treating systemic hypertension include demonstrable improvement in measurable signs, symptoms, and other variables clinically relevant to systemic hypertension. Such improvements include reduction of blood pressure below 140/90 mm Hg, improvement in signs of hypertensive retinopathy, improved blood supply to the organs, reduction of headaches, heart hypertrophy, drowsiness, confusion, numbness, tingling in the hands and feet, coughing blood, nosebleeds and reductions in shortness of breath. Some of the signs of improved organ function can be a reduction in blurred or reduced vision acuity as well as reduced pain upon urination. An additional sign of efficacy is a reduction in occlusions or emboli that can lead to strokes of the brain or myocardial infarctions of the heart.

### Cardiac Hypertrophy

The inventors have discovered that Compounds of Formula I or II inhibit cytokinesis, cytokine and chemokine secretion, proliferation and cell motility. Therefore, compounds of Formula I or II are useful in regulating the inflammation and remodeling that occur in cardiac hypertrophy.

The present invention is directed to a method of treating cardiac hypertrophy. The method comprises the steps of first identifying a subject suffering from cardiac hypertrophy, then administering to the subject an effective amount of a compound of Formula I or II to treat cardiac hypertrophy.

Indicia of efficacy for treating cardiac hypertrophy by the present method include demonstrable improvement in measurable signs, symptoms and other variables clinically relevant to cardiac hypertrophy. Such signs of improvement include rapid regression or complete reversal of cardiac hypertrophy over a period of a few months, gradual regression of cardiac hypertrophy over a period of 2-3 years, the relative reduction in left ventricular mass index, cardiac function after regression of cardiac hypertrophy, reduced number of ventricular premature beats, reduced vulnerability to inducible ventricular fibrillation, reduced evidence of diastolic dysfunction, reduced risk of cardiovascular morbidity, reduced risk of cardiovascular mortality, and increase in general quality of life. Parameters for measurement of efficacy include the reduction in left ventricular mass measured by echocardiography, reduction in wall thickness values obtained from M-mode or 2D images from the parasternal views, number of ventricular premature beats.

### Sexual Dysfunction

The inventors have discovered that compounds of Formula I or II, which inhibit Rho kinase activity, inhibit vasoconstriction leading to the relaxation of smooth muscles in male or female erectile tissue. The inventors have therefore discovered that compounds of Formula I or II provide a method of preventing or treating male and female sexual dysfunction.

As used herein, sexual dysfunction includes erectile dysfunction in men, where erectile dysfunction is defined as the inability to achieve and maintain sufficient rigidity of the penis to permit penetration of the sexual partner during intercourse. In females, sexual dysfunction includes a failure of clitoral erection and/or a failure to attain (or maintain) sexually stimulated congestion of blood in the walls of the vagina, which results in inadequate vaginal lubrication. Thus, sexual dysfunction comprises both male and female sexual dysfunction which is due, at least in part, to lack of necessary blood flow in the erectile tissue of sexual organs.

The present invention is directed to a method of treating male and female sexual dysfunction. The method comprises the steps of first identifying a subject suffering from dysfunction, then administering to the subject an effective amount of a compound of Formula I or II to treat sexual dysfunction in the subject.

The present invention is suitable for treating sexual dysfunction which arises due to a variety of causes. Sexual dysfunction (in both men and women) may arise as a result of reduced hormonal levels, psychological reasons, or physiological factors. For example, hypertension is often associated with a high prevalence of erectile dysfunction, and the drugs used to treat hypertension may cause erectile dysfunction.

The present invention targets inhibition of vasoconstrictors, and thereby provides an alternate approach for inducing smooth muscle relaxation of sexual organs.

An effective amount of a Formula I or II compound is administered to a patient in need of such treatment. The patient either already has the symptoms of at least one above-mentioned disease, or is identified as being at risk of at least one above-mentioned disease. The compound is administered at a frequency that achieves desired efficacy. What constitutes desired efficacy is determined by a physician or other health-care professional. Whether or not sufficient efficacy has been reached is determined by indicia of efficacy for the specific disease. After an initial dose, additional doses are optionally administered if judged to be necessary by a health-care professional.

### Methods of Administration

The present invention is particularly effective in treating cardiovascular diseases or conditions such as stent restenosis and thrombosis, vascular thrombosis, cerebral vasospasm, atherosclerosis, systemic hypertension, cardiac hypertrophy, and sexual dysfunction. Any method of delivering the compound to the target tissues, including systemic administration, is suitable for the present invention.

In one embodiment, the active compound is delivered by systemic administration; the compound first reaches plasma and then distributes into the target tissues. Examples of systemic administration include oral ingestion, or intravenous or subcutaneous or intraperitoneal or intrathecal or intramuscular administration.

Additional method of systemic administration of the active compound to a subject involves administering a suppository form of the active compound, such that a therapeutically effective amount of the compound reaches the target sites via systemic absorption and circulation.

Another method of systemically administering the active compounds to the subject involves administering a liquid/liquid suspension in the form of eye drops or eye wash or nasal drops of a liquid formulation, or a nasal spray of respirable particles that the subject inhales. Liquid pharmaceutical compositions of the active compound for producing a nasal spray or nasal or eye drops can be prepared by combining the active compound with a suitable vehicle, such as sterile pyrogen free water or sterile saline by techniques known to those skilled in the art.

The active compounds can also be systemically administered to the subject through absorption by the skin using transdermal patches or pads. The active compounds are absorbed into the bloodstream through the skin. Plasma concentration of the active compounds can be controlled by using patches containing different concentrations of active compounds.

For systemic administration, plasma concentrations of active compounds delivered can vary according to compounds; but are generally 1×10⁻¹⁰-1×10⁻⁴ moles/liter, and preferably 1×10⁻⁸-1×10⁻⁵ moles/liter.

Dosage levels about 0.01-140 mg per kg, preferably 0.1-100 mg/kg of body weight per day are useful in the treatment or preventions of conditions involving an inflammatory response (about 0.5 mg to about 7 g per patient per day). Preferred dosage levels are about 0.05-25, or 0.1-10 mg/kg body weight per day. The amount of active ingredient that can be combined with the carrier materials to produce a single dosage form will vary depending upon the host treated and the particular mode of administration. Dosage unit forms will generally contain between from about 1 mg to about 500 mg of an active ingredient.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more can be administered to achieve adequate steady state levels. The maximum total dose in general does not exceed about 2 g/day for a 40 to 80 kg human patient.

Frequency of dosage can also vary depending on the compound used and the particular disease treated. However, for treatment of most disorders, a dosage regimen of p.r.n, 4 times daily, three times daily, or less is preferred, with a dosage regimen of once daily or 2 times daily being particularly preferred.

In another embodiment, the active compound is delivered by inhalation, topical application, or targeted drug delivery to the target tissue. Methods of inhalation include liquid instillation, instillation as a pressurized fluid preparation via metered dose inhaler or equivalent, or inhalation of an aerosolized solution via nebulizer (preferred), inhalation of dry powder (more preferred), and directing soluble or dried material into the air stream during mechanical ventilation (also more preferred).

One administration method is administering to a subject an aerosol suspension of respirable particles comprising the active compound by inhalation. The respirable particles can be liquid or solid, with a particle size sufficiently small to pass through the mouth and larynx upon inhalation; in general, particles ranging from about 1 to 10 microns, but more preferably 1-5 microns, in size are considered respirable. The surface concentrations of active compounds delivered via inhalation can vary according to compounds; but are generally 1×10⁻¹°-1×10⁻⁴ moles/liter, and preferably 1×10⁻⁸-1×10⁻⁵ moles/liter.

It is understood, however, that the specific dose level for any particular patient will depend upon a variety of factors including the activity of the specific compound employed, the age, body weight, general health, sex, diet, time of administration, route of administration, and rate of excretion, drug combination (i.e., other drugs being administered to the patient), the severity of the particular disease undergoing therapy, and other factors, including the judgment of the prescribing medical practitioner.

Preferred compounds of the invention will have favorable pharmacological properties. Such properties include, but are not limited to bioavailability, low toxicity, low serum protein binding and desirable in vitro and in vivo half-life.

An example of targeted drug delivery is enclosure of the compound within a liposome, where the liposome is coated with a specific antibody whose antigen is expressed in the targeted lung tissue. It can be advantageous to construe a controlled delivery system of the compounds since such an inhaled product targets the site of action, presents the compound of interest in small regimented quantities and reduces/minimizes any unwanted side effects.

Another example of a delivery system includes microparticulate compositions of the compound. In such a case, the compound is formulated as a microparticulate wherein the carrier is loaded with the compound; such a preparation is then filtered through a fine porous membrane or suitable filtering medium or is exposed to solvent interchanges to produce nanoparticles. Such preparations can be freeze dried or held in suspension in an aqueous or physiologically compatible medium. The preparation so obtained can be inhaled by suitable means.

Another example of a suitable preparation includes a reconstitutable preparation. In this case, the compound is formulated in a preparation to contain the necessary adjuvant to make it physiologically compatible. Such a preparation can be reconstituted by addition of water for injection or suitable physiological fluids, admixed by simple agitation and inhaled using appropriate techniques described above.

The compounds described above can also be prepared into dry powder or equivalent inhalation powders using the well known art of super critical fluid technology. In such a case, the compound is admixed with appropriate excipients and milled into a homogenous mass using suitable solvents or adjuvants. Following this, this mass is subjected to mixing using super critical fluid technology and suitable particle size distribution achieved. The particles in the formulation need to be of a desired particle size range such that the particles can be directly inhaled into the lungs using a suitable inhalation technique or introduced into the lungs via a mechanical ventilator. Alternatively, a formulation can be designed such that the particles are large enough in size thereby offering sufficient surface area to dissolve completely in a suitable fluid when admixed together or to dissolve sufficiently enough prior to nebulization into the lungs.

The invention is illustrated further by the following examples that are not to be construed as limiting the invention in scope to the specific procedures described in them.

### EXAMPLES

### Example 1

### Rho kinase Inhibition Assay

### Relevance:

This assay demonstrates a compound's ability to inhibit ROCK2 and ROCK1 in an in vitro setting using the isolated enzyme. Compounds having ROCK2 IC₅₀ values on the order of 2 μM or below have been shown to possess efficacy in many studies using in vivo models of the disease processes described in this application.

### Protocol

Inhibition of ROCK2 and ROCK1 activity was determined using the IMAP™ Screening Express Kit (Molecular Devices product number #8073). ROCK2 enzyme (Upstate/Chemicon #14-451), ROCK1 (Upstate/Chemicon #14-601) and Flourescein tagged substrate peptide Fl-AKRRRLSSLRA (Molecular Devices product number R7184) was pre-incubated with a test compound (a Formula I or II compound or other Rho kinase compound such as fasudil, H-1152, H7, Y-27632, Y-39983) for 5 minutes in buffer containing 10 mM Tris-HCl pH 7.2, 10 mM MgCl₂, and 0.1% BSA. Following the pre-incubation, 10 μM ATP was added to initiate the reaction. After 60 minutes at room temperature, Molecular Devices IMAP™ binding solution was added to bind phosphorylated substrate. After 30 minutes of incubation in the presence of the IMAP™ beads, the fluorescence polarization was read and the ratio was reported as mP. Ki values for compounds and EC₅₀ values for ATP were calculated using the Prism software from Graphpad.

### Results:

TABLE 1
Rho kinase I and II Potency Data

| | ROCK1 Ki, | ROCK1 Ki, | ROCK2 Ki, | ROCK2 Ki, |
|---|---|---|---|---|
| Compound | Avg, nM | StdDev, nM | Avg, nM | StdDev, nM |
| 1.008 | 30.5 | 0.8 | 3.9 | 0.1 |
| 1.034 | 36.0 | 22.2 | 5.3 | 2.6 |
| 1.039 | 208.6 | 109.0 | 24.7 | 8.4 |
| 1.051 | 37.2 | 4.0 | 3.8 | 0.0 |
| 1.072 | 33.7 | 22.1 | 5.6 | 3.1 |
| 1.074 | 40.1 | 3.3 | 4.1 | 1.5 |
| 1.075 | 48.7 | 2.8 | 4.4 | 0.3 |
| 1.076 | 14.3 | 5.4 | 2.6 | 0.6 |
| 1.077 | 76.1 | 30.9 | 11.1 | 5.8 |
| 1.078 | 36.3 | 10.1 | 3.6 | 0.9 |
| 1.079 | 71.5 | 9.1 | 4.7 | 1.1 |
| 1.080 | 130.8 | 42.6 | 15.2 | 4.4 |
| 1.087 | 84.1 | 11.1 | 15.4 | 1.4 |
| 1.090 | 281.0 | 103.7 | 24.9 | 7.9 |
| 1.091 | 71.4 | 22.0 | 3.3 | 1.0 |
| 1.092 | 190.5 | 42.2 | 28.4 | 10.6 |
| 1.093 | 64.5 | 21.9 | 7.7 | 5.2 |
| 1.095 | 274.8 | 88.0 | 49.5 | 35.9 |
| 1.098 | 205.6 | 69.4 | 25.0 | 6.4 |
| 1.106 | 223.4 | 82.0 | 15.1 | 4.9 |
| 1.107 | 233.7 | 137.2 | 14.0 | 8.5 |
| 1.108 | 25.6 | 3.2 | 6.5 | 0.3 |
| 1.109 | 58.8 | 25.8 | 9.6 | 2.5 |
| 1.110 | 59.0 | 4.1 | 11.2 | 0.3 |
| 1.115 | 89.7 | 17.5 | 20.6 | 1.7 |
| 1.116 | 257.8 | 45.6 | 48.9 | 5.5 |
| 1.117 | 208.0 | 1.9 | 35.8 | 2.3 |
| 1.118 | 461.7 | 28.3 | 81.7 | 52.7 |
| 1.123 | 82.3 | 11.0 | 9.6 | 4.3 |
| 1.124 | 64.5 | 7.9 | 3.3 | 0.8 |
| 1.125 | 557.1 | 1.7 | 50.9 | 16.8 |
| 1.126 | 76.2 | 16.7 | 17.2 | 3.9 |
| 1.127 | 96.6 | 11.6 | 11.2 | 0.4 |
| 1.130 | 577.1 | 340.0 | 142.0 | 38.1 |
| 1.131 | 19.7 | 5.9 | 3.8 | 0.9 |
| 1.132 | 22.5 | 6.5 | 3.5 | 0.4 |
| 1.133 | 25.0 | 7.2 | 4.3 | 1.1 |
| 1.134 | 22.4 | 6.0 | 4.4 | 0.6 |
| 1.136 | 40.3 | 15.3 | 5.4 | 0.4 |
| 1.137 | 25.8 | 10.7 | 5.1 | 1.2 |
| 1.138 | 36.3 | 12.2 | 7.2 | 1.1 |
| 1.139 | 200.3 | 26.3 | 23.2 | 9.6 |
| 1.140 | 236.1 | 199.3 | 32.9 | 24.9 |
| 1.141 | 28.5 | 11.1 | 3.8 | 1.1 |
| 1.142 | 104.2 | 26.6 | 12.0 | 4.4 |
| 1.143 | 49.7 | 30.8 | 12.6 | 11.9 |
| 1.144 | 97.6 | 65.0 | 19.5 | 13.0 |
| 1.145 | 35.0 | 13.5 | 6.4 | 0.9 |
| 1.146 | 39.8 | 10.9 | 10.7 | 1.5 |
| 1.147 | 58.3 | 15.6 | 45.7 | 52.0 |
| 1.148 | 24.3 | 13.7 | 3.6 | 0.9 |
| 1.149 | 46.8 | 21.3 | 4.2 | 2.2 |
| 1.150 | 33.2 | 17.5 | 3.2 | 1.2 |
| 1.151 | 22.8 | 6.0 | 2.9 | 0.5 |
| 1.152 | 19.8 | 13.3 | 3.3 | 0.9 |
| 1.153 | 62.8 | 8.7 | 4.2 | 0.8 |
| 1.154 | 52.7 | 9.5 | 6.6 | 1.0 |
| 1.155 | 45.4 | 14.7 | 7.0 | 2.0 |
| 1.156 | 135.8 | 34.3 | 13.0 | 3.0 |
| 1.157 | 263.8 | 73.9 | 8.8 | 1.6 |
| 1.158 | 64.1 | 20.1 | 5.1 | 1.0 |
| 1.159 | 48.1 | 9.2 | 10.1 | 2.6 |
| 1.160 | 218.3 | 28.3 | 49.4 | 13.4 |
| 1.161 | 9.9 | 3.4 | 2.5 | 0.5 |
| 1.162 | 15.2 | 1.5 | 2.8 | 0.8 |
| 1.163 | 33.6 | 5.8 | 2.9 | 0.4 |
| 1.164 | 42.4 | 7.2 | 6.1 | 1.2 |
| 1.165 | 50.7 | 4.4 | 3.4 | 0.6 |
| 1.166 | 95.2 | 8.6 | 8.0 | 0.8 |
| 1.167 | 118.6 | 17.1 | 18.5 | 1.7 |
| 1.168 | 162.2 | 68.3 | 22.9 | 10.4 |
| 1.169 | 256.2 | 132.7 | 33.8 | 20.0 |
| 1.170 | 80.0 | 25.9 | 12.5 | 6.1 |
| 1.171 | 109.2 | 60.1 | 16.0 | 8.4 |
| 1.172 | 103.0 | 40.6 | 20.5 | 7.3 |
| 1.173 | 15.1 | 6.8 | 3.6 | 1.0 |
| 1.175 | 65.9 | 28.3 | 7.6 | 1.5 |
| 1.176 | 314.3 | 77.6 | 11.2 | 3.2 |
| 1.177 | 156.1 | 55.0 | 18.2 | 5.5 |
| 1.178 | 137.6 | 58.0 | 24.9 | 17.6 |
| 1.179 | 292.0 | 70.7 | 19.3 | 4.4 |
| 1.180 | 138.5 | 46.5 | 23.1 | 4.8 |
| 1.181 | 567.8 | 191.3 | 32.8 | 3.5 |
| 1.182 | 408.3 | 106.6 | 30.6 | 4.3 |
| 1.183 | 165.1 | 46.3 | 16.8 | 3.7 |
| 1.184 | 843.1 | 53.0 | 90.9 | 13.9 |
| 1.185 | 81.6 | 33.0 | 12.6 | 6.4 |
| 1.186 | 129.3 | 42.2 | 11.9 | 4.9 |
| 1.187 | 296.2 | 78.8 | 17.3 | 5.8 |
| 1.188 | 3468.8 | | 652.7 | |
| 1.189 | 187.9 | 62.0 | 34.3 | 5.1 |
| 1.190 | 325.6 | 38.9 | 71.8 | 9.0 |
| 1.191 | 147.3 | 24.7 | 33.4 | 2.0 |
| 1.192 | 158.4 | 33.5 | 37.7 | 4.7 |
| 1.193 | 64.9 | 4.2 | 14.8 | 1.2 |
| 1.194 | 175.7 | 6.3 | 20.2 | 2.4 |
| 1.195 | 196.2 | 58.0 | 10.3 | 3.6 |
| 1.196 | 710.7 | 191.7 | 39.8 | 15.0 |
| 1.197 | 120.2 | 36.0 | 5.0 | 1.4 |
| 1.198 | 584.5 | 139.5 | 24.7 | 9.9 |
| 1.199 | 1856.6 | | 213.0 | 34.4 |
| 1.200 | 76.5 | 17.9 | 5.9 | 0.9 |
| 1.201 | 1585.4 | | 229.5 | |
| 1.202 | 203.5 | 40.9 | 33.0 | 2.1 |
| 1.203 | 329.4 | 67.4 | 41.6 | 6.4 |
| 1.204 | 196.1 | 42.0 | 31.9 | 2.2 |
| 1.205 | 498.1 | 95.2 | 46.4 | 3.7 |
| 1.206 | 64.4 | 15.1 | 9.1 | 3.8 |
| 1.207 | 516.3 | 27.5 | 43.7 | 1.1 |
| 1.208 | 54.2 | 25.0 | 12.9 | 2.8 |
| 1.209 | 4591.0 | | 469.6 | 58.3 |
| 1.210 | 95.1 | 18.2 | 25.5 | 3.8 |
| 1.211 | 395.5 | 58.5 | 57.6 | 0.6 |
| 1.212 | 44.2 | 11.2 | 3.9 | 0.2 |
| 1.213 | 106.3 | 10.9 | 3.0 | 0.5 |
| 1.214 | 546.5 | 10.9 | 143.0 | 7.0 |
| 1.215 | 102.8 | 5.8 | 3.5 | 0.3 |
| 1.216 | 1885.4 | | 402.9 | 79.5 |
| 1.217 | 70.1 | 9.5 | 12.1 | 1.1 |
| 1.218 | 401.8 | 34.4 | 30.7 | 3.0 |
| 1.219 | 343.6 | 37.6 | 15.4 | 2.3 |
| 1.221 | 264.4 | 41.6 | 30.0 | 2.6 |
| 1.222 | 228.8 | 41.9 | 75.5 | 1.2 |
| 1.223 | 239.5 | 21.5 | 15.7 | 1.9 |
| 1.224 | 487.0 | 151.5 | 77.5 | 23.0 |
| 1.225 | 605.0 | 133.2 | 189.4 | 48.9 |
| 1.226 | 91.7 | 31.5 | 8.8 | 2.6 |
| 1.227 | 47.5 | 2.8 | 5.3 | 0.4 |
| 1.228 | 1883.4 | 681.9 | 139.6 | 28.2 |
| 1.229 | 121.4 | 86.2 | 18.4 | 5.8 |
| 1.230 | 345.9 | 85.2 | 35.3 | 9.8 |
| 1.231 | 305.1 | 62.8 | 60.3 | 18.2 |
| 1.232 | 136.6 | 41.1 | 20.8 | 8.8 |
| 1.233 | 47.2 | 7.2 | 1.3 | 0.1 |
| 1.234 | 1735.2 | 179.0 | 166.4 | 11.6 |
| 1.235 | 1386.4 | 173.1 | 335.4 | 29.4 |
| 1.236 | 49.3 | 7.1 | 2.1 | 0.1 |
| 1.237 | 286.7 | 55.0 | 4.0 | 0.4 |
| 1.238 | 61.2 | 22.1 | 1.5 | 0.3 |
| 1.239 | 282.6 | 36.2 | 6.3 | 0.6 |
| 1.240 | 624.8 | 74.2 | 60.1 | 9.3 |
| 1.241 | 65.1 | 11.8 | 21.0 | 6.4 |
| 1.242 | 71.4 | 14.1 | 17.5 | 1.8 |
| 1.243 | 219.3 | 29.7 | 84.3 | 17.2 |
| 1.244 | 683.1 | 80.9 | 138.7 | 25.4 |
| 1.245 | 199.0 | 27.7 | 49.5 | 7.9 |
| 1.246 | 92.1 | 6.3 | 11.2 | 0.8 |
| 1.247 | 1312.4 | 268.7 | 242.6 | 53.1 |
| 1.248 | 2349.7 | | 890.6 | 509.8 |
| 1.249 | 91.7 | 25.0 | 8.6 | 3.8 |
| 1.250 | 247.0 | 63.7 | 45.8 | 13.8 |
| 1.251 | 206.8 | 44.0 | 49.2 | 10.5 |
| 1.252 | 30.5 | 1.5 | 4.5 | 0.4 |
| 1.253 | 59.9 | 7.4 | 1.7 | 0.2 |
| 1.254 | 116.0 | 19.4 | 39.0 | 8.7 |
| 1.255 | 3559.3 | 1202.9 | 358.9 | 99.3 |
| 1.256 | 700.1 | 179.5 | 85.5 | 18.8 |
| 1.257 | 1273.7 | 237.3 | 168.0 | 35.4 |
| 1.258 | 9.5 | 3.5 | 1.3 | 0.4 |
| 1.259 | 19.5 | 11.6 | 2.1 | 0.3 |
| 1.260 | 70.9 | 48.0 | 7.1 | 1.9 |
| 1.261 | 307.4 | 139.0 | 14.8 | 6.5 |
| 1.262 | 54.9 | 13.3 | 4.0 | 0.7 |
| 1.263 | 2130.5 | 673.5 | 453.4 | 105.3 |
| 1.264 | 494.5 | 1.1 | 59.4 | 9.5 |
| 1.265 | 161.7 | 25.9 | 21.6 | 0.8 |
| 1.266 | 53.8 | 15.1 | 17.1 | 2.8 |
| 1.267 | 98.8 | 21.6 | 23.9 | 6.2 |
| 1.268 | 403.6 | 78.8 | 40.7 | 7.5 |
| 1.269 | 239.1 | 62.6 | 22.8 | 9.0 |
| 1.270 | 130.5 | 45.0 | 9.9 | 0.6 |
| 1.271 | 332.1 | 99.9 | 77.7 | 5.8 |
| 1.272 | 1823.7 | 1294.6 | 194.3 | 17.0 |
| 1.273 | 31.3 | 8.3 | 8.2 | 1.0 |
| 1.274 | 223.4 | 46.3 | 10.7 | 1.1 |
| 1.275 | 401.7 | 44.9 | 14.1 | 2.0 |
| 1.276 | 64.2 | 5.2 | 12.3 | 2.5 |
| 1.277 | 42.3 | 10.4 | 4.6 | 1.3 |
| 1.278 | 80.2 | 10.5 | 10.2 | 1.8 |
| 1.279 | 455.9 | 20.3 | 34.2 | 1.6 |
| 1.280 | 746.0 | 58.3 | 38.0 | 4.0 |
| 1.281 | 71.8 | | 7.4 | |
| 2.007 | 390.4 | | 179.1 | |
| 2.016 | 100.5 | 14.8 | 42.4 | 10.2 |
| 2.020 | 100.5 | 13.1 | 36.5 | 4.7 |
| 2.022 | 44.8 | 6.9 | 15.3 | 1.1 |
| 2.025 | 6.9 | 1.3 | 2.9 | 0.5 |
| 2.026 | 38.0 | 15.2 | 13.0 | 4.1 |
| 2.027 | 15.7 | 3.8 | 7.4 | 2.3 |
| 2.031 | 14.6 | 4.9 | 5.3 | 1.2 |
| 2.034 | 1002.6 | 392.4 | 221.1 | 312.7 |
| 2.035 | 601.0 | | 201.9 | |
| 2.036 | 579.5 | 139.9 | 232.8 | |
| 2.037 | 920.8 | | 182.2 | |
| 2.038 | 28.9 | 4.5 | 6.3 | 1.0 |
| 2.039 | 18.8 | 9.6 | 6.7 | 1.9 |
| 2.040 | 59.6 | 10.7 | 25.4 | 5.0 |
| 2.041 | 30.8 | 2.6 | 9.6 | 2.6 |
| 2.043 | 49.4 | 9.5 | 21.5 | 2.4 |
| 2.044 | 81.4 | 20.2 | 24.1 | 3.7 |
| 2.045 | 90.6 | 64.6 | 88.0 | 57.3 |
| 2.046 | 16.7 | 1.1 | 5.6 | 0.8 |
| 2.047 | 26.4 | 3.6 | 7.0 | 2.3 |
| 2.048 | 71.5 | 22.8 | 34.6 | 9.7 |
| 2.049 | 113.0 | 42.1 | 48.0 | 17.1 |
| 2.050 | 367.7 | 115.4 | 250.7 | |
| 2.051 | 1437.2 | 595.4 | 1179.8 | |
| 2.052 | 508.5 | 169.1 | 142.6 | |
| 2.053 | 951.6 | 157.1 | 182.4 | |
| 2.054 | 17.1 | 2.3 | 3.7 | 0.1 |
| 2.055 | 16.0 | 5.3 | 6.4 | 1.2 |
| 2.056 | 106.6 | 12.7 | 48.7 | 26.5 |
| 2.057 | 6.2 | 1.3 | 3.7 | 0.7 |
| 2.058 | 15.3 | 2.8 | 3.3 | 0.6 |
| 2.059 | 3.9 | 0.3 | 2.7 | 0.2 |
| 2.060 | 4.9 | 0.3 | 3.2 | 0.1 |
| 2.061 | 10.5 | 3.2 | 1.8 | 0.4 |
| 2.062 | 63.4 | 25.1 | 30.5 | 2.2 |
| 2.063 | 206.2 | 88.8 | 73.9 | 3.5 |
| 2.064 | 4.1 | 1.8 | 2.2 | 0.4 |
| 2.065 | 4.1 | 1.4 | 1.8 | 0.2 |
| 2.066 | 10.2 | 3.4 | 2.3 | 0.4 |
| 2.067 | 19.6 | 5.8 | 4.2 | 0.5 |
| 2.068 | 8.0 | 2.0 | 5.8 | 0.4 |
| 2.069 | 16.7 | 4.9 | 2.4 | 0.3 |
| 2.070 | 285.9 | 122.0 | 48.4 | 6.1 |
| 2.071 | 21.2 | 2.7 | 11.9 | 0.5 |
| 2.072 | 7.5 | 1.4 | 4.4 | 0.5 |
| 2.073 | 12.7 | 2.6 | 4.2 | 0.4 |
| 2.074 | 133.3 | 31.1 | 36.4 | 7.7 |
| 2.075 | 123.0 | 25.7 | 21.7 | 1.5 |
| 2.076 | 8.0 | 1.8 | 2.4 | 0.3 |
| 2.077 | 33.7 | 12.5 | 5.0 | 0.8 |
| 2.078 | 18.3 | 4.4 | 2.6 | 0.0 |
| 2.079 | 18.5 | 5.5 | 2.3 | 0.2 |
| 2.080 | 213.7 | 18.5 | 125.9 | 17.7 |
| 2.081 | 1446.1 | 317.4 | 1111.2 | 989.8 |
| 2.082 | 131.7 | 30.1 | 9.0 | 2.9 |
| 2.083 | 1882.9 | 380.5 | 857.6 | 706.9 |
| 2.084 | 1174.6 | 172.9 | 349.6 | 116.2 |
| 2.085 | 2391.7 | 219.6 | 812.0 | 417.7 |
| 2.086 | 1246.0 | 57.7 | 358.0 | 28.5 |
| 2.087 | 896.4 | 67.0 | 59.3 | 6.2 |
| 2.088 | 38.7 | 6.1 | 13.6 | 1.6 |
| 2.089 | 102.1 | 3.7 | 32.9 | 3.1 |
| 2.090 | 53.3 | 10.2 | 19.5 | 2.4 |
| 2.091 | 776.1 | 94.2 | 236.7 | 16.1 |
| 2.092 | 1132.5 | 128.2 | 458.0 | 73.1 |
| 2.093 | 576.3 | 99.5 | 127.7 | 19.5 |
| 2.094 | 16570.6 | 1465.6 | | |
| 2.096 | 70.2 | 9.7 | 9.6 | 1.5 |
| 2.097 | 35.4 | 2.1 | 2.8 | 0.8 |
| 2.098 | 382.5 | 13.6 | 73.5 | 3.6 |
| 2.099 | 15.0 | | 3.8 | |
| fasudil | 346.3 | 17.6 | 96.4 | 6.4 |
| H-1152 | 18.5 | 5.3 | 2.0 | 0.3 |
| H7 | | | 124.7 | 5.6 |
| Y-27632 | 197.2 | 50.6 | 60.9 | 16.9 |
| Y-39983 | 34.7 | 11.1 | 3.6 | 0.9 |

### Conclusion

Most of the compounds studied inhibited ROCK2 with a Kᵢ below 600 nM, many of these values below 60 nM. The most potent compounds in this assay showed Kᵢ values below 15 nM.

### Example 2

### NIH/3T3 Cell Morphology Assay

### Relevance

The assay demonstrates that a compound's in vitro ROCK inhibition activity manifests itself in morphology changes, such as actin stress fiber disassembly and alteration in focal adhesions in intact cells leading to inhibition of acto-myosin driven cellular contraction. These morphology changes provide the basis for the beneficial pharmacological effects sought in the setting of the disease processes described in this application, specifically the disruption of the actin stress fibers and its impact on smooth muscle contractility; cell mobility (Howard et. al. The J. of Cell Biology 98:1265-1271, 1984); and endothelial and epithelial permeability (Stephens et al., Am. Rev. Respir. Dis. 137:4220-5, 1988 and Vandenbroucke et al., Ann. N.Y. Acad. Sci. 1123: 134-145, 2008.)

### Protocol

NIH/3T3 cells were grown in DMEM-H containing glutamine and 10% Colorado Calf Serum. Cells were passaged regularly prior to reaching confluence. Eighteen to 24 hours prior to experimentation, the cells were plated onto Poly-L-Lysine-coated glass bottom 24-well plates. On the day of experimentation, the cell culture medium was removed and was replaced with the same medium containing from 10 nM to 25 μM of the test compound, and the cells were incubated for 60 minutes at 37° C. The culture medium was then removed and the cells were washed with warmed PBS and fixed for 10 minutes with warmed 4% paraformaldehyde. The cells were permeabilized with 0.5% Triton-X, stained with TRITC-conjugated phalloidin and imaged using a Nikon Eclipse E600 epifluorescent microscope to determine the degree of actin disruption. Results were expressed as a numerical score indicating the observed degree of disruption of the actin cytoskeleton at the test concentration, ranging from 0 (no effect) to 4 (complete disruption), and were the average of at least 2 determinations.

All compounds tested show measurable activity in the cell morphology assay, with most of the compounds providing substantial effects (score of ≧2 at 1 μM) on the actin cytoskeleton at the testing concentration (see Table 2).

TABLE 2
Cell Morphology Assay Data

| Compound | Cell score at 1 μM |
|---|---|
| 1.002 | 1.4 |
| 1.004 | 1.8 |
| 1.005 | 1.3 |
| 1.006 | 2 |
| 1.008 | 2 |
| 1.024 | 2.4 |
| 1.025 | 2 |
| 1.034 | 2 |
| 1.039 | 2 |
| 1.041 | 2.5 |
| 1.046 | 2.5 |
| 1.048 | 1.5 |
| 1.051 | 2.5 |
| 1.052 | 2.8 |
| 1.062 | 2.3 |
| 1.066 | 2 |
| 2.002 | 1.8 |
| 2.006 | 2.8 |
| 2.008 | 1 |
| 2.016 | 1.8 |
| 2.017 | 2 |
| 2.018 | 1.8 |
| 2.026 | 2 |

### Example 3

### Human Neutrophil Chemotaxis

Neutrophils are recruited to sites of injury and can contribute to the pathogenic features of inflammation through generation of cytokines, reactive oxygen intermediates, elastolytic enzymes, metalloproteases, and myeloperoxidase. This assay is an in vitro assay of neutrophil chemotaxis that can be used to evaluate the ability of Rho Kinase inhibitor compounds of Formula I or II to inhibit the migration of human neutrophils.

Peripheral blood from healthy human volunteers was collected and the neutrophils were isolated by Ficoll-paque density centrifugation followed by dextran sedimentation and hypotonic lysis of the red blood cells. Neutrophil chemotaxis was assessed using a modified Boyden Chamber (Neuroprobe, 96-well) with a 3 μm pore polycarbonate membrane. The ability of the tested compounds to block chemotaxis induced by a 1 μM fMLP challenge during a one hour incubation at 37° C. with 5% CO₂ was assessed in a dose response manner. The results are shown in Table 3.

The results demonstrate that Rho kinase inhibition by Formula I or II compounds inhibited human neutrophil migration toward a chemotactic stimulant in vitro with IC₅₀ potencies ranging from less than 1 μM to nearly 24 μM (Table 3)

TABLE 3
Inhibition of fMLP-induced neutrophil
chemotaxis by Rho kinase inhibitors.

| | Chemotaxis | |
|---|---|---|
| Compound | Avg. IC₅₀ | Chemotaxis |
| Number | (nM) | SEM (nM) |
| 2.038 | 734 | 367 |
| Y-39983 | 1,390 | 803 |
| 1.131 | 1,587 | 916 |
| 2.039 | 1,643 | 949 |
| 2.025 | 1,650 | 636 |
| 1.138 | 1,850 | 212 |
| 1.091 | 2,332 | 2,077 |
| 1.136 | 2,600 | 424 |
| 1.092 | 2,747 | 1,586 |
| 2.036 | 2,767 | 1,597 |
| 1.123 | 3,050 | 778 |
| 1.124 | 3,402 | 1,964 |
| 2.026 | 3,800 | 2,970 |
| H-1152 | 4,350 | 1,202 |
| 1.087 | 4,500 | 2,598 |
| 2.034 | 4,733 | 2,733 |
| 1.034 | 5,601 | 3,234 |
| 2.035 | 6,600 | 3,811 |
| Y-27632 | 6,765 | 1,747 |
| Fasudil | 23,800 | 13,741 |

### Example 4

### Human and Murine Eosinophil Chemotaxis

Eosinophils are known to play a pivotal role in the pathogenesis of allergic asthma. Eosinophils are a major source of growth factors, lipids, basic granule proteins, cytokines and chemokines that contribute to the asthmatic disease state. Although infiltration and activation of other inflammatory cells actively contribute, it is the chemotaxis of eosinophils that is considered to be the single most important event in the pathogenesis of allergic inflammation. (See Adachi, T et. al., The Journal of Immunology. 167: 4609-4615, 2001.)

In a murine model of asthma, when a Rho kinase inhibitor was administered to ovalbumin challenged mice, reductions in eosinophil recruitment to the airways was demonstrated (Taki, F. et. al., Clinical and Experimental Allergy. 37: 599-607, 2007). Likewise, it has also been shown in vitro that Rho kinase is critical for eosinophil chemotaxis and inhibition of ROCK results in a dose-dependent inhibition of eotaxin-induced chemotaxis of human eosinophils (Alblas, J et. al., Molecular Biology of the Cell. 12: 2137-2145).

Human Eosinophil Isolation:

Peripheral blood from healthy human volunteers was collected and the PMNs separated via Ficoll-paque density centrifugation followed by hypotonic lysis of the red blood cells. Subsequently, the human eosinophils were isolated from the cell suspension via StemCell Technologies Human Eosinophil Enrichment kit (Cat. No 19256) according to the manufacturer's recommendations. Briefly, unwanted cells were specifically labeled with dextran-coated magnetic nanoparticles using bispecific Tetrameric Antibody Complexes (TAC) directed against cell surface antigens on human blood cells: CD2, CD3, CD14, CD16, CD19, CD20, CD36, CD56, CD123, glycophorin A and dextran. The unwanted cells are then separated from the unlabelled eosinophils using the EasySep® magnetic isolation procedure.

Mouse Eosinophil Isolation:

Bronchoalveolar lavage was collected from ovalbumin sensitized and challenged mice in a volume of 2.5 mL lavage buffer. The lavage buffer was 0.9% saline with 10% fetal bovine serum. The pooled lavages were maintained on ice until use. The murine eosinophils were isolated using MACS cell separation (Miltenyi Biotech) by depletion of B cells and T cells by positive selection following incubation with antibody conjugated magnetic beads specific for CD45-R (B220) and CD90 (Thy 1.2), which bind B cells and T cells, respectively.

In Vitro Chemotaxis:

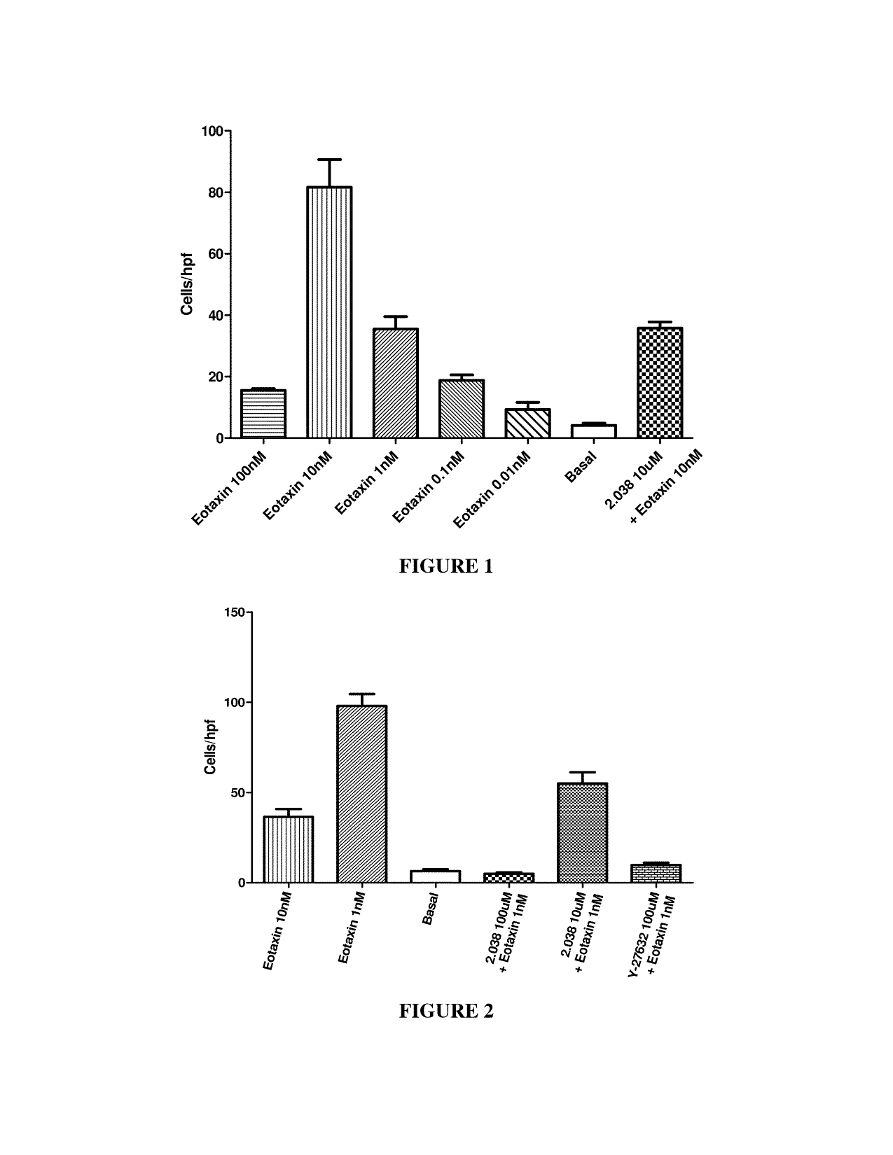

Eosinophil chemotaxis was assessed using a modified Boyden Chamber (Neuroprobe, 96-well) with a 5 μm pore membrane. The ability of the tested compounds to block chemotaxis induced by a 10 nM eotaxin challenge (mouse) or 1 nM eotaxin challenge (human) during one hour incubation at 37° C. with 5% CO₂ was assessed. Chemotaxis was quantified via microscopy by counting the number of migrated cells in at least 3 view fields per treatment. The results are shown in FIGS. 1 and 2. FIG. 1 demonstrates that chemotaxis was induced by eotaxin in murine eosinophils; the chemotactic response was subsequently inhibited by Rho kinase inhibitor Compound 2.038. FIG. 2 demonstrates that chemotaxis was induced by eotaxin in human eosinophils. The chemotactic response was subsequently inhibited by Rho kinase inhibitor Compound 2.038.

### Example 5

### Suppression of Proliferation Ability of Vascular Smooth Muscle Cells (VSMC) by Formula I or II Compounds

Smooth muscle proliferation and remodeling play a role in the pathophysiology of thrombosis.

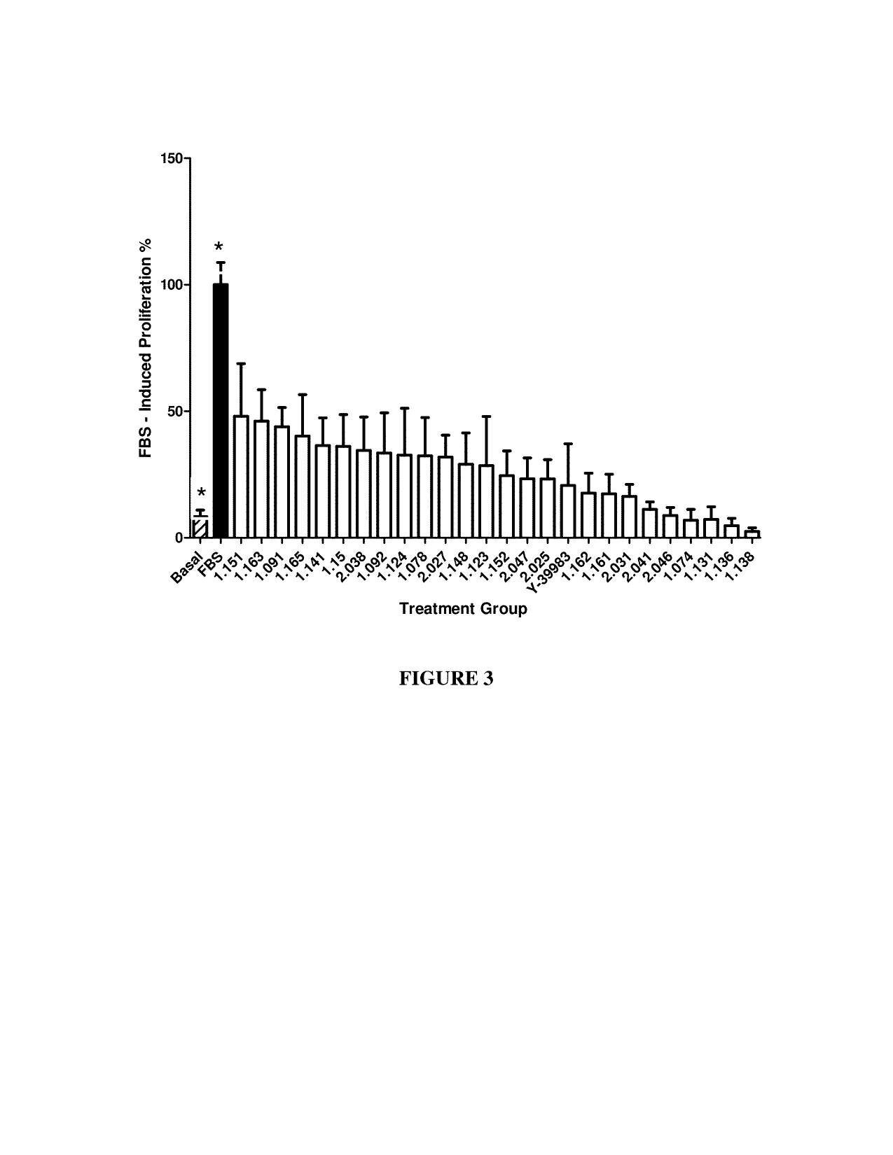

Effects of Formula I or II compounds on cell proliferation were measured using a radiographic technique know as [³H] thymidine incorporation. A-10 rat thoracic aorta cells (ATCC #CRL 1476) were grown on 24-well plates in Dulbecco's Modified Eagles Medium-High Glucose (Gibco cat. #11995-065) containing 10% Fetal Bovine Serum (Sigma EC #232-690-6) for 24 hrs in an incubator at 37° C. Growth media was then removed, the cells were washed with warmed PBS (Gibco cat #14190-144) and warmed serum free media containing 0.1% BSA in order to force the cells into a quiescent state. 24 hours later the media was removed and replaced with warmed serum free media containing from 10 nM to 30 uM of test compound. The cells were incubated for 60 min at 37° C. The cells were then stimulated with either 10% FBS or 10 ng/mL PDGF (BD Biosciences cat #354051) and placed in an incubator at 37° C. for 18 hrs. [³H] thymidine (Perkin Elmer NET027A001MC) was then added to the cells at a final concentration of 3 uCi/mL and placed in an incubator at 37° C. for 24 hrs. The media was removed and the cells were washed with warmed PBS twice. 500 uL of warmed trypsin (Gibco cat #25300-054) was added to each well and they were place in an incubator at 37° C. for 15 min. To precipitate the DNA, 500 uL of ice cold 20% TCA (MP Biomedicals cat #152592) was added to each well. The resulting suspension was filtered using a vacuum manifold and glass fiber filters (Whatman cat #1827-025). The fiber filters were then counted using a liquid scintillation counter (Wallac 1409). Results were normalized to the total signal of the challenge, graphed using Graphpad Prism (Ver. 5.00) and reported as % challenge stimulated proliferation. The results are shown in FIG. 3.

The results demonstrate that the tested Rho kinase inhibitors of Formula I or II compounds reduced the smooth muscle cell proliferation in vitro. Majority of the tested compounds decreased the proliferation to less than 50% of the normal rate at a concentration of 30 uM.

### Example 6

### Coating a Stent with a Polymer Incorporating a Compound of Formula I or II

A stent is coated with a Rho kinase inhibitor compound with procedures modified from that described in Example 4 of U.S. Pat. No. 6,908,624 (Hossainy). A stent is suspended in isopropanol and cleaned in an ultrasonic bath for 30 minutes. The stent is dried and cleaned in a plasma chamber. A poly(ethylene-vinyl alcohol) solution is made by dissolving one part poly(ethylene-vinyl alcohol) in seven parts dimethylsulfoxide, with stiffing and shaking at 60° C. for 24 hours. A Rho kinase inhibitor compound (typically in the range of 2-10% by weight of the total) is added to the poly(ethylene-vinyl alcohol)/dimethyl sulfoxide solution and the solution is mixed, vortexed and placed in a tube. The stent is attached to a mandrel wire and dipped into the solution. The coated stent is briefly passed over a hotplate at 60° C., then is cured for 6 hours at ambient temperature, after which it is dried for 24 hours in a vacuum oven at 40-60° C. The above process is repeated two or three times to give two or three layers. Following final drying, the stent is optionally sterilized by electron beam radiation.

### Example 7

### Compounds of Formula I or II Suppress the Proliferation of Regenerated Intima after Balloon Injury of Carotid Artery in Rat

A 2F Fogarty catheter is inserted from the outer left carotid artery of 8-week-old male WKY rats under anesthesia and inflated in the left common carotid artery, whereby intima is detached in the entire length. Physiological saline is consecutively administered to a control group, and Rho kinase inhibitors at 1 to 30 mg/kg) are consecutively administered to test groups, wherein both control and treated groups undergo intraperitoneal administration starting from 3 days before operation. The rats free of the intima detachment treatment are used as a sham group. At 14 days after the operation, left carotid artery is subjected to perfusion fixation and removed thereafter, stained with HE, and a new intima thickness/medial thickness (I/M) ratio is measured. The left carotid arteries removed and stained with HE are photographed.

In the control group, an increase in proliferation of new intima (measured as an increase of the I/M ratio) mainly consisting of VSMC proliferation is observed when compared with the sham group, thus evidencing lumen constriction. In contrast, in the Rho kinase inhibitor treated group, a decrease in the TIM ratio is observed due to suppression of neogenesis of intima.

### Example 8

### Preventing Restenosis by Using Formula I or II Compound-Eluting Stent in Human Patients

Patients in need of a drug eluting stent are prepared according to established procedures known by those skilled in the art. Patients are administered nitrates. Balloon predilation of the target lesion is performed before delivery of 1 or more stents coated with Rho kinase inhibitor of sufficient length to completely cover the target lesions using procedures known by those skilled in the art. The size of the Rho kinase inhibitor-eluting stent range between 8 mm and 33 mm in length and between 2 mm and 3 mm in diameter.

Quantitative Coronary Angiography is performed in the patients before, during and after the implantation of the stent and angiographic images using edge-detection techniques are obtained (Morice M-C et al. JAMA 295: 895-904, 2006). Coronary luminal diameter and degree of stenosis (as a percentage of the diameter) are measured before dilatation, at the end of the procedure, and at 30 days and 8, 12, 18, and 24 months after the procedure. Restenosis is defined as the presence of a more than 50% luminal-diameter stenosis. Late loss is calculated as the difference between minimum luminal diameter (MLD) immediately after the procedure and MLD measured after 8 months. The target lesion is defined as the stent segment and 5 mm proximal and distal to the edge of the stent.

### Example 9

### Effects of Formula I or II Compound on Platelet Aggregation In Vivo

To evaluate the ability of compounds of Formula I or II to inhibit platelet aggregation in vivo, an experimental protocol similar to the method of R. G. Humphries et al. (Br. J. Pharmacol. 115:1110-1116, 1995) is performed.

### Surgical Preparation and Instrumentation:

Male Sprague-Dawley rats are anesthetized. Body temperature is maintained at 37° C. with a heating lamp. Animals breathe spontaneously and a tracheotomy is performed to ensure a patent airway. A cannula containing heparinized saline is introduced into the left femoral artery and connected to a transducer to record blood pressure and heart rate. Cannulae containing non-heparinized saline are introduced into the left common carotid artery and left jugular vein for withdrawal of arterial blood samples and intravenous administration of compounds, respectively.

### Experimental Protocol

Either compound of Formula I or II or vehicle is administered to each animal as an infusion. Blood samples are taken immediately prior to the first infusion, at the end of each infusion and 20 min after cessation of the final infusion for measurement of platelet aggregation ex vivo. Immediately after sampling, platelet rich plasma is obtained and agonist-induced platelet aggregation is measured. Plasma samples are incubated at 37° C. for 4 min. For the final minute of this period, cuvettes are transferred to a lumi-aggregometer and the sample stirred. Platelet agonist is added in a volume of 20 μl and the aggregation response is recorded. Treatment with Rho kinase inhibitors decrease the shape change and aggregation induced by thrombin receptor agonists.

### Example 10

### Inhibition of Thrombus Formation in Anesthetized Rats

To evaluate the effect of compound of Formula I or II on thrombus formation in vivo, the following experimental protocol is performed.

Rats (CD-1; male; approximately 350 grams; Charles River, Raleigh, N.C.), are anesthetized with sodium pentobarbital (70 mg/kg i.p.). The abdomens are shaved and a 22 gauge intravenous catheter is inserted into a lateral tail vein. A midline incision is made and the intestines are wrapped in saline-soaked gauze and positioned so the abdominal aorta is accessible. The inferior vena cava and abdominal aorta are carefully isolated and a section (approximately 1 cm) of the abdominal aorta (distal to the renal arteries proximal to the bifurcation) is dissected. All branches from the aorta in this section are ligated with 4-0 silk suture. A 2.5 mm diameter flow probe connected to a Transonic flow meter is placed on the artery and a baseline (pre-stenosis) flow is recorded. Two clips are placed around the artery decreasing the vessel diameter by approximately 80%. A second baseline flow measurement is taken (post-stenosis) and the hyperemic response is tested. Animals are then treated with either Rho kinase inhibitor compound or saline intravenously via tail vein catheter. Thrombosis is induced five minutes after treatment by repeated external compressions of the vessel with hemostatic forceps. Two minutes post-injury, the vessel compressions are repeated and a 10 minute period of flow monitoring is started. Animals are monitored continuously for a minimum of the first ten minutes post-injury. After twenty minutes (post-injury), a flow measurement is repeated and the animals are euthanized. The section of the aorta that includes the injured section is harvested and placed in 10% formalin for histological evaluation. Treatment with compound of Formula I or II results in a decrease in the vessel injury-induced flow reduction and histological evidence of thrombosis.

### Example 11

### Inhibition of Thrombus Formation in Anesthetized Dogs

To evaluate the effect of compound of Formula I or II on dynamic thrombus formation in vivo, the following experimental protocol, similar to the method of J. L. Romson et al. (Thromb. Res. 17:841-853, 1980), is performed.

### Surgical Preparation and Instrumentation

Briefly, purpose-bred dogs are anesthetized, intubated and ventilated with room air. The heart is exposed by a left thoracotomy in the fifth intercostal space and suspended in a pericardial cradle. A 2-3 cm segment of the left circumflex coronary artery (LCCA) is isolated by blunt dissection. The artery is instrumented from proximal to distal with a flow probe, a stimulation electrode, and a Goldblatt clamp. The flow probe monitors the mean and phasic LCCA blood flow velocities. The stimulation electrode and its placement in the LCCA and the methodology to induce an occlusive coronary thrombus have been described previously (J. K. Mickelson et al., Circulation 81:617-627, 1990; R. J. Shebuski et al., Circulation 82:169-177, 1990; J. F. Tschopp et al., Coron. Artery Dis. 4:809-817, 1993).

### Experimental Protocol:

Dogs are randomized to one of four treatment protocols in which the control group receives saline intravenously and the three drug-treated groups are administered Rho kinase inhibitor compound intravenously. Upon stabilization from the surgical interventions, dogs receive either saline or compound at different concentrations. After approximately 30 minutes, an anodal current is applied to the LCCA for 180 min. The number and frequency of cyclic flow variations (CFV) that precede formation of an occlusive thrombus are recorded. These cyclic phenomena are caused by platelet thrombi that form in the narrowed lumen as a result of platelet aggregation (J. D. Folts et al., Circulation 54:365-370, 1976; Bush et al., Circulation 69:1161-1170, 1984). Zero flow in the LCCA for a minimum of 30 minutes indicates a lack of antithrombotic efficacy (L. G. Frederick et al., Circulation 93:129-134, 1996). Treatment with compound of Formula I or II significantly increases the number and frequency of cyclic flow variations that precede the formation of an occlusive thrombus.

### Example 12

### An Animal Model of Cerebral Vasospasm

### Protocol

The model is produced as described in Kimura et al. Stroke, 33:593-599, 2002 and Tosaka et al. Stroke, 32:2913-2919, 2001 and Satoh et al. J Clin Neurosci 6:394-39, 1999.

### In Vitro Assessment of Basilar Artery Contraction

Rings of canine basilar arteries are obtained from adult dogs after pentobarbital sodium anesthesia (30 mg/kg IV) and placed in HEPES-buffered Krebs solution at pH 7.4. The 4-mm-long strips are placed in organ baths containing Krebs-Ringer bicarbonate solution and continuously bubbled with 95% O2 and 5% CO2 (pH 7.4). The strips are set at 1.0 g of resting tension between a hook and an isometric force transducer connecting an amplifier and a multipen recorder (LR4220E, Nihon Kohden Ltd). The media in the organ baths are warmed at 37° C. by using a thermal circulator. Strips are equilibrated for at least 90 minutes before data collection. After equilibration, the strips are exposed to 40 mmol/L KCl until the contractile responses were stabilized. The rings were incubated with an efficacious dose of a contractile stimulant such as oxyhemoglobin, sphingosine-1-phosphate, endothelin or serotonin to obtain a stable plateau contraction, and then increasing doses of test compound of Formula I or II, or vehicle control are applied. Alternatively, the rings are first incubated with test compound of Formula I or II or vehicle control for 15 minutes, and then contractile agonist-induced contractions are measured.

The contractile responses are measured over time. Return of contractile responses to basal values or inhibition of contractile responses is seen in Formula I or II treated groups when compared to the control group.

### In Vivo Experimental Model and Angiographic Assessment of Basilar Artery Contraction

Dogs are anesthetized by pentobarbital sodium. Endotracheal intubation is performed, and respiration is mechanically controlled by use of a respirator (tidal volume 200 mL, respiratory rate 14 cycles/min). Each dog is placed in a supine position, the head is fixed, the left vertebral artery is catheterized via the right femoral artery, and control vertebral angiography is performed by using 5 mL of a contrast agent (iomeprol). The cisterna magna is atraumatically punctured by using a 21-gauge spinal needle. Cerebrospinal fluid (CSF, 4 ml) fluid is removed an equivalent amount of arterial blood is withdrawn from the femoral artery and immediately injected into the cisterna magna. This first injection is considered the day 0 subarachnoid hemorrhage (SAH). On day 2, this blood injection procedure is repeated. Six hours after this injection, the first i.v. infusion of vehicle or Formula I or II compound (1 to 100 mg/kg) is administered over 30 min. Administration of Rho kinase inhibitor or vehicle continues twice daily until the morning of day 7. All dogs are killed by an overdose of pentobarbital on day 7. During the experiments, the animals were maintained on a standard diet of pellets and water. Angiograms are obtained at 24, 48, 72, and 96 hours after day 2 SAH. Injection is performed with the animals reanesthetized, intubated, and under controlled ventilation. The diameters of basilar arteries on films were measured 3 cm from the bifurcation of the basilar artery by using a surgical scope.

### Morphology

The basilar arteries are fixed in 10% buffered formalin and carefully removed from the brain stem. The basilar arteries are stained with hematoxylin and eosin for light microscopy. Narrowing of the vessel lumen, corrugation of the lamina elastica and endothelium, and thickness of the vessel wall are observed under light microscopy.

### Results

The diameter of the arteries and morphological endpoints are measured at 24, 48, 72 and 96 hours after day 2 SAH and compared in compound-treated versus vehicle-treated dogs. At least one of the following improvements is observed in the Rho kinase inhibitor treated group: (1) Attenuation of SAH-induced arterial constriction; (2) attenuation of SAH-induced corrugation of the lamina elastica and endothelium; and (3) attenuation of SAH-induced thickening of the vessel wall.

### Example 13

### Pulmonary Arterial and Aortal Relaxation Assay

This assay is a model for selecting compounds for treating diseases that involve constriction of arterial smooth muscle, such as atherosclerosis and systemic hypertension. The effects of compounds to induce relaxation of pre-contracted rat pulmonary artery and rat aorta were determined.

Male Sprague-Dawley rats weighing 301-325 gm were sacrificed by asphyxiation in a CO₂ chamber. Pulmonary artery or aorta were excised, cleaned of connective tissue and cut into cylindrical segments of 2-3 mm length. The preparations were mounted in a tissue bath by tying two threads of surgical silk through the lumen of the vessel. One silk was used to anchor the tissue to a metal wire in the bath and the other silk was connected to a force transducer. Preparations were mounted in 5 ml water-jacketed organ baths (Radnoti Glass Technology) filled with Kreb buffer (95 mM NaCl, 5 mM KCl, 2.6 mM CaCl₂, 1.2 mM MgSO₄, 24.9 mM NaHCO₃, 1.2 mM KH₂PO₄, 10 mM glucose) maintained at 37° C. and gassed with 95% O₂ and 5% CO₂. Contractile tensions were measured using an isometric force transducer (Grass Instruments) and signals were analyzed using specialized software (Chart v5.5, ADInstruments). The preparations were allowed to equilibrate at a resting tension of 0.1 to 0.2 gm for pulmonary artery and 2.0 gm for aorta prior to two challenges with 80 mM KCl to assess tissue viability. After washing, tissues were treated with 100 nM norepinephrine for 5 to 10 minutes to induce a contractile response. For pulmonary artery, compounds were added cumulatively to the bath every 30 minutes and reductions in tension were recorded. Basal tension was subtracted from all values and data was reported as a percentage of the maximal norephinephrine-induced contraction. Data were fit to the Hill equation using GraphPad Prism v5 software. For aorta, a single dose of compound was added and reductions in tension were recorded.

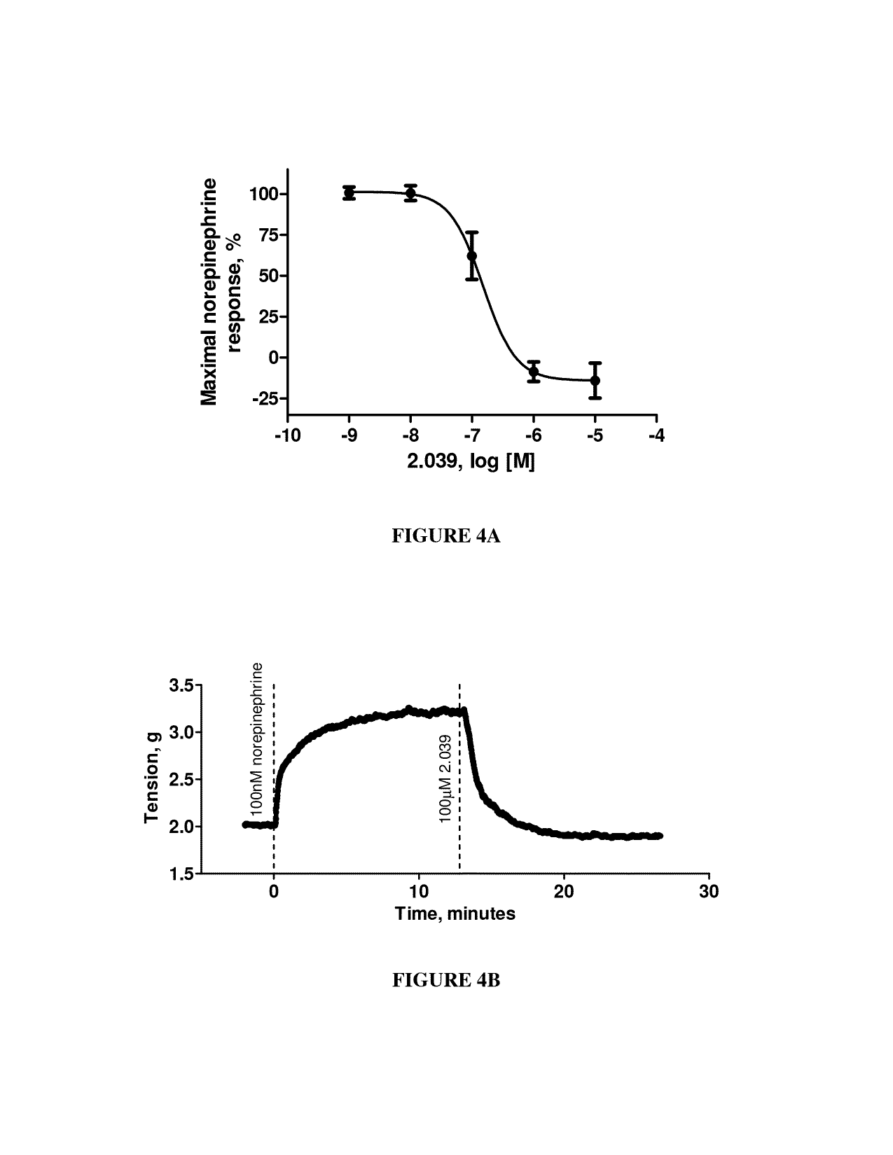

FIG. 4A shows the dose response relationship for a representative compound to induce a relaxant response in precontracted pulmonary artery. The representative compound fully relaxed the pre-contracted pulmonary artery. The IC₅₀ for compound-induced relaxation was 151 nM. These data demonstrate that compounds of this class are able to induce a relaxant response in arterial smooth muscle. FIG. 4B shows the reduction in tension after addition of 100 μM compound in precontracted aorta. Tension returned to basal values upon addition of compound to the norepinephrine precontracted aortal rings. Smooth muscle contractile responses mediate hypertensive disorder and currently marketed therapeutics for hypertensive disorders, such as iloprost, demonstrate efficacy in norepinephrine pre-contracted pulmonary arteries (Walch et al, Brit J Pharmacol 126:859-866 (1999)). Therefore, the results indicate that the compounds are good candidates for treating diseases that involve constriction of arterial smooth muscle, such as atherosclerosis and systemic hypertension.

### Example 14

### Regression of Arteriosclerotic Coronary Lesions in a Porcine Model In Vivo

This example illustrates the efficacy of compounds of this invention in treatment of atherosclerotic coronary lesions in a porcine model with interleukin (IL)-1β (Shimokawa H et al. Cardiovascular Research 51:169-177, 2001).

### Protocol

Segments of the left porcine coronary artery are chronically treated from the adventitia with IL-1β. Two weeks after the procedure, coronary stenotic lesions with constrictive remodeling and vasospastic response to serotonin are noted at the IL-1β-treated site. Then, animals are randomly divided into two groups; one group is treated with 1-100 mg/kg (p.o. or i.p.) of a Rho kinase inhibitor for 8 weeks followed by 1 or 4 weeks of washout period and another group serves as a control.

### Results

In the group treated by compound of Formula I or II, coronary stenosis and vasospastic response are progressively reduced in vivo, while the coronary hyperreactivity is abolished both in vivo and in vitro. The histological examination demonstrates a marked regression of the coronary constrictive remodeling.

### Example 15

### Reduction of Neointimal Inflammation in a Rabbit Model In Vivo

This example illustrates the efficacy of compounds of this invention in treatment of neointimal inflammation in a rabbit model (Carmen Bustos M A et al. J Am Coll Cardiol 32:2057-2064, 1998).

### Protocol

Twenty-five New Zealand male rabbits are housed in individual cages and quarantined for 7 days before use. Atherosclerosis is induced in each femoral artery by endothelial desiccation with nitrogen, followed by 4 weeks of atherogenic diet. After that, a control angiography is performed to discard those animals with a complete occlusion of the artery, and they are switched to standard chow and randomized to receive 1-100 mg/kg/d of a compound of Formula I or II (p.o. or i.p.) or no treatment. The control of the drug intake is done daily and the dietary regime consists of feeding 50 g of standard chow the first week, 100 g the second week and 150 g the last 2 weeks. After 4 weeks all animals are euthanized. Five control animals fed standard chow and with no experimental intervention are also studied. Q

### Angiography

Animals are anesthetized and given antibiotics. After the medial laparotomy, the abdominal aorta is reached and exposed. A ligature is placed to control the bleeding, and then cannulated and nitroglycerin is infused to avoid spasm. After 1 min, and under microscope, 2 mL of contrast is infused. Hemostasis is achieved by local pressure and the wound is closed.

### Example 16

### IL-1β Monocyte Secretion Assay

IL-1β plays a major role in a number of inflammatory diseases. In the presence of increased IL-1β levels, certain tissues show an up-regulation of adhesion molecules, increased vascular permeability, and increased extravasation of leukocytes including neutrophils, macrophages, and lymphocytes. In this assay, lipopolysaccharide (LPS) was used as the inflammatory stimulus to induce cytokine production in human monocytes, and ATP was used to stimulate release of the pro-inflammatory cytokine IL-1β. Monocytes are known to orchestrate the innate immunity response to LPS by expressing a variety of inflammatory cytokines including IL-1β, TNF-α, IL-6, and many others (Gua M, et al., Cellular Signalling. 13: 85-94, 2001).

Peripheral blood from healthy human volunteers was collected and the monocytes isolated via Ficoll-paque density centrifugation. The resultant pellet was re-suspended in media containing 1 ng/mL lipopolysaccharide (LPS) and plated at a density of 500,000 cells/mL. After 3 hours of incubation (37° C., 5% CO₂, humidified air), monocytes were selected by adherence to the tissue culture plastic by washing wells with media. Following the media wash, cells were incubated for 2 minutes with the Rho kinase inhibitors (10 μM) prior to the addition of 1 mM ATP. Cells were allowed to incubate with compounds for 30 minutes at 37° C. after which the supernatant was removed for immediate determination of IL-1β concentration. The concentration of IL-1β in cell supernatants was measured using the Human IL-1β kit and Bio-Plex system (Bio-Rad) according to manufacture's instructions.

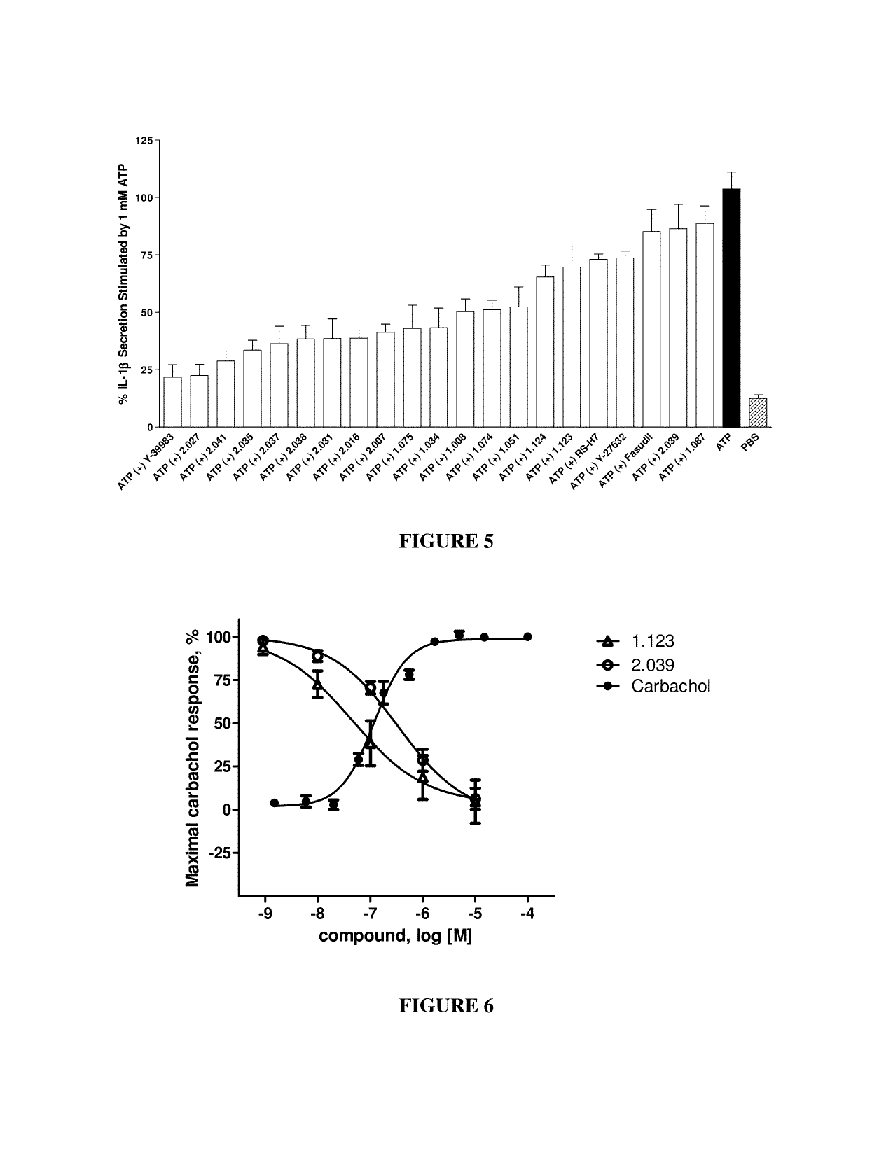

FIG. 5 shows percent inhibition of IL-1β secretion in human monocytes by Rho kinase inhibitors. The tested Rho kinase inhibitors of Formula I or II at a 10 μM concentration demonstrated a varying efficacy range. Many compounds effectively reduced IL-1β secretion to low level.

### Example 17

### Rat In Vivo Hypertension Model

The protocol is similar to the studies of Doe et al (J Pharmacol Exp Ther 320: 89-98 (2007)). Spontaneously hypertensive rats (SHR) are obtained from National Institutes of Health (Bethesda, Md.) and age-matched normotensive rats (Wistar-Kyoto and Sprague-Dawley) to be used as the control group are purchased from Charles River Laboratories, Inc. (Wilmington, Mass.). Experiments are conducted in accordance with the Guide for Care and Use of Laboratory Animals (NIH Publication 85-23).

Blood pressure measurements are preformed using a telemetry system as described previously (Ju et al, J Pharmacol Exp Ther 307: 932-938 (2003)). Male SHR and normotensive rats, 8-10 weeks of age maintained on a normal powdered diet, are anesthetized with 2% isoflurane anesthesia and a telemetry transmitter (Data Sciences International, St. Paul, Minn.) is implanted. The transmitter catheter is inserted into the femoral artery and advanced into the lower abdominal aorta. Baseline measurements of systolic and diastolic blood pressure, heart rate, and activity are obtained 1 week before experiments. Recordings are obtained each week thereafter for a continuous period of 24 hours with data acquisition of 10-s averages every 5 min. Rho kinase inhibitors of Formula I or II compounds are administered via oral gavage at 0.1 mg/kg to 100 mg/kg of body weight and blood pressure responses are monitored immediately following drug administration. Four to six animals are examined for each dose in treated and vehicle groups.

Maximal blood pressure changes are analyzed for statistical significance. Oral administration of Rho kinase inhibitors of Formula I or II compounds induces a dose-dependent reduction in blood pressure in spontaneously hypertensive rats (SHR). The reduction of blood pressure is accompanied by an increase in heart rate. Therefore, Rho kinase inhibitors of Formula I or II compounds provide a method to modulate vasodilation due to reduction of total peripheral vascular resistance.

### Sample Collection

Animals are anesthetized and both femoral arteries and the aorta are exposed. One of the femoral arteries and a piece of the aorta are removed, the adventitial layer is carefully peeled off and immediately snap-frozen in liquid nitrogen. The animals are euthanized with an overdose of pentobarbital and a liver sample is obtained and frozen. The other femoral artery is cannulated, fixed in situ with 4% buffered formaldehyde at 100 mm Hg pressure, removed and embedded in paraffin. Plasma samples are collected 24 h postmeal at the beginning of the study, at the moment of randomization and at death. Plasma cholesterol, LDL and HDL cholesterol and triglycerides are measured by enzymatic techniques.

### Results

The Rho kinase inhibitors of Formula I or II compounds induce a significant reduction in serum lipids and in lesion size. Arterial macrophage infiltration is abolished by the treatment, and monocyte chemoattractant protein-1 (MCP-1) is significantly diminished in the neointima and in the media. Nuclear factor kappa-B (NF-kB) is activated in the lesions, both in macrophages and vascular smooth muscle cells (VSMC), of the untreated group more so than in the treated group. NF-kB activity is also lower in the uninjured aorta and liver of treated compared with untreated rats. In a rabbit atherosclerosis model, compounds of Formula I or II diminish the neointimal inflammation, and this contributes to the stabilization of the atherosclerotic plaque.

### Example 18

### Prevention of Cardiac Hypertrophy with Rho Kinase Inhibitor in ApoE-KO Mice

Apolipoprotein E deficient knock out (apoE-KO) mice (6 months old) are infused with angiotensin II (1.44 mg/kg per day) for 30 days in the presence or absence of a compound of Formula I or II using the methods described previously (Deng et al. Circ. Res., 92(2):510-517, 2003; Wang et al. Am. J. Pathol., 159(4):1455-1464, 2001). A solution of a compound of Formula I or II is prepared by dissolving Rho kinase inhibitor in water (at a dose level from 1 to 200 mg/kg in water) and provided to apoE-KO mice ad libitum. Daily water consumption is measured and the average daily dose of the Formula I or II compound is calculated. Both untreated angiotensin II-infused mice and angiotensin II-infused mice treated with the Formula I or II compound consume similar quantities of water during the course of the experiment.

Angiotensin II treatment causes cardiac hypertrophy, accompanied by up-regulation of gene expression of ANP and collagen III in the heart of apoE-KO mice. To determine the effect of treatment on cardiac hypertrophy, hearts are removed and wet weights are measured. Then the heart tissue is prepared and examined. Treatment of apoE-KO mice with the Formula I or II compound significantly reduces cardiac hypertrophy as measured by heart weight and cardiomyocyte size. In addition, the Formula I or II compound reduces perivascular fibrosis, improves cardiac function, and normalizes gene expression of ANP and collagen III in mice.

### Example 19

### Animal Model for Treating Erectile Dysfunction

Rats which have been rendered severely hypogonadal by surgical castration show a diminished erectile response. Traces of the erectile response to graded stimulation (1-5 V) of the major pelvic ganglion before and after intracavenosal injection of the Formula I or II compound are examined for each of the applied voltages (based on the value for 2 minutes of stimulation at a given voltage) for several animals.

When castrated rats that display an impaired erectile response are treated with a compound of Formula I or II at 2.0 to 400 nmol/kg body weight, the erectile response is restored to levels similar to those in age-matched intact animals. The results suggest that Formula I or II compound can reverse the erectile dysfunction associated with augmented vasoconstrictor activity, and restore the erectile response to normal.

### Example 20

### Efficacy of Compounds in Treating Pulmonary Arterial Hypertension Protocol

The experiment is conducted essentially as in Abe K et al. Circ. Res. 94: 385-393, 2004. Male Sprague Dawley rats are administered either monocrotaline or vehicle. Each MCT-treated rat receives a single subcutaneous injection (right or left flank) of MCT (60 mg/kg body weight) on day 0. Control animals receive a single subcutaneous injection of vehicle. A compound of this invention is administered daily starting on day 0 and continued until necropsy. Groups of animals are sacrificed on Days 21, 28, and 63. A compound of Formula I or II is administered i.p. or p.o. at 1-100 mg/kg of body weight.

### Right Ventricle (RV) Hypertrophy

The RV is dissected from the left ventricle (LV) plus the septum (S) and weighed to determine the extent of RV hypertrophy (RVH) as follows: RV/(LV+S)(Cowan K N et al. Nat. Med. 6:698-702, 2000).

### Survival Analysis

The effects of a compound of this invention on the survival of MCT-injected rats are examined. The day of MCT injection is defined as day 0. This survival analysis covers the entire experimental period to day 63.

### Hemodynamic Measurements

After the animals are anesthetized with sodium pentobarbital (30 mg/kg, IP), polyethylene catheters are inserted into the RV through the jugular vein and into the carotid artery for hemodynamic measurements. RV systolic pressure (RVSP) is measured with a polygraph system (AP-601G, Nihon Kohden).

### Morphometric Analysis of Pulmonary Arteries

After the hemodynamic measurements, lung tissue is prepared for morphometric analysis by using the barium injection method (Cowan K N et al. Nat. Med. 6:698-702, 2000). All barium-filled arteries of 15 to 50 μm in diameter, which are nonmuscular under normal conditions, are evaluated for muscularization of pulmonary microvessels (Cowan K N et al. Nat. Med. 6:698-702, 2000). For each artery, the median wall thickness (MWT) is expressed as follows: percent wall thickness=[(medial thickness×2)/external diameter]×100 (Cowan K N et al. Nat. Med. 6:698-702, 2000).

### Results

The survival over the course of treatment from day 0 to day 63 after the MCT administration and the right ventricular hypertrophy, RVSP, MWT at day 21, 28 and 63 after the MCT administration are measured and compared in the compound-treated MCT-exposed rats vs. saline-treated MCT-exposed rats. Improvement in at least one of the above-mentioned endpoints is observed for at least one of the time points.

### Example 21

### Tracheal Relaxation Assay

### Relevance

These data demonstrate that inhibition of Rho kinase with the described compounds induces relaxation of smooth muscle. Although the model described is from tracheal preparations, these data demonstrate the general smooth muscle relaxant properties of these compounds. Therefore, the activity of the present compounds in this ex vivo model supports the use of these agents in diseases associated with constriction of smooth muscle such as vasoconstriction.

### Protocol

The effects of compounds to induce relaxation of pre-contracted rat trachealis were determined. Male Sprague-Dawley rats weighing 301-325 gm were sacrificed by asphyxiation in a CO₂ chamber. Trachea were excised, cleaned of connective tissue and cut into cylindrical segments of 2-3 mm length. Two stainless steel wires were guided through the lumen of the tracheal ring. One wire was fixed in the tissue bath and the other was connected to a force transducer via surgical silk. Preparations were mounted in 5 ml water-jacketed organ baths (Radnoti Glass Technology) filled with Krebs buffer (95 mM NaCl, 5 mM KCl, 2.6 mM CaCl₂, 1.2 mM MgSO₄, 24.9 mM NaHCO₃, 1.2 mM KH₂PO₄, 10 mM glucose) maintained at 37° C. and gassed with 95% O₂ and 5% CO₂. Indomethacin (1 μM), a cyclooxygenase inhibitor, was added to the Krebs buffer and was present throughout the experiments. Contractile tensions were measured using an isometric force transducer (Grass Instruments) and signals were analyzed using specialized software (Chart v5.5, ADInstruments). The preparations were allowed to equilibrate at a resting tension of 0.3 to 0.5 gm prior to two challenges with 60 mM KCl to assess tissue viability. After washing, tissues were treated with 1 μM carbachol for 10 to 15 minutes to induce a contractile response. Test Compounds were added cumulatively to the bath every 30 minutes and reductions in tension were recorded. Basal tension was subtracted from all values and data were reported as a percentage of the maximal carbachol-induced contraction. Data were fit to the Hill equation using GraphPad Prism v5 software.

FIG. 6 shows the dose response relationship for carbachol-induced contraction and the dose response relationship for representative compounds to induce a relaxant response in precontracted tracheal rings.

Table 4 shows (i) the IC₅₀ values of the listed compounds to induce a relaxant response in precontracted tracheal rings, and (ii) the efficacy at 10 μM of the listed compound reported as a percent of the maximal carbachol-induced contraction response.

TABLE 4
ROCK Inhibitor Potency and Efficacy in Tracheal Ring Relaxation
IC₅₀ values and efficacy of 10 μM compounds are shown as a percent
of the carbachol-induced contraction of rat trachea rings.

| | | Percentage of | Percentage of |
|---|---|---|---|
| | IC₅₀ | carbachol-induced | carbachol-induced |
| | Avg, | contraction at 10 μM | contraction at 10 μM |
| Compound | nM | compound, Avg % | compound, StdDev % |
| 1.074 | 729 | −15.1% | 3.4% |
| 1.091 | 103 | 14.3% | 20.0% |
| 1.092 | 453 | −2.9% | 22.5% |
| 1.107 | 1241 | 9.9% | 10.6% |
| 1.123 | 45 | 4.6% | 24.8% |
| 1.124 | 21 | 19.7% | 15.3% |
| 1.131 | 243 | 14.2% | 13.5% |
| 1.136 | 861 | 19.7% | 9.6% |
| 2.026 | 2859 | 17.1% | 18.7% |
| 2.037 | 2115 | 21.3% | 4.8% |
| 2.038 | 272 | 10.2% | 4.9% |
| 2.039 | 343 | 6.2% | 18.2% |
| 2.041 | 162 | −5.4% | 12.2% |
| 2.045 | 2723 | 13.6% | 15.4% |
| fasudil | | 43.0% | 40.1% |
| H-1152 | 164 | −6.5% | 12.0% |
| Y-27632 | 4783 | 23.4% | 17.8% |
| Y-39983 | 190 | −6.0% | 13.7% |

With the exception of fasudil, all compounds tested induced a relaxant response in carbachol precontracted tissue to values that are <25% of the maximal carbachol response and displayed IC₅₀ values of <5 Fasudil was the least efficacious compound at the highest tested concentration of 10 μM. Due to the lack of potency and efficacy of fasudil, an IC₅₀ value could not be obtained with the tested concentrations.

Table 5 shows the efficacy at 1 μM of the listed compound reported as a percent of the maximal carbachol-induced contraction response. Y-27632 induced a relaxant response that was 83.7% of the carbachol-induced contraction. Most of the compound of Formula I or II displayed greater efficacy than Y-27632.

TABLE 5
ROCK Inhibitor Efficacy in Tracheal Ring Relaxation
Efficacy of 1 μM compounds are shown as a percent of the
carbachol-induced contraction of rat trachea rings.

| | Percentage of | Percentage of |
|---|---|---|
| | carbachol-induced | carbachol-induced |
| | contraction at 1 μM | contraction at 1 μM |
| Compound | compound, Avg % | compound, StdDev % |
| 1.072 | 33.1 | 10.1 |
| 1.074 | 39.6 | 11.8 |
| 1.075 | 39.1 | 10.5 |
| 1.078 | 39.6 | 17.5 |
| 1.091 | 35.4 | 21.2 |
| 1.092 | 67.7 | 7.6 |
| 1.123 | 46.7 | 13.1 |
| 1.124 | 37.8 | 12.1 |
| 1.131 | 37.4 | 8.7 |
| 1.132 | 42.4 | 13.0 |
| 1.136 | 45.0 | 18.1 |
| 1.141 | 35.3 | 17.0 |
| 1.148 | 51.2 | 8.0 |
| 1.149 | 34.4 | 14.5 |
| 1.150 | 39.7 | 12.8 |
| 1.151 | 33.1 | 16.2 |
| 1.152 | 34.4 | 13.4 |
| 1.153 | 40.9 | 16.4 |
| 1.161 | 40.3 | 20.9 |
| 1.162 | 34.4 | 21.5 |
| 1.163 | 35.4 | 6.4 |
| 1.165 | 29.8 | 15.7 |
| 1.173 | 32.8 | 14.1 |
| 1.184 | 88.6 | 6.6 |
| 1.196 | 96.2 | 8.0 |
| 1.197 | 41.7 | 17.7 |
| 1.200 | 91.5 | 6.1 |
| 1.212 | 40.1 | 20.8 |
| 1.213 | 29.3 | 11.0 |
| 1.215 | 40.6 | 15.5 |
| 2.025 | 33.3 | 14.8 |
| 2.038 | 57.2 | 14.7 |
| Y-27632 | 83.7 | 7.4 |
| Y-39983 | 26.5 | 11.7 |

### Example 22

### Effect of Inflammatory Cytokines on Tracheal Relaxation

### Relevance

Inflammatory cytokines can alter tissue function and may limit the efficacy of therapeutic interventions. Demonstration of compound efficacy as smooth muscle relaxants in tissue that has been exposed to inflammatory cytokines in vitro supports the utility of these compounds as smooth muscle relaxants in diseases that are accompanied by inflammation in vivo. Therefore, these compounds will prevent vasoconstriction under conditions of inflammation.

### Protocol

Male Sprague-Dawley rats weighing 301-325 gm were sacrificed by asphyxiation in a CO₂ chamber. Trachea were excised, cleaned of connective tissue and cut into cylindrical segments of 2-3 mm length. Tissues were treated for 18 hours at 37° C. in F12 media with penicillin-streptomycin and 0.1% BSA alone or with 10 ng/ml IL-1β and 100 ng/ml TNF-α. Tissues were then washed free of cytokines with Krebs buffer. Contractile tensions were measured using an isometric force transducer (Grass Instruments) as described for Example 3 and signals were analyzed using specialized software (Chart v5.5, ADInstruments). Tissues were treated with 300 nM carbachol for 10 to 15 minutes to induce a contractile response. Test Compounds were added cumulatively to the bath every 30 minutes and reductions in tension were recorded. Basal tension was subtracted from all values and data were reported as a percentage of the maximal carbachol-induced contraction. Data were fit to the Hill equation using GraphPad Prism v5 software.

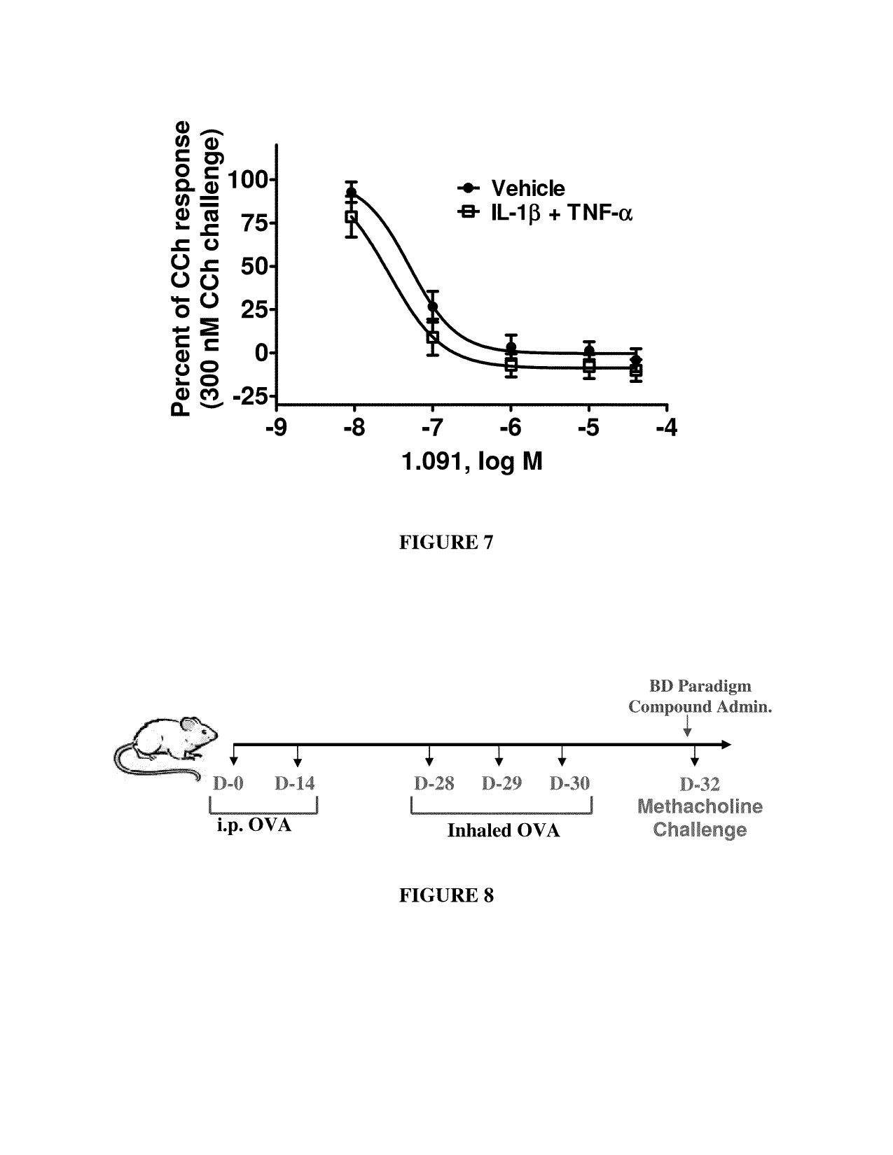

FIG. 7 shows the dose response relationship for representative compounds to induce a relaxant response in vehicle-pretreated and cytokine-pretreated tissues. Compound 1.091 is fully efficacious in relaxing tracheal rings from both vehicle-pretreated and cytokine-pretreated tissues and is slightly more potent in cytokine-pretreated tissues.

### Example 23

### Bronchodilator Assay in Ovalbumin-Sensitized Mice

A mouse model of asthma via ovalbumin sensitization was used to evaluate bronchodilator efficacy of compounds of Formula I or II. The bronchodilator efficacy of these compounds is due to the smooth muscle relaxant properties of these compounds. These data demonstrate the in vivo efficacy of these compounds as smooth muscle relaxants and support the use of these compounds in diseases associated with constriction of smooth muscle such as vasoconstriction

Male BALB/c mice were ordered from Charles River Laboratories (Raleigh, N.C.). The animals were approximately 19 to 21 grams at time of receipt. Upon arrival, the animals were randomized into groups of five males per cage and assigned to a dosing group. Animals were quarantined for 7 days under test conditions. They were observed daily for general health status and ability to adapt to the water bottles.

Animals were sensitized on day 0 and 14 of study by an intraperitoneal injection with 20 μg of ovalbumin (ova) and 2.0 mg aluminum hydroxide (alum) which initiates the development of a specific T-helper (Th) cells type 2 resulting in asthmatic animals. One group of animals received an injection of saline to use as non-asthmatic control animals. All animals were challenged with aerosolized 1% ova once daily for 25 minutes on days 28, 29, and 30 (Zosky, et al. Respiratory Research. 2004; 5:15). Aerosol challenge consists of using an Aerogen Aeroneb nebulizer and controller with a particle size of 4-6 μm mass median aerodynamic diameter (MMAD) with a distribution of 400 μl per minute. This aerosol challenge is necessary to target the Th2-driven allergic inflammation in the lower airways.

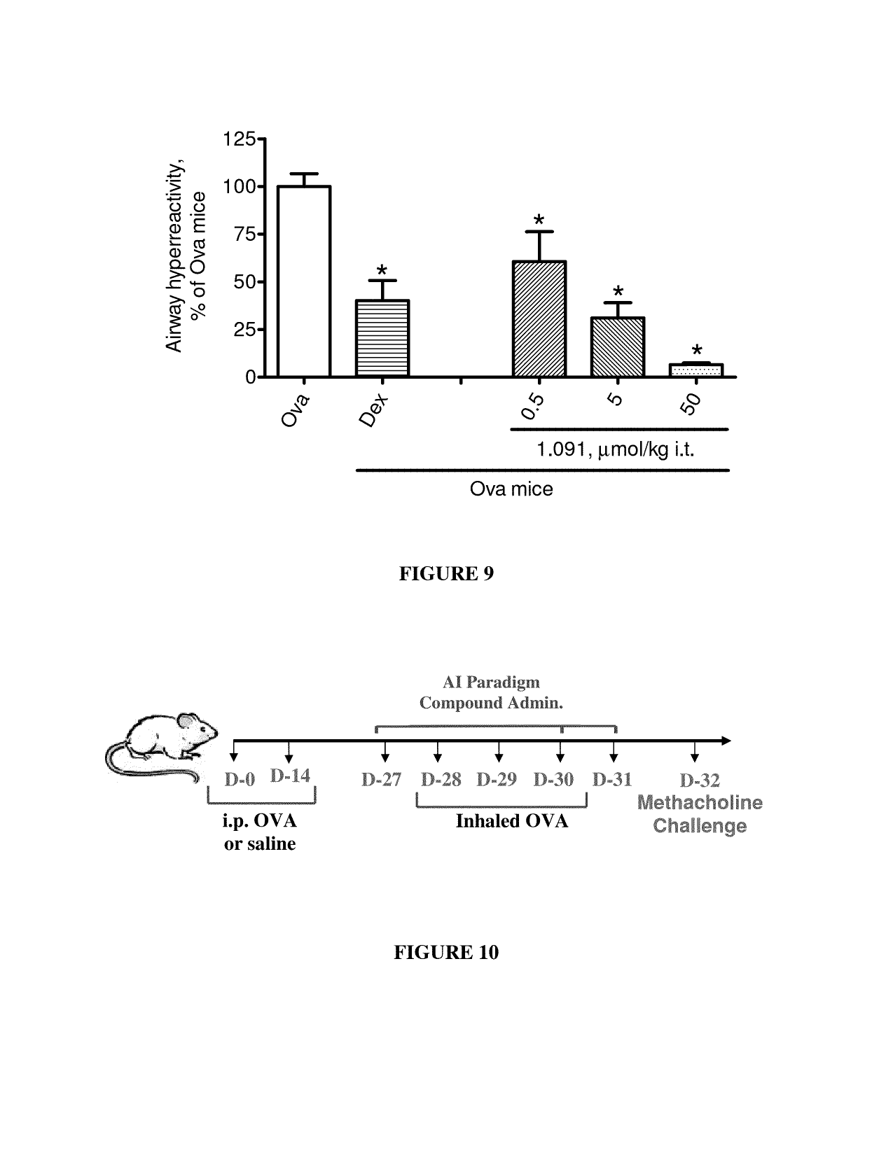

The test compounds and the control vehicle were administered to animals on the day of airway hyperreactivity evaluation 30 minutes to 1 hour before the evaluation to determine the bronchodilator effects of the compounds according to the bronchodilator dosing paradigm (FIG. 8). Compounds were administered p.o. (orally), i.p. (intraperitoneally) at 15 μMol/kg unless otherwise noted (Table 6). Alternatively, compounds were administered i.t. (intratracheally) at varying doses as shown (FIG. 9). On day 32 of the experiment, airway hyperreactivity was evaluated by placing conscious, unrestrained animals in a whole body plethysmometer (Buxco Wilmington, N.C.) and exposing them to escalating doses of nebulized methacholine, a known bronchial constrictor which acts through the muscarinic receptors of the lungs, (doses: 0.325-50 mg/ml). Exposure to the methacholine doses consisted of a 3 minute period during which a nebulizer was aerosolizing the methacholine and an additional 3 minute period following the cessation of nebulization. Over this 6 minute period, the plethysmometer monitors and generates numerical values for all parameters of the breath pattern. Enhanced pause (Penh), a unitless index of airway hyperreactivity, is derived from the expiratory side of the respiratory waveform measured via the plethysmograph and is used as an indirect measure of airway resistance and hyperreactivity. Penh is an indicator of changes in resistance within the airways and has been shown to be a valid marker for airway responsiveness to allergen challenge (Hamelmann, et al. Am J Respir Crit. Care Med. 1997; 156:768-775). Following the methacholine dose response, all animals were anesthetized, bled and euthanized.

### Statistical Methods

Within each experiment, a mouse was given a single compound and exposed to increasing doses of methacholine [0 (baseline), 0.375, 0.75, 1.5, 3, 6, 12, 25, 50 mg/ml]. The Penh value at each of the dose levels of methacholine represents the 6-minute average response. Change from baseline (CFB) in Penh was calculated at each methacholine dose and the area under the curve (AUC) for these CFB values was calculated using the trapezoidal rule. This same approach was applied for each mouse across multiple experiments.

For statistical analyses, a linear mixed-effects model where the response was the log 10 transformed value of AUC described above was used. Data from equal experimental conditions across experiments performed on different days were pooled for statistical analysis and data reporting. The various compounds were compared adjusting for the log 10-transformed baseline value of Penh and the chamber (1 of 10) of the plethysmometer each mouse was contained in during an experiment. A random intercept for each experiment was assumed to account for possible similarities of the results obtained from a given experiment (i.e., as a “blocking effect”). Pairwise comparisons of the compounds were performed using approximate t-tests to test the null hypothesis of no compound difference of the least-squares means of log 10(AUC). p values of less than 0.05 were considered statistically significant. Computations were performed using PROC MIXED (SAS Version 9.1).

For Table 6 and Table 7, Penh values are reported as log 10 transformed AUC values. For FIG. 9 and FIG. 16, linear AUC values from compound treated mice were reported as a percent of linear AUC values from vehicle-treated ovalbumin-sensitized, ovalbumin-challenged (asthmatic) mice.

### Results

Evaluation of the pulmonary mechanics data shows a methacholine dose response trend of increased Penh levels. The ova-sensitized, ova-challenged (asthmatic) animals showed a heightened response to the methacholine, which indicated hyperresponsivness to the smooth muscle constricting agent when compared to the nonsensitized control animals exposed to inhaled ovalbumin or completely naïve animals.

Treating animals with oral doses of potent ROCK2 inhibitors of Formula I or II, which after oral dosing reached high plasma and lung tissue concentrations such as Compounds 1.131 or 2.038 (Table 13 and Table 15), yielded a statistically significant reduction in airway hyperresponsivness (Table 6). Conversely, treating animals with oral doses of potent ROCK2 inhibitors of Formula I or II, which after oral dosing did not reach a detectable plasma concentration in the rat (Table 13) or low concentrations in the mouse (Table 15), such as Compound 1.136 or 1.091, did not yield a significant reduction in airway hyperresponsivness (Table 6). Dosing compounds such as Compound 1.136 or 1.091, which have poor oral bioavailability, via the i.p. route of administration yielded an enhanced reduction in airway hyperresponsiveness when compared to oral dosing (Table 6). Direct application of compounds such as Compound 1.091 to the lung by intratracheal administration resulted in a robust bronchodilatory response (Table 6 and FIG. 9).

TABLE 6
Bronchodilator Efficacy: Statistical Analysis of
the AUC for Average Penh Values Determined During
Experiment Normalized to Baseline for Each Animal

| | | Number | | | |
|---|---|---|---|---|---|
| | Dosing | of | | | |
| | concentration/ | animals | | | Student |
| | route of | per | log10AUC | Standard | t-test |
| Compound | administration | group | (penh) | Error | p-value |
| asthmatic | Vehicle/all | 209 | 2.3205 | 0.02806 | |
| | routes | | | | |
| 1.136 | 15 μmol/kg/ | 10 | 2.2490 | 0.1023 | 0.4853 |
| | oral | | | | |
| 1.091 | 15 μmol/kg/ | 20 | 2.2309 | 0.07207 | 0.2123 |
| | oral | | | | |
| 1.136 | 15 μmol/kg/ | 10 | 2.1379 | 0.1017 | 0.0731 |
| | intraperitoneal | | | | |
| 1.215 | 15 μmol/kg/ | 10 | 2.0081 | 0.1014 | 0.0022 |
| | intraperitoneal | | | | |
| 2.025 | 15 μmol/kg/ | 10 | 1.9667 | 0.1014 | 0.0005 |
| | intraperitoneal | | | | |
| 1.235 | 15 μmol/kg/ | 10 | 1.9362 | 0.1017 | 0.0002 |
| | intraperitoneal | | | | |
| 1.162 | 15 μmol/kg/ | 10 | 1.7001 | 0.1017 | <.0001 |
| | intraperitoneal | | | | |
| 2.038 | 15 μmol/kg/ | 25 | 2.0813 | 0.06628 | 0.0003 |
| | oral | | | | |
| 1.091 | 15 μmol/kg/ | 59 | 2.0096 | 0.04474 | <.0001 |
| | intraperitional | | | | |
| 1.131 | 15 μmol/kg/ | 40 | 2.0283 | 0.05314 | <.0001 |
| | oral | | | | |
| Naïve | | 20 | 1.9978 | 0.07286 | <.0001 |
| 1.161 | 15 μmol/kg/ | 10 | 1.9042 | 0.1014 | <.0001 |
| | intraperitional | | | | |
| 1.091 | 5 μmol/kg/ | 10 | 1.8127 | 0.09980 | <.0001 |
| | intratracheal | | | | |
| non- | Vehicle/oral | 100 | 1.9283 | 0.03624 | <.0001 |
| asthmatic | | | | | |
| Y-27632 | 30 μmol/kg/ | 59 | 2.0135 | 0.04523 | <.0001 |
| | oral | | | | |

The t-test was conducted for the comparison of compound-treated to vehicle-treated “asthmatic groups”..

### Example 24

### In Vivo Anti-Inflammatory Assay in Ovalbumin-Sensitized Mice

### Relevance

The mouse ovalbumin sensitization model has been developed by investigators to study malfunction of the immune system, cellular infiltration composed primarily of eosinophils and neutrophils, acute and chronic inflammation, and fluid accumulation (edema), especially in asthma. Although this model is mostly utilized in the context of asthma, this model can be utilized to demonstrate the in vivo anti-inflammatory properties of Compounds of Formula I or II.

### Protocol and Results

A mouse model of asthma via ovalbumin sensitization was created as described in Example 23. The anti-inflammatory dosing paradigm (FIG. 10) was utilized to evaluate the anti-inflammatory effects of experimental compounds. The anti-inflammatory dosing paradigm consists of dosing the animals once a day starting on day 27 and finishing on either day 30 or 31 (1 hr prior to the aerosolized ovalbumin challenges on days 28 to 30) but not on day 32 when hyperreactivity evaluation occurs.

Bronchoalveolar lavage fluid (BALF) was collected by infusing 3.0 ml of saline with 10% fetal calf serum into the lungs via the trachea and then withdrawing the fluid. The total amount of cells/ml of BALF fluid was determined via manual cell count on hemocytometer. The BALF was centrifuged, supernatant removed and analyzed for cytokine concentrations as described below, and cell pellet reconstituted in 500 μL of fluid. Cytospin slides were prepared from the cell pellet using 100 μL of fluid and spinning samples for 5 minutes at 5000 rpms in a cytospin centrifuge. Following Hema3 stain, relative percentages of individual leukocytes were determined on a 200 cell count for each sample. The final concentration of individual leukocyte cell types per ml of BALF was determined by multiplication of the relative percentage of individual leukocytes with the total amount of cells/ml of BALF fluid.

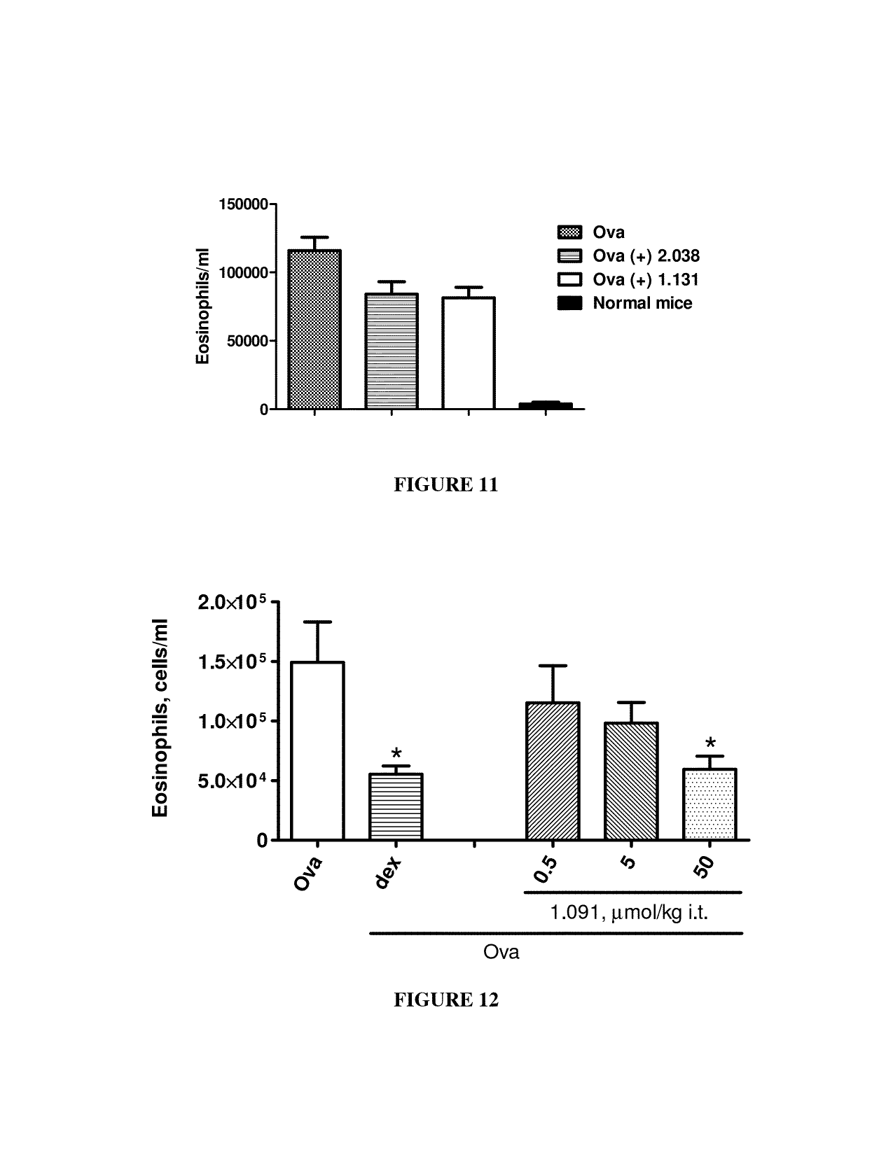

Evaluation of the differential counts performed on these samples showed an increased number of inflammatory cells in the ova-challenged, ova-sensitized animals. FIG. 11 shows the eosinophils per ml of BALF in ova-sensitized, ova-challenged mice, mice treated with Compound 2.038, mice treated with Compound 1.131 and normal mice. Compounds were dosed orally to day 31 according to the anti-inflammatory dosing paradigm shown in FIG. 10. Airway eosinophil infiltration was reduced in animals treated with the two tested compounds (FIG. 11). As shown in FIG. 12, Compound 1.091 generates a reduction of eosinophils when dosed i.t. to day 30 according to the anti-inflammatory dosing paradigm shown in FIG. 10.

The concentrations of cytokines in the BALF samples were determined using commercially available Bio-plex kits (Bio-Rad) for the detection of mouse IL-5, IL-13, and Eotaxin. The analysis of cytokine levels was measured using the Bio-Plex 200 (Bio-Rad) system according to the manufacturer's instructions. Substantial evidence suggests that cytokines play an important role in orchestrating and regulating inflammatory processes through the involvement of T-helper type 2 lymphocytes.

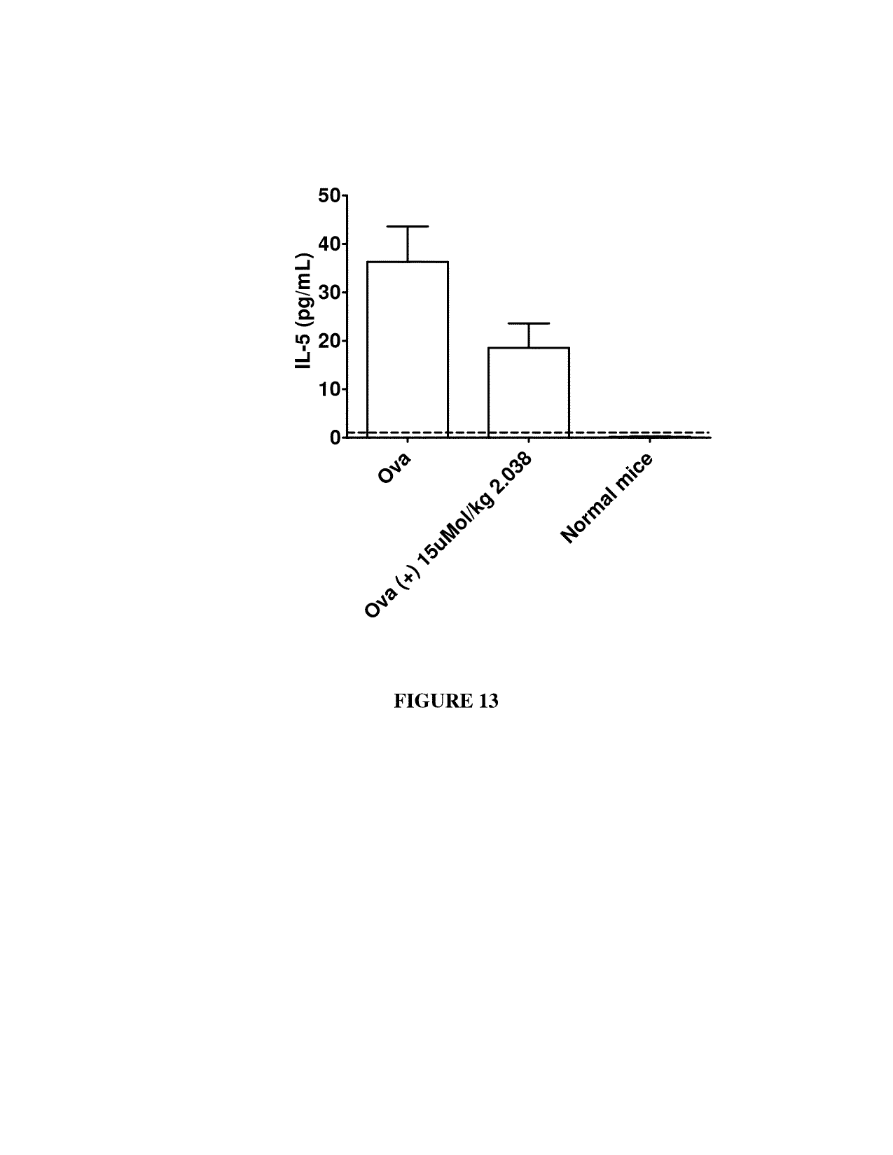

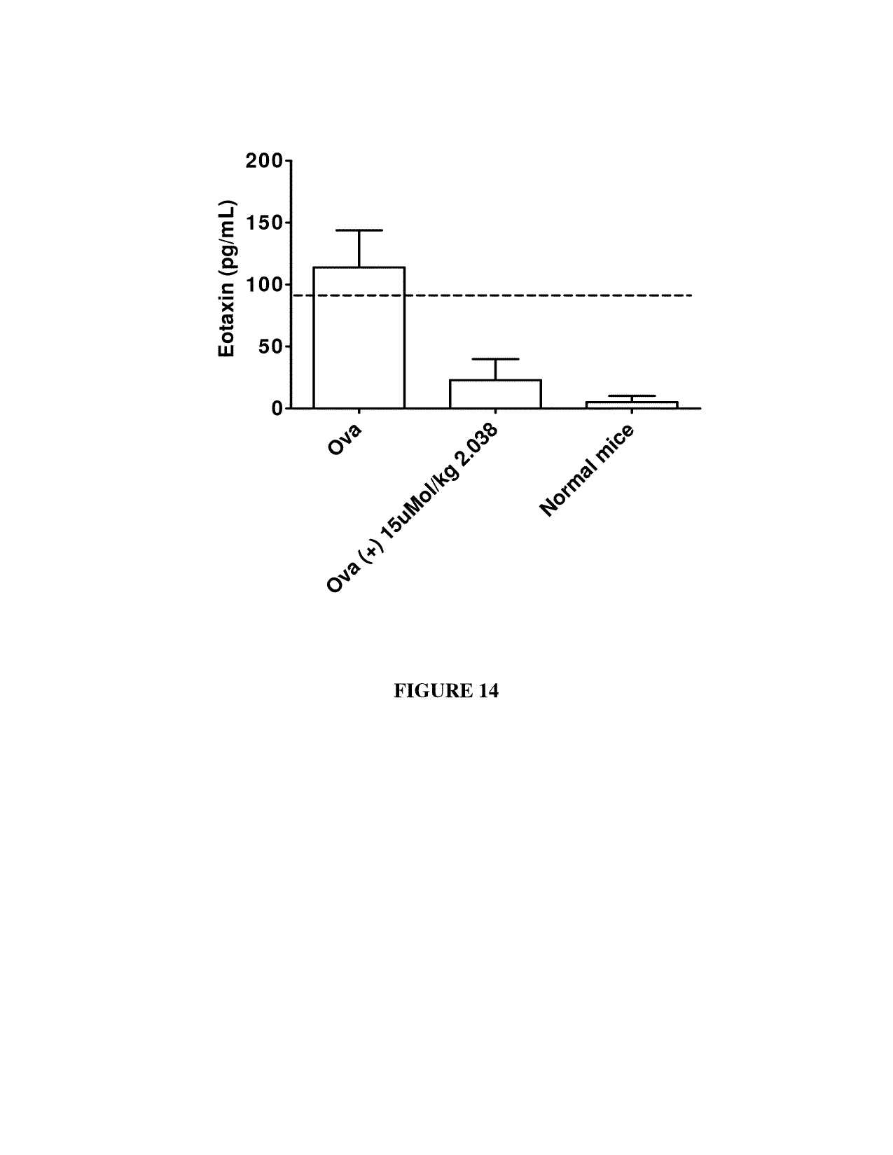

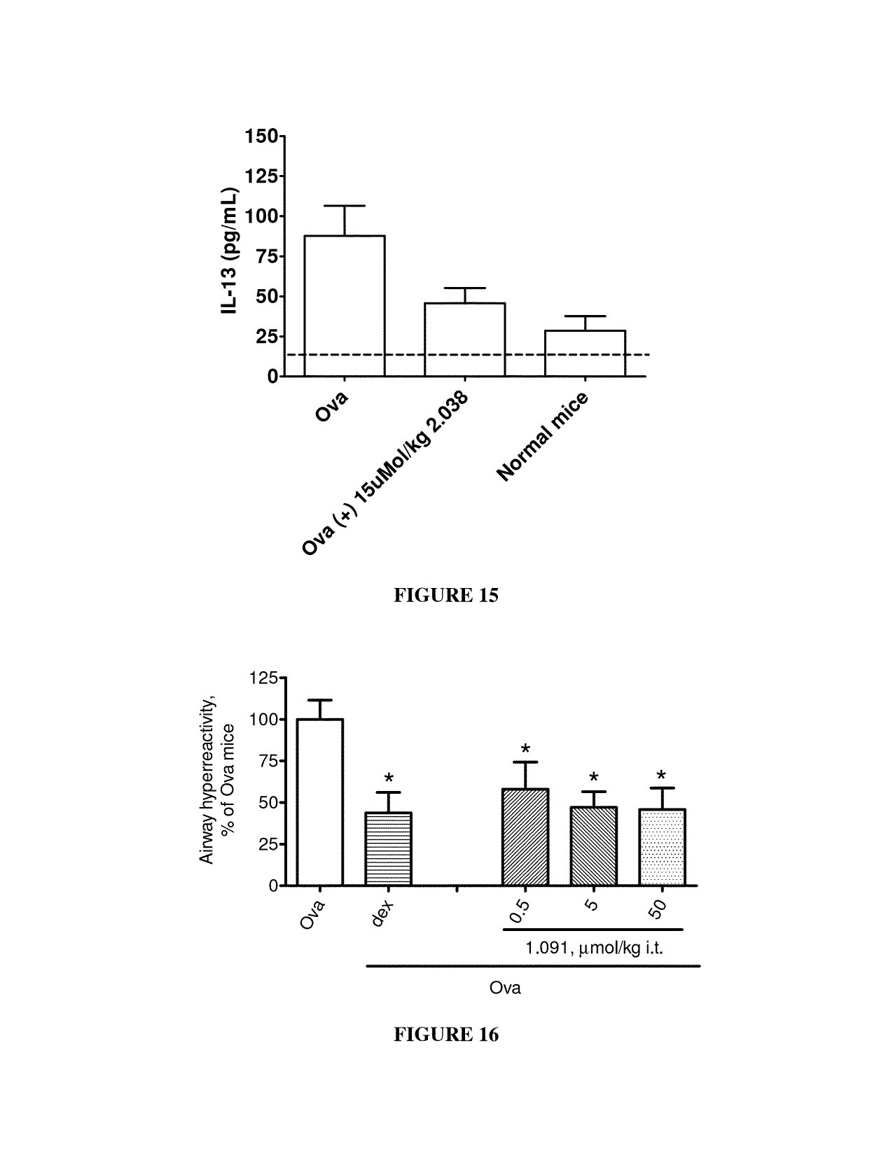

FIGS. 13-15 show the concentration of IL-5, Eotaxin, and IL-13 in (1) ova-sensitized, ova-challenged mice, (2) ova-sensitized, ova-challenged mice treated with Compound 2.038 (15 μmol/kg/oral on days 27 to 31), and (3) normal, saline-sensitized mice. The results showed that ova-sensitized, ova-challenged mice treated with Compound 2.038 had reduced levels of IL-5, Eotaxin, and IL-13.

### Example 25

### Prevention of Airway Hyperreactivity Development Via Decrease in Pulmonary Inflammation

### Relevance

Airway hyperreactivity is a downstream physiologic effect of inflammation in the mouse ovalbumin sensitization model. The objective of the experiment was to answer whether the decrease in inflammation due to ROCK inhibitor anti-inflammatory dosing results in the prevention of downstream physiological consequences as measured by Penh. Although this concept is demonstrated in a model of airway hyperreactivity due to pulmonary inflammation, these data support the general use of these compounds as anti-inflammatory agents to prevent the downstream physiological consequences of inflammation in an in vivo model.

### Protocol

Mouse model of asthma via ovalbumin sensitization was created as described in Example 23. The anti-inflammatory dosing paradigm was utilized as described in Example 10 to evaluate the prevention of airway hyperreactivity due to the anti-inflammatory effects of experimental compounds. The objective of the experiment was to answer whether the decrease in pulmonary inflammation due to ROCK inhibitor anti-inflammatory dosing results in prevention of airway hyperreactivity/decrease in bronchial constriction as measured by Penh, as described in Example 23. Statistical analysis was performed as described in Example 23.

The oral administration of 15 μMol/kg of Compound 1.131 or 2.038 once a day during days 27 to 31 resulted in prevention of airway hyperreactivity to methacholine dosed on Day 32 (Table 7). As shown in FIG. 16 and Table 7, intratracheal administration of Compound 1.091 once a day during days 27 to 30 (FIG. 16) or Compounds 1.161, 2.066 or 2.059 once a day during days 27 to 31 (Table 7) according to the anti-inflammatory dosing paradigm shown in FIG. 10 resulted in prevention of airway hyperreactivity. Compound 1.091, 1.161, 2.066 or 2.059 had similar efficacy to dexamethasone, a corticosteroid anti-inflammatory control. These data support the use of these compounds to prevent the downstream physiologic consequences of inflammation.

TABLE 7
Anti-inflammatory dosing: Statistical Analysis of
the AUC for Average Penh Values Determined During
Experiment Normalized to Baseline for Each Animal

| | | Number | | | |
|---|---|---|---|---|---|
| | Dosing | of | | | |
| | concentration/ | animals | | | Student |
| | route of | per | log10AUC | Standard | t-test |
| | administration | group | (Penh) | Error | p-value |
| asthmatic | Vehicle/oral | 70 | 2.3354 | 0.04751 | |
| 1.131 | 15 μmol/kg/ | 10 | 2.0674 | 0.1061 | 0.0133 |
| | oral | | | | |
| 2.038 | 15 μmol/kg/ | 20 | 1.8981 | 0.07966 | <0.0001 |
| | oral | | | | |
| 1.161 | 0.5 μmol/kg/ | 10 | 2.0405 | 0.1083 | 0.0077 |
| | intratracheal | | | | |
| 2.066 | 0.5 μmol/kg/ | 10 | 2.0248 | 0.1091 | 0.0055 |
| | intratracheal | | | | |
| 2.059 | 0.5 μmol/kg/ | 10 | 1.9979 | 0.1084 | 0.0024 |
| | intratracheal | | | | |
| Y-27632 | 30 μmol/kg/ | 10 | 1.9942 | 0.1062 | 0.0017 |
| | oral | | | | |
| Dexa- | 1 mg/kg/oral | 30 | 2.0216 | 0.06546 | <0.0001 |
| methasone | | | | | |
| non- | Vehicle/oral | 20 | 1.7810 | 0.07973 | <0.0001 |
| asthmatic | | | | | |

Compounds were administered on days 27 to 31 according to the anti-inflammatory dosing paradigm. The t-test was conducted for the comparison of compound-treated to vehicle-treated “asthmatic groups” based on the vehicle which was run in every study.

### Example 26

### Human Monocyte Cytokine Secretion Assay

### Relevance:

This assay demonstrates a compound's ability to inhibit the secretion of multiple pro-inflammatory cytokines from human monocytes. Reduction in the levels of pro-inflammatory cytokines is associated with improvement in disorders with an inflammatory component.

### Protocol

Peripheral blood from healthy human volunteers was collected and the monocytes isolated via Ficoll-paque density centrifugation. Monocytes were purified via an Easy Sep© Monocyte Enrichment Kit (Product number 19059) according to the manufacturer's instructions. The purified monocytes were then plated in 96-well plates at a density of 300,000 cells/mL in RPMI 1640±10% heat inactivated FBS media. The cells were allowed to pre-incubate with test compound at the indicated concentration for 30 minutes (37° C., 5% CO₂, humidified air); after which the supernatant was removed and media containing compound and 1 ng/mL LPS was added. Cells were allowed to incubate with compounds and LPS for 4 hours at 37° C. after which the supernatant was removed and stored at −80° C. Cytokine concentrations in the supernatant were determined using commercially available Bio-Rad Bio-plex™ kits according the manufacturer's instructions.

### Results:

Compounds of Formulae I and II inhibit the release of multiple cytokines from human monocytes when incubated at 10 μM concentration in vitro, as shown in Table 8. Shown further in Table 9, potency determinations on compounds 2.059 and 2.066, both potent inhibitors of ROCK1 and ROCK2 and both of the chemical class in which R₂ is R₂-2, dose-dependently reduced the secretion of IL-1β, TNF-α and IL-9 from LPS-stimulated human monocytes, with potencies ranging from approximately 170 nM to

TABLE 8
Percent inhibition values for inhibition of
cytokine secretion at 10 μM of test compound

| Compound | IL-1β % | IL-6% | TNF-α % |
|---|---|---|---|
| 1.072 | 98.2 | 96.1 | 83.8 |
| 1.074 | 43.9 | 96.0 | 87.7 |
| 1.075 | 49.7 | 73.9 | 51.6 |
| 1.076 | 51.0 | 81.2 | 78.9 |
| 1.077 | 30.3 | 43.3 | 52.3 |
| 1.078 | 60.4 | 111.0 | 88.1 |
| 1.079 | 59.3 | 31.1 | 56.5 |
| 1.091 | 165.5 | 108.2 | 104.6 |
| 1.093 | 109.0 | 49.7 | 76.1 |
| 1.106 | 121.5 | 95.0 | 80.6 |
| 1.107 | 111.3 | 122.1 | 83.1 |
| 1.108 | 131.3 | 89.8 | 116.7 |
| 1.109 | 190.5 | 312.9 | 118.3 |
| 1.110 | 133.6 | 111.7 | 118.6 |
| 1.123 | 82.6 | 64.7 | 62.7 |
| 1.124 | 99.5 | 101.4 | 61.5 |
| 1.127 | 198.0 | 67.3 | 97.3 |
| 1.131 | 48.3 | 68.6 | 85.2 |
| 1.132 | 58.6 | 72.5 | 80.3 |
| 1.133 | 54.5 | 70.7 | 66.2 |
| 1.134 | 43.2 | 74.6 | 69.1 |
| 1.135 | 57.0 | 123.2 | 108.0 |
| 1.136 | 66.3 | 95.0 | 71.5 |
| 1.137 | 40.3 | 46.2 | 58.0 |
| 1.138 | 257.4 | 76.6 | 130.9 |
| 1.141 | 50.4 | 71.7 | 75.7 |
| 1.142 | 82.8 | 40.7 | 68.6 |
| 1.143 | 76.8 | 130.5 | 66.4 |
| 1.145 | 129.2 | 95.1 | 88.9 |
| 1.146 | 85.2 | 128.0 | 97.7 |
| 1.148 | 63.9 | 78.6 | 56.1 |
| 1.149 | 69.8 | 121.5 | 119.9 |
| 1.150 | 78.2 | 89.2 | 94.4 |
| 1.151 | 84.5 | 114.1 | 88.9 |
| 1.152 | 74.7 | 94.7 | 120.1 |
| 1.153 | 64.1 | 106.2 | 74.3 |
| 1.154 | 52.3 | 104.4 | 86.4 |
| 1.155 | 76.7 | 121.8 | 79.7 |
| 1.156 | 60.7 | 92.5 | 70.5 |
| 1.157 | 121.4 | 92.6 | 65.1 |
| 1.158 | 80.8 | 133.1 | 86.6 |
| 1.159 | 97.1 | 84.8 | 76.1 |
| 1.161 | 87.7 | 86.3 | 153.5 |
| 1.162 | 95.5 | 99.8 | 158.7 |
| 1.163 | 166.7 | 140.9 | 91.6 |
| 1.164 | 80.1 | 109.5 | 89.0 |
| 1.165 | 129.9 | 114.3 | 103.5 |
| 1.166 | 107.0 | 87.2 | 82.2 |
| 1.170 | 80.6 | 72.7 | 67.8 |
| 1.171 | 78.9 | 91.8 | 72.2 |
| 1.173 | 86.1 | 79.5 | 80.1 |
| 1.175 | 29.3 | 38.2 | 47.4 |
| 1.176 | 95.2 | 112.4 | 72.4 |
| 1.183 | 68.7 | 123.3 | 76.5 |
| 1.185 | 39.8 | 63.0 | 66.6 |
| 1.186 | 64.1 | 105.3 | 68.2 |
| 1.195 | 115.4 | 94.4 | 67.7 |
| 1.197 | 179.1 | 128.8 | 83.3 |
| 1.200 | 0.0 | 0.0 | 0.2 |
| 1.206 | 88.7 | 164.0 | 97.3 |
| 1.208 | 62.0 | 109.0 | 92.0 |
| 1.212 | 116.3 | 111.0 | 108.1 |
| 1.213 | 111.1 | 81.7 | 77.4 |
| 1.215 | 136.7 | 63.2 | 60.4 |
| 1.217 | 118.6 | 73.8 | 71.3 |
| 1.219 | 138.9 | 127.7 | 82.1 |
| 1.223 | 117.0 | 88.5 | 60.7 |
| 1.226 | 99.3 | 52.2 | 66.6 |
| 1.227 | 69.4 | 66.7 | 79.3 |
| 1.229 | 44.9 | 63.2 | 50.7 |
| 1.233 | 78.5 | 78.9 | 79.0 |
| 1.236 | 75.2 | 93.0 | 98.0 |
| 1.237 | 97.1 | 100.9 | 70.6 |
| 1.238 | 101.1 | 62.9 | 73.2 |
| 1.239 | 39.4 | 84.7 | 58.5 |
| 1.246 | 103.0 | 108.3 | 79.0 |
| 1.249 | 133.8 | 56.2 | 60.0 |
| 1.252 | 139.2 | 68.3 | 101.6 |
| 1.253 | 160.6 | 228.6 | 126.8 |
| 1.258 | 104.1 | 83.5 | 94.0 |
| 1.262 | 145.7 | 156.6 | 135.3 |
| 2.026 | 166.0 | 180.7 | 109.1 |
| 2.031 | 49.0 | 89.3 | 66.4 |
| 2.038 | 90.8 | 79.7 | 70.2 |
| 2.039 | 49.8 | 70.3 | 47.8 |
| 2.054 | 24.0 | 56.8 | 37.9 |
| 2.058 | 1.2 | 1.3 | 10.6 |
| 2.059 | 0.3 | 0.0 | 6.9 |
| 2.060 | 5.9 | 19.6 | 33.0 |
| 2.064 | 14.3 | 45.7 | 66.2 |
| 2.066 | 0.0 | 0.0 | 25.2 |

TABLE 9
IC₅₀ values for inhibition of cytokine secretion

| | IL-1β (nM) | TNF-α (nM) | IL-9 (nM) |
|---|---|---|---|
| Compound 2.059 | 169.4 ± 13.0 | 207.1 ± 17.0 | 268.6 ± 28.1 |
| Compound 2.066 | 346.2 ± 182.3 | 610.6 ± 154.1 | 934.9 ± 407.5 |

### Example 27

### LPS-Induced Neutrophilia and Cytokine Production Assay

### Relevance

Marked neutrophilia can occur upon tissue inflammation. The LPS-induced neutrophilia model is often used to determine the potential efficacy of therapeutic approaches to limit inflammatory responses. This assay is an in vivo assay of neutrophil accumulation and cytokine production that can be used to evaluate the activity of Rho Kinase inhibitor compounds of Formula I or II as anti-inflammatory agents in a whole animal model. Neutrophil accumulation and cytokine production are indicative of an inflammatory response and the activity of compounds to decrease neutrophil accumulation and cytokine production in this assay supports the use of these compounds to treat disorders with an inflammatory component

### Protocol

Male BALB/c mice, approximately 19 to 21 grams, were ordered from Charles River Laboratories (Raleigh, N.C.). All animals were challenged with aerosolized LPS (10 μg/ml) for 25 minutes on study day 0. LPS aerosol was generated using an Aerogen Aeroneb nebulizer and controller providing a flow of 400 μl/min and a particle size of 2-4 μm MMAD. Rolipram was administered i.p at 20 mg/kg. Compound 1.091 or Compound 2.059 was administered intratracheally (i.t.) at 0.5-50 μmol/kg body weight one hour prior to LPS challenge. Four hours following LPS challenge, BALF was collected using a total of 3 ml of 0.9% sodium chloride containing 10% fetal calf serum. Total cell counts were determined using the Coulter Counter. For differential evaluations, BALF was centrifuged and cytospin slides prepared and stained with Hema3 stain. Manual leukocyte counts were then completed on 200 cells. The final concentration of individual leukocyte cell types per ml of BALF was determined by multiplication of the relative percentage of individual leukocytes with the total amount of cells/ml of BALF fluid. The concentration of IL-1β in the BALF samples was determined using commercially available Bio-plex kits (Bio-Rad). The analysis of cytokine levels was measured using the Bio-Plex 200 (Bio-Rad) system according to the manufacturer's instructions.

### Results

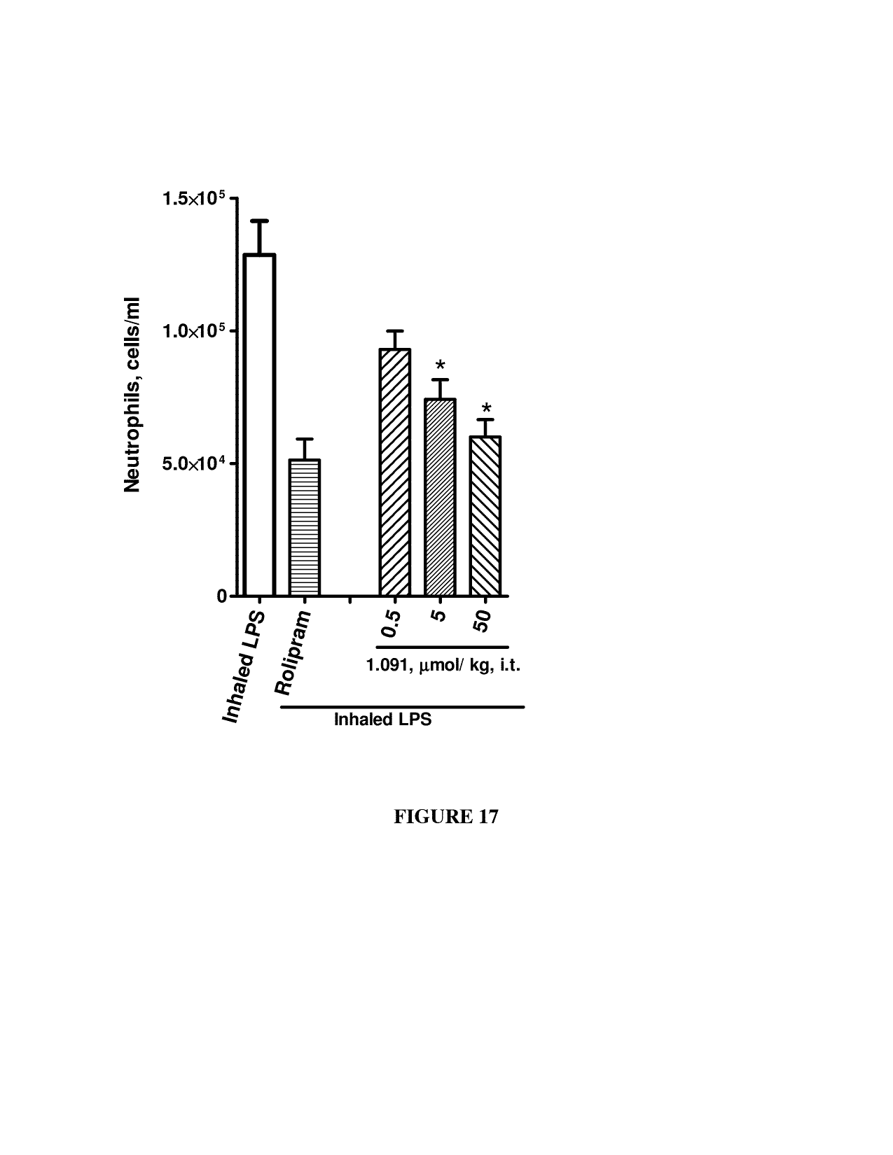

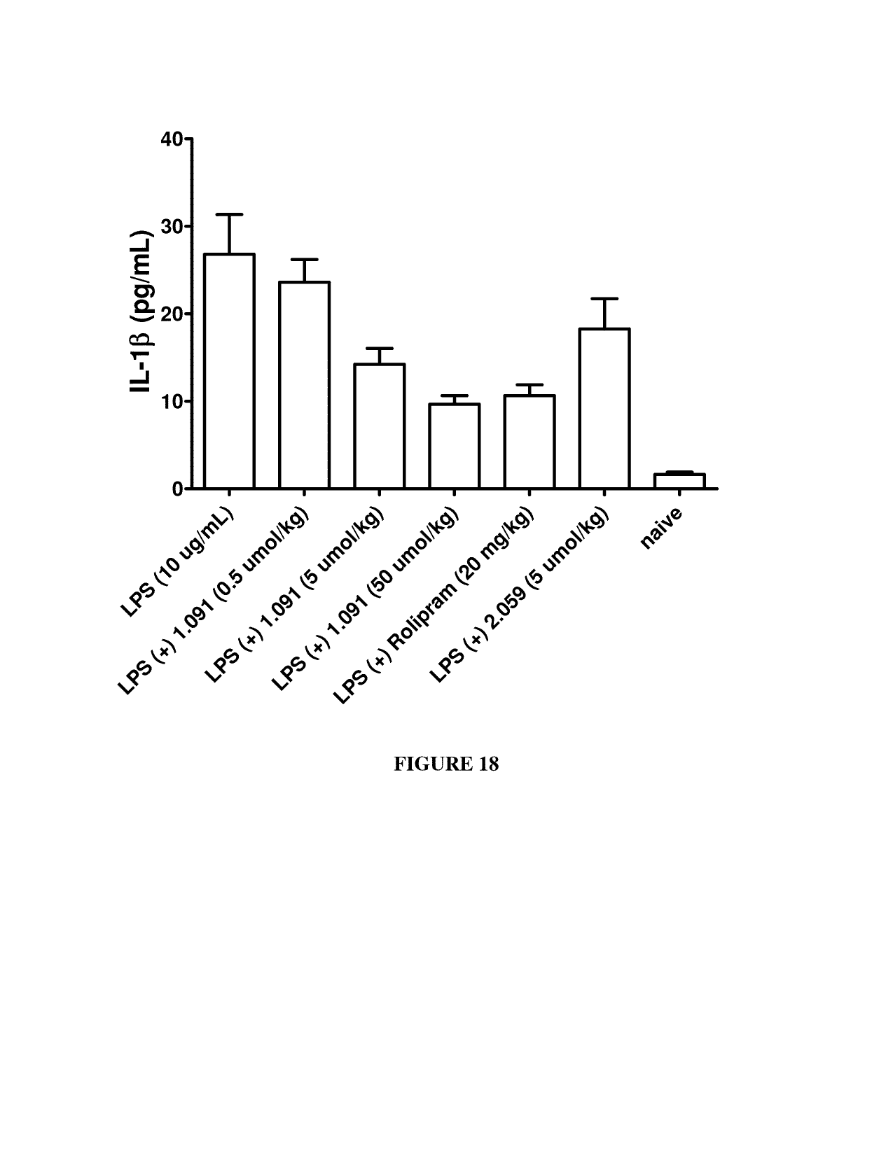

FIG. 17 shows a significant reduction in pulmonary neutrophilia influx after intratracheal dosing of Compound 1.091. The efficacy of Compound 1.091 when dosed intratracheally is similar to the efficacy of the control compound rolipram dosed i.p. FIG. 18 shows the reduction in IL-1β after intratracheal administration of Compound 1.091 or Compound 2.059. These data demonstrate the efficacy of Rho kinase inhibitors of Formula I or II to inhibit inflammatory responses in vivo.

### Example 28

### PDGF-Stimulated Smooth Muscle Cell Proliferation Assay

### Relevance:

This assay demonstrates a compound's ability to inhibit cellular proliferation induced by platelet derived growth factor (PDGF). Activity of compounds in the assay demonstrates the anti-proliferative properties of these compounds and supports the use of these compounds in the treatment of disorders associated with a proliferative component.

### Protocol

Effects on cell proliferation were measured using a bromodeoxyuridine (BrdU) incorporation assay. A-10 rat thoracic aorta cells (ATCC #CRL 1476) were plated at 1000 cells per well in 96-well plates in Dulbecco's Modified Eagles Medium-High Glucose (Gibco cat. #11995-065) containing 10% Fetal Bovine Serum (Sigma EC #232-690-6) and allowed to grow for 24 hrs in an incubator at 37° C. Growth media was then removed and the cells were washed with warmed PBS (Gibco cat #14190-144). Serum free media containing 0.1% BSA was added to the cells. 24 hours later the media was removed and replaced with warmed serum free media. Cells were treated with either 1 μM or 10 μM of test compound and incubated for 60 min at 37° C. prior to the addition of 10 ng/mL PDGF (BD Biosciences cat. #354051) and placed in an incubator at 37° C. for 18 hrs with both compound and stimulant present. Proliferation was then monitored using the BrdU Cell Proliferation Assay, HTS (Calbiochem cat. #HTS01). BrdU was allowed to incorporate into cells for 24 hours prior to the addition of fixative/denaturing solution and the fluorometric detection of incorporated BrdU using a BrdU antibody as per manufacturer's directions. Data are reported as a percent of the PDGF-stimulated BrdU incorporation.

### Results:

As shown in Table 10, compounds of Formulae I and II reduced PDGF-stimulated proliferation of A10 cells with efficacy ranging from 10-80% inhibition when dosed in vitro at 1 μM.

TABLE 10
Reduction of PDGF-stimulated proliferation of A-10 cells as
a percent of the total challenge- stimulated proliferation.

| | Percent of | Percent of | Percent of | Percent of |
|---|---|---|---|---|
| | PDGF | PDGF | PDGF | PDGF |
| | Induced | Induced | Induced | Induced |
| | Proliferation | Proliferation | Proliferation | Proliferation |
| Com- | at 10 μM | at 10 μM | at 1 μM | at 1 μM |
| pound | Avg | SEM | Avg | SEM |
| 1.074 | 46.9 | 3.5 | 79.9 | 9.7 |
| 1.076 | 53.7 | 4.1 | 84.0 | 8.5 |
| 1.091 | 69.3 | 5.5 | 85.7 | 5.3 |
| 1.108 | 43.7 | 1.6 | 83.1 | 6.7 |
| 1.124 | 61.6 | 2.6 | 68.5 | 3.1 |
| 1.131 | 36.6 | 2.4 | 61.7 | 4.8 |
| 1.132 | 30.3 | 1.3 | 48.9 | 3.4 |
| 1.135 | 35.0 | 3.9 | 52.6 | 4.9 |
| 1.136 | 39.8 | 2.6 | 71.4 | 1.3 |
| 1.138 | 27.0 | 1.7 | 46.3 | 1.5 |
| 1.148 | 63.5 | 3.0 | 56.9 | 2.7 |
| 1.151 | 63.8 | 4.1 | 51.0 | 2.1 |
| 1.161 | 33.4 | 0.9 | 50.0 | 3.7 |
| 1.162 | 42.5 | 1.6 | 55.6 | 2.3 |
| 1.165 | 57.9 | 1.2 | 74.8 | 6.1 |
| 1.167 | 52.7 | 4.6 | 78.8 | 4.5 |
| 1.173 | 35.8 | 2.8 | 55.4 | 4.2 |
| 1.175 | 49.0 | 2.5 | 58.2 | 2.3 |
| 1.180 | 64.8 | 5.0 | 92.4 | 7.9 |
| 1.197 | 48.9 | 2.8 | 52.5 | 1.5 |
| 1.204 | 42.8 | 5.3 | 79.3 | 3.0 |
| 1.206 | 51.1 | 2.1 | 77.5 | 5.8 |
| 1.213 | 52.3 | 3.6 | 70.1 | 2.3 |
| 1.215 | 54.0 | 5.3 | 70.8 | 4.0 |
| 1.237 | 51.4 | 4.8 | 63.5 | 5.2 |
| 1.238 | 48.6 | 3.2 | 40.7 | 1.9 |
| 1.239 | 37.8 | 1.6 | 41.7 | 2.7 |
| 1.253 | 47.9 | 2.0 | 44.8 | 3.1 |
| 1.258 | 43.4 | 4.7 | 50.5 | 3.3 |
| 2.009 | 56.5 | 3.9 | 128.9 | 13.4 |
| 2.022 | 39.4 | 1.1 | 89.7 | 4.5 |
| 2.025 | 68.0 | 4.1 | 69.8 | 4.6 |
| 2.026 | 52.0 | 2.5 | 74.5 | 6.5 |
| 2.027 | 64.4 | 5.8 | 79.4 | 5.6 |
| 2.031 | 52.6 | 2.8 | 90.3 | 9.9 |
| 2.038 | 62.7 | 3.5 | 58.6 | 1.2 |
| 2.041 | 61.5 | 3.1 | 81.8 | 4.8 |
| 2.046 | 32.1 | 1.4 | 57.4 | 1.2 |
| 2.047 | 53.8 | 3.2 | 65.3 | 3.0 |
| 2.054 | 84.6 | 6.4 | 68.2 | 4.0 |
| 2.059 | 25.5 | 1.1 | 75.0 | 5.7 |
| 2.064 | 56.2 | 3.9 | 53.1 | 1.9 |
| 2.066 | 19.8 | 0.7 | 20.0 | 0.7 |

### Example 29

### Akt3 and p70S6K Inhibition Assay

### Relevance:

This assay demonstrates a compound's ability to inhibit the kinases Akt3 and p70S6K in vitro. Both kinases are known to play a role in proliferation pathways.

### Protocol

Inhibition of Akt3 and p70S6K activity was determined using the IMAP™ FP Progressive Binding Kit (Molecular Devices product number R8127). Akt3 human enzyme (Upstate Chemicon #14-502), or p70S6K human enzyme (Upstate Chemicon #14-486), and Flourescein tagged substrate peptide (Molecular Devices product number R7110) or (Molecular Devices product number R7184), for Akt3 and p70S6K respectively, was pre-incubated with test compound for 5 minutes in buffer containing 10 mM Tris-HCL pH 7.2, 10 mM MgCl₂, 1 mM DTT and 0.1% BSA. Following the pre-incubation, 30 μM ATP was added to initiate the reaction. After 60 minutes at RT, Molecular Devices IMAP™ binding solution was added to bind phosphorylated substrate. After 30 minutes of incubation in the presence of the IMAP™ beads the fluorescence polarization was read and the ratio was reported as mP. IC₅₀ results were calculated using the Prism software from Graphpad. The Kᵢ values were determined according to the following formula: Kᵢ=IC₅₀/(1+([ATP Challenge]/EC₅₀ ATP)).

### Results:

As shown in Table 11, many compounds of Formulae I and II show sub-micromolar inhibitory potencies against both Akt3 and p70S6K.

TABLE 11
Akt3 and p70S6K potency data

| | Akt3 Ki, | Akt3 Ki, | p70S6K Ki, | p70S6K Ki, |
|---|---|---|---|---|
| Compound | Avg, nM | StdDev, nM | Avg, nM | StdDev, nM |
| 1.072 | 4752.1 | 617.1 | 1130.3 | 263.7 |
| 1.074 | 437.4 | 13.2 | 548.3 | 170.9 |
| 1.075 | 5321.5 | 61.8 | 974.6 | 166.8 |
| 1.076 | 240.9 | 6.2 | 414.3 | 162.7 |
| 1.077 | 5253.2 | 1422.9 | 715.5 | 291.5 |
| 1.078 | 3267.4 | 150.9 | 1678.1 | 640.4 |
| 1.079 | 7191.7 | 445.6 | 3012.8 | 963.8 |
| 1.091 | 5388.5 | 171.6 | 1420.4 | 78.5 |
| 1.093 | 1824.9 | 27.9 | 2025.6 | 356.8 |
| 1.106 | 3914.9 | 257.1 | 1329.1 | 268.0 |
| 1.107 | 16304.0 | 1575.9 | 3356.5 | 701.7 |
| 1.108 | 205.0 | 2.2 | 510.6 | 106.0 |
| 1.109 | 5190.9 | 318.3 | 2495.5 | 314.8 |
| 1.110 | 462.6 | 2.3 | 1298.2 | 175.9 |
| 1.123 | 2406.9 | 287.1 | 2810.7 | 597.6 |
| 1.124 | 7868.0 | 909.4 | 3325.3 | 542.0 |
| 1.127 | 975.4 | 126.4 | 2065.5 | 54.3 |
| 1.131 | 282.6 | 2.0 | 502.8 | 112.4 |
| 1.132 | 81.8 | 8.2 | 514.6 | 111.1 |
| 1.133 | 148.3 | 3.7 | 531.8 | 45.6 |
| 1.134 | 150.7 | 22.1 | 519.7 | 81.1 |
| 1.135 | 444.2 | 32.9 | 588.6 | 142.4 |
| 1.136 | 289.7 | 12.5 | 1236.7 | 413.1 |
| 1.137 | 197.9 | 10.3 | 353.6 | 132.2 |
| 1.138 | 91.3 | 48.3 | 443.5 | 36.3 |
| 1.141 | 1263.0 | 133.1 | 387.5 | 5.8 |
| 1.142 | 8268.5 | 702.6 | 2524.8 | 882.2 |
| 1.143 | 706.5 | 130.5 | 538.2 | 173.7 |
| 1.145 | 1190.5 | 63.5 | 2296.4 | 602.2 |
| 1.146 | 204.9 | 24.7 | 741.5 | 272.3 |
| 1.148 | 1131.4 | 161.7 | 435.5 | 138.0 |
| 1.149 | 7395.9 | 410.0 | 1888.4 | 661.8 |
| 1.150 | 3183.1 | 98.7 | 1273.8 | 106.7 |
| 1.151 | 708.9 | 112.8 | 530.7 | 69.6 |
| 1.152 | 1976.2 | 155.8 | 523.5 | 295.5 |
| 1.153 | 9950.2 | 2150.4 | 2376.1 | 553.3 |
| 1.154 | 4947.5 | 541.2 | 1130.1 | 355.3 |
| 1.155 | 5680.5 | 644.8 | 1751.6 | 502.8 |
| 1.156 | 8772.6 | 427.6 | 3244.6 | 675.0 |
| 1.157 | 29192.3 | 10235.1 | 8693.4 | 2357.4 |
| 1.158 | 5905.2 | 343.4 | 1971.7 | 454.0 |
| 1.159 | 1232.9 | 459.5 | 2061.8 | 271.7 |
| 1.161 | 63.5 | 3.6 | 129.4 | 73.5 |
| 1.162 | 92.0 | 0.9 | 387.4 | 217.4 |
| 1.163 | 4423.8 | 182.3 | 1875.2 | 496.6 |
| 1.164 | 4306.8 | 26.6 | 1957.4 | 729.2 |
| 1.165 | 4140.0 | 293.7 | 1627.1 | 584.4 |
| 1.166 | 18132.9 | 4816.3 | 5163.5 | 1419.0 |
| 1.167 | 8247.3 | 802.7 | 1071.0 | 516.6 |
| 1.170 | 7814.3 | 82.1 | 2046.3 | 580.9 |
| 1.171 | 9326.9 | 448.0 | 3419.0 | 841.6 |
| 1.173 | 157.0 | 0.5 | 339.7 | 204.4 |
| 1.175 | 2820.2 | 294.6 | 853.0 | 92.0 |
| 1.176 | 20941.5 | 4664.9 | 8755.7 | 3209.3 |
| 1.178 | 711.4 | 5.8 | 1116.2 | 637.4 |
| 1.180 | 12022.9 | 416.9 | 1029.2 | 139.1 |
| 1.183 | 9007.8 | 1662.8 | 2477.1 | 1431.3 |
| 1.185 | 4216.6 | 403.6 | 1152.2 | 761.8 |
| 1.186 | 10237.7 | 1867.1 | 1612.5 | 982.8 |
| 1.195 | 21975.8 | 379.4 | 2731.0 | 1192.9 |
| 1.197 | 64051.2 | 47694.4 | 8688.8 | 366.2 |
| 1.200 | 10608.5 | 131.2 | 3903.1 | 3979.1 |
| 1.204 | 1908.2 | 34.3 | 926.8 | 122.9 |
| 1.206 | 529.1 | 22.0 | 314.4 | 209.6 |
| 1.208 | 345.7 | 19.4 | 720.6 | 705.8 |
| 1.212 | 390.2 | 3.8 | 894.0 | 580.3 |
| 1.213 | 3207.8 | 140.6 | 2097.2 | 112.7 |
| 1.215 | 14753.0 | 1613.1 | 1285.8 | 108.5 |
| 1.217 | 10301.1 | 93.6 | 3501.9 | 3691.2 |
| 1.219 | 38297.7 | 11679.7 | 4969.9 | 1893.5 |
| 1.223 | 11139.0 | 1467.2 | 3101.9 | 1629.9 |
| 1.226 | 531.0 | 1.1 | 1348.5 | 1389.6 |
| 1.227 | 3476.0 | 196.6 | 1580.9 | 623.5 |
| 1.229 | 24557.8 | 17008.1 | 3128.5 | 322.4 |
| 1.233 | 2628.6 | 182.4 | 2004.9 | 815.1 |
| 1.236 | 3716.5 | 474.9 | 2755.4 | 2914.8 |
| 1.237 | 7910.2 | 217.5 | 9873.2 | 7272.6 |
| 1.238 | 4171.1 | 173.1 | 2609.6 | 1573.2 |
| 1.239 | 17657.7 | 4393.7 | 10026.9 | 8534.5 |
| 1.246 | 1096.1 | 9.5 | 1879.2 | 1883.4 |
| 1.249 | 1599.7 | 63.8 | 937.5 | 226.8 |
| 1.252 | 205.0 | 11.9 | 170.7 | 84.1 |
| 1.253 | 2597.1 | 29.9 | 2515.0 | 1464.8 |
| 1.258 | 315.2 | 94.1 | 531.5 | 229.6 |
| 1.262 | 861.0 | 1.0 | 5436.6 | 49.5 |
| 2.009 | 3725.8 | 198.3 | 1280.8 | 361.0 |
| 2.022 | 4115.1 | 209.4 | 501.1 | 6.9 |
| 2.025 | 966.4 | 103.5 | 498.8 | 74.2 |
| 2.026 | 2076.0 | 196.5 | 536.0 | 4.6 |
| 2.027 | 657.7 | 58.8 | 509.0 | 70.6 |
| 2.031 | 1357.9 | 0.6 | 326.4 | 52.7 |
| 2.038 | 2553.9 | 184.2 | 1397.0 | 345.6 |
| 2.039 | 1988.0 | 66.7 | 1010.3 | 195.5 |
| 2.041 | 3443.4 | 187.8 | 2095.1 | 161.9 |
| 2.046 | 1975.4 | 142.9 | 758.9 | 401.2 |
| 2.047 | 1942.1 | 163.1 | 437.5 | 184.9 |
| 2.054 | 414.8 | 5.7 | 438.9 | 207.3 |
| 2.055 | 977.5 | 72.3 | 311.6 | 180.9 |
| 2.058 | 1936.0 | 136.7 | 212.6 | 44.7 |
| 2.059 | 119.8 | 24.5 | 207.9 | 173.8 |
| 2.060 | 328.8 | 10.3 | 181.3 | 102.7 |
| 2.064 | 382.0 | 6.7 | 178.2 | 103.4 |
| 2.066 | 2510.4 | 30.5 | 368.3 | 133.1 |

### Example 30

### Kinase Panel Screen

### Relevance:

This assay demonstrates a compound's ability to inhibit members of a panel of kinases known to be involved in signaling pathways connected to inflammatory processes.

### Protocol

Compounds of Formulae I and II were examined for activity against a selected panel of kinases using the KinaseProfiler™ enzyme profiling services (Upstate, Millipore Bioscience Division). Percent kinase activity at 10 μM and 1 μM test compound and 10 μM ATP was determined against 40 wild-type recombinant human kinases according to Upstate's standard protocol: ASK1, BTK, CSK, c-RAF, GCK, GSK3β, IKKα, IKKβ, IRAK1, IRAK4, JNK1α1, JNK2α2, JNK3, ERK1, ERK2, MAPKAP-K2, MAPKAP-K3, MEK1, MKK4, MKK6, MKK7β, Mnk2, MSK1, PAK3, PDK1, PRAK, ROCK1, Rsk2, SAPK2a, SAPK2b, SAPK3, SAPK4, SRPK1, SRPK2, Syk, TAK1, TBK1, PI3-Kβ, PI3-Kγ, PI3-Kδ.

### Results:

Percent inhibition results are reported in Table 12 for four compounds against six kinases in the panel. Only compounds in which R₂ is R₂-2 were found to inhibit significantly GCK, ERK1/2, Mnk2 and IRAK1/2. Only ERK1/2 were inhibited by ˜50% at 1 μM by both compounds 2.059 and 2.066.

TABLE 12
Percent inhibition data for six of the tested kinases

| | Compound | | Compound | | Compound | | Compound | |
|---|---|---|---|---|---|---|---|---|
| | 2.059 | | 2.066 | | 1.161 | | 1.162 | |
| | 1 | 10 | 1 | 10 | 1 | 10 | 1 | 10 |
| | μM | μM | μM | μM | μM | μM | μM | μM |
| ERK1 | 37 | 4 | 52 | 15 | 97 | 75 | 84 | 50 |
| ERK2 | 56 | 12 | 50 | 12 | 104 | 92 | 89 | 60 |
| Mnk2 | 49 | 12 | 99 | 54 | 108 | 106 | 111 | 65 |
| IRAK4 | 63 | 22 | 77 | 25 | 96 | 109 | 105 | 88 |
| IRAK1 | 87 | 30 | 74 | 32 | 106 | 99 | 100 | 97 |
| GCK | 75 | 34 | 39 | 7 | 96 | 91 | 93 | 75 |

### Example 31

### Rodent Pharmacokinetic Analyses of ROCK Inhibitors

Plasma (EDTA K2 anticoagulant) was collected from male, cannulated, CD Sprague Dawley rats to determine the pharmacokinetics of formulations containing compound inhibitors of Rho kinase. Each animal was dosed orally with a 4 ml/kg solution or suspension of each test compound in 10 mM acetate buffered saline, pH 4.5 at a final concentration range of 20-30 μmol/kg. Blood was collected at 0.25, 0.5, 1, 2, 4, 6, 8, and 24 hours. Plasma samples were assayed for the concentration of the test compound using an on-line, solid phase extraction LC/MS/MS analysis system.

Samples were analyzed on a QSTAR Elite, hybrid quadrupole time-of-flight mass spectrometer (Applied Biosystems, Framingham, Mass.) coupled with a Symbiosis Pharma integrated, on-line SPE-HPLC system (Spark Holland Inc., Plainsboro, N.J.). Analyst QS 2.0 software was used for instrument control, data acquisition and processing. An aliquot of each sample was injected onto a Luna C18 column (50×2 mm, 4 um, 80A, Phenomenex, Torrance, Calif.), and elution was carried out using a gradient from 2-98% acetonitrile. Mobile Phase A consisted of 0.1% ammonium hydroxide in water and Mobile Phase B consisted of 0.1% formic acid in acetonitrile. Pharmacokinetic analyses were performed using WinNonlin software version 5.2 (Pharsight Corporation, Mountain View, Calif.).

The pharmacokinetic results based on the observed plasma concentrations of the test compounds in rats are shown in Table 13.

TABLE 13
Pharmacokinetic results from rat oral PK studies
(mean plasma values for n = 3 rats)

| | Tmax | Cmax | AUC (0-last) | t½ | Vz_F |
|---|---|---|---|---|---|
| Compound | (hr) | (nM) | (nM*hr) | (hr) | (L/kg) |
| 1.131 | 0.83 | 5610 | 10825 | 1.55 | 6.8 |
| 1.092 | 0.25 | 2101 | 1849 | 1.74 | 19.0 |
| 1.123 | 0.33 | 2044 | 2064 | 0.9 | 14.8 |
| 2.038 | 0.5 | 1037 | 1283 | 0.71 | 22.5 |
| 2.039 | 0.33 | 783 | 905 | 1.13 | 59.4 |
| 1.074 | 0.42 | 735 | 1167 | 0.86 | 45.7 |
| 1.107 | 1.67 | 544 | 1586 | 1.28 | 36.3 |
| 1.124 | 0.5 | 415 | 535 | 1.39 | 93.4 |
| 2.045 | 0.67 | 223 | 456 | 1.59 | 226 |
| 1.108 | 0.83 | 209 | 415 | 1.36 | 116 |
| 1.091 | BLQ | BLQ | BLQ | BLQ | BLQ |
| 2.026 | BLQ | BLQ | BLQ | BLQ | BLQ |
| 1.136 | BLQ | BLQ | BLQ | BLQ | BLQ |

BLQ indicates that the compound was below the limit of quantitation in the assay

As determined from the plasma concentration versus time curves, the time to peak and peak exposure are represented by the values Tmax and Cmax, respectively. The AUC values (nM*hr) shown were calculated as the areas under the plasma concentration versus time curves from time zero through the time of the last observable value and represent the total exposure of the compound over the course of the study. Half-life values or the amount of time required for the plasma levels of the compound to decline to half the initial value are represented as t1/2. The volume of distribution (Vz_F expressed in L/kg) relates the amount of theoretical volume needed to account for the observed concentration of a given dose of a compound. For rats, the total body water content is approximately 0.15 L/kg. Calculated volumes of distribution below 0.15 L/kg are considered low, whereas values between 5 and 100 L/kg are considered high. The volume of distribution varies depending on the degree of plasma protein binding as well as partitioning of the compound into fat and tissues. Table 13 provides evidence that our ROCK inhibiting compounds have a varying degree of pharmacokinetic properties that would allow them to be optimized for multiple routes of administration. These compounds are quickly absorbed, as indicated by a Tmax of generally less than 1 hour, with varying degrees of peak and total exposure as indicated by Cmax and AUC, with higher values indicating greater exposure. Regardless of exposure, these compounds demonstrate a similar clearance, t1/2.

Additionally, compound concentrations were determined in the plasma and lungs of male, ovalbumin-sensitized, Balb/c mice from a murine model of asthma. Test compounds were formulated in water or 1% polysorbate 80 and dosed at 15 μmol/kg for intraperitoneal (IP) or oral (PO) administration or formulated for intratracheal (IT) administration and dosed at 5 μmol/kg, which directly targets the lungs. Following completion of the in vivo study, mice were euthanized and blood and plasma collected approximately 2.5-3 hours post administration of test compound for bronchodialator (BD) studies and 24 hours post administration for anti-inflamatory (AI) studies. Lungs were homogenized in Matrix A lysing tubes using a FastPrep 24 tissue and cell homogenizer (MP Biomedicals, Solon, Ohio). Both plasma samples and lung extracts were assayed for compound concentrations using an on-line, solid phase extraction LC/MS/MS system. The actual lung tissue concentrations of each compound in mouse were extrapolated from the lung and plasma concentrations, data are shown in Table 14. The results of a set of experiments using unsensitized mice and collecting only plasma 15 minutes post administration of test compounds are shown in Table 15.

TABLE 14
Compound concentrations in ova-sensitized, ova-challenged
mice lungs post IP, PO and IT administration (mean plasma
corrected lung values for n = 9 or 10 mice)

| | Efficacy | | Time Point, | Lung, |
|---|---|---|---|---|
| Compound | Model | Route | h | nM¹ |
| 1.131 | BD | PO | 3 | 7353 |
| 2.038 | BD | PO | 3 | 440 |
| 1.092 | BD | PO | 3 | 152 |
| 1.091 | BD | IP | 3 | 117 |
| 1.091 | BD | IT | 2.5 | 123 |
| 1.131 | AI | PO | 24 | 33 |
| 2.038 | AI | PO | 24 | 11 |

¹for calculation of lung concentrations, it was assumed that 22.6% of the lung mass was plasma (R. H. Storey, Cancer Research, 943-947, 1951)

TABLE 15
Compound concentrations in mice at 15 min post administration
(mean plasma values for n = 3 mice)

| | Plasma Mean | Plasma |
|---|---|---|
| | Concentration, | Concentration |
| Compound | nM | StdDev, nM |
| 1.072 | 1770.9 | 320.9 |
| 1.074 | 506.1 | 407.9 |
| 1.075 | 348.0 | 83.9 |
| 1.076 | 1715.0 | 474.9 |
| 1.077 | 25.9 | 0.2 |
| 1.078 | 1018.8 | 75.8 |
| 1.079 | 2442.5 | 302.9 |
| 1.090 | 5.9 | 5.2 |
| 1.091 | 333.8 | 82.7 |
| 1.092 | 314.3 | 60.4 |
| 1.093 | 362.6 | 148.7 |
| 1.106 | 441.4 | 146.7 |
| 1.107 | 211.1 | 129.5 |
| 1.108 | 394.5 | 9.0 |
| 1.109 | 187.2 | 36.0 |
| 1.110 | 792.0 | 311.9 |
| 1.123 | 71.4 | 11.8 |
| 1.124 | 118.0 | 2.4 |
| 1.126 | 0.0 | 0.0 |
| 1.127 | 980.2 | 757.5 |
| 1.131 | 444.5 | 130.0 |
| 1.132 | 982.4 | 207.7 |
| 1.133 | 1097.9 | 234.3 |
| 1.134 | 1550.8 | 623.9 |
| 1.135 | 656.8 | 115.4 |
| 1.136 | 25.9 | 6.3 |
| 1.137 | 556.9 | 279.8 |
| 1.138 | 1863.8 | 378.7 |
| 1.141 | 1643.1 | 368.6 |
| 1.142 | 329.7 | 171.6 |
| 1.143 | 274.5 | 68.8 |
| 1.145 | 109.0 | 117.9 |
| 1.146 | 1255.7 | 703.5 |
| 1.148 | 767.1 | 63.9 |
| 1.149 | 1559.4 | 789.6 |
| 1.150 | 1392.3 | 1278.3 |
| 1.151 | 478.6 | 173.6 |
| 1.152 | 435.4 | 44.5 |
| 1.153 | 521.5 | 61.3 |
| 1.154 | 1039.5 | 447.9 |
| 1.155 | 32.4 | 36.3 |
| 1.156 | 88.0 | 37.5 |
| 1.157 | 357.2 | 131.9 |
| 1.158 | 101.6 | 54.4 |
| 1.159 | 250.5 | 343.2 |
| 1.161 | 392.5 | 14.9 |
| 1.162 | 76.1 | 12.9 |
| 1.163 | 10.1 | 1.1 |
| 1.164 | 1504.3 | 580.6 |
| 1.165 | 93.5 | 49.6 |
| 1.166 | 342.4 | 118.1 |
| 1.168 | 587.5 | 258.9 |
| 1.170 | 638.6 | 154.7 |
| 1.171 | 368.8 | 208.9 |
| 1.172 | 111.1 | 32.0 |
| 1.173 | 144.4 | 72.6 |
| 1.175 | 1126.5 | 112.5 |
| 1.176 | 89.1 | 69.1 |
| 1.177 | 283.1 | 125.6 |
| 1.182 | 452.5 | 297.7 |
| 1.183 | 708.5 | 359.6 |
| 1.185 | 1023.6 | 492.8 |
| 1.186 | 2169.4 | 1599.1 |
| 1.191 | 260.0 | 58.8 |
| 1.193 | 55.4 | 26.0 |
| 1.194 | 355.0 | 133.5 |
| 1.195 | 107.9 | 23.1 |
| 1.197 | 453.1 | 354.0 |
| 1.198 | 643.2 | 112.1 |
| 1.200 | 0.0 | 0.0 |
| 1.202 | 129.7 | 71.9 |
| 1.203 | 1134.7 | 44.2 |
| 1.204 | 549.1 | 183.6 |
| 1.206 | 671.5 | 80.9 |
| 1.208 | 281.1 | 45.4 |
| 1.210 | 285.8 | 122.9 |
| 1.212 | 863.4 | 104.1 |
| 1.213 | 396.4 | 135.1 |
| 1.215 | 2651.2 | 529.0 |
| 1.217 | 292.5 | 176.0 |
| 1.219 | 1678.9 | 516.3 |
| 1.223 | 12.8 | 0.6 |
| 1.226 | 526.1 | 157.9 |
| 1.227 | 1859.4 | 603.7 |
| 1.229 | 1453.9 | 465.0 |
| 1.233 | 41.1 | 11.6 |
| 1.234 | 239.6 | 79.4 |
| 1.236 | 47.7 | 18.1 |
| 1.237 | 178.4 | 64.6 |
| 1.238 | 48.3 | 29.6 |
| 1.239 | 258.9 | 111.8 |
| 1.241 | 991.4 | 134.5 |
| 1.242 | 579.8 | 314.0 |
| 1.245 | 1524.0 | 127.5 |
| 1.246 | 587.4 | 299.7 |
| 1.249 | 2147.1 | 688.2 |
| 1.252 | 1259.2 | 1210.0 |
| 1.253 | 240.0 | 20.3 |
| 1.258 | 567.5 | 223.5 |
| 1.259 | 264.4 | 39.1 |
| 1.260 | 291.2 | 120.7 |
| 1.262 | 285.2 | 76.2 |
| 2.025 | 73.7 | 21.2 |
| 2.026 | 629.5 | 94.6 |
| 2.027 | 502.6 | 248.5 |
| 2.031 | 1430.4 | 139.2 |
| 2.034 | 664.7 | 649.4 |
| 2.036 | 1343.9 | 1603.3 |
| 2.038 | 728.9 | 222.8 |
| 2.039 | 92.0 | 47.6 |
| 2.041 | 986.5 | 287.0 |
| 2.043 | 60.8 | 24.7 |
| 2.046 | 488.1 | 96.1 |
| 2.047 | 3.0 | 1.7 |
| 2.054 | 765.5 | 214.3 |
| 2.055 | 656.1 | 172.6 |
| 2.056 | 1257.0 | 230.6 |
| 2.057 | 431.2 | 41.5 |
| 2.058 | 193.6 | 167.4 |
| 2.059 | 89.6 | 21.5 |
| 2.060 | 307.6 | 157.6 |
| 2.061 | 73.2 | 21.1 |
| 2.062 | 659.9 | 582.8 |
| 2.063 | 347.9 | 248.5 |
| 2.064 | 201.6 | 78.7 |
| 2.065 | 236.4 | 29.8 |
| 2.066 | 491.6 | |

The results of these quantitative analyses have enabled the selection of compounds for additional studies based on desirable pharmacokinetic profiles. We have identified compounds which possess high bioavailability and efficacy against airway hyperreactivity when dosed orally, as well as compounds that are efficacious when administered intraperitoneally or intratracheally, but do not reach systemic levels when dosed orally and thus are not efficacious by the oral route. Characterization of the pharmacokinetic properties and distribution of these Rho Kinase inhibitors is an essential part of the selection of compounds for drug development.

### Example 32

### Efficacy of Compounds of Formula I or II to Inhibit Proliferation of Primary Smooth-Muscle Like Cells Derived from Human LAM Patients

### Relevance

This assay measures the ability of a compound to directly inhibit the proliferation of primary smooth-muscle like cells derived from human LAM patients. Activity of compounds in this assay supports the use of these compounds for the treatment of diseases with a proliferative component.

### Protocol

LAM cells were dissociated from LAM nodules from the lung of patients with LAM who have undergone lung transplant. In brief, cells were dissociated by enzymatic digestion in M199 medium containing 0.2 mM CaCl₂, 2 mg/ml collagenase D, 1 mg/ml trypsin inhibitor, and 3 mg/ml elastase. The cell suspension was filtered and then washed with equal volumes of cold DF8 medium, consisting of equal amounts of Ham's F-12 and Dulbecco's modified Eagle's medium supplemented with 1.6×10⁻⁶ M ferrous sulfate, 1.2×10⁻⁵ U/ml vasopressin, 1.0×10⁻⁹ M triiodothyronine, 0.025 mg/ml insulin, 1.0×10⁻⁸ M cholesterol, 2.0×10⁻⁷ M hydrocortisone, 10 pg/ml transferrin, and 10% fetal bovine serum. The cells were cultured in DF8 medium and were passaged twice per week. All LAM cells had a high degree of proliferative activity in the absence of any stimuli. Two separate LAM cell lines were tested and denoted as LAM1 or LAM2 cells. LAM cells in subculture during the 3rd through 12th cell passages were used. DNA synthesis was measured using a [³H]thymidine incorporation assay. In brief, near-confluent cells that were serum-deprived for 48 h were incubated with 10 μM of compound or with vehicle (control). After 18 h of incubation, cells were labeled with [methyl-³H]thymidine for 24 hours. The cells were then scraped and lysed, and DNA was precipitated with 10% trichloroacetic acid. The precipitants were aspirated on glass filters and extensively washed and dried, and [³H]thymidine incorporation was counted (Goncharova et al., Mol Pharmacol 73:778-788, 2008)

### Results

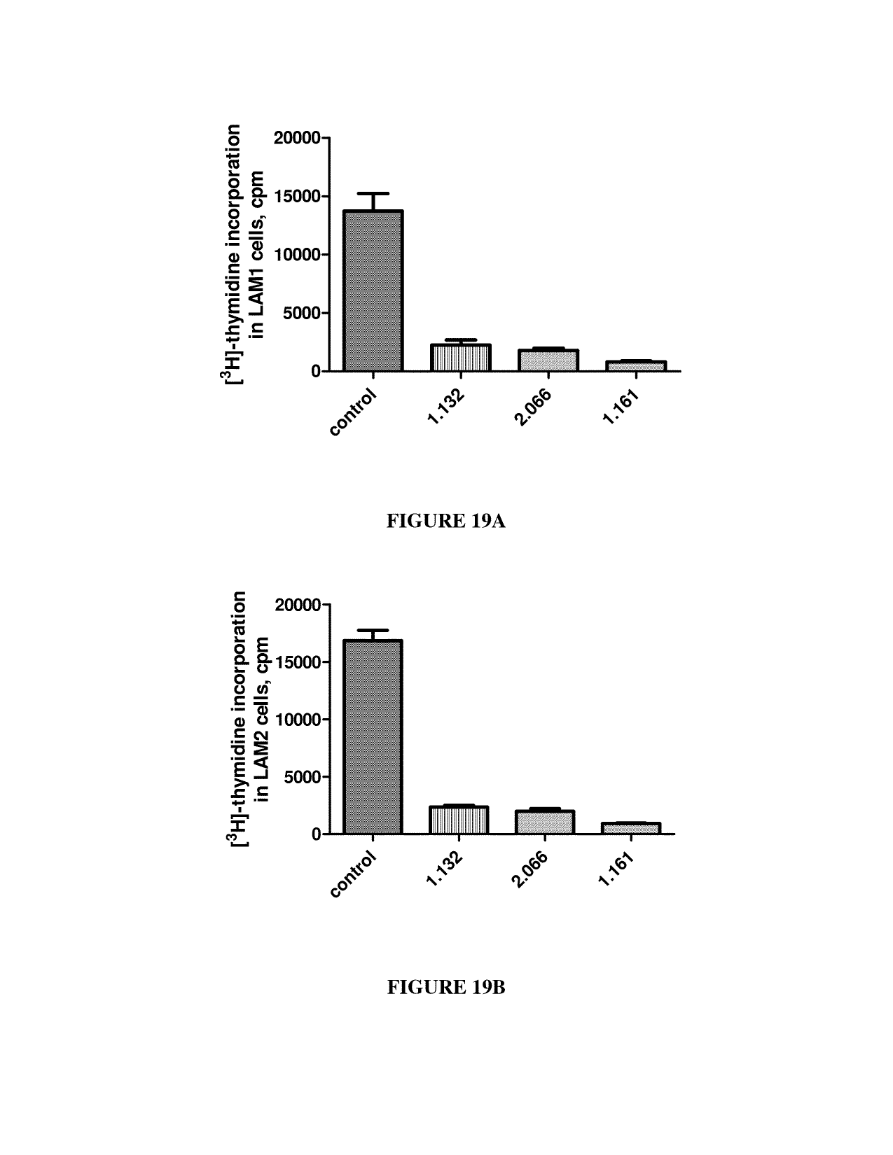

As shown in FIGS. 19A and 19B, compounds of Formula I and II reduced proliferation of LAM1 (FIG. 19A) and LAM2 (FIG. 19B) cells when dosed in vitro at 10 μM. These results demonstrate that Compounds of Formula I and II are efficacious in inhibiting the proliferation of primary cells.

### Example 33

### Summary of Data of Preferred Compounds

Principal biological data describing the preferred compounds of the invention have been collected into Table 16. Displayed in this table are ROCK1 and ROCK2 average Ki values in nM (as detailed in Example 1), Akt3 and p70S6K average Ki values in nM (as detailed in Example 29), average percent of PDGF stimulated proliferation at 10 and 1 μM of test compound (as detailed in Example 28), average percent of stimulated IL-1β, IL-6, and TNF-αsecretion from human monocytes at 10 μM of test compound (as detailed in Example 26), average IC₅₀ for inhibition of fMLP-induced neutrophil chemotaxis in μM (as detailed in Example 3), mean compound plasma concentrations in mice at 15 minutes post oral administration (as detailed in Example 31), and the average percentage of carbachol-induced rat trachael ring contraction at 1 μM of test compound (as detailed in Example 21).

TABLE 16
Summary of Data of Preferred Compounds

| | ROCK1 | ROCK2 | Akt3 | p70S6K | Proliferation | Proliferation | IL-1β | IL-6, | TNF-α | Chemotaxis | Mouse Oral | Trachael Ring |
|---|---|---|---|---|---|---|---|---|---|---|---|---|
| Compound | Ki, nM | Ki, nM | Ki, nM | Ki, nM | at 10 μM, % | at 1 μM, % | % | % | % | IC50, μM | PK, nM | Contraction, % |
| 1.074 | 40.1 | 4.1 | 437.4 | 548.3 | 46.9 | 79.9 | 43.9 | 96.0 | 87.7 | | 506 | 40 |
| 1.075 | 48.7 | 4.4 | 5321.5 | 974.6 | | | 49.7 | 73.9 | 51.6 | | 348 | 39 |
| 1.076 | 14.3 | 2.6 | 240.9 | 414.3 | 53.7 | 84.0 | 51.0 | 81.2 | 78.9 | | 1715 | |
| 1.077 | 76.1 | 11.1 | 5253.2 | 715.5 | | | 30.3 | 43.3 | 52.3 | | 26 | |
| 1.079 | 71.5 | 4.7 | 7191.7 | 3012.8 | | | 59.3 | 31.1 | 56.5 | | 2443 | |
| 1.091 | 71.4 | 3.3 | 5388.5 | 1420.4 | 69.3 | 85.7 | 165.5 | 108.2 | 104.6 | 2.3 | 334 | 35 |
| 1.093 | 64.5 | 7.7 | 1824.9 | 2025.6 | | | 109.0 | 49.7 | 76.1 | | 363 | |
| 1.108 | 25.6 | 6.5 | 205.0 | 510.6 | 43.7 | 83.1 | 131.3 | 89.8 | 116.7 | | 395 | |
| 1.109 | 58.8 | 9.6 | 5190.9 | 2495.5 | | | 190.5 | 312.9 | 118.3 | | 187 | |
| 1.123 | 82.3 | 9.6 | 2406.9 | 2810.7 | | | 82.6 | 64.7 | 62.7 | 3.1 | 71 | 47 |
| 1.124 | 64.5 | 3.3 | 7868.0 | 3325.3 | 61.6 | 68.5 | 99.5 | 101.4 | 61.5 | 3.4 | 118 | 38 |
| 1.126 | 76.2 | 17.2 | | | | | | | | | 0 | |
| 1.131 | 19.7 | 3.8 | 282.6 | 502.8 | 36.6 | 61.7 | 48.3 | 68.6 | 85.2 | 1.6 | 445 | 37 |
| 1.132 | 22.5 | 3.5 | 81.8 | 514.6 | 30.3 | 48.9 | 58.6 | 72.5 | 80.3 | | 982 | 42 |
| 1.133 | 25.0 | 4.3 | 148.3 | 531.8 | | | 54.5 | 70.7 | 66.2 | | 1098 | |
| 1.134 | 22.4 | 4.4 | 150.7 | 519.7 | | | 43.2 | 74.6 | 69.1 | | 1551 | |
| 1.135 | 40.3 | 5.4 | 444.2 | 588.6 | 35.0 | 52.6 | 57.0 | 123.2 | 108.0 | | 657 | |
| 1.136 | 25.8 | 5.1 | 289.7 | 1236.7 | 39.8 | 71.4 | 66.3 | 95.0 | 71.5 | 2.6 | 26 | 45 |
| 1.137 | 36.3 | 7.2 | 197.9 | 353.6 | | | 40.3 | 46.2 | 58.0 | | 557 | |
| 1.138 | 41.1 | 6.3 | 91.3 | 443.5 | 27.0 | 46.3 | 257.4 | 76.6 | 130.9 | 1.9 | 1864 | |
| 1.141 | 28.5 | 3.8 | 1263.0 | 387.5 | | | 50.4 | 71.7 | 75.7 | | 1643 | 35 |
| 1.148 | 24.3 | 3.6 | 1131.4 | 435.5 | 63.5 | 56.9 | 63.9 | 78.6 | 56.1 | | 767 | 51 |
| 1.149 | 46.8 | 4.2 | 7395.9 | 1888.4 | | | 69.8 | 121.5 | 119.9 | | 1559 | 34 |
| 1.150 | 33.2 | 3.2 | 3183.1 | 1273.8 | | | 78.2 | 89.2 | 94.4 | | 1392 | 40 |
| 1.152 | 19.8 | 3.3 | 1976.2 | 523.5 | | | 74.7 | 94.7 | 120.1 | | 435 | 34 |
| 1.153 | 62.8 | 4.2 | 9950.2 | 2376.1 | | | 64.1 | 106.2 | 74.3 | | 522 | 41 |
| 1.155 | 45.4 | 7.0 | 5680.5 | 1751.6 | | | 76.7 | 121.8 | 79.7 | | 32 | |
| 1.156 | 135.8 | 13.0 | 8772.6 | 3244.6 | | | 60.7 | 92.5 | 70.5 | | 88 | |
| 1.157 | 263.8 | 8.8 | 29192.3 | 8693.4 | | | 121.4 | 92.6 | 65.1 | | 357 | |
| 1.158 | 64.1 | 5.1 | 5905.2 | 1971.7 | | | 80.8 | 133.1 | 86.6 | | 102 | |
| 1.161 | 9.9 | 2.5 | 63.5 | 129.4 | 33.4 | 50.0 | 87.7 | 86.3 | 153.5 | | 392 | 40 |
| 1.162 | 15.2 | 2.8 | 92.0 | 387.4 | 42.5 | 55.6 | 95.5 | 99.8 | 158.7 | | 76 | 34 |
| 1.163 | 33.6 | 2.9 | 4423.8 | 1875.2 | | | 166.7 | 140.9 | 91.6 | | 10 | 35 |
| 1.164 | 42.4 | 6.1 | 4306.8 | 1957.4 | | | 80.1 | 109.5 | 89.0 | | 1504 | |
| 1.165 | 50.7 | 3.4 | 4140.0 | 1627.1 | 57.9 | 74.8 | 129.9 | 114.3 | 103.5 | | 94 | 30 |
| 1.166 | 95.2 | 8.0 | 18132.9 | 5163.5 | | | 107.0 | 87.2 | 82.2 | | 342 | |
| 1.171 | 109.2 | 16.0 | 9326.9 | 3419.0 | | | 78.9 | 91.8 | 72.2 | | 369 | |
| 1.173 | 15.1 | 3.6 | 157.0 | 339.7 | 35.8 | 55.4 | 86.1 | 79.5 | 80.1 | | 144 | 33 |
| 1.175 | 65.9 | 7.6 | 2820.2 | 853.0 | 49.0 | 58.2 | 29.3 | 38.2 | 47.4 | | 1126 | |
| 1.176 | 314.3 | 11.2 | 20941.5 | 8755.7 | | | 95.2 | 112.4 | 72.4 | | 89 | |
| 1.186 | 129.3 | 11.9 | 10237.7 | 1612.5 | | | 64.1 | 105.3 | 68.2 | | 2169 | |
| 1.193 | 64.9 | 14.8 | | | | | | | | | 55 | |
| 1.195 | 196.2 | 10.3 | 21975.8 | 2731.0 | | | 115.4 | 94.4 | 67.7 | | 108 | |
| 1.197 | 120.2 | 5.0 | 64051.2 | 8688.8 | 48.9 | 52.5 | 179.1 | 128.8 | 83.3 | | 453 | 42 |
| 1.200 | 76.5 | 5.9 | 10608.5 | 3903.1 | | | 0.0 | 0.0 | 0.2 | | 0 | 92 |
| 1.206 | 64.4 | 9.1 | 529.1 | 314.4 | 51.1 | 77.5 | 88.7 | 164.0 | 97.3 | | 672 | |
| 1.212 | 44.2 | 3.9 | 390.2 | 894.0 | | | 116.3 | 111.0 | 108.1 | | 863 | 40 |
| 1.213 | 106.3 | 3.0 | 3207.8 | 2097.2 | 52.3 | 70.1 | 111.1 | 81.7 | 77.4 | | 396 | 29 |
| 1.215 | 102.8 | 3.5 | 14753.0 | 1285.8 | 54.0 | 70.8 | 136.7 | 63.2 | 60.4 | | 2651 | 41 |
| 1.217 | 70.1 | 12.1 | 10301.1 | 3501.9 | | | 118.6 | 73.8 | 71.3 | | 293 | |
| 1.219 | 343.6 | 15.4 | 38297.7 | 4969.9 | | | 138.9 | 127.7 | 82.1 | | 1679 | |
| 1.223 | 239.5 | 15.7 | 11139.0 | 3101.9 | | | 117.0 | 88.5 | 60.7 | | 13 | |
| 1.233 | 47.2 | 1.3 | 2628.6 | 2004.9 | | | 78.5 | 78.9 | 79.0 | | 41 | |
| 1.236 | 49.3 | 2.1 | 3716.5 | 2755.4 | | | 75.2 | 93.0 | 98.0 | | 48 | |
| 1.237 | 286.7 | 4.0 | 7910.2 | 9873.2 | 51.4 | 63.5 | 97.1 | 100.9 | 70.6 | | 178 | |
| 1.238 | 61.2 | 1.5 | 4171.1 | 2609.6 | 48.6 | 40.7 | 101.1 | 62.9 | 73.2 | | 48 | |
| 1.239 | 282.6 | 6.3 | 17657.7 | 10026.9 | 37.8 | 41.7 | 39.4 | 84.7 | 58.5 | | 259 | |
| 1.249 | 91.7 | 8.6 | 1599.7 | 937.5 | | | 133.8 | 56.2 | 60.0 | | 2147 | |
| 1.252 | 30.5 | 4.5 | 205.0 | 170.7 | | | 139.2 | 68.3 | 101.6 | | 1259 | |
| 1.253 | 59.9 | 1.7 | 2597.1 | 2515.0 | 47.9 | 44.8 | 160.6 | 228.6 | 126.8 | | 240 | |
| 1.258 | 9.5 | 1.3 | 315.2 | 531.5 | 43.4 | 50.5 | 104.1 | 83.5 | 94.0 | | 567 | |
| 1.259 | 19.5 | 2.1 | | | | | | | | | 264 | |
| 1.260 | 70.9 | 7.1 | | | | | | | | | 291 | |
| 1.261 | 307.4 | 14.8 | | | | | | | | | | |
| 1.262 | 54.9 | 4.0 | 861.0 | 5436.6 | | | 145.7 | 156.6 | 135.3 | | 285 | |
| 1.270 | 130.5 | 9.9 | | | | | | | | | | |
| 1.273 | 31.3 | 8.2 | | | | | | | | | | |
| 1.275 | 401.7 | 14.1 | | | | | | | | | | |
| 1.277 | 42.3 | 4.6 | | | | | | | | | | |
| 1.281 | 71.8 | 7.4 | | | | | | | | | | |
| 2.025 | 6.9 | 2.9 | 966.4 | 498.8 | 68.0 | 69.8 | | | | 1.7 | 74 | 33 |
| 2.026 | 38.0 | 13.0 | 2076.0 | 536.0 | 52.0 | 74.5 | 166.0 | 180.7 | 109.1 | 3.8 | 629 | |
| 2.031 | 14.6 | 5.3 | 1357.9 | 326.4 | 52.6 | 90.3 | 49.0 | 89.3 | 66.4 | | 1430 | |
| 2.038 | 28.9 | 6.3 | 2553.9 | 1397.0 | 62.7 | 58.6 | 90.8 | 79.7 | 70.2 | 0.7 | 729 | 57 |
| 2.039 | 18.8 | 6.7 | 1988.0 | 1010.3 | | | 49.8 | 70.3 | 47.8 | 1.6 | 92 | |
| 2.041 | 30.8 | 9.6 | 3443.4 | 2095.1 | 61.5 | 81.8 | | | | | 987 | |
| 2.046 | 16.7 | 5.6 | 1975.4 | 758.9 | 32.1 | 57.4 | | | | | 488 | |
| 2.047 | 26.4 | 7.0 | 1942.1 | 437.5 | 53.8 | 65.3 | | | | | 3 | |
| 2.054 | 17.1 | 3.7 | 414.8 | 438.9 | 84.6 | 68.2 | 24.0 | 56.8 | 37.9 | | 765 | |
| 2.055 | 16.0 | 6.4 | 977.5 | 311.6 | | | | | | | 656 | |
| 2.057 | 6.2 | 3.7 | | | | | | | | | 431 | |
| 2.058 | 15.3 | 3.3 | 1936.0 | 212.6 | | | 1.2 | 1.3 | 10.6 | | 194 | |
| 2.059 | 3.9 | 2.7 | 119.8 | 207.9 | 25.5 | 75.0 | 0.3 | 0.0 | 6.9 | | 90 | |
| 2.060 | 4.9 | 3.2 | 328.8 | 181.3 | | | 5.9 | 19.6 | 33.0 | | 308 | |
| 2.061 | 10.5 | 1.8 | | | | | | | | | 73 | |
| 2.064 | 4.1 | 2.2 | 382.0 | 178.2 | 56.2 | 53.1 | 14.3 | 45.7 | 66.2 | | 202 | |
| 2.065 | 4.1 | 1.8 | | | | | | | | | 236 | |
| 2.066 | 10.2 | 2.3 | 2510.4 | 368.3 | 19.8 | 20.0 | 0.0 | 0.0 | 25.2 | | 492 | |
| 2.067 | 19.6 | 4.2 | | | | | | | | | | |
| 2.068 | 8.0 | 5.8 | | | | | | | | | | |
| 2.069 | 16.7 | 2.4 | | | | | | | | | | |
| 2.072 | 7.5 | 4.4 | | | | | | | | | | |
| 2.073 | 12.7 | 4.2 | | | | | | | | | | |
| 2.076 | 8.0 | 2.4 | | | | | | | | | | |
| 2.077 | 33.7 | 5.0 | | | | | | | | | | |
| 2.078 | 18.3 | 2.6 | | | | | | | | | | |
| 2.079 | 18.5 | 2.3 | | | | | | | | | | |
| 2.082 | 131.7 | 9.0 | | | | | | | | | | |
| 2.096 | 70.2 | 9.6 | | | | | | | | | | |
| 2.097 | 35.4 | 2.8 | | | | | | | | | | |
| 2.099 | 15.0 | 3.8 | | | | | | | | | | |

Although the invention has been described with reference to the presently preferred embodiments, it should be understood that various modifications could be made without departing from the scope of the invention.

What is claimed:

1. A method of treating systemic hypertension, comprising the steps of first identifying a subject suffering from systemic hypertension, then administering to the subject an effective amount of a compound of Formula II; 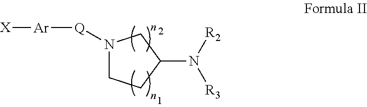
wherein:
Q is C═O, SO₂, or (CR₄R₅)ₙ₃;
n₁ is 1, 2, or 3;
n₂ is for 2;
n₃ is 0, 1, 2, or 3;
wherein the ring represented by 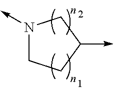
is optionally substituted by alkyl, halo, oxo, OR₆, NR₆R₇, or SR₆;
R₂ is R₂-1 or R₂-2, optionally substituted: 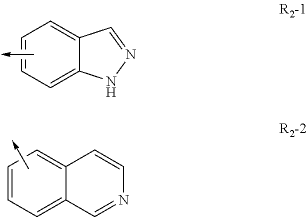
Ar is a monocyclic or bicyclic aryl or heteroaryl ring;
X is from 1 to 3 substituents on Ar, and each is independently selected from the group consisting of OR₈, NR₈R₉, SR₈, SOR₈, SO₂R₈, SO₂NR₈R₉, NR₈SO₂R₉, CONR₈R₉, NR₈C(═O)R₉, NR₈C(═O)OR₉, OC(═O)NR₈R₉, and NR₈C(═O)NR₉R₁₀,
R₃-R₇ are independently H, alkyl, alkenyl, alkynyl, cycloalkyl, cycloalkenyl, cycloalkylalkyl, cycloalkylalkenyl, or cycloalkylalkynyl, optionally substituted;
R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, and NR₁₁C(═O)NR₁₂R₁₃;
R₉ and R₁₀ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₄, NR₁₄R₁₅, NO₂, SR₁₄, SOR₁₄, SO₂R₁₄, SO₂NR₁₄R₁₅, NR₁₄SO₂R₁₅, OCF₃, CONR₁₄OR₁₅, NR₁₄C(═O)R₁₅, NR₁₄C(═O)OR₁₅, OC(═O)NR₁₄R₁₅, and NR₁₄C(═O)NR₁₅R₁₆;
wherein any two of the groups R₈, R₉ and R₁₀ are optionally joined with a link selected from the group consisting of bond, —O—, —S—, —SO—, —SO₂—, and —NR₁₇— to form a ring;
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle;
with the first proviso that if X is acyclic and is connected to Ar by a carbon atom, then X contains at least one nitrogen or sulfur atom,
with the second proviso that if X is acyclic and is connected to Ar by an oxygen or nitrogen atom, then X contains at least one additional oxygen, nitrogen or sulfur atom, and
with the third proviso that if X is connected to Ar by a —SO₂— linkage, then R₂ is not nitrogen- or oxygen-substituted R₂-2.

2. The method according to claim 1, wherein said compound of Formula II is a compound of Formula IIa, IIb, or IIc: 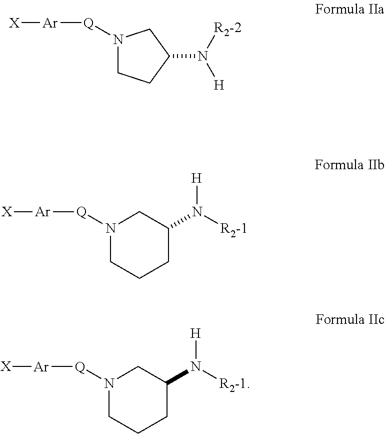
wherein Ar is phenyl, a 6,5-fused bicyclic heteroaryl ring, or a 6,6-fused bicyclic heteroaryl ring; Ar is substituted by 1 or 2 substituents X, and Q is CH₂.

3. The method according to claim 2, wherein Ar is 3-substituted phenyl; 4-substituted phenyl; 3,4-disubstituted phenyl; or 2,3-disubstituted phenyl.

4. The method according to claim 2, wherein Ar is benzofuran, benzothiophene, indole, and benzimidazole.

5. The method according to claim 1, wherein said compound is Compound 1.074, which is (R)—N-(1-(4-(methylthio)benzyl)piperidin-3-yl)-1H-indazol-5-amine; Compound 1.075, which is (S)—N-(1-(4-(methylthio)benzyl)piperidin-3-yl)-1H-indazol-5-amine; Compound 1.091, which is (S)—N-(3-((3-(1H-indazol-5-ylamino)piperidin-1-yl)methyl)phenyl)methanesulfonamide; Compound 1.093, which is (R)—N-(3-((3-(1H-indazol-5-ylamino)piperidin-1-yl)methyl)phenyl)methanesulfonamide; Compound 1.123, which is (R)—N-(3-((3-(1H-indazol-5-ylamino)piperidin-1-yl)methyl)phenyl)ethanesulfonamide; Compound 1.124, which is (S)—N-(3-((3-(1H-indazol-5-ylamino)piperidin-1-yl)methyl)phenyl)ethanesulfonamide; Compound 1.126, which is (R)-2-(3-((3-(1H-indazol-5-ylamino)piperidin-1-yl)methyl)phenoxy)-N-(pyridin-3-yl)acetamide; Compound 1.152, which is (S)-2-(5-((3-(1H-indazol-5-ylamino)piperidin-1-yl)methyl)-2-methylphenoxy)ethanol; Compound 1.158, which is (S)—N-(1-(3-(methylthio)benzyl)piperidin-3-yl)-1H-indazol-5-amine; Compound 1.161, which is (R)-2-(5-((3-(1H-indazol-5-ylamino)piperidin-1-yl)methyl)-2-methylphenoxy)ethanol; Compound 1.195, which is (S)-2-(3-((3-(1H-indazol-5-ylamino)piperidin-1-yl)methyl)phenoxy)acetamide; Compound 1.200, which is (S)-ethyl 2-(3-((3-(1H-indazol-5-ylamino)piperidin-1-yl)methyl)phenoxy)acetate; Compound 1.212, which is (R)—N-(5-((3-(1H-indazol-5-ylamino)piperidin-1-yl)methyl)-2-chlorophenyl)methanesulfonamide; Compound 1.213, which is (S)—N-(5-((3-(1H-indazol-5-ylamino)piperidin-1-yl)methyl)-2-chlorophenyl)methanesulfonamide; Compound 1.215, which is (S)-3-(3-(1H-indazol-5-ylamino)piperidin-1-yl)methyl)benzenesulfonamide; Compound 1.219, which is (S)-3-((3-(1H-indazol-5-ylamino)piperidin-1-yl)methyl)benzamide; Compound 1.233, which is (S)—N-(5-((3-(1H-indazol-5-ylamino)piperidin-1-yl)methyl)-2-methylphenyl)methanesulfonamide; Compound 1.236, which is (S)—N-(5-((3-(1H-indazol-5-ylamino)piperidin-1-yl)methyl)-2-methylphenyl)butane-1-sulfonamide; Compound 1.237, which is (S)—N-(2-((3-(1H-indazol-5-ylamino)piperidin-1-yl)methyl)-5-methylphenyl)-N′,N′ dimethylaminosulfamide; Compound 1.238, which is (S)—N-(5-((3-(1H-indazol-5-ylamino)piperidin-1-yl)methyl)-2-methylphenyl)propane-1-sulfonamide; Compound 1.239, which is (S)—N-(5-((3-(1H-indazol-5-ylamino)piperidin-1-yl)methyl)-2-methylphenyl)-4-methylbenzenesulfonamide; Compound 1.249, which is (R)-3-(3-(1H-indazol-5-ylamino)piperidin-1-yl)methyl)benzenesulfonamide; Compound 1.253, which is (S)—N-(5-((3-(1H-indazol-5-ylamino)piperidin-1-yl)methyl)-2-methylphenyl)ethanesulfonamide; Compound 1.258, which is (R)—N-(5-((3-(1H-indazol-5-ylamino)piperidin-1-yl)methyl)-2-methylphenyl)methanesulfonamide; Compound 1.259, which is (R)—N-(5-((3-(1H-indazol-5-ylamino)piperidin-1-yl)methyl)-2-methylphenyl)ethanesulfonamide; Compound 1.260, which is (R)—N-(5-((3-(1H-indazol-5-ylamino)piperidin-1-yl)methyl)-2-methylphenyl)-4-methylbenzenesulfonamide; Compound 1.261, which is (S)—N-(3-((3-(1H-indazol-5-ylamino)piperidin-1-yl)methyl)phenyl)-N′,N′ dimethylaminosulfamide; Compound 1.262, which is (R)—N-(2-((3-(1H-indazol-5-ylamino)piperidin-1-yl)methyl)-5-methylphenyl)-N′,N′ dimethylaminosulfamide; Compound 1.270, which is (S)—N-(3-((3-(1H-indazol-5-ylamino)piperidin-1-yl)methyl)phenyl)piperidine-1-sulfonamide; Compound 1.275, which is (S)—N-(3-((3-(1H-indazol-5-ylamino)piperidin-1-yl)methyl)-2-methylphenyl)-N′,N′ dimethylaminosulfamide; Compound 1.281, which is (R)-2-(5-((3-(1H-indazol-5-ylamino)piperidin-1-yl)methyl)-2-methylphenyl1H-indazol-5-ylamino)piperidin-1-yl)methyl)-2-methylphenoxy)acetamide; Compound 2.026, which is (R)—N-(1-(4-(methylthio)benzyl)pyrrolidin-3-yl)isoquinolin-5-amine; Compound 2.038, which is (R)—N-(3-((3-(isoquinolin-5-ylamino)pyrrolidin-1-yl)methyl)phenyl)methanesulfonamide; Compound 2.039, which is (R)-2-(3-((3-(isoquinolin-5-ylamino)pyrrolidin-1-yl)methyl)phenoxy)ethanol; Compound 2.041, which is (R)—N-(3-((3-(isoquinolin-5-ylamino)pyrrolidin-1-yl)methyl)phenyl)ethanesulfonamide; Compound 2.054, which is (R)—N-(5-((3-(isoquinolin-5-ylamino)pyrrolidin-1-yl)methyl)-2-methylphenyl)ethanesulfonamide; Compound 2.064, which is (R)-2-(5-((3-(isoquinolin-5-ylamino)pyrrolidin-1-yl)methyl)-2-methylphenoxy)ethanol; Compound 2.067, which is (R)-2-(5-((3-(isoquinolin-5-ylamino)pyrrolidin-1-yl)methyl)-2-methoxyphenoxy)ethanol; Compound 2.068, which is (R)-2-(2-fluoro-5-((3-(isoquinolin-5-ylamino)pyrrolidin-1-yl)methyl)phenoxy)ethanol; Compound 2.069, which is (R)—N-(3-((3-(isoquinolin-5-ylamino)pyrrolidin-1-yl)methyl)phenyl)piperidine-1-sulfonamide; Compound 2.073, which is (R)-2-(5-((3-(isoquinolin-5-ylamino)pyrrolidin-1-yl)methyl)-2-methylphenoxy)acetic acid; Compound 2.076, which is (R)—N-(5-((3-(isoquinolin-5-ylamino)pyrrolidin-1-yl)methyl)-2-methylphenyl)methanesulfonamide; Compound 2.077, which is (R)—N-(5-((3-(isoquinolin-5-ylamino)pyrrolidin-1-yl)methyl)-2-methylphenyl)-N′,N′ dimethylaminosulfamide; Compound 2.078, which is (R)—N-(3-((3-(isoquinolin-5-ylamino)pyrrolidin-1-yl)methyl)-2-methylphenyl)methanesulfonamide; Compound 2.079, which is (R)—N-(3-((3-(isoquinolin-5-ylamino)pyrrolidin-1-yl)methyl)-2-methylphenyl)-N′,N′ dimethylaminosulfamide; Compound 2.082, which is (R)—N-(1-((2-(methylthio)pyrimidin-4-yl)methyl)pyrrolidin-3-yl)isoquinolin-5-amine; Compound 2.096, which is (R)—N-(3-((3-(isoquinolin-5-ylamino)pyrrolidin-1-yl)methyl)-2-methoxyphenyl)methanesulfonamide; Compound 2.097, which is (R)—N-(3-((3-(isoquinolin-5-ylamino)pyrrolidin-1-yl)methyl)-2-methoxyphenyl)-N′,N′ dimethylaminosulfamide; or Compound 2.099, which is (R)-2-(5-((3-(isoquinolin-5-ylamino)pyrrolidin-1-yl)methyl)-2-methylphenoxy)acetamide.

6. A method of treating systemic hypertion, the method comprises the steps of first identifying a subject suffering from systemic hypertion, then administering to the subject an effective amount of a compound of Formula II; 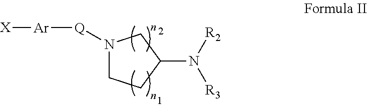
wherein:
Q is C═O, SO₂, or (CR₄R₅)ₙ₃;
n₁ is 1, 2, or 3;
n₂ is for 2;
n₃ is 0, 1, 2, or 3;
wherein the ring represented by 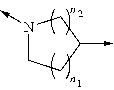
is optionally substituted by alkyl, halo, oxo, OR₆, NR₆R₇, or SR₆;
R₂ is R₂-1 or R₂-2, optionally substituted: 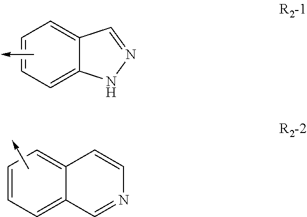
Ar is a monocyclic or bicyclic aryl or heteroaryl ring;
X is from 1 to 3 substituents on Ar, each independently in the form Y—Z, in which Z is attached to Ar;
Y is one or more substituents on Z, and each is independently selected from the group consisting of H, halogen, OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, and NR₈C(═O)NR₉R₁₀;
Z is alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, and (heterocycle)alkynyl;
R₃-R₇ are independently H, alkyl, alkenyl, alkynyl, cycloalkyl, cycloalkenyl, cycloalkylalkyl, cycloalkylalkenyl, or cycloalkylalkynyl, optionally substituted;
R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, and NR₁₁C(═O)NR₁₂R₁₃;
R₉ and R₁₀ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₄, NR₁₄R₁₅, NO₂, SR₁₄, SOR₁₄, SO₂R₁₄, SO₂NR₁₄R₁₅, NR₁₄SO₂OR₁₅, OCF₃, CONR₁₄R₁₅, NR₁₄C(═O)R₁₅, NR₁₄C(═O)OR₁₅, OC(═O)NR₁₄R₁₅, and NR₁₄C(═O)NR₁₅R₁₆;
wherein any two of the groups R₈, R₉ and R₁₀ are optionally joined with a link selected from the group consisting of bond, —O—, —S—, —SO—, —SO₂—, and —NR₁₇— to form a ring; and
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle.

7. The method according to claim 6, wherein said compound of Formula II is a compound of Formula IIa, IIb, or IIc: 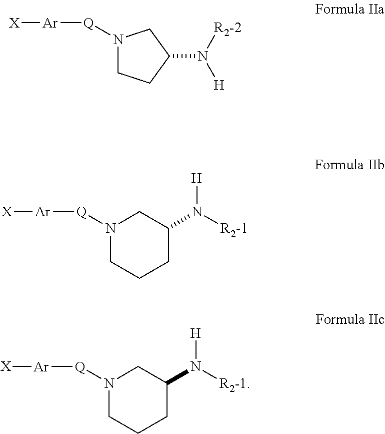
wherein Ar is phenyl, a 6,5-fused bicyclic heteroaryl ring, or a 6,6-fused bicyclic heteroaryl ring; Ar is substituted by 1 or 2 substituents X, and Q is CH₂.

8. The method according to claim 6, wherein said compound is Compound 1.076, which is (R)—N-(1-(4-ethynylbenzyl)piperidin-3-yl)-1H-indazol-5-amine; Compound 1.077, which is (S)—N-(1-(4-ethynylbenzyl)piperidin-3-yl)-1H-indazol-5-amine; Compound 1.153, which is (S)—N-(1-(3-ethynylbenzyl)piperidin-3-yl)-1H-indazol-5-amine; Compound 1.186, which is (S)—N-(1-(3-cyclopropylbenzyl)piperidin-3-yl)-1H-indazol-5-amine; Compound 1.193, which is (R)—N-(1-(3-ethynylbenzyl)piperidin-3-yl)-1H-indazol-5-amine; Compound 1.206, which is (R)—N-(1-(4-cyclopropylbenzyl)piperidin-3-yl)-1H-indazol-5-amine; or Compound 2.031, which is (R)—N-(1-(4-ethynylbenzyl)pyrrolidin-3-yl)isoquinolin-5-amine.

9. A method of treating systemic hypertion, the method comprises the steps of first identifying a subject suffering from systemic hypertion, then administering to the subject an effective amount of a compound of Formula II; 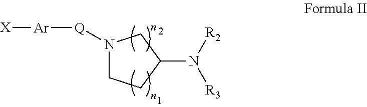
wherein:
Q is C═O, SO₂, or (CR₄R₅)ₙ₃;
n₁ is 1, 2, or 3;
n₂ is for 2;
n₃ is 0, 1, 2, or 3;
wherein the ring represented by 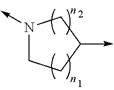
is optionally substituted by alkyl, halo, oxo, OR₆, NR₆R₇, or SR₆;
R₂ is R₂-1 or R₂-2, optionally substituted: 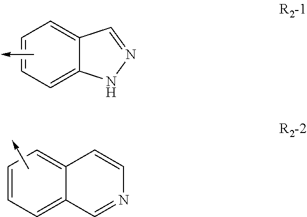
Ar is a monocyclic or bicyclic aryl or heteroaryl ring;
X is from 1 to 3 substituents on Ar, each independently in the form Y—Z, in which Z is attached to Ar;
Y is one or more substituents on Z, and each is independently OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀,
Z is alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl;
R₃-R₇ are independently H, alkyl, alkenyl, alkynyl, cycloalkyl, cycloalkenyl, cycloalkylalkyl, cycloalkylalkenyl, or cycloalkylalkynyl, optionally substituted;
R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, and NR₁₁C(═O)NR₁₂R₁₃;
R₉ and R₁₀ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₄, NR₁₄R₁₅, NO₂, SR₁₄, SOR₁₄, SO₂R₁₄, SO₂NR₁₄R₁₅, NR₁₄SO₂R₁₅, OCF₃, CONR₁₄R₁₅, NR₁₄C(═O)R₁₅, NR₁₄C(═O)OR₁₅, OC(═O)NR₁₄R₁₅, or NR₁₄C(═O)NR₁₅R₁₆;
wherein any two of the groups R₈, R₉ and R₁₀ are optionally joined with a link selected from the group consisting of bond, —O—, —S—, —SO—, —SO₂—, and —NR₁₇— to form a ring; and
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle;
with the proviso that when Z is selected from the group consisting of alkyl, alkenyl, and alkynyl, and Y falls on the carbon by which Z is attached to Ar, then Y contains at least one nitrogen or sulfur atom.

10. The method according to claim 9, wherein Ar is a heteroaryl.

11. The method according to claim 9, wherein said compound of Formula II is a compound of Formula IIa, IIb, or IIc: 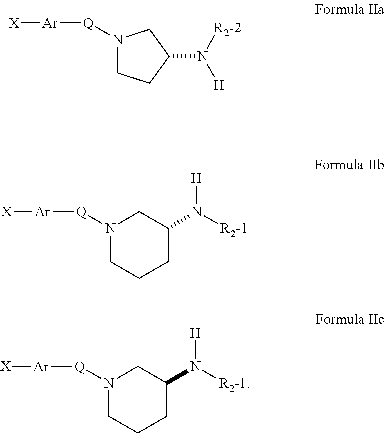
wherein Ar is phenyl, a 6,5-fused bicyclic heteroaryl ring, or a 6,6-fused bicyclic heteroaryl ring; Ar is substituted by 1 or 2 substituents X, and Q is CH₂.

12. The method according to claim 9, wherein said compound is Compound 1.108, which is (R)-2-(6-((3-(1H-indazol-5-ylamino)piperidin-1-yl)methyl)-1H-indol-1-yl)ethanol; Compound 1.109, which is (S)-2-(6-((3-(1H-indazol-5-ylamino)piperidin-1-yl)methyl)-1H-indol-1-yl)ethanol; Compound 1.162, which is (R)-2-(5-((3-(1H-indazol-5-ylamino)piperidin-1-yl)methyl)-1H-indol-1-yl)acetamide; Compound 1.165, which is (S)-2-(5-((3-(1H-indazol-5-ylamino)piperidin-1-yl)methyl)-1H-indol-1-yl)acetamide; Compound 1.176, which is (S)-tert-butyl 3-((4-(1H-indazol-5-ylamino)piperidin-1-yl)methyl)benzylcarbamate; Compound 1.197, which is (S)—N-(1H-indazol-5-ylamino)piperidin-1-yl)methyl)benzyl)acetamide; Compound 1.217, which is (S)-2-(6-((3-(1H-indazol-5-ylamino)piperidin-1-yl)methyl)indolin-1-yl)ethanol; Compound 1.223, which is (S)-(4-((3-(1H-indazol-5-ylamino)piperidin-1-yl)methyl)phenyl)methanol; Compound 1.273, which is (R)-2-(3-((3-(1H-indazol-5-ylamino)piperidin-1-yl)methyl)-1H-indol-1-yl)ethanol; Compound 2.058, which is (R)-2-(6-((3-(isoquinolin-5-ylamino)pyrrolidin-1-yl)methyl)-1H-indol-1-yl)acetamide; Compound 2.059, which is (R)-2-(5-((3-(isoquinolin-5-ylamino)pyrrolidin-1-yl)methyl)-1H-indol-1-yl)acetamide; Compound 2.060, which is (R)-2-(6-((3-(isoquinolin-5-ylamino)pyrrolidin-1-yl)methyl)-1H-indol-1-yl)ethanol; Compound 2.066, which is (R)-2-(5-((3-(isoquinolin-5-ylamino)pyrrolidin-1-yl)methyl)-1H-indol-1-yl)ethanol; or Compound 2.072, which is (R)-2-(3-((3-(isoquinolin-5-ylamino)pyrrolidin-1-yl)methyl)-1H-indol-1-yl)ethanol.

13. A method of treating systemic hypertion, the method comprises the steps of first identifying a subject suffering from systemic hypertion, then administering to the subject an effective amount of a compound of Formula II; 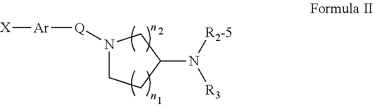
wherein:
Q is C═O, SO₂, or (CR₄R₅)ₙ₃;
n₁ is 1, 2, or 3;
n₂ is for 2;
n₃ is 0, 1, 2, or 3;
wherein the ring represented by 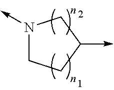
is optionally substituted by alkyl, halo, oxo, OR₆, NR₆R₇, or SR₆;
R₂-5 is 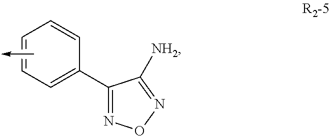
optionally substituted;
Ar is a monocyclic or bicyclic aryl or heteroaryl ring;
X is from 1 to 3 substituents on Ar, each independently in the form Y—Z, in which Z is attached to Ar;
Y is one or more substituents on Z, and each is independently selected from the group consisting of H, halogen, OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, and NR₈C(═O)NR₉R₁₀;
Z is independently selected from the group consisting of absent, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, heterocycle, (heterocycle)alkyl, (heterocycle)alkenyl, and (heterocycle)alkynyl;
R₃-R₇ are independently H, alkyl, alkenyl, alkynyl, cycloalkyl, cycloalkenyl, cycloalkylalkyl, cycloalkylalkenyl, or cycloalkylalkynyl, optionally substituted;
R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, and NR₁₁C(═O)NR₁₂R₁₃;
R₉ and R₁₀ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₄, NR₁₄R₁₅, NO₂, SR₁₄, SOR₁₄, SO₂R₁₄, SO₂NR₁₄R₁₅, NR₁₄SO₂R₁₅, OCF₃, CONR₁₄R₁₅, NR₁₄C(═O)R₁₅, NR₁₄C(═O)OR₁₅, OC(═O)NR₁₄R₁₅, and NR₁₄C(═O)NR₁₅R₁₆;
wherein any two of the groups R₈, R₉ and R₁₀ are optionally joined with a link selected from the group consisting of bond, —O—, —S—, —SO—, —SO₂—, and —NR₁₇— to form a ring; and
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle.

14. A method of treating systemic hypertion, the method comprises the steps of first identifying a subject suffering from systemic hypertion, then administering to the subject an effective amount of a compound of Formula Ia, Ib, or Ic; 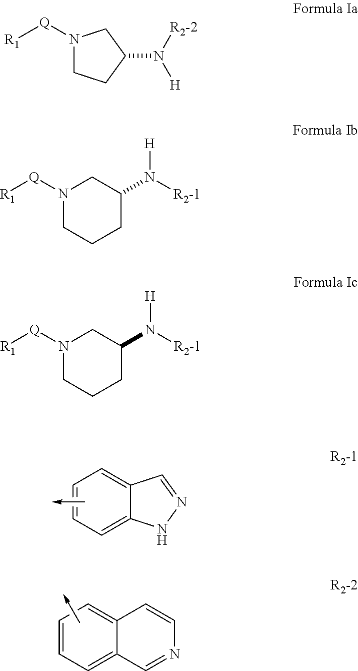
wherein R₁ is phenyl, thiophene, 6,5-fused bicyclic heteroaryl ring, or 6,6-fused bicyclic heteroaryl ring, R₁ is either unsubstituted or is optionally substituted with 1, 2 or 3 substituents independently selected from halogen, methyl, ethyl, hydroxyl, methoxy, or ethoxy;
Q is C═O, SO₂, or (CR₄R₅)ₙ₃;
R₂-1 and R₂-2 are optionally substituted;
R₄ and R₅ are independently H, alkyl, cycloalkyl, optionally substituted.

15. The method according to claim 14, wherein R₁ is 3-substituted phenyl, 4-substituted phenyl, 3,4-disubstituted phenyl, or 6,5-fused bicyclic heteroaryl ring.

16. The method according to claim 14, wherein said compound of Formula Ia is Compound 2.025, which is (R)—N-(1-(4-methylbenzyl)pyrrolidin-3-yl)isoquinolin-5-amine; Compound 2.046, which is (R)—N-(1-benzylpyrrolidin-3-yl)isoquinolin-5-amine; Compound 2.047, which is (R)—N-(1-(4-methoxybenzyl)pyrrolidin-3-yl)isoquinolin-5-amine; Compound 2.055, which is (R)—N-(1-(benzofuran-5-ylmethyl)pyrrolidin-3-yl)isoquinolin-5-amine; Compound 2.057, which is (R)—N-(1-((1H-indol-6-yl)methyl)pyrrolidin-3-yl)isoquinolin-5-amine; Compound 2.061, which is (R)-3-((3-(isoquinolin-5-ylamino)pyrrolidin-1-yl)methyl)phenol; or Compound 2.065, which is (R)—N-(1-((1H-indol-5-yl)methyl)pyrrolidin-3-yl)isoquinolin-5-amine.

17. The method according to claim 14, wherein said compound of Formula Ic is Compound 1.079, which is (S)—N-(1-(4-methoxybenzyl)piperidin-3-yl)-1H-indazol-5-amine; Compound 1.141, which is (S)—N-(1-(4-chlorobenzyl)piperidin-3-yl)-1H-indazol-5-amine; Compound 1.148, which is (S)—N-(1-((1H-indol-6-yl)methyl)piperidin-3-yl)-1H-indazol-5-amine; Compound 1.149, which is (S)—N-(1-((1H-indol-5-yl)methyl)piperidin-3-yl)-1H-indazol-5-amine; Compound 1.150, which is (S)—N-(1-(benzofuran-5-ylmethyl)piperidin-3-yl)-1H-indazol-5-amine; Compound 1.155, which is (S)—N-(1-(2,4-dimethylbenzyl)piperidin-3-yl)-1H-indazol-5-amine; Compound 1.156, which is (S)—N-(1-(2,3-dimethylbenzyl)piperidin-3-yl)-1H-indazol-5-amine; Compound 1.163, which is (S)-3-((3-(1H-indazol-5-ylamino)piperidin-1-yl)methyl)phenol; Compound 1.164, which is (S)—N-(1-(4-fluorobenzyl)piperidin-3-yl)-1H-indazol-5-amine; Compound 1.166, which is (S)—N-(1-((2,3-dihydrobenzo[b][1,4]dioxin-6-yl)methyl)piperidin-3-yl)-1H-indazol-5-amine; Compound 1.171, which is (S)—N-(1-(3-methylbenzyl)piperidin-3-yl)-1H-indazol-5-amine; Compound 1.175, which is (S)—N-(1-(benzo[b]thiophen-5-ylmethyl)piperidin-3-yl)-1H-indazol-5-amine; or Compound 1.277, which is (S)—N-(1-(thiophen-3-ylmethyl)piperidin-3-yl)-1H-indazol-5-amine.

18. The method according to claim 14, wherein said compound of Formula Ib is Compound 1.131, which is (R)—N-(1-(benzofuran-5-ylmethyl)piperidin-3-yl)-1H-indazol-5-amine; Compound 1.132, which is (R)—N-(1-(4-chlorobenzyl)piperidin-3-yl)-1H-indazol-5-amine; Compound 1.133, which is (R)—N-(1-(4-methylbenzyl)piperidin-3-yl)-1H-indazol-5-amine; Compound 1.134, which is (R)—N-(1-(4-bromobenzyl)piperidin-3-yl)-1H-indazol-5-amine; Compound 1.135, which is (R)—N-(1-(4-ethylbenzyl)piperidin-3-yl)-1H-indazol-5-amine; Compound 1.136, which is (R)—N-(1-(2,4-dimethylbenzyl)piperidin-3-yl)-1H-indazol-5-amine; Compound 1.137, which is (R)—N-(1-(benzo[b]thiophen-5-ylmethyl)piperidin-3-yl)-1H-indazol-5-amine; Compound 1.138, which is (R)—N-(1-((1H-indol-6-yl)methyl)piperidin-3-yl)-1H-indazol-5-amine; Compound 1.173, which is (R)-5-(3-(1H-indazol-5-ylamino)piperidin-1-yl)methyl)-2-methylphenol; or Compound 1.252, which is (R)—N-(1-((1H-indol-3-yl)methyl)piperidin-3-yl)-1H-indazol-5-amine.
